(19) United States
(12) Patent Application Publication
(10) Pub. No.: US 2009/0221642 A1
(43) Pub. Date: Sep. 3, 2009

(54) MUSCARINIC RECEPTOR AGONISTS, COMPOSITIONS, METHODS OF TREATMENT THEREOF, AND PROCESSES FOR PREPARATION THEREOF-176

(75) Inventors: Shujuan Jin, Saint Laurent (CA); Miroslaw Jerzy Tomaszewski, Saint Laurent (CA); Vijayaratnam Santhakumar, Saint Laurent (CA)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 12/392,293

(22) Filed: Feb. 25, 2009

Related U.S. Application Data
(60) Provisional application No. 61/033,082, filed on Mar. 3, 2008.

(51) Int. Cl. A61K 31/454; C07D 211/32; A61P 27/06

(52) U.S. Cl. 514/322; 546/199

(57) ABSTRACT

Compounds of Formula 1, or pharmaceutically acceptable salts thereof:

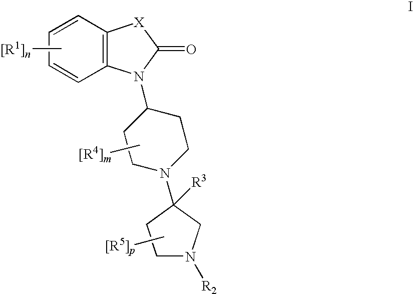

wherein X, R¹, R², R³, R⁴, R⁵, n, m, and p are as defined in the specification as well as salts and pharmaceutical compositions including the compounds are prepared. They are useful in therapy, in particular in the management of pain.

### BACKGROUND OF THE INVENTION

1. Field of the invention

The present invention relates to agonists of muscarinic receptors. The present invention also provides compositions comprising such agonists, and methods therewith for treating muscarinic receptor mediated diseases. Particularly, the present invention is related to compounds that may be effective in treating pain, Alzheimer's disease, glaucoma, and/or schizophrenia.

2. Discussion of Technology

The neurotransmitter acetylcholine binds to two types of cholinergic receptors: the ionotropic family of nicotinic receptors and the metabotropic family of muscarinic receptors. Muscarinic receptors belong to the large superfamily of plasma membrane-bound G protein coupled receptors (GPCRs) and show a remarkably high degree of homology across species and receptor subtype. These M1-M5 muscarinic receptors are predominantly expressed within the parasympathetic nervous system which exerts excitatory and inhibitory control over the central and peripheral tissues and participate in a number of physiologic functions, including heart rate, arousal, cognition, sensory processing, and motor control.

Muscarinic agonists such as muscarine and pilocarpine, and antagonists, such as atropine have been known for over a century, but little progress has been made in the discovery of receptor subtype-selective compounds, thereby making it difficult to assign specific functions to the individual receptors. See, e.g., DeLapp, N. et al., “Therapeutic Opportunities for Muscarinic Receptors in the Central Nervous System,” J. Med. Chem., 43(23), pp. 4333-4353 (2000); Hulme, E. C. et al., “Muscarinic Receptor Subtypes,” Ann. Rev. Pharmacol. Toxicol., 30, pp. 633-673 (1990); Caulfield, M. P. et al., “Muscarinic Receptors-Characterization, Coupling, and Function,” Pharmacol. Ther., 58, pp. 319-379 (1993); Caulfield, M. P. et al., International Union of Pharmacology. XVII. Classification of Muscarinic Acetylcholine Receptors,” Pharmacol. Rev., 50, pp. 279-290 (1998).

The Muscarinic family of receptors is the target of a large number of pharmacological agents used for various diseases, including leading drugs for COPD, asthma, urinary incontinence, glaucoma, schizophrenia, Alzheimer's (AchE inhibitors), and Pain.

For example, direct acting muscarinic receptor agonists have been shown to be antinociceptive in a variety of animal models of acute pain (Bartolini A., Ghelardini C., Fantetti L., Malcangio M., Malmberg-Aiello P., Giotti A. Role of muscarinic receptor subtypes in central antinociception. Br. J. Pharmacol. 105:77-82, 1992.; Capone F., Aloisi A. M., Carli G., Sacerdote P., Pavone F. Oxotremorine-induced modifications of the behavioral and neuroendocrine responses to formalin pain in male rats. Brain Res. 830:292-300, 1999.).

A few studies have examined the role of muscarinic receptor activation in chronic or neuropathic pain states. In these studies, the direct and indirect elevation of cholinergic tone was shown to ameliorate tactile allodynia after intrathecal administration in a spinal ligation model of neuropathic pain in rats and these effects again were reversed by muscarinic antagonists (Hwang J.-H., Hwang K.-S., Leem J.-K., Park P.-H., Han S.-M., Lee D.-M. The antiallodynic effects of intrathecal cholinesterase inhibitors in a rat model of neuropathic pain. Anesthesiology 90:492-494, 1999; Lee E. J., Sim J. Y, Park J. Y., Hwang J. H., Park P. H., Han S. M. Intrathecal carbachol and clonidine produce a synergistic antiallodynic effect in rats with a nerve ligation injury. Can J Anaesth 49:178-84, 2002.). Thus, direct or indirect activation of muscarinic receptors has been shown to elicit both acute analgesic activity and to ameliorate neuropathic pain. Muscarinic agonists and ACHE-Is are not widely used clinically owing to their propensity to induced a plethora of adverse events when administered to humans. The undesirable side effects include excessive salivation and sweating, enhanced gastrointestinal motility, and bradycardia among other adverse events. These side effects are associated with the ubiquitous expression of the muscarinic family of receptors throughout the body.

To date, five subtypes of muscarinic receptors (M1-M5) have been cloned and sequenced from a variety of species, with differential distributions in the body. Therefore, it was desirable to provide molecules would permit selective modulation, for example, of muscarinic receptors controlling central nervous function without also activating muscarinic receptors controlling cardiac, gastrointestinal or glandular functions.

There is also a need for methods for treating muscarinic receptor-mediated diseases.

There is also a need for modulators of muscarinic receptors that are selective as to subtypes M1-M5.

### DESCRIPTION OF THE EMBODIMENTS

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “n-membered” where n is an integer typically describes the number of ring-forming atoms in a moiety where the number of ring-forming atoms is n. For example, piperidinyl is an example of a 6-membered heterocycloalkyl ring and 1,2,3,4-tetrahydro-naphthalene is an example of a 1 0-membered cycloalkyl group.

For compounds of the invention in which a variable appears more than once, each variable can be a different moiety independently selected from the group defining the variable. For example, where a structure is described having two R groups that are simultaneously present on the same compound, the two R groups can represent different moieties independently selected from the group defined for R. In another example, when an optionally multiple substituent is designated in the form:

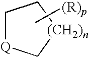

then it is understood that substituent R can occur p number of times on the ring, and R can be a different moiety at each occurrence. It is understood that each R group may replace any hydrogen atom attached to a ring atom, including one or both of the (CH₂)ₙ hydrogen atoms. Further, in the above example, should the variable Q be defined to include hydrogens, such as when Q is said to be CH₂, NH, etc., any floating substituent such as R in the above example, can replace a hydrogen of the Q variable as well as a hydrogen in any other non-variable component of the ring.

For compounds of the invention in which a variable appears more than once, each variable can be a different moiety independently selected from the group defining the variable. For example, where a structure is described having two R groups that are simultaneously present on the same compound, the two R groups can represent different moieties independently selected from the group defined for R.

As used herein, the phrase “optionally substituted” means unsubstituted or substituted. As used herein, the term “substituted” means that a hydrogen atom is removed and replaced by a substitutent. As used herein, the phrase “substituted by oxo” means that two hydrogen atoms are removed from a carbon atom and replaced by an oxygen bound by a double bond to the carbon atom. It is understood that the number of substituents for a given atom is limited by its valency.

Throughout the definitions, the term “Cₙ₋ₘ” is referred to indicate C₁₋₄, C₁₋₆, and the like, wherein n and m are integers and indicate the number of carbons, wherein n-m indicates a range which includes the endpoints.

As used herein, the term “Cₙ₋ₘ alkyl”, employed alone or in combination with other terms, refers to a saturated hydrocarbon group that may be straight-chain or branched, having n to m carbons. In some embodiments, the alkyl group contains from 1 to 7 carbon atoms, from 1 to 6 carbon atoms, from 1 to 4 carbon atoms, from 1 to 3 carbon atoms, or 1 to 2 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, n-heptyl, n-octyl, and the like.

As used herein, the term “alkylene” refers to a divalent alkyl linking group. Examples of alkylene groups include, but are not limited to, ethan-1,2-diyl, propan-1,3-diyl, propan-1,2-diyl, butan-1,4-diyl, butan-1,3-diyl, butan-1,2-diyl, 2-methyl-propan-1,3-diyl, and the like.

As used herein, “Cₙ₋ₘ alkenyl”, employed alone or in combination with other terms, refers to an alkyl group having one or more double carbon-carbon bonds and having n to m carbons. In some embodiments, the alkynyl moiety contains 2 to 6 or to 2 to 5 carbon atoms. Example alkenyl groups include, but are not limited to, ethenyl, n-propenyl, isopropenyl, n-butenyl, sec-butenyl, and the like.

As used herein, the term “alkenylene”, employed alone or in combination with other terms, refers to a divalent alkenyl group. Example alkenylene groups include, but are not limited to, ethen-1,2-diyl, propen-1,3-diyl, propen-1,2-diyl, buten-1,4-diyl, buten-1,3-diyl, buten-1,2-diyl, 2-methyl-propen-1,3-diyl, and the like.

As used herein, “Cₙ₋ₘ alkynyl”, employed alone or in combination with other terms, refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6 or 2 to 5 carbon atoms.

As used herein, the term “alkynylene”, employed alone or in combination with other terms, refers to a divalent alkynyl group. In some embodiments, the alkynylene moiety contains 2 to 12 carbon atoms. In some embodiments, the alkynylene moiety contains 2 to 6 carbon atoms. Example alkynylene groups include, but are not limited to, ethyn-1,2-diyl, propyn-1,3,-diyl, 1-butyn-1,4-diyl, 1-butyn-1,3-diyl, 2-butyn-1,4-diyl, and the like.

As used herein, the term “Cₙ₋ₘ alkoxy”, employed alone or in combination with other terms, refers to an group of formula —O-alkyl, wherein the alkyl group has n to m carbons. Example alkoxy groups include methoxy, ethoxy, propoxy (e.g., n-propoxy and isopropoxy), t-butoxy, and the like.

As used herein, the term “Cₙ₋ₘ aryl”, employed alone or in combination with other terms, refers to a monocyclic or polycyclic (e.g., having 2, 3 or 4 fused or covalently linked rings), aromatic hydrocarbon having n to m carbons, such as, but not limited to, phenyl, 1-naphthyl, 2-naphthyl, anthracenyl, phenanthrenyl, and the like. In some embodiments, aryl groups have from 6 to 20 carbon atoms, from 6 to 10 carbon atoms, or from 6 to 8 carbons atoms. In some embodiments, the aryl group is phenyl.

As used herein, the term “Cₙ₋ₘ aryl-Cₙ₋ₘalkyl” refers to a group of formula-alkylene-aryl, wherein the alkyl and aryl portions each has, independently, n to m carbon atoms. In some embodiments, the alkyl portion has 1 to 4, 1 to 3, 1 to 2, or 1 carbon atom(s). In some embodiments, the alkyl portion of the arylalkyl group is methyl or ethyl. In some embodiments, the arylalkyl group is benzyl.

As used herein, the term “Cₙ₋ₘ cycloalkyl”, employed alone or in combination with other terms, refers to a non-aromatic cyclic hydrocarbon moiety, which may optionally contain one or more alkenylene or alkynylene groups as part of the ring structure and which has n to m carbons. Cycloalkyl groups can include mono- or polycyclic (e.g., having 2, 3 or 4 fused or covalently linked rings) ring systems. Also included in the definition of cycloalkyl are moieties that have one or more aromatic rings fused (i.e., having a bond in common with) to the cycloalkyl ring, for example, benzo derivatives of pentane, pentene, hexane, and the like. In some embodiments, the cycloalkyl group is monocyclic and has 3 to 14 ring members, 3 to 10 ring members, 3 to 8 ring members, or 3 to 7 ring members. One or more ring-forming carbon atoms of a cycloalkyl group can be oxidized to form carbonyl linkages. Exemplary cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. In some embodiments, the cycloalkyl group is cyclopropyl, cyclobutyl, cyclopentyl, or cyclohexyl.

As used herein, the term “Cₙ₋ₘ cycloalkyl-Cₙ₋ₘalkyl” refers to a group of formula-alkylene-cycloalkyl, wherein the alkyl and cycloalkyl portions each has, independently n to m carbon atoms. In some embodiments, the alkyl portion has 1 to 4, 1 to 3, 1 to 2, or 1 carbon atom(s).

As used herein, “Cₙ₋ₘ haloalkoxy”, employed alone or in combination with other terms, refers to a group of formula —O-haloalkyl having n to m carbon atoms. An example haloalkoxy group is OCF₃. In some embodiments, the haloalkoxy group is fluorinated only.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only.

As used herein, the term “fluorinated Cₙ₋ₘ haloalkyl” refers to a Cₙ₋ₘ haloalkyl wherein the halogen atoms are selected from fluorine. In some embodiments, fluorinated Cₙ₋ₘ haloalkyl is fluoromethyl, difluoromethyl, or trifluoromethyl.

As used herein, the terms “halo” and “halogen”, employed alone or in combination with other terms, refer to fluoro, chloro, bromo, and iodo. In some embodiments, halogen is fluoro, bromo, or chloro. In some embodiments, halogen is fluoro or chloro.

As used herein, the term “Cₙ₋ₘ heteroaryl”, “Cₙ₋ₘ heteroaryl ring”, or “Cₙ₋ₘ heteroaryl group”, employed alone or in combination with other terms, refers to a monocyclic or polycyclic (e.g., having 2, 3 or 4 fused or covalently linked rings) aromatic hydrocarbon moiety, having one or more heteroatom ring members selected from nitrogen, sulfur and oxygen, and having n to m carbon atoms. In some embodiments, the heteroaryl group has 1, 2, 3, or 4 heteroatoms. In some embodiments, the heteroaryl group has 1, 2, or 3 heteroatoms. In some embodiments, the heteroaryl group has 1 or 2 heteroatoms. In some embodiments, the heteroaryl group has 1 heteroatom. When the heteroaryl group contains more than one heteroatom ring member, the heteroatoms may be the same or different. Example heteroaryl groups include, but are not limited to, pyrrolyl, azolyl, oxazolyl, thiazolyl, imidazolyl, furyl, thienyl, quinolinyl, isoquinolinyl, indolyl, benzothienyl, benzofuranyl, benzisoxazolyl, imidazo[1,2-b]thiazolyl or the like. In some embodiments, the heteroaryl group has 5 to 10 carbon atoms.

As used herein, the term “Cₙ₋ₘ heteroaryl-Cₙ₋ₘalkyl” refers to a group of formula-alkylene-heteroaryl, wherein the alkyl and heteroaryl portions each has, independently, n to m carbon atoms. In some embodiments, the alkyl portion has 1 to 4, 1 to 3, 1 to 2, or 1 carbon atom(s).

As used herein, the term “Cₙ₋ₘ heterocycloalkyl”, “Cₙ₋ₘ heterocycloalkyl ring”, or “Cₙ₋ₘ heterocycloalkyl group”, employed alone or in combination with other terms, refers to non-aromatic ring system, which may optionally contain one or more alkenylene or alkynylene groups as part of the ring structure, and which has at least one heteroatom ring member selected from nitrogen, sulfur and oxygen, and which has n to m carbon atoms. In some embodiments, the heteroaryl group has 1, 2, 3, or 4 heteroatoms. In some embodiments, the heteroaryl group has 1, 2, or 3 heteroatoms. In some embodiments, the heteroaryl group has 1 or 2 heteroatoms. In some embodiments, the heteroaryl group has 1 heteroatom. In some embodiments, the heteroaryl group has 1 or 2 heteroatoms. When the heterocycloalkyl groups contains more than one heteroatom, the heteroatoms may be the same or different. Heterocycloalkyl groups can include mono- or polycyclic (e.g., having 2, 3 or 4 fused or covalently bonded rings) ring systems. Also included in the definition of heterocycloalkyl are moieties that have one or more aromatic rings fused (i.e., having a bond in common with) to the non-aromatic ring, for example, 1,2,3,4-tetrahydro-quinoline and the like. In some embodiments, the heterocycloalkyl group has 3 to 20 ring-forming atoms, 3 to 10 ring-forming atoms, or about 3 to 8 ring forming atoms. The carbon atoms or heteroatoms in the ring(s) of the heterocycloalkyl group can be oxidized to form a carbonyl, or sulfonyl group (or other oxidized linkage) or a nitrogen atom can be quaternized. In some embodiments, the heterocycloalkyl group is a monocyclic or bicyclic ring. In some embodiments, the heterocycloalkyl group is a monocyclic ring, wherein the ring comprises from 3 to 6 carbon atoms and from 1 to 3 heteroatoms, referred to herein as C₃₋₆heterocycloalkyl.

Examples of heterocycloalkyl groups include pyrrolidinyl, pyrrolidino, piperidinyl, piperidino, piperazinyl, piperazino, morpholinyl, morpholino, thiomorpholinyl, thiomorpholino, and pyranyl.

A five-membered ring heteroaryl is a heteroaryl with a ring having five ring atoms wherein 1, 2 or 3 ring atoms are independently selected from N, O, and S.

Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered ring heteroaryl is a heteroaryl with a ring having six ring atoms wherein 1, 2 or 3 ring atoms are independently selected from N, O, and S. Exemplary six-membered ring heteroaryls are pyridyl, pyrazinyl, pyrimidinyl, triazinyl and pyridazinyl.

As used herein, the term “Cₙ₋ₘ heterocycloalkyl-Cₙ₋ₘalkyl” refers to a group of formula-alkylene-heterocycloalkyl, wherein the alkyl and heterocycloalkyl portions each has, independently, n to m carbon atoms. In some embodiments, the alkyl portion of the heterocycloalkylalkyl group is methylene. In some embodiments, the alkyl portion has 1-4, 1-3, 1-2, or 1 carbon atom(s).

As used herein, the moiety “C(O)” indicates a divalent carbonyl group of formula C(═O).

As used herein, the term “hydroxyl-C₁₋₆alkyl” refers to a group of formula —C₁₋₆alkylene-OH.

As used herein, the term “Cₙ₋ₘ alkylene bridge” refers to an alkylene group having n to m carbon atoms which bridges two carbon atoms to which the group is attached, thereby forming a bridge between the two carbon atoms.

As used herein, the symbol “*” indicates a multiplication sign.

As used herein, an “isolated enantiomer” means a compound containing more than 50% of the enantiomer of the compound, preferably containing at least 75% of the enantiomer of the compound, more preferably containing at least 90% of the enantiomer of the compound, even more preferably containing at least 95% of the enantiomer of the compound.

### Compounds

In one aspect, the present invention provides a compound of Formula I:

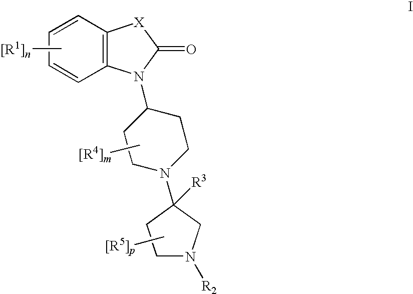

or pharmaceutically acceptable salt thereof;
wherein:

X is —CR⁶R⁷—, —NR⁸—, —O—, or —S—;

each R¹ is, independently, hydrogen, halogen, cyano, nitro, C₁₋₆alkyl, C₁₋₆haloalkyl, C₂₋₆alkenyl, C₂₋₆ alkynyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀aryl-C₁₋₃alkyl, C₃₋₉ heteroaryl, C₃₋₉heteroaryl-C₁₋₃alkyl, —SR^{e}, —OR^{f}, —O(CH₂)ᵣ—OR^{f}, —C(═O)—R^{e}, —C(═O)OR^{f}, —C(═O)NR^{g}R^{h}, —SO₂R^{e}, —SO₂NR^{g}R^{h}, —NR^{g}R^{h}, or —(CH₂)ᵣNR^{g}R^{h};

R² is selected from —C(═O)OR^{a}, —C(═O)NR^{c}R^{d};

R³ is C₁₋₆ alkyl or C₁₋₆ haloalkyl;

each R⁴ is, independently, halogen, C₁₋₆alkyl, C₁₋₆haloalkyl, C₁₋₆alkoxy, hydroxyl-C₁₋₆alkyl-, —CH₂—OR, -or —C(═O)NR₂; or

any two of R⁴ are linked together to form a C₁₋₄ alkylene bridge and the other R⁴, if any, are each, independently, halogen, C₁₋₆alkyl, C₁₋₆haloalkyl, C₁₋₆alkoxy, hydroxyl-C₁₋₆alkyl-, —CH₂—OR, -or —C(═O)NR₂;

each R⁵ is, independently, halogen, C₁₋₆alkyl, C₁₋₆haloalkyl, C₁₋₆alkoxy, hydroxyl-C₁₋₆alkyl-, —CH₂—OR, -or —C(═O)NR₂;

R⁶, R⁷, and R⁸ are each, independently, hydrogen, C₁₋₆alkyl, C₂₋₆alkenyl, or C₁₋₆haloalkyl;

each R⁹, R¹⁰, and R¹¹ is, independently, phenyl, C₃₋₆ cycloalkyl, C₂₋₅ heterocycloalkyl, C₃₋₅ heteroaryl, halogen, cyano, nitro, —SR^{w}, —OR^{x}, —O(CH₂)ᵣ—OR^{x}, R^{x}, —C(═O)—R^{w}, —C(═O)OR^{x}, —C(═O)NR^{y}R^{z}, —SO₂R^{w}, —SO₂NR^{y}R^{z}, —NR^{y}R^{z}, or —(CH₂)ᵣNR^{u}R^{z};

R^{a}, R^{c}, and R^{d} are each, independently, hydrogen, C₁₋₇ alkyl, C₁₋₇ haloalkyl, C₂₋₆ alkenyl, C₂₋₆ alkynyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein the C₁₋₇ alkyl, C₂₋₆ alkenyl, C₂₋₆ alkynyl, and C₁₋₇ haloalkyl are each optionally substituted by 1, 2, or 3 independently selected R⁹ groups; wherein the C₃₋₇ cycloalkyl, C₃₋₇ cycloalkyl-C₁₋₃ alkyl, C₃₋₇ heterocycloalkyl, and C₃₋₇ heterocycloalkyl-C₁₋₃ alkyl are each optionally substituted by 1, 2, 3, or 4 independently selected R¹⁰ groups; and wherein the C₆₋₁₀aryl, C₆₋₁₀aryl-C₁₋₃alkyl, C₃₋₉ heteroaryl, and C₃₋₉heteroaryl-C₁₋₃ alkyl are each optionally substituted by 1, 2, 3, or 4 independently selected R¹¹ groups, with a proviso that R^{a} is not hydrogen;

each R^{e}, R^{f}, R^{g}, R^{h}, R^{w}, R^{x}, R^{y}, R^{z}, and R is, independently hydrogen, C₁₋₆ alkyl, C₂₋₆ alkenyl, or C₁₋₆ haloalkyl;

r is 1, 2, 3, or 4;

n is 1, 2, 3, or 4;

m is 0, 1, 2, 3, 4, 5, 6, 7, or 8; and

p is an integer from 0 to 6.

In some embodiments, X is —CR⁶R⁷— or —NR⁸—.

In some embodiments, X is —NR⁸—.

In some embodiments, X is —NH.

In some embodiments, R⁶, R⁷, and R⁸ are each, independently, hydrogen, C₁₋₆alkyl, or C₁₋₆ haloalkyl.

In some embodiments, R⁶, R⁷, and R⁸ are each, independently, hydrogen or C₁₋₆alkyl.

In some embodiments, R² is —C(═O)OCH₃, —C(═O)OCH₂CH₃, —C(═O)OCH(CH₃)₂, is —C(═O)OCH₂CH₂F, —C(═O)OCH₂—C≡CH, or —C(═O)NHCH₂CH₃.

In some embodiments, R² is —C(═O)OR^{a}; and R^{a} is C₁₋₇ alkyl, C₂₋₆ alkenyl, C₂₋₆ alkynyl, C₁₋₇ haloalkyl, C₃₋₇ cycloalkyl, C₃₋₇ cycloalkyl-C₁₋₃alkyl, C₃₋₇ heterocycloalkyl-C₁₋₃ alkyl, C₆₋₁₀ aryl-C₁₋₃alkyl, or C₃₋₉ heteroaryl-C₁₋₃alkyl. In some further embodiments R^{a} is C₁₋₇ alkyl, C₂₋₆ alkenyl, C₂₋₆ alkynyl, C₁₋₇ haloalkyl, C₃₋₇ cycloalkyl, or C₃₋₇ cycloalkyl-C₁₋₃alkyl. In still further embodiments, R^{a} is C₁₋₇ alkyl, C₂₋₆ alkynyl, or C₁₋₇ haloalkyl.

In some embodiments, R² is —C(═O)NR^{c}R^{d}; and R^{c} and R^{d} are each, independently, hydrogen, C₁₋₇ alkyl, C₂₋₆ alkenyl, C₂₋₆ alkynyl, C₁₋₇ haloalkyl, C₃₋₇ cycloalkyl, or C₃₋₇ cycloalkyl-C₁₋₃alkyl. In some further embodiments, one of R^{c} and R^{d} is H and the other is C₁₋₇ alkyl, C₂₋₆ alkenyl, C₂₋₆ alkynyl, C₁₋₇ haloalkyl, C₃₋₇ cycloalkyl, or C₃₋₇ cycloalkyl-C₁₋₃alkyl. In some further embodiments, one of R^{c} and R^{d} is H and the other is C₁₋₇ alkyl, C₂₋₆ alkenyl, C₂₋₆ alkynyl, or C₁₋₇ haloalkyl. In still further embodiments, one of R^{c} and R^{d} is H and the other is C₁₋₇ alkyl or C₁₋₇ haloalkyl.

In some embodiments, R^{c} and R^{d} are each, independently, hydrogen, C₁₋₇ alkyl, C₂₋₆ alkenyl, C₂₋₆ alkynyl, C₁₋₇ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉heteroaryl, or C₃₋₉ heteroaryl-C₁₋₃alkyl; wherein the C₁₋₇ alkyl, C₂₋₆ alkenyl, C₂₋₆ alkynyl, and C₁₋₇ haloalkyl are each optionally substituted by 1 or 2 independently selected R⁹ groups; wherein the C₃₋₇ cycloalkyl, C₃₋₇ cycloalkyl-C₁₋₃ alkyl, C₃₋₇ heterocycloalkyl, and C₃₋₇ heterocycloalkyl-C₁₋₃ alkyl are each optionally substituted by 1, 2, or 3 independently selected R¹⁰ groups; and wherein the C₆₋₁₀aryl, C₆₋₁₀aryl-C₁₋₃alkyl, C₃₋₉ heteroaryl, and C₃₋₉heteroaryl-C₁₋₃ alkyl are each optionally substituted by 1, 2, or 3 independently selected R¹¹ groups.

In some embodiments, R^{c} and R^{d} are each, independently, hydrogen, C₁₋₇ alkyl, C₂₋₆ alkenyl, C₂₋₆ alkynyl, C₁₋₇ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein the C₁₋₇ alkyl, C₂₋₆ alkenyl, C₂₋₆ alkynyl, and C₁₋₇ haloalkyl are each optionally substituted by 1 independently selected R⁹ group; wherein the C₃₋₇ cycloalkyl, C₃₋₇ cycloalkyl-C₁₋₃ alkyl, C₃₋₇ heterocycloalkyl, and C₃₋₇ heterocycloalkyl-C₁₋₃ alkyl are each optionally substituted by 1 or 2 independently selected R¹⁰ groups; and wherein the C₆₋₁₀aryl, C₆₋₁₀aryl-C₁₋₃alkyl, C₃₋₉ heteroaryl, and C₃₋₉heteroaryl-C₁₋₃alkyl are each optionally substituted by 1 or 2 independently selected R¹¹ groups.

In some embodiments, R^{c} and R^{d} are each, independently, hydrogen, C₁₋₇ alkyl, C₂₋₆ alkenyl, C₂₋₆ alkynyl, C₁₋₇ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl.

In some embodiments, R^{c} and R^{d} are each, independently, hydrogen, C₁₋₇ alkyl, C₂₋₆ alkenyl, C₂₋₆ alkynyl, or C₁₋₇ haloalkyl; wherein the C₁₋₇ alkyl, C₂₋₆ alkenyl, C₂₋₆ alkynyl, and C₁₋₇ haloalkyl are each optionally substituted by 1, 2, or 3 independently selected R⁹ groups.

In some embodiments, R^{c}, and R^{d} are each, independently, hydrogen, C₁₋₇ alkyl, C₂₋₆ alkynyl, or C₁₋₇ haloalkyl.

In some embodiments, R^{c}, and R^{d} are each, independently, hydrogen, C₁₋₇ alkyl, C₂₋₆ alkynyl, or fluorinated C₁₋₇ haloalkyl.

In some embodiments, R^{c}, and R^{d} are each, independently, hydrogen, methyl, ethyl, isopropyl, prop-2-ynyl, or 2-fluoroethyl.

In some embodiments, R^{a} is C₁₋₇ alkyl, C₂₋₆ alkenyl, C₂₋₆ alkynyl, C₁₋₇ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃ alkyl, wherein the C₁₋₇ alkyl, C₂₋₆ alkenyl, C₂₋₆ alkynyl, and C₁₋₇ haloalkyl are each optionally substituted by 1 or 2 independently selected R⁹ groups; wherein the C₃₋₇ cycloalkyl, C₃₋₇ cycloalkyl-C₁₋₃ alkyl, C₃₋₇ heterocycloalkyl, and C₃₋₇ heterocycloalkyl-C₁₋₃ alkyl are each optionally substituted by 1, 2, or 3 independently selected R¹⁰ groups; and wherein the C₆₋₁₀aryl, C₆₋₁₀aryl-C₁₋₃alkyl, C₃₋₉ heteroaryl, and C₃₋₉heteroaryl-C₁₋₃ alkyl are each optionally substituted by 1, 2, or 3 independently selected R¹¹ groups.

In some embodiments, R^{a} is C₁₋₇ alkyl, C₂₋₆ alkenyl, C₂₋₆ alkynyl, C₁₋₇ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein the C₁₋₇ alkyl, C₂₋₆ alkenyl, C₂₋₆ alkynyl, and C₁₋₇ haloalkyl are each optionally substituted by 1 independently selected R⁹ group; wherein the C₃₋₇ cycloalkyl, C₃₋₇ cycloalkyl-C₁₋₃ alkyl, C₃₋₇ heterocycloalkyl, and C₃₋₇ heterocycloalkyl-C₁₋₃ alkyl are each optionally substituted by 1 or 2 independently selected R¹⁰ groups; and wherein the C₆₋₁₀aryl, C₆₋₁₀aryl-C₁₋₃alkyl, C₃₋₉ heteroaryl, and C₃₋₉heteroaryl-C₁₋₃ alkyl are each optionally substituted by 1 or 2 independently selected R¹¹ groups.

In some embodiments, R^{a} is C₁₋₇ alkyl, C₂₋₆ alkenyl, C₂₋₆ alkynyl, C₁₋₇ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ C₆₋₁₀aryl, C₆₋₁₀ aryl-C₁₋₃alkyl, C₃₋₉ heteroaryl, or C₃₋₉ heteroaryl-C₁₋₃alkyl.

In some embodiments, R^{a} is C₁₋₇ alkyl, C₂₋₆ alkenyl, C₂₋₆ alkynyl, or C₁₋₇ haloalkyl; wherein the C₁₋₇ alkyl, C₂₋₆ alkenyl, C₂₋₆ alkynyl, and C₁₋₇ haloalkyl are each optionally substituted by 1, 2, or 3 independently selected R⁹ groups.

In some embodiments, R^{a} is C₁₋₇ alkyl, C₂₋₆ alkynyl, or C₁₋₇ haloalkyl.

In some embodiments, R^{a} is C₁₋₇ alkyl, C₂₋₆ alkynyl, or fluorinated C₁₋₇ haloalkyl.

In some embodiments, R^{a} is methyl, ethyl, isopropyl, prop-2-ynyl, or 2-fluoroethyl.

In some embodiments, each R¹ is, independently, hydrogen, halogen, cyano, nitro, C₁₋₆alkyl, C₁₋₆haloalkyl, C₂₋₆alkenyl, —OR^{f}, —C(═O)OR^{f}, or —C(═O)NR^{g}R^{h}.

In some embodiments, each R¹ is, independently, hydrogen, halogen, cyano, C₁₋₆alkyl, C₂₋₆alkenyl, —C(═O)OR^{f}, —C(═O)NR^{g}R^{h}, hydroxyl, or C₁₋₆alkoxy.

In some embodiments, each R¹ is, independently, hydrogen, halogen, cyano, C₁₋₆alkyl, C₁₋₆haloalkyl, hydroxyl, or C₁₋₆alkoxy.

In some embodiments, each R¹ is, independently, hydrogen, halogen, or C₁₋₆alkyl.

In some embodiments, each R¹ is, independently, hydrogen, fluoro, or methyl.

In some embodiments, R³ is C₁₋₆ alkyl.

In some embodiments, R³ is methyl.

In some embodiments, each R⁴ and R⁵ is, independently, halogen, C₁₋₆alkyl, C₁₋₆haloalkyl, C₁₋₆alkoxy, hydroxyl-C₁₋₆alkyl-, —CH₂—OR, -or —C(═O)NR₂.

In some embodiments, each R⁴ and R⁵ is, independently, halogen, C₁₋₆alkyl, or C₁₋₆haloalkyl.

In some embodiments, each R⁴ and R⁵ is, independently, C₁₋₆alkyl.

In some embodiments, each R⁴ and R⁵ is, independently, C₁₋₃alkyl.

In some embodiments, each R⁴ and R⁵ is, independently, methyl.

In some embodiments, each R⁹, R¹⁰, and R¹¹ is, independently, halogen, cyano, nitro, —SR^{w}, —OR^{x}, —O(CH₂)ᵣ—OR^{x}, R^{x}, —C(═O)—R^{w}, —C(═O)OR^{x}, —C(═O)NR^{y}R^{z}, —SO₂R^{w}, SO₂NR^{y}R^{z}, —NR^{y}R^{z}, or —(CH₂)ᵣNR^{y}R^{z}.

In some embodiments, each R⁹, R¹⁰, and R¹¹ is, independently, halogen, cyano, nitro, —OR^{x}, R^{x}, —SO₂R^{w}, —NR^{y}R^{z}, or —(CH₂)ᵣNR^{y}R^{z}.

In some embodiments, each R⁹, R¹⁰, and R¹¹ is, independently, halogen, cyano, nitro, —OR^{x}, R^{z}, or —NR^{y}R^{z}.

In some embodiments, each R⁹, R¹⁰, and R¹¹ is, independently, halogen, —OR^{x}, R^{x}, or —NR^{y}R^{z}.

In some embodiments, each R⁹, R¹⁰, and R¹¹ is, independently, halogen, —OR^{x}, or R^{x}.

In some embodiments, r is 1, 2, or 3. In some embodiments, r is 1 or 2. In some embodiments, r is 1. In some embodiments, r is 2, 3, or 4.

In some embodiments, m is 0, 1, 2, 3, or 4. In some embodiments, m is 0, 1, 2, or 3. In some embodiments, m is 0, 1 or 2. In some embodiments, m is 0 or 1. In some embodiments, m is 0.

In some embodiments, p is an integer from 0 to 4. In some embodiments, p is 0, 1, 2, or 3. In some embodiments, p is 0, 1 or 2. In some embodiments, p is 0 or 1. In some embodiments, p is 0.

In some embodiments, n is 1, 2, or 3. In some embodiments, n is 1 or 2. In some embodiments, n is 1.

In some embodiments, m and p are each, independently, 0, 1, or 2; and n is 1 or 2.

In some embodiments, m and p are each 0; and n is 1 or 2.

In some embodiments, m and p are each 0; and n is 1.

In some embodiments, the compound is a compound of Formula II:

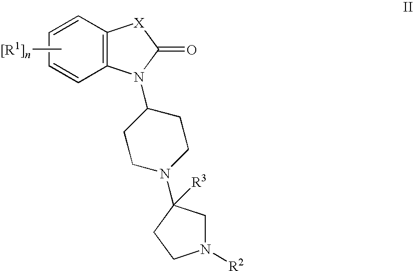

or pharmaceutically acceptable salt thereof; wherein R¹, R², R³, X, and n are defined the same as in any of the embodiments above, or combination thereof.

In some embodiments the compound is a compound of Formula III:

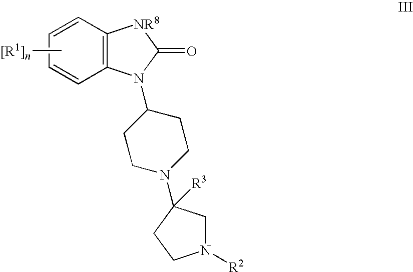

or pharmaceutically acceptable salt thereof; R¹, R², R³, R⁸, X, and n are defined the same as in any of the embodiments above, or combination thereof.

In some embodiments, the compound has the structure of Formula IV or V:

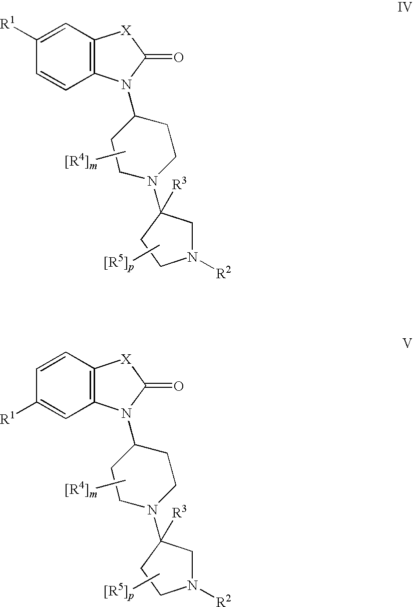

or is pharmaceutically acceptable salt thereof, wherein R¹, R², R³, R⁴, R⁵, X, m, and p are defined the same as in any of the embodiments above, or combination thereof.

In some embodiments, the compound has the structure of Formula VI or VII:

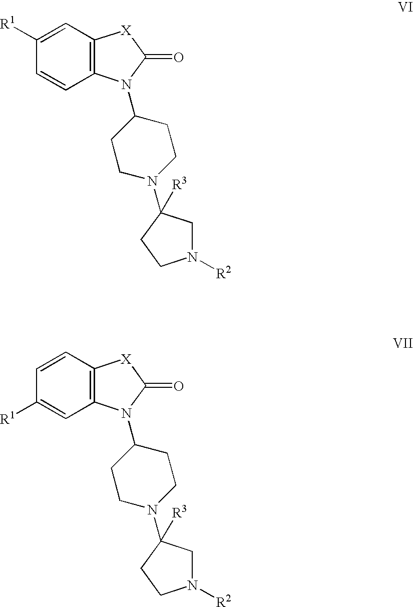

or is pharmaceutically acceptable salt thereof, wherein R¹, R², R³, and X are defined the same as in any of the embodiments above, or combination thereof.

In some embodiments, the compound has the structure of Formula VIII or IX:

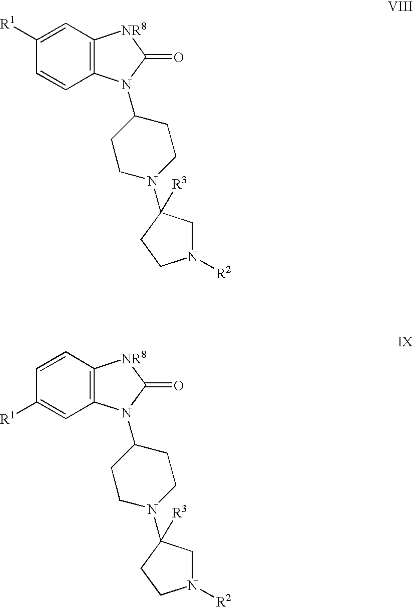

or is pharmaceutically acceptable salt thereof, wherein R¹, R², R³, and R⁸ are defined the same as in any of the embodiments above, or combination thereof.

In some embodiments:

X is —CR⁶R⁷—, —NR⁸—, —O—, or —S—;

each R¹ is, independently, hydrogen, halogen, cyano, nitro, C₁₋₆alkyl, C₁₋₆haloalkyl, C₂₋₆alkenyl, C₂₋₆ alkynyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀aryl-C₁₋₃alkyl, C₃₋₉ heteroaryl, C₃₋₉heteroaryl,-C₁₋₃alkyl, —SR^{e}, —OR^{f}, —O(CH₂)ᵣ—OR^{f}, —C(═O)—R^{e}, —C(═O)OR^{f}, —C(═O)NR^{g}R^{h}, —SO₂R^{e}, —SO2NR^{g}R^{h}, —NR^{g}R^{h}, or —(CH₂)ᵣNR^{g}R^{h};

R² is —C(═O)OR^{a} or —C(═O)NR^{c}R^{d};

R³ is C₁₋₆ alkyl or C₁₋₆ haloalkyl;

each R⁴ is, independently, C₁₋₆alkyl;

each R⁵ is, independently, C₁₋₆alkyl;

R⁶, R⁷, and R³ are each, independently, hydrogen;

each R⁹, R¹⁰, and R¹¹ is, independently, phenyl, C₃₋₆cycloalkyl, C₂₋₅heterocycloalkyl, C₃₋₅ heteroaryl, halogen, cyano, nitro, —SR^{w}, —OR^{x}, —O(CH₂)ᵣ—OR^{x}, R^{x}, —C(═O)—R^{w}, —C(═O)OR^{x}, —C(═O)NR^{y}R^{z}, —SO₂R^{w}, —SO₂NR^{y}R^{z}, —NR^{y}R^{z}, or —(CH₂)ᵣNR^{y}R^{z};

R^{a}, R^{c}, and R^{d} are each, independently, hydrogen, C₁₋₇ alkyl, C₁₋₇ haloalkyl, C₂₋₆ alkenyl, C₂₋₆ alkynyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ -C₁₋₃alkyl; wherein the C₁₋₇ alkyl, C₂₋₆ alkenyl, C₂₋₆ alkynyl, and C₁₋₇ haloalkyl are each optionally substituted by 1, 2, or 3 independently selected R⁹ groups; wherein the C₃₋₇ cycloalkyl, C₃₋₇ cycloalkyl-C₁₋₃ alkyl, C₃₋₇ heterocycloalkyl, and C₃₋₇ heterocycloalkyl-C₁₋₃ alkyl are each optionally substituted by 1, 2, 3, or 4 independently selected R¹⁰ groups; and wherein the C₆₋₁₀aryl, C₆₋₁₀aryl-C₁₋₃alkyl, C₃₋₉ heteroaryl, and C₃₋₉heteroaryl-C₁₋₃ alkyl are each optionally substituted by 1, 2, 3, or 4 independently selected R¹¹ groups, with a proviso that R^{a} is not hydrogen;

each R^{e}, R^{f}, R^{g}, R^{h}, R^{w}, R^{x}, R^{y}, R^{z}, and R is, independently hydrogen, C₁₋₆ alkyl, C₂₋₆ alkenyl, or C₁₋₆ haloalkyl;

r is 1, 2, 3, or 4;

n is 1, 2, 3, or 4;

m is 0, 1, 2, 3, 4, 5, 6, 7, or 8; and

p is an integer from 0 to 6.

In some embodiments:

X is —NR⁸—;

each R¹ is, independently, hydrogen, halogen, cyano, nitro, C₁₋₆alkyl, C₁₋₆haloalkyl, C₂₋₆alkenyl, —OR^{f}, —C(═O)OR^{f}, or —C(═O)NR^{g}R^{h};

R² is —C(═O)OR^{a}, —C(═O)NR^{c}R^{d}, C₁₋₇ alkyl, C₁₋₇ haloalkyl, C₂₋₆ alkynyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀aryl-C₁₋₃alkyl, C₃₋₉ heteroaryl, or C₃₋₉heteroaryl-C₁₋₃alkyl; wherein the C₁₋₇ alkyl, C₂₋₆ alkynyl, and C₁₋₇ haloalkyl are each optionally substituted by 1, 2, or 3 independently selected R⁹ groups; wherein the C₃₋₇ cycloalkyl, C₃₋₇ cycloalkyl-C₁₋₃ alkyl, C₃₋₇ heterocycloalkyl, and C₃₋₇ heterocycloalkyl-C₁₋₃ alkyl are each optionally substituted by 1, 2, 3, or 4 independently selected R¹⁰ groups; and wherein the C₆₋₁₀aryl, C₆₋₁₀aryl-C₁₋₃alkyl, C₃₋₉ heteroaryl, and C₃₋₉heteroaryl-C₁₋₃ alkyl are each optionally substituted by 1, 2, 3, or 4 independently selected R¹¹ groups;

R³ is C₁₋₆ alkyl or C₁₋₆ haloalkyl;

each R⁴ and R⁵ is, independently, C₁₋₆alkyl;

R⁸ is hydrogen or C₁₋₆alkyl;

each R⁹, R¹⁰, and R¹¹ is, independently, halogen, cyano, nitro, —OR^{x}, R^{x}, —SO₂R^{w}, —NR^{y}R^{z}, or —(CH₂)ᵣNR^{y}R^{z};

R^{a} is C₁₋₇ alkyl, C₂₋₆ alkenyl, C₂₋₆ alkynyl, C₁₋₇ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein the C₁₋₇ alkyl, C₂₋₆ alkenyl, C₂₋₆ alkynyl, and C₁₋₇ haloalkyl are each optionally substituted by 1 or 2 independently selected R⁹ groups; wherein the C₃₋₇ cycloalkyl, C₃₋₇ cycloalkyl-C₁₋₃ alkyl, C₃₋₇ heterocycloalkyl, and C₃₋₇ heterocycloalkyl-C₁₋₃ alkyl are each optionally substituted by 1, 2, or 3 independently selected R¹⁰ groups; and wherein the C₆₋₁₀aryl, C₆₋₁₀aryl-C₁₋₃alkyl, C₃₋₉ heteroaryl, and C₃₋₉heteroaryl-C₁₋₃ alkyl are each optionally substituted by 1, 2, or 3 independently selected R¹¹ groups;

R^{c} and R^{d} are each, independently, hydrogen, C₁₋₇ alkyl, C₂₋₆ alkenyl, C₂₋₆ alkynyl, C₁₋₇ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein the C₁₋₇ alkyl, C₂₋₆ alkenyl, C₂₋₆ alkynyl, and C₁₋₇ haloalkyl are each optionally substituted by 1 or 2 independently selected R⁹ groups; wherein the C₃₋₇ cycloalkyl, C₃₋₇ cycloalkyl-C₁₋₃ alkyl, C₃₋₇ heterocycloalkyl, and C₃₋₇ heterocycloalkyl-C₁₋₃ alkyl are each optionally substituted by 1, 2, or 3 independently selected R¹⁰ groups; and wherein the C₆₋₁₀aryl, C₆₋₁₀aryl-C₁₋₃alkyl, C₃₋₉ heteroaryl, and C₃₋₉heteroaryl-C₁₋₃ alkyl are each optionally substituted by 1, 2, or 3 independently selected R¹¹ groups;

each R^{f}, R^{g}, R^{h}, R^{w}, R^{x}, R^{y}, and R^{z} is, independently hydrogen, C₁₋₆ alkyl, or C₁₋₆ haloalkyl;

r is 1, 2, 3, or 4;

n is 1, 2, 3, or 4;

m is 0, 1, 2, 3, or 4; and

p is an integer from 0 to 4.

In some embodiments:

X is —NR⁸—;

each R¹ is, independently, hydrogen, halogen, cyano, nitro, C₁₋₆alkyl, C₁₋₆haloalkyl, C₂₋₆alkenyl, —OR^{f}, —C(═O)OR^{f}, or —C(═O)NR^{g}R^{h};

R² is —C(═O)OR^{a} or —C(═O)NR^{c}R^{d};

R³ is C₁₋₆ alkyl or C₁₋₆ haloalkyl;

each R⁴ and R⁵ is, independently, C₁₋₆alkyl;

R⁸ is hydrogen or C₁₋₆alkyl;

each R⁹, R¹⁰, and R¹¹ is, independently, halogen, cyano, nitro, —OR^{x}, R^{x}, —SO₂R^{w}, —NR^{y}R^{z}, or —(CH₂)ᵣNR^{y}R^{z};

R^{a} is C₁₋₇ alkyl, C₂₋₆ alkenyl, C₂₋₆ alkynyl, C₁₋₇ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein the C₁₋₇ alkyl, C₂₋₆ alkenyl, C₂₋₆ alkynyl, and C₁₋₇ haloalkyl are each optionally substituted by 1 or 2 independently selected R⁹ groups; wherein the C₃₋₇ cycloalkyl, C₃₋₇ cycloalkyl-C₁₋₃ alkyl, C₃₋₇ heterocycloalkyl, and C₃₋₇ heterocycloalkyl-C₁₋₃ alkyl are each optionally substituted by 1, 2, or 3 independently selected R¹⁰ groups; and wherein the C₆₋₁₀aryl, C₆₋₁₀aryl-C₁₋₃alkyl, C₃₋₉ heteroaryl, and C₃₋₉heteroaryl-C₁₋₃ alkyl are each optionally substituted by 1, 2, or 3 independently selected R¹¹ groups;

R^{c} and R^{d} are each, independently, hydrogen, C₁₋₇ alkyl, C₂₋₆ alkenyl, C₂₋₆ alkynyl, C₁₋₇ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein the C₁₋₇ alkyl, C₂₋₆ alkenyl, C₂₋₆ alkynyl, and C₁₋₇ haloalkyl are each optionally substituted by 1 independently selected R⁹ groups; wherein the C₃₋₇ cycloalkyl, C₃₋₇ cycloalkyl-C₁₋₃ alkyl, C₃₋₇ heterocycloalkyl, and C₃₋₇ heterocycloalkyl-C₁₋₃ alkyl are each optionally substituted by 1, 2, or 3 independently selected R¹⁰ groups; and wherein the C₆₋₁₀aryl, C₆₋₁₀aryl-C₁₋₃alkyl, C₃₋₉ heteroaryl, and C₃₋₉heteroaryl-C₁₋₃ alkyl are each optionally substituted by 1, 2, or 3 independently selected R¹¹ groups;

each R^{f}, R^{g}, R^{h}, R^{w}, R^{x}, R^{y}, and R^{z} is, independently hydrogen, C₁₋₆ alkyl, or C₁₋₆ haloalkyl;

r is 1, 2, 3, or 4;

n is 1, 2, 3, or 4;

m is 0, 1, or 2; and

p is 0, 1, or 2.

In some embodiments:

X is —NR⁸—;

each R¹ is, independently, hydrogen, halogen, cyano, C₁₋₆alkyl, C₂₋₆alkenyl, —C(═O)OR^{f}, —C(═O)NR^{g}R^{h}, hydroxyl, or C₁₋₆alkoxy;

R² is —C(═O)OR^{a} or —C(═O)NR^{c}R^{d};

R³ is C₁₋₆ alkyl or C₁₋₆ haloalkyl;

R³ is hydrogen or C₁₋₆alkyl;

each R⁹, R¹⁰, and R¹¹ is, independently, halogen, cyano, nitro, —OR^{x}, R^{x}, —SO₂R^{w}, —NR^{y}R^{z}, or —(CH₂)ᵣNR^{y}R^{z};

R^{a} is C₁₋₇ alkyl, C₂₋₆ alkenyl, C₂₋₆ alkynyl, C₁₋₇ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein the C₁₋₇ alkyl, C₂₋₆ alkenyl, C₂₋₆ alkynyl, and C₁₋₇ haloalkyl are each optionally substituted by 1 or 2 independently selected R⁹ groups; wherein the C₃₋₇ cycloalkyl, C₃₋₇ cycloalkyl-C₁₋₃ alkyl, C₃₋₇ heterocycloalkyl, and C₃₋₇ heterocycloalkyl-C₁₋₃ alkyl are each optionally substituted by 1, 2, or 3 independently selected R¹⁰ groups; and wherein the C₆₋₁₀aryl, C₆₋₁₀aryl-C₁₋₃alkyl, C₃₋₉ heteroaryl, and C₃₋₉heteroaryl-C₁₋₃ alkyl are each optionally substituted by 1, 2, or 3 independently selected R¹¹ groups;

R^{c} and R^{d} are each, independently, hydrogen, C₁₋₇ alkyl, C₂₋₆ alkenyl, C₂₋₆ alkynyl, C₁₋₇ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein the C₁₋₇ alkyl, C₂₋₆ alkenyl, C₂₋₆ alkynyl, and C₁₋₇ haloalkyl are each optionally substituted by 1 or 2 independently selected R⁹ groups; wherein the C₃₋₇ cycloalkyl, C₃₋₇ cycloalkyl-C₁₋₃ alkyl, C₃₋₇ heterocycloalkyl, and C₃₋₇ heterocycloalkyl-C₁₋₃ alkyl are each optionally substituted by 1, 2, or 3 independently selected R¹⁰ groups; and wherein the C₆₋₁₀aryl, C₆₋₁₀aryl-C₁₋₃alkyl, C₃₋₉ heteroaryl, and C₃₋₉heteroaryl-C₁₋₃ alkyl are each optionally substituted by 1, 2, or 3 independently selected R¹¹ groups;

each R^{w}, R^{x}, R^{y}, and R^{z} is, independently hydrogen, C₁₋₆ alkyl, or C₁₋₆ haloalkyl;

r is 1, 2, 3, or 4;

n is 1, 2, 3, or 4;

m and p are each 0.

In some embodiments:

X is —NR⁸—;

each R¹ is, independently, hydrogen, halogen, cyano, C₁₋₆alkyl, C₂₋₆alkenyl, —C(═O)OR^{f}, —C(═O)NR^{g}R^{h}, hydroxyl, or C₁₋₆ alkyl;

R² is —C(═O)OR^{a} or —C(═O)NR^{c}R^{d};

R³ is C₁₋₆ alkyl or C₁₋₆ haloalkyl;

R⁸ is hydrogen, C₁₋₆alkyl, C₂₋₆ alkenyl, or C₁₋₆ haloalkyl;

each R⁹, R¹⁰, and R¹¹ is, independently, phenyl, C₃₋₆cycloalkyl, C₂₋₅heterocycloalkyl, C₃₋₅ heteroaryl, halogen, cyano, nitro, —SR^{w}, —OR^{x}, —O(CH₂)ᵣ—OR^{x}, R^{x}, —C(═O)—R^{w}, —C(═O)OR^{x}, —C(═O)NR^{y}R^{z}, —SO₂R^{w}, —SO₂NR^{y}R^{z}, —NR^{y}R^{z}, or —(CH₂)ᵣNR^{y}R^{z};

R^{a} is C₁₋₇ alkyl, C₂₋₆ alkenyl, C₂₋₆ alkynyl, C₁₋₇ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein the C₁₋₇ alkyl, C₂₋₆ alkenyl, C₂₋₆ alkynyl, and C₁₋₇ haloalkyl are each optionally substituted by 1 or 2 independently selected R⁹ groups; wherein the C₃₋₇ cycloalkyl, C₃₋₇ cycloalkyl-C₁₋₃ alkyl, C₃₋₇ heterocycloalkyl, and C₃₋₇ heterocycloalkyl-C₁₋₃ alkyl are each optionally substituted by 1, 2, or 3 independently selected R¹⁰ groups; and wherein the C₆₋₁₀aryl, C₆₋₁₀aryl-C₁₋₃alkyl, C₃₋₉ heteroaryl, and C₃₋₉heteroaryl-C₁₋₃ alkyl are each optionally substituted by 1, 2, or 3 independently selected R¹¹ groups;

R^{c} and R^{d} are each, independently, hydrogen, C₁₋₇alkyl, C₂₋₆alkenyl, C₂₋₆alkynyl, C₁₋₇ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein the C₁₋₇ alkyl, C₂₋₆ alkenyl, C₂₋₆ alkynyl, and C₁₋₇ haloalkyl are each optionally substituted by 1 or 2 independently selected R⁹ groups; wherein the C₃₋₇ cycloalkyl, C₃₋₇ cycloalkyl-C₁₋₃ alkyl, C₃₋₇ heterocycloalkyl, and C₃₋₇ heterocycloalkyl-C₁₋₃ alkyl are each optionally substituted by 1, 2, or 3 independently selected R¹⁰ groups; and wherein the C₆₋₁₀ aryl, C₆₋₁₀aryl-C₁₋₃alkyl, C₃₋₉ heteroaryl, and C₃₋₉heteroaryl-C₁₋₃ alkyl are each optionally substituted by 1, 2, or 3 independently selected R¹¹ groups;

each R^{f}, R^{g}, R^{h}, R^{w}, R^{x}, R^{y}, and R^{z} is, independently hydrogen, C₁₋₆ alkyl, or C₁₋₆ haloalkyl;

r is 1, 2, 3, or 4;

n is 1, 2, 3, or 4; and

m and p are each 0.

In some embodiments:

X is —NR⁸—;

each R¹ is, independently, hydrogen, halogen, cyano, C₁₋₆alkyl, C₂₋₆alkenyl, —C(═O)OR^{f}, —C(═O)NR^{g}R^{h}, hydroxyl, or C₁₋₆alkoxy;

R² is —C(═O)OR^{a} or —C(═O)NR^{c}R^{d};

R³ is C₁₋₆ alkyl or C₁₋₆ haloalkyl;

R⁸ is hydrogen;

each R⁹, R¹⁰, and R¹¹ is, independently, halogen, cyano, nitro, —OR^{x}, R^{x}, —SO₂R^{w}, —NR^{y}R^{z}, or —(CH₂)ᵣNR^{y}R^{z};

R^{a} is C₁₋₇ alkyl, C₂₋₆ alkynyl, C₁₋₇ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein the C₁₋₇ alkyl, C₂₋₆ alkynyl, and C₁₋₇ haloalkyl are each optionally substituted by 1 or 2 independently selected R⁹ groups; wherein the C₃₋₇ cycloalkyl, C₃₋₇ cycloalkyl-C₁₋₃ alkyl, C₃₋₇ heterocycloalkyl, and C₃₋₇ heterocycloalkyl-C₁₋₃ alkyl are each optionally substituted by 1, 2, or 3 independently selected R¹⁰ groups; and wherein the C₆₋₁₀aryl, C₆₋₁₀aryl-C₁₋₃alkyl, C₃₋₉ heteroaryl, and C₃₋₉heteroaryl-C₁₋₃ alkyl are each optionally substituted by 1, 2, or 3 independently selected R¹¹ groups;

R^{c} and R^{d} are each, independently, hydrogen, C₁₋₇ alkyl, C₂₋₆ alkynyl, C₁₋₇ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein the C₁₋₇alkyl, C₂₋₆ alkynyl, and C₁₋₇ haloalkyl are each optionally substituted by 1 or 2 independently selected R⁹ groups; wherein the C₃₋₇ cycloalkyl, C₃₋₇ cycloalkyl-C₁₋₃ alkyl, C₃₋₇ heterocycloalkyl, and C₃₋₇ heterocycloalkyl-C₁₋₃ alkyl are each optionally substituted by 1, 2, or 3 independently selected R¹⁰ groups; and wherein the C₆₋₁₀aryl, C₆₋₁₀aryl-C₁₋₃alkyl, C₃₋₉ heteroaryl, and C₃₋₉heteroaryl-C₁₋₃ alkyl are each optionally substituted by 1, 2, or 3 independently selected R¹¹ groups;

each R^{f}, R^{g}, R^{h}, R^{w}, R^{x}, R^{y}, and R^{z} is, independently hydrogen, C₁₋₆ alkyl, or C₁₋₆ haloalkyl;

r is 1, 2, 3, or 4;

n is 1, 2, 3, or 4; and

m and p are each 0.

In some embodiments:

X is —NR⁸—;

each R¹ is, independently, hydrogen, halogen, cyano, C₁₋₆alkyl, hydroxyl, or C₁₋₆alkoxy;

R² is —C(═O)OR^{a} or —C(═O)NR^{c}R^{d};

R³ is C₁₋₆ alkyl or C₁₋₆ haloalkyl;

R⁸ is hydrogen;

each R⁹, R¹⁰, and R¹¹ is, independently, halogen, cyano, nitro, —OR^{x}, R^{x}, —SO₂R^{w}, —NR^{y}R^{z}, or —(CH₂)ᵣNR^{y}R^{z};

R^{a} is C₁₋₇ alkyl, C₂₋₆ alkynyl, C₁₋₇ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein the C₁₋₇ alkyl, C₂₋₆ alkynyl, and C₁₋₇ haloalkyl are each optionally substituted by 1 or 2 independently selected R⁹ groups; wherein the C₃₋₇ cycloalkyl, C₃₋₇ cycloalkyl-C₁₋₃ alkyl, C₃₋₇ heterocycloalkyl, and C₃₋₇ heterocycloalkyl-C₁₋₃ alkyl are each optionally substituted by 1, 2, or 3 independently selected R¹⁰ groups; and wherein the C₆₋₁₀aryl, C₆₋₁₀aryl-C₁₋₃alkyl, C₃₋₉ heteroaryl, and C₃₋₉heteroaryl-C₁₋₃ alkyl are each optionally substituted by 1, 2, or 3 independently selected R¹¹ groups;

R^{c} and R^{d} are each, independently, hydrogen, C₁₋₇ alkyl, C₂₋₆ alkynyl, C₁₋₇ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein the C₁₋₇ alkyl, C₂₋₆ alkynyl, and C₁₋₇ haloalkyl are each optionally substituted by 1 or 2 independently selected R⁹ groups; wherein the C₃₋₇ cycloalkyl, C₃₋₇ cycloalkyl-C₁₋₃ alkyl, C₃₋₇ heterocycloalkyl, and C₃₋₇ heterocycloalkyl-C₁₋₃ alkyl are each optionally substituted by 1, 2, or 3 independently selected R¹⁰ groups; and wherein the C₆₋₁₀aryl, C₆₋₁₀aryl-C₁₋₃alkyl, C₃₋₉ heteroaryl, and C₃₋₉heteroaryl-C₁₋₃ alkyl are each optionally substituted by 1, 2, or 3 independently selected R¹¹ groups;

each R^{t}, R^{w}, R^{x}, R^{y}, and R^{z} is, independently hydrogen, C₁₋₆alkyl, or C₁₋₆ haloalkyl;

r is 1, 2, 3, or 4;

n is 1, 2, 3, or 4; and

m and p are each 0.

In some embodiments:

X is —NR⁸—;

each R¹ is, independently, hydrogen, halogen, C₁₋₆alkyl, or C₁₋₆haloalkyl;

R² is —C(═O)OR^{a} or —C(═O)NR^{c}R^{d};

R³ is C₁₋₆ alkyl;

R⁸ is hydrogen;

R^{a}, R^{c}, and R^{d} are each, independently, hydrogen, C₁₋₇alkyl, C₂₋₆ alkenyl, C₂₋₆alkynyl, or C₁₋₆ haloalkyl;

n is 1, 2, 3, or 4; and

m and p are each 0.

In some embodiments:

X is —NR⁸—;

each R¹ is, independently, hydrogen, halogen, C₁₋₆alkyl, or C₁₋₆haloalkyl;

R² is —C(═O)OR^{a} or —C(═O)NR^{c}R^{d};

R³ is C₁₋₆ alkyl;

R⁸ is hydrogen;

R^{a} is C₁₋₇ alkyl, C₂₋₆ alkenyl, C₂₋₆ alkynyl, or C₁₋₆ haloalkyl;

R^{c} and R^{d} are each, independently, hydrogen, C₁₋₇ alkyl, C₂₋₆ alkenyl, C₂₋₆ alkynyl, or C₁₋₆ haloalkyl;

n is 1 or 2; and

m and p are each 0.

In some embodiments:

X is —NR⁸—;

each R¹ is, independently, hydrogen, fluoro, or methyl;

R² is —C(═O)OR^{a} or —C(═O)NR^{c}R^{d};

R³ is methyl;

R⁸ is hydrogen;

R^{a}, R^{c}, and R^{d} are each, independently, methyl, ethyl, isopropyl, 2-fluoroethyl, or prop-2-ynyl;

n is 1; and

m and p are each 0.

In some embodiments, the compound is selected from:

ethyl 3-methyl-3-(4-(2-oxo-2,3-dihydro-1H-benzo[d]imidazol-1-yl)piperidin-1-yl)pyrrolidine-1-carboxylate;

methyl 3-(4-(2-oxo-2,3-dihydro-1H-benzo[d]imidazol-1-yl)piperidin-1-yl)pyrrolidine-1-carboxylate;

isopropyl 3-methyl-3-(4-(2-oxo-2,3-dihydro-1H-benzo[d]imidazol-1-yl)piperidin-1-yl)pyrrolidine-1-carboxylate;

2-fluoroethyl 3-methyl-3-(4-(2-oxo-2,3-dihydro-1H-benzo[d]imidazol-1-yl)piperidin-1-yl)pyrrolidine-1-carboxylate;

N-ethyl-3-methyl-3-(4-(2-oxo-2,3-dihydro-1H-benzo[d]imidazol-1-yl)piperidin-1-yl)pyrrolidine-1-carboxamide;

ethyl 3-(4-(5-fluoro-2-oxo-2,3-dihydro-1H-benzo[d]imidazol-1-yl)piperidin-1-yl)-3-methylpyrrolidine-1-carboxylate;

methyl 3-(4-(5-fluoro-2-oxo-2,3-dihydro-1H-benzo[d]imidazol-1-yl)piperidin-1-yl)-3-methylpyrrolidine-1-carboxylate;

isopropyl 3-(4-(5-fluoro-2-oxo-2,3-dihydro-1H-benzo[d]imidazol-1-yl)piperidin-1-yl)-3-methylpyrrolidine-1-carboxylate;

2-fluoroethyl 3-(4-(5-fluoro-2-oxo-2,3-dihydro-1H-benzo[d]imidazol-1-yl)piperidin-1-yl)-3-methylpyrrolidine-1-carboxylate;

prop-2-ynyl 3-(4-(5-fluoro-2-oxo-2,3-dihydro-1H-benzo[d]imidazol-1-yl)piperidin-1-yl)-3-methylpyrrolidine-1-carboxylate;

ethyl 3-methyl-3-(4-(5-methyl-2-oxo-2,3-dihydro-1H-benzo[d]imidazol-1-yl)piperidin-1-yl)pyrrolidine-1-carboxylate;

isopropyl 3-methyl-3-(4-(5-methyl-2-oxo-2,3-dihydro-1H-benzo[d]imidazol-1-yl)piperidin-1-yl)pyrrolidine-1-carboxylate;

ethyl 3-methyl-3-(4-(6-methyl-2-oxo-2,3-dihydro-1H-benzo[d]imidazol-1-yl)piperidin-1-yl)pyrrolidine-1-carboxylate; and

methyl 3-methyl-3-(4-(6-methyl-2-oxo-2,3-dihydro-1H-benzo[d]imidazol-1-yl)piperidin-1-yl)pyrrolidine-1-carboxylate;

but-2-ynyl 3-methyl-3-(4-(6-methyl-2-oxo-2,3-dihydro-1H-benzo[d]imidazol-1-yl)piperidin-1-yl)pyrrolidine-1-carboxylate;

but-2-ynyl 3-methyl-3-(4-(5-methyl-2-oxo-2,3-dihydro-1H-benzo[d]imidazol-1-yl)piperidin-1-yl)pyrrolidine-1-carboxylate;

but-2-ynyl 3-(4-(5-fluoro-2-oxo-2,3-dihydro-1H-benzo[d]imidazol-1-yl)piperidin-1-yl)-3-methylpyrrolidine-1-carboxylate;

methyl 3-(4-(5-fluoro-2-oxo-2,3-dihydro-1H-benzo[d]imidazol-1-yl)piperidin-1-yl)-3-methylpyrrolidine-1-carboxylate;

2-fluoroethyl 3-(4-(5-fluoro-2-oxo-2,3-dihydro-1H-benzo[d]imidazol-1-yl)piperidin-1-yl)-3-methylpyrrolidine-1-carboxylate;

prop-2-ynyl 3-(4-(5-fluoro-2-oxo-2,3-dihydro-1H-benzo[d]imidazol-1-yl)piperidin-1-yl)-3-methylpyrrolidine-1-carboxylate;

2-fluoroethyl 3-methyl-3-(4-(6-methyl-2-oxo-2,3-dihydro-1H-benzo[d]imidazol-1-yl)piperidin-1-yl)pyrrolidine-1-carboxylate;

2-fluoroethyl 3-methyl-3-(4-(5-methyl-2-oxo-2,3-dihydro-1H-benzo[d]imidazol-1-yl)piperidin-1-yl)pyrrolidine-1-carboxylate;

methyl 3-methyl-3-(4-(2-oxo-2,3-dihydro-1H-benzo[d]imidazol-1-yl)piperidin-1-yl)pyrrolidine-1-carboxylate;

2-fluoroethyl 3-methyl-3-(4-(2-oxo-2,3-dihydro-1H-benzo[d]imidazol-1-yl)piperidin-1-yl)pyrrolidine-1-carboxylate;

but-2-ynyl 3-methyl-3-(4-(2-oxo-2,3-dihydro-1H-benzo[d]imidazol-1-yl)piperidin-1-yl)pyrrolidine-1-carboxylate;

but-2-ynyl 3-(4-(5-fluoro-2-oxo-2,3-dihydro-1H-benzo[d]imidazol-1-yl)piperidin-1-yl)-3-methylpyrrolidine-1-carboxylate;

prop-2-ynyl 3-(4-(5-fluoro-2-oxo-2,3-dihydro-1H-benzo[d]imidazol-1-yl)piperidin-1-yl)-3-methylpyrrolidine-1-carboxylate;

methyl 3-(4-(5-fluoro-2-oxo-2,3-dihydro-1H-benzo[d]imidazol-1-yl)piperidin-1-yl)-3-methylpyrrolidine-1-carboxylate;

2-fluoroethyl 3-(4-(5-fluoro-2-oxo-2,3-dihydro-1H-benzo[d]imidazol-1-yl)piperidin-1-yl)-3-methylpyrrolidine-1-carboxylate;

isopropyl 3-methyl-3-(4-(6-methyl-2-oxo-2,3-dihydro-1H-benzo[d]imidazol-1-yl)piperidin-1-yl)pyrrolidine-1-carboxylate;

prop-2-ynyl 3-methyl-3-(4-(6-methyl-2-oxo-2,3-dihydro-1H-benzo[d]imidazol-1-yl)piperidin-1-yl)pyrrolidine-1-carboxylate;

prop-2-ynyl 3-methyl-3-(4-(5-methyl-2-oxo-2,3-dihydro-1H-benzo[d]imidazol-1-yl)piperidin-1-yl)pyrrolidine-1-carboxylate;

methyl 3-methyl-3-(4-(5-methyl-2-oxo-2,3-dihydro-1H-benzo[d]imidazol-1-yl)piperidin-1-yl)pyrrolidine-1-carboxylate;

methyl 3-methyl-3-(4-(6-methyl-2-oxo-2,3-dihydro-1H-benzo[d]imidazol-1-yl)piperidin-1-yl)pyrrolidine-1-carboxylate;

ethyl 3-(4-(6-fluoro-2-oxo-2,3-dihydro-1H-benzo[d]imidazol-1-yl)piperidin-1-yl)-3-methylpyrrolidine-1-carboxylate;

methyl 3-(4-(6-fluoro-2-oxo-2,3-dihydro-1H-benzo[d]imidazol-1-yl)piperidin-1-yl)-3-methylpyrrolidine-1-carboxylate;

2-fluoroethyl 3-(4-(6-fluoro-2-oxo-2,3-dihydro-1H-benzo[d]imidazol-1-yl)piperidin-1-yl)-3-methylpyrrolidine-1-carboxylate;

ethyl 3-(4-(5-fluoro-6-methyl-2-oxo-2,3-dihydro-1H-benzo[d]imidazol-1-yl)piperidin-1-yl)-3-methylpyrrolidine-1-carboxylate;

ethyl 3-(4-(5,6-difluoro-2-oxo-2,3-dihydro-1H-benzo[d]imidazol-1-yl)piperidin-1-yl)-3-methylpyrrolidine-1-carboxylate;

methyl 3-(4-(5,6-difluoro-2-oxo-2,3-dihydro-1H-benzo[d]imidazol-1-yl)piperidin-1-yl)-3-methylpyrrolidine-1-carboxylate;

2-fluoroethyl 3-(4-(5,6-difluoro-2-oxo-2,3-dihydro-1H-benzo[d]imidazol-1-yl)piperidin-1-yl)-3-methylpyrrolidine-1-carboxylate;

propyl 3-(4-(6-fluoro-2-oxo-2,3-dihydro-1H-benzo[d]imidazol-1-yl)piperidin-1-yl)-3-methylpyrrolidine-1-carboxylate;

isopropyl 3-(4-(6-fluoro-2-oxo-2,3-dihydro-1H-benzo[d]imidazol-1-yl)piperidin-1-yl)-3-methylpyrrolidine-1-carboxylate;

methyl 3-methyl-3-(4-(6-methyl-2-oxoindolin-1-yl)piperidin-1-yl)pyrrolidine-1-carboxylate;

ethyl 3-methyl-3-(4-(2-oxoindolin-1-yl)piperidin-1-yl)pyrrolidine-1-carboxylate;

methyl 3-methyl-3-(4-(2-oxoindolin-1-yl)piperidin-1-yl)pyrrolidine-1-carboxylate;

ethyl 3-methyl-3-(4-(2-oxoindolin-1-yl)piperidin-1-yl)pyrrolidine-1-carboxylate;

ethyl 3-(4-(6-fluoro-2-oxoindolin-1-yl)piperidin-1-yl)-3-methylpyrrolidine-1-carboxylate;

methyl 3-(4-(6-fluoro-2-oxoindolin-1-yl)piperidin-1-yl)-3-methylpyrrolidine-1-carboxylate;

methyl 3-(4-(5-fluoro-2-oxoindolin-1-yl)piperidin-1-yl)-3-methylpyrrolidine-1-carboxylate;

ethyl 3-(4-(5-fluoro-2-oxoindolin-1-yl)piperidin-1-yl)-3-methylpyrrolidine-1-carboxylate;

ethyl 3-(4-(5-fluoro-6-methyl-2-oxoindolin-1-yl)piperidin-1-yl)-3-methylpyrrolidine-1-carboxylate;

methyl 3-(4-(5-fluoro-6-methyl-2-oxoindolin-1-yl)piperidin-1-yl)-3-methylpyrrolidine-1-carboxylate;

ethyl 3-(4-(4-tert-butyl-6-fluoro-2-oxoindolin-1-yl)piperidin-1-yl)-3-methylpyrrolidine-1-carboxylate;

methyl 3-(4-(4-tert-butyl-6-methyl-2-oxoindolin-1-yl)piperidin-1-yl)-3-methylpyrrolidine-1-carboxylate;

ethyl 3-(4-(6′-fluoro-2′-oxospiro[cyclopropane-1,3′-indoline]-1′-yl)piperidin-1-yl)-3-methylpyrrolidine-1-carboxylate;

and isolated enantiomers thereof, and pharmaceutically acceptable salt thereof.

It will be understood that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of a compound of Formula I to X The optically active forms of the compound of the invention may be prepared, for example, by chiral chromatographic separation of a racemate, by synthesis from optically active starting materials or by asymmetric synthesis based on the procedures described thereafter.

Optical isomers can be obtained in pure form by standard procedures known to those skilled in the art, and include, but are not limited to, diastereomeric salt formation, kinetic resolution, and asymmetric synthesis. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, IN 1972), each of which is incorporated herein by reference in their entireties. It is also understood that this invention encompasses all possible regioisomers, and mixtures thereof, which can be obtained in pure form by standard separation procedures known to those skilled in the art, and include, but are not limited to, column chromatography, thin-layer chromatography, and high-performance liquid chromatography.

It will also be appreciated that certain compounds of the present invention may exist as geometrical isomers, for example E and Z isomers of alkenes. The present invention includes any geometrical isomer of a compound of Formula I to X. It will further be understood that the present invention encompasses tautomers of the compounds of the Formula I to X.

It will also be understood that certain compounds of the present invention may exist in solvated, for example hydrated, as well as unsolvated forms. It will further be understood that the present invention encompasses all such solvated forms of the compounds of the Formula I to X.

Within the scope of the invention are also salts of the compounds of the Formula I to X. Generally, pharmaceutically acceptable salts of compounds of the present invention may be obtained using standard procedures well known in the art, for example by reacting a sufficiently basic compound, for example an alkyl amine with a suitable acid, for example, HCl or acetic acid, to afford a physiologically acceptable anion. It may also be possible to make a corresponding alkali metal (such as sodium, potassium, or lithium) or an alkaline earth metal (such as a calcium) salt by treating a compound of the present invention having a suitably acidic proton, such as a carboxylic acid or a phenol with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques.

In one embodiment, the compound of Formula I to X above may be converted to a pharmaceutically acceptable salt or solvate thereof, particularly, an acid addition salt such as a hydrochloride, hydrobromide, sulfate, phosphate, acetate, fumarate, maleate, tartrate, citrate, methanesulphonate or p-toluenesulphonate.

In some embodiments, the compounds of Formula I to IX are prodrugs. As used herein, “prodrug” refers to a moiety that releases a compound of the invention when administered to a patient. Prodrugs can be prepared by modifying functional groups present in the compounds in such a way that the modifications are cleaved, either in routine manipulation or in vivo, to the parent compounds. Examples of prodrugs include compounds of the invention as described herein that contain one or more molecular moieties appended to a hydroxyl, amino, sulfhydryl, or carboxyl group of the compound, and that when administered to a patient, cleaves in vivo to form the free hydroxyl, amino, sulfhydryl, or carboxyl group, respectively. Examples of prodrugs include, but are not limited to, acetate, formate and benzoate derivatives of alcohol and amine functional groups in the compounds of the invention. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are incorporated herein by reference in their entireties.

### Compositions, Methods and Uses

We have now found that many of the compounds of the invention tested have activity as pharmaceuticals, in particular as agonists of Ml receptors. More particularly, many of the compounds of the invention tested exhibit selective activity as agonist of the M1 receptors and are useful in therapy, especially for relief of various pain conditions such as chronic pain, neuropathic pain, acute pain, cancer pain, pain caused by rheumatoid arthritis, migraine, visceral pain etc. This list should however not be interpreted as exhaustive. Additionally, compounds of the present invention may be useful in other disease states in which dysfunction of M1 receptors is present or implicated. Furthermore, the compounds of the invention may be used to treat cancer, multiple sclerosis, Parkinson's disease, Huntington's chorea, schizophrenia, Alzheimer's disease, anxiety disorders, depression, obesity, gastrointestinal disorders and cardiovascular disorders.

In some embodiments, the compounds may be used to treat schizophrenia or Alzheimer's disease.

In another embodiment, the compounds may be used to treat pain.

In another particular embodiment, the compounds may be used to treat neuropathic pain.

Compounds of the invention may be useful as immunomodulators, especially for autoimmune diseases, such as arthritis, for skin grafts, organ transplants and similar surgical needs, for collagen diseases, various allergies, for use as anti-tumour agents and anti viral agents.

Compounds of the invention may be useful in disease states where degeneration or dysfunction of M1 receptors is present or implicated in that paradigm. This may involve the use of isotopically labeled versions of the compounds of the invention in diagnostic techniques and imaging applications such as positron emission tomography (PET).

Compounds of the invention may be useful for the treatment of diarrhea, depression, anxiety and stress-related disorders such as post-traumatic stress disorder, panic disorder, generalized anxiety disorder, social phobia, and obsessive compulsive disorder, urinary incontinence, premature ejaculation, various mental illnesses, cough, lung oedema, various gastro-intestinal disorders, e.g. constipation, functional gastrointestinal disorders such as Irritable Bowel Syndrome and Functional Dyspepsia, Parkinson's disease and other motor disorders, traumatic brain injury, stroke, cardioprotection following miocardial infarction, obesity, spinal injury and drug addiction, including the treatment of alcohol, nicotine, opioid and other drug abuse and for disorders of the sympathetic nervous system for example hypertension.

Compounds of the invention may be useful as an analgesic agent for use during general anaesthesia and monitored anaesthesia care. Combinations of agents with different properties are often used to achieve a balance of effects needed to maintain the anaesthetic state (e.g. amnesia, analgesia, muscle relaxation and sedation). Included in this combination are inhaled anaesthetics, hypnotics, anxiolytics, neuromuscular blockers, and opioids.

A further aspect of the invention is a method for the treatment of a subject suffering from any of the conditions discussed above, whereby an effective amount of a compound according to the Formula I above, is administered to a patient in need of such treatment.

The present invention further provides the use of any of the compounds according to the Formula I above, for the manufacture of a medicament for the treatment of any of the conditions discussed above.

The present invention further provides a compound of Formula I, or pharmaceutically acceptable salt or solvate thereof, as hereinbefore defined for use in therapy.

In a further aspect, the present invention provides the use of a compound of Formula I, or a pharmaceutically acceptable salt or solvate thereof, as hereinbefore defined in the manufacture of a medicament for use in therapy.

In the context of the present specification, the term “therapy” also includes “prophylaxis” unless there are specific indications to the contrary. The term “therapeutic” and “therapeutically” should be construed accordingly. The term “therapy” within the context of the present invention further encompasses to administer an effective amount of a compound of the present invention, to mitigate either a pre-existing disease state, acute or chronic, or a recurring condition. The term “therapy” within the context of the present invention encompasses (a) inhibiting a disease, condition or disorder in an individual who is experiencing or displaying the pathology or symptomatology of the disease, condition or disorder (i.e., arresting further development of the pathology and/or symptomatology); (b) retarding a disease, condition or disorder in an individual who is experiencing or displaying the pathology or symptomatology of the disease, condition or disorder (i.e., slowing down the development of the pathology and/or symptomatology); and (c) ameliorating the disease; for example, ameliorating a disease, condition or disorder in an individual who is experiencing or displaying the pathology or symptomatology of the disease, condition or disorder (i.e., reversing the pathology and/or symptomatology). This definition also encompasses prophylactic therapies for prevention of recurring conditions and continued therapy for chronic disorders.

In some embodiments, the patient, mammal or human who is administered any compound or composition of the invention is “in need thereof”. Similarly, in some embodiments, the patient, individual, mammal or human may have been diagnosed with a particular disease or condition or may be suspected of having a particular disease or conditions.

The phrase “therapeutically effective amount” refers to the amount of a compound of the invention that elicits the biological or medicinal response in a tissue, system, animal, individual, patient, or human that is being sought by a researcher, veterinarian, medical doctor or other clinician. The desired biological or medicinal response may include preventing the disorder in an individual (e.g., preventing the disorder in an individual that may be predisposed to the disorder, but does not yet experience or display the pathology or symptomatology of the disease). The desired biological or medicinal response may also include inhibiting the disorder in an individual that is experiencing or displaying the pathology or symptomatology of the disorder (i.e., arresting or slowing further development of the pathology and/or symptomatology). The desired biological or medicinal response may also include ameliorating the disorder in an individual that is experiencing or displaying the pathology or symptomatology of the disease (i.e., reversing the pathology or symptomatology).

The therapeutically effective amount provided in the treatment of a specific disorder will vary depending the specific disorder(s) being treated, the size, age, and response pattern of the individual the severity of the disorder(s), the judgment of the attending clinician, the manner of administration, and the purpose of the administration, such as prophylaxis or therapy. In general, effective amounts for daily oral administration may be about 0.01 to 1000 mg/kg, 0.01 to 50 mg/kg, about 0.1 to 10 mg/kg and effective amounts for parenteral administration may be about 0.01 to 10 mg/kg, or about 0.1 to 5 mg/kg.

The compounds of the present invention may be useful in therapy, especially for the therapy of various pain conditions including, but not limited to: acute pain, chronic pain, neuropathic pain, back pain, cancer pain, and visceral pain. In a particular embodiment, the compounds may be useful in therapy for neuropathic pain. In an even more particular embodiment, the compounds may be useful in therapy for chronic neuropathic pain.

In use for therapy in a warm-blooded animal such as a human, the compound of the invention may be administered in the form of a conventional pharmaceutical composition by any route including orally, intramuscularly, subcutaneously, topically, intranasally, intraperitoneally, intrathoracially, intravenously, epidurally, intrathecally, transdermally, intracerebroventricularly and by injection into the joints.

In one embodiment of the invention, the route of administration may be oral, intravenous or intramuscular.

The dosage will depend on the route of administration, the severity of the disease, age and weight of the patient and other factors normally considered by the attending physician, when determining the individual regimen and dosage level at the most appropriate for a particular patient.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or table disintegrating agents; it can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture in then poured into convenient sized moulds and allowed to cool and solidify.

Suitable carriers are magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Depending on the mode of administration, the pharmaceutical composition will preferably include from 0.05% to 99% w/w (per cent by weight), more preferably from 0.10 to 50% w/w, of the compound of the invention, all percentages by weight being based on total composition.

Within the scope of the invention is the use of any compound of Formula I as defined above for the manufacture of a medicament.

Also within the scope of the invention is the use of any compound of Formula I for the manufacture of a medicament for the therapy of pain.

Additionally provided is the use of any compound according to Formula I for the manufacture of a medicament for the therapy of various pain conditions including, but not limited to: acute pain, chronic pain, neuropathic pain, back pain, cancer pain, and visceral pain.

A further aspect of the invention is a method for therapy of a subject suffering from any of the conditions discussed above, whereby an effective amount of a compound according to the Formula I above, is administered to a patient in need of such therapy.

Additionally, there is provided a pharmaceutical composition comprising a compound of Formula I or a pharmaceutically acceptable salt thereof, in association with a pharmaceutically acceptable carrier.

Particularly, there is provided a pharmaceutical composition comprising a compound of Formula I or a pharmaceutically acceptable salt thereof, in association with a pharmaceutically acceptable carrier for therapy, more particularly for therapy of pain.

Further, there is provided a pharmaceutical composition comprising a compound of Formula I or a pharmaceutically acceptable salt thereof, in association with a pharmaceutically acceptable carrier use in any of the conditions discussed above.

In a further embodiment, a compound of the present invention, or a pharmaceutical composition or formulation comprising a compound of the present invention may be administered concurrently, simultaneously, sequentially or separately with one or more pharmaceutically active compound(s) selected from the following:

(i) antidepressants such as, for example, amitriptyline, amoxapine, bupropion, citalopram, clomipramine, desipramine, doxepin duloxetine, elzasonan, escitalopram, fluvoxamine, fluoxetine, gepirone, imipramine, ipsapirone, maprotiline, nortriptyline, nefazodone, paroxetine, phenelzine, protriptyline, reboxetine, robalzotan, sertraline, sibutramine, thionisoxetine, tranylcypromaine, trazodone, trimipramine, venlafaxine and equivalents and pharmaceutically active isomer(s) and metabolite(s) thereof;

(ii) atypical antipsychotics including, for example, quetiapine and pharmaceutically active isomer(s) and metabolite(s) thereof; amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, lithium, loxapine, mesoridazine, olanzapine, paliperidone, perlapine, perphenazine, phenothiazine, phenylbutlypiperidine, pimozide, prochlorperazine, risperidone, quetiapine, sertindole, sulpiride, suproclone, suriclone, thioridazine, trifluoperazine, trimetozine, valproate, valproic acid, zopiclone, zotepine, ziprasidone and equivalents thereof;

(iii) antipsychotics including, for example, amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutlypiperidine, pimozide, prochlorperazine, risperidone, sertindole, sulpiride, suproclone, suriclone, thioridazine, trifluoperazine, trimetozine, valproate, valproic acid, zopiclone, zotepine, ziprasidone and equivalents and pharmaceutically active isomer(s) and metabolite(s) thereof;

(iv) anxiolytics including, for example, alnespirone, azapirones, benzodiazepines, barbiturates such as adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, zolazepam and equivalents and pharmaceutically active isomer(s) and metabolite(s) thereof;

(v) anticonvulsants including, for example, carbamazepine, valproate, lamotrogine, gabapentin and equivalents and pharmaceutically active isomer(s) and metabolite(s) thereof;

(vi) Alzheimer's therapies including, for example, donepezil, memantine, tacrine and equivalents and pharmaceutically active isomer(s) and metabolite(s) thereof;

(vii) Parkinson's therapies including, for example, deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase and equivalents and pharmaceutically active isomer(s) and metabolite(s) thereof;

(viii) migraine therapies including, for example, almotriptan, amantadine, bromocriptine, butalbital, cabergoline, dichloralphenazone, eletriptan, frovatriptan, lisuride, naratriptan, pergolide, pramipexole, rizatriptan, ropinirole, sumatriptan, zolmitriptan, zomitriptan, and equivalents and pharmaceutically active isomer(s) and metabolite(s) thereof;

(ix) stroke therapies including, for example, abciximab, activase, NXY-059, citicoline, crobenetine, desmoteplase, repinotan, traxoprodil and equivalents and pharmaceutically active isomer(s) and metabolite(s) thereof;

(x) over active bladder urinary incontinence therapies including, for example, darafenacin, falvoxate, oxybutynin, propiverine, robalzotan, solifenacin, tolterodine and and equivalents and pharmaceutically active isomer(s) and metabolite(s) thereof;

(xi) neuropathic pain therapies including, for example, gabapentin, lidoderm, pregablin and equivalents and pharmaceutically active isomer(s) and metabolite(s) thereof;

(xii) nociceptive pain therapies such as, for example, celecoxib, etoricoxib, lumiracoxib, rofecoxib, valdecoxib, diclofenac, loxoprofen, naproxen, paracetamol and equivalents and pharmaceutically active isomer(s) and metabolite(s) thereof;

(xiii) insomnia therapies including, for example, allobarbital, alonimid, amobarbital, benzoctamine, butabarbital, capuride, chloral, cloperidone, clorethate, dexclamol, ethchlorvynol, etomidate, glutethimide, halazepam, hydroxyzine, mecloqualone, melatonin, mephobarbital, methaqualone, midaflur, nisobamate, pentobarbital, phenobarbital, propofol, roletamide, triclofos,secobarbital, zaleplon, zolpidem and equivalents and pharmaceutically active isomer(s) and metabolite(s) thereof; and

(xiv) mood stabilizers including, for example, carbamazepine, divalproex, gabapentin, lamotrigine, lithium, olanzapine, quetiapine, valproate, valproic acid, verapamil, and equivalents and pharmaceutically active isomer(s) and metabolite(s) thereof.

Such combinations employ the compounds of this invention within the dosage range described herein and the other pharmaceutically active compound or compounds within approved dosage ranges and/or the dosage described in the publication reference.

In an even further embodiment, a compound of the present invention, or a pharmaceutical composition or formulation comprising a compound of the present invention may be administered concurrently, simultaneously, sequentially or separately with one or more pharmaceutically active compound(s) selected from buprenorphine; dezocine; diacetylmorphine; fentanyl; levomethadyl acetate; meptazinol; opioids such as morphine; oxycodone; oxymorphone; remifentanil; sufentanil; and tramadol.

In a particular embodiment, it may be particularly effective to administrate a combination containing a compound of the invention and a second active compound selected from buprenorphine; dezocine; diacetylmorphine; fentanyl; levomethadyl acetate; meptazinol; morphine; oxycodone; oxymorphone; remifentanil; sufentanil; and tramadol to treat chronic nociceptive pain. The efficacy of this therapy may be demonstrated using a rat SNL heat hyperalgesia assay described below.

The methods, uses, compounds for use in therapy, and pharmaceutical compositions may utilize any of the embodiments of the compounds of Formulas I to IX, or any combination thereof.

In another aspect, the invention provides a method of treating ocular hypertension or glaucoma by administering to a patient in need thereof one of the compounds of formula I, optionally, in combination with a β-adrenergic blocking agent such as timolol, carbonic anhydrase inhibitor such as dorzolamide, acetazolamide, methazolamide or brinzolamide, potassium channel blocker, a prostaglandin such as latanoprost, isopropyl unoprostone, S1033 or a prostaglandin derivative such as a hypotensive lipid derived from PGF2α prostaglandins. An example of a hypotensive lipid (the carboxylic acid group on the α-chain link of the basic prostaglandin structure is replaced with electrochemically neutral substituents) is that in which the carboxylic acid group is replaced with CH₂—OR group such as CH₂OCH₃ (PGF₂ₐ 1-OCH₃), or a CH₂OH group (PGF₂ₐ 1-OH). Preferred potassium channel blockers for use in combination with the Ml agonist are calcium activated potassium channel blockers. More preferred potassium channel blockers are high conductance, calcium activated potassium (Maxi-K) channel blockers.

Macular edema is swelling within the retina within the critically important central visual zone at the posterior pole of the eye. An accumulation of fluid within the retina tends to detach the neural elements from one another and from their local blood supply, creating a dormancy of visual function in the area.

Glaucoma is characterized by progressive atrophy of the optic nerve and is frequently associated with elevated intraocular pressure (IOP). It is possible to treat glaucoma, however, without necessarily affecting IOP by using drugs that impart a neuroprotective effect. See Arch. Ophthalmol. Vol. 112, January 1994, pp. 37-44; Investigative Ophthalmol. & Visual Science, 32, 5, April 1991, pp. 1593-99. It is believed that Ml agonist which lower IOP are useful for providing a neuroprotective effect. They are also believed to be effective for treating macular edema and/or macular degeneration, increasing retinal and optic nerve head blood velocity and increasing retinal and optic nerve oxygen by lowering IOP, which when coupled together benefits optic nerve health. As a result, this invention further relates to a method for treating macular edema and/or macular degeneration, increasing retinal and optic nerve head blood velocity, increasing retinal and optic nerve oxygen tension as well as providing a neuroprotective effect or a combination thereof.

Also within the scope of the invention is the use of any of the compounds according to the Formula I above, for the manufacture of a medicament for the treatment of any of the conditions discussed above.

A further aspect of the invention is a method for the treatment of a subject suffering from any of the conditions discussed above, whereby an effective amount of a compound according to the Formula I above, is administered to a patient in need of such treatment.

Thus, the invention provides a compound of Formula I or pharmaceutically acceptable salt or solvate thereof, as hereinbefore defined for use in therapy.

In a further aspect, the present invention provides the use of a compound of Formula I or a pharmaceutically acceptable salt or solvate thereof, as hereinbefore defined in the manufacture of a medicament for use in therapy.

In the context of the present specification, the term “therapy” also includes “prophylaxis” unless there are specific indications to the contrary. The term “therapeutic” and “therapeutically” should be construed accordingly. The term “therapy” within the context of the present invention further encompasses to administer an effective amount of a compound of the present invention, to mitigate either a pre-existing disease state, acute or chronic, or a recurring condition. This definition also encompasses prophylactic therapies for prevention of recurring conditions and continued therapy for chronic disorders.

In another aspect, the invention provides an ophthalmic pharmaceutical composition containing an effective amount of a compound of formula I. The ophthalmic pharmaceutical compositions may be adapted for topical administration to the eye in the form of solutions, suspensions, ointments, creams or as a solid insert. Ophthalmic formulations of this compound may contain from 0.01 to 5% and especially 0.1 to 2% of medicament. Higher dosages as, for example, about 10% or lower dosages can be employed provided the dose is effective in reducing intraocular pressure, treating glaucoma, increasing blood flow velocity or oxygen tension. For a single dose, from between 0.001 to 5.0 mg, preferably 0.005 to 2.0 mg, and especially 0.005 to 1.0 mg of the compound can be applied to the human eye.

The pharmaceutical preparation that contains the compound may be conveniently admixed with a non-toxic pharmaceutical organic carrier, or with a non-toxic pharmaceutical inorganic carrier. Typical of pharmaceutically acceptable carriers are, for example, water, mixtures of water and water-miscible solvents such as lower alkanols or aralkanols, vegetable oils, polyalkylene glycols, petroleum based jelly, ethyl cellulose, ethyl oleate, carboxymethyl-cellulose, polyvinylpyrrolidone, isopropyl myristate and other conventionally employed acceptable carriers. The pharmaceutical preparation may also contain non-toxic auxiliary substances such as emulsifying, preserving, wetting agents, bodying agents and the like, as for example, polyethylene glycols 200, 300, 400 and 600, carbowaxes 1,000, 1,500, 4,000, 6,000 and 10,000, antibacterial components such as quaternary ammonium compounds, phenylmercuric salts known to have cold sterilizing properties and which are non-injurious in use, thimerosal, methyl and propyl paraben, benzyl alcohol, phenyl ethanol, buffering ingredients such as sodium borate, sodium acetates, gluconate buffers, and other conventional ingredients such as sorbitan monolaurate, triethanolamine, oleate, polyoxyethylene sorbitan monopalmitate, dioctyl sodium sulfosuccinate, monothioglycerol, thiosorbitol, ethylenediamine tetracetic acid, and the like. Additionally, suitable ophthalmic vehicles can be used as carrier media for the present purpose including conventional phosphate buffer vehicle systems, isotonic boric acid vehicles, isotonic sodium chloride vehicles, isotonic sodium borate vehicles and the like. The pharmaceutical preparation may also be in the form of a microparticle formulation. The pharmaceutical preparation may also be in the form of a solid insert. For example, one may use a solid water soluble polymer as the carrier for the medicament. The polymer used to form the insert may be any water soluble non-toxic polymer, for example, cellulose derivatives such as methylcellulose, sodium carboxymethyl cellulose, (hydroxyloweralkyl cellulose), hydroxyethyl cellulose, hydroxypropyl cellulose, hydroxypropylmethyl cellulose; acrylates such as polyacrylic acid salts, ethylacrylates, polyactylamides; natural products such as gelatin, alginates, pectins, tragacanth, karaya, chondrus, agar, acacia; the starch derivatives such as starch acetate, hydroxymethyl starch ethers, hydroxypropyl starch, as well as other synthetic derivatives such as polyvinyl alcohol, polyvinyl pyrrolidone, polyvinyl methyl ether, polyethylene oxide, neutralized carbopol and xanthan gum, gellan gum, and mixtures of said polymer.

Suitable subjects for the administration of the formulation of the present invention include primates, man and other animals, particularly man and domesticated animals such as cats and dogs.

The pharmaceutical preparation may contain non-toxic auxiliary substances such as antibacterial components which are non-injurious in use, for example, thimerosal, benzalkonium chloride, methyl and propyl paraben, benzyldodecinium bromide, benzyl alcohol, chlorhexidine, or phenylethanol; buffering ingredients such as sodium borate, sodium acetate, sodium citrate, or gluconate buffers; and other conventional ingredients such as sodium chloride, sorbitan monolaurate, triethanolamine, polyoxyethylene sorbitan monopalmitate, ethylenediaminetetraacetic acid, and the like.

The ophthalmic solution or suspension may be administered as often as necessary to maintain an acceptable IOP level in the eye. It is contemplated that administration to the mammalian eye will be once to three times daily.

For topical ocular administration the novel formulations of this invention may take the form of solutions, gels, ointments, suspensions or solid inserts, formulated so that a unit dosage comprises a therapeutically effective amount of the active component or some multiple thereof in the case of a combination therapy.

### Syntheses and Processes

The compounds of the present invention can be prepared in a variety of ways known to one skilled in the art of organic synthesis. The compounds of the present invention can be synthesized using the methods as hereinafter described below, together with synthetic methods known in the art of synthetic organic chemistry or variations thereon as appreciated by those skilled in the art.

The compounds of present invention can be conveniently prepared in accordance with the procedures outlined in the schemes below, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures. Those skilled in the art of organic synthesis will recognize that the nature and order of the synthetic steps presented may be varied for the purpose of optimizing the formation of the compounds of the invention.

The processes described herein can be monitored according to any suitable method known in the art. For example, product formation can be monitored by spectroscopic means, such as nuclear magnetic resonance spectroscopy (e.g., ¹H or ¹³C NMR) infrared spectroscopy, spectrophotometry (e.g., UV-visible), or mass spectrometry, or by chromatography such as high performance liquid chromatography (HPLC) or thin layer chromatography.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 4d. Ed., Wiley & Sons, 2007, which is incorporated herein by reference in its entirety. Adjustments to the protecting groups and formation and cleavage methods described herein may be adjusted as necessary in light of the various substituents.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

The compounds of the present invention may be made by a variety of methods, as described herein. For example, compounds of Formula I, wherein X is —NR⁸— may be made as shown in Scheme I. Accordingly, a appropriate protected (e.g. using BOC as the amine protecting group, i.e., R^{P} is t-butyl) 4-oxopiperidine, 3-oxopyrrolidine, or 4-oxoazepane (1) is reacted with a 4-hydroxypiperidine (2) in the presence of titanium isopropoxide in a solvent at room temperature for approximately 24 hours. The product may then be treated in situ with cyanodiethylaluminum in a solvent such as toluene for approximately 24 hours. The R³ group can be added by reacting the product of the previous reaction with a Grignard reagent of formula R³MgBr in a solvent such an ether (such as ethyl ether, butyl ether, or THF) to give the hydroxyl compound (3). The hydroxyl compound is then oxidized, e.g., via a Swern oxidation to give the ketone (4) (e.g. reaction with oxalyl dichloride in a solvent such as dichloromethane at a lower temperature, such as −78° C., followed by quenching with a base such as a tertiary amine such as triethylamine). The ketone (4) can then be reacted with an unsubstituted or substituted benzene-1,2-diamine (5) in a solvent such as dichloromethane with the addition of sodium triacetoxyhydroboarate, followed by the addition of acetic acid to give the amine (6). The amine (6) can then be reacted with phosgene or a phosgene equivalent, such as triphosgene, to give the protected compound (7). The compound of Formula I is then formed by removing the BOC protecting group from compound (7) to give the amine (8). The amine (8) may be reacted in situ or after isolation to add the R² group by processes such in Schemes I-A and I-B. The processes vary depending on the type of R² group. After addition of the R² group, the compounds may be purified by preparative HPLC to separate the desired regioisomer from the other regioisomer, if necessary.

In Scheme I-A, the amine (8) may be converted to a carbamate (10) using a compound of formula “R^{a}OC(O)-L” [e.g. R^{a}OC(O)Cl] wherein L is a leaving group such as halogen or —OR^{a}, generally in the presence of a base such as a tertiary amine (e.g., triethylamine or diisopropylethylamine), imidazole, N,N-dimethyl-4-aminopyridine, or the like, in a solvent such as dichloromethane (DCM).

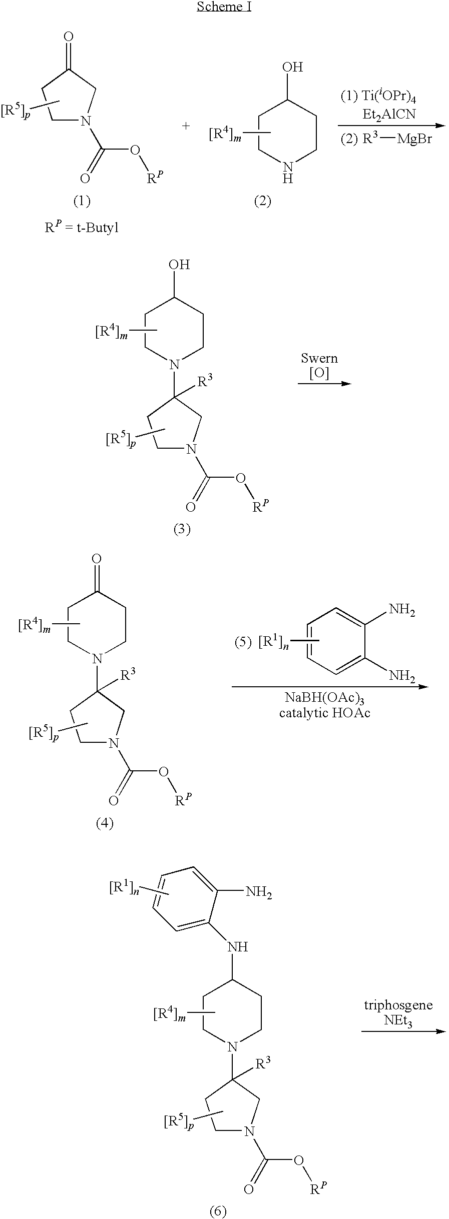

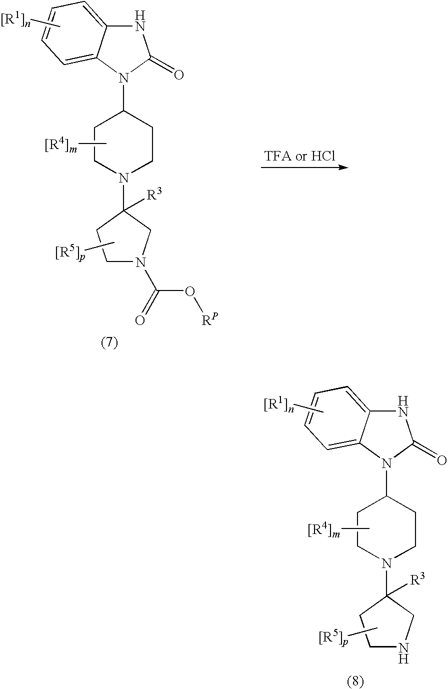

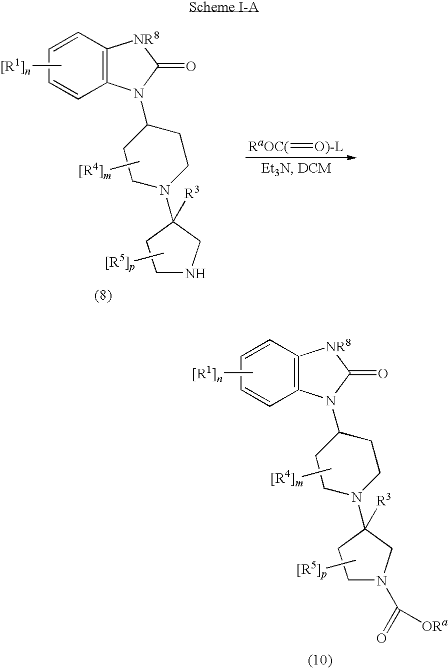

The amine (8) may be converted to a urea (11) by methods known to those skilled in the organic synthesis. Form example, as shown in Scheme I-C, the amine (8) can be reacted with a compound having the formula of R′OC(═O)-L wherein L is a leaving group such as halogen or —OR′ to form a carbamate (wherein R′ is methyl, ethyl or the like). The carbamate can be reacted with an amine of formula “HNR^{c}R^{d}” to form the urea (11). Alternatively, a urea (11), wherein R^{d} is hydrogen, may be formed by reacting (8) with an isocyanate of formula “R^{c}—N═C═O”.

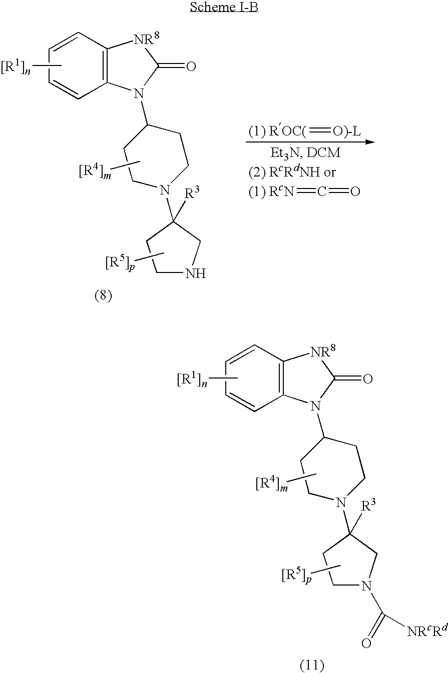

Alternatively, compounds of Formula I, wherein X is —NR⁸— may be made as shown in Scheme II (such as those wherein R¹ is C₁₋₆alkyl). Accordingly, the ketone (14) is converted to an amine (15), by reacting with ammonia and titanium(IV) isopropoxide, followed by addition of sodium borohydride at room temperature. The amine (15) may then be reacted with an unsubstituted or substituted 1-fluoro-2-nitrobenzene (16) in the presence of a base such as potassium carbonate to form the nitro compound (17). The nitro compound (17) is then reduced to the amine (18) under catalytic hydrogenation conditions (e.g., palladium on carbon and hydrogen gas). Protecting groups may be used if necessary to protect any substitutents prior to hydrogenation. The amine (18) is then reacted with phosgene or phosgene equivalent (e.g., triphosphene) to give compound (19). After removal of the BOC protecting group, R² group may be added by the methods analogous to those illustrated in Schemes I-A to I-B and the surrounding text. After addition of the R² group, the compounds may be purified by preparative HPLC to separate the desired regioisomer from the other regioisomer, if necessary.

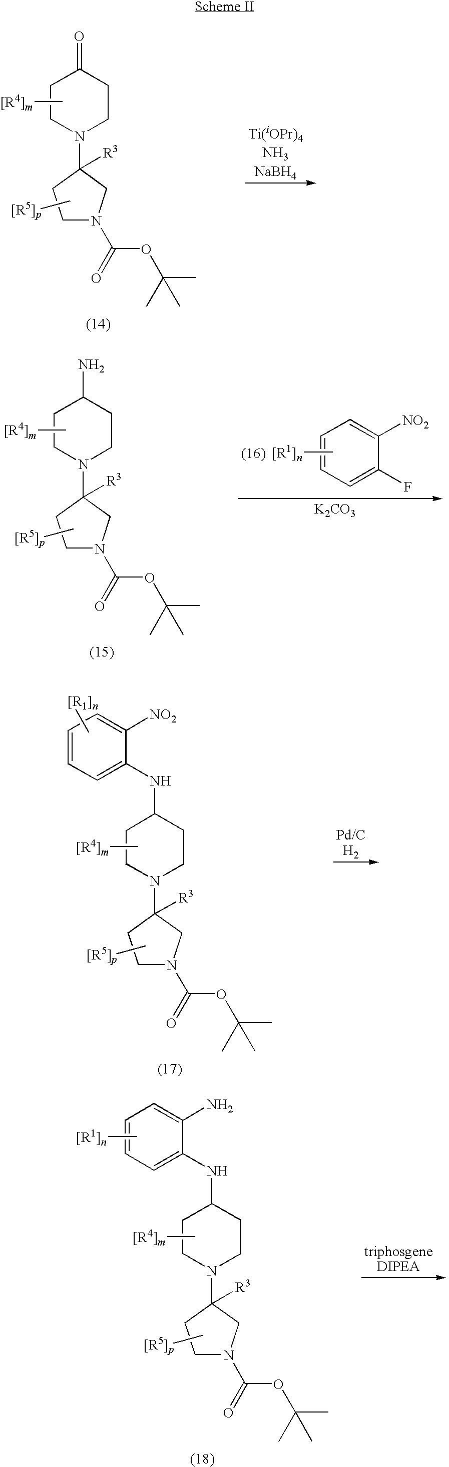

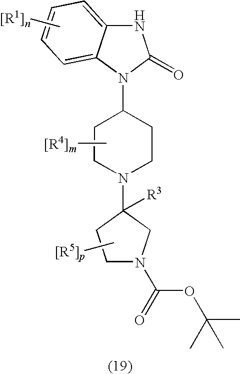

Alternatively, compounds of Formula I, wherein X is —NR⁸— may be made as shown in Scheme III (such as those wherein R¹ is C₁₋₆ alkyl). One of the amino groups of an unsubstituted or substituted diamine (20) may be protected, for example using di(t-butyl) dicarbonate to give the BOC-protected amine (21). The protected amine (21) may then be reacted with compound (22) in the presence of sodium cyanoborohydride and zinc chloride to give compound (23). Compound (23) may then be cyclized in the presence of a base such as potassium t-butoxide to give compound (24). After removal of the BOC protecting group, R² group may be added by the methods analogous to those illustrated in Schemes I-A to I-B and the surrounding text. After addition of the R² group, the compounds may be purified by preparative HPLC to separate the desired regioisomer from the other regioisomer, if necessary.

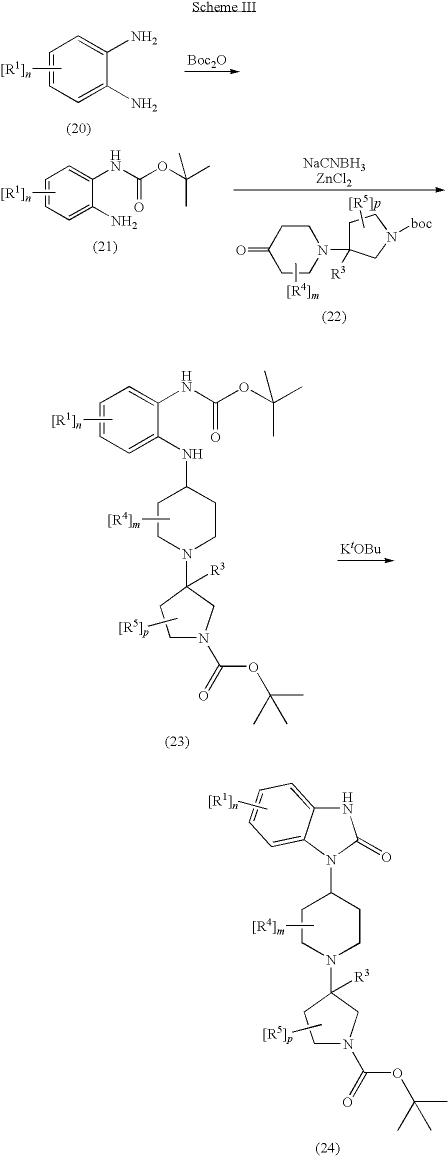

Compounds of Formula I, where X is —NR⁸— and R⁸ is other than hydrogen, may be formed by reacting compound (9), (10), or (11), with sodium hydride in DMF, followed by addition of a compound of formula “R⁸-L”, wherein L is a leaving group, such as a halogen atom (e.g., bromine or iodine). Protecting groups may be used as necessary to protect particular substituent groups.

Alternatively, compounds of Formula I, where X is —NR⁸— and R⁸ is other than hydrogen, may be formed by protecting the unsubstituted or substituted benzene-1,2-diamine (e.g., compound (5) of Scheme I) with a protecting group, such as a BOC group, to form compound (5a), as shown in Scheme III-A. The correct regioisomer may then be isolated by preparative HPLC, if necessary. The R⁸ group may then be added to other amine group of compound (5a) by reacting (5a) with a compound of formula “R⁸-L” (such as R⁸I, e.g. methyl iodide) wherein L is a leaving group, such as a halogen atom. The protecting group may then be removed under standard deprotection conditions, as HCl in dioxane, to yield compound (5b). Compound (5b) may then be substituted for compound (5) in Scheme I. Alternatively, the compound of formula “R⁸-L” may be added directly to compound (5) and the regioisomers separated by preparative HPLC.

Similarly, compounds of Formula I, where X is —NR⁸— and R⁸ is other than hydrogen, may be formed by reacting compound (6) of Scheme I or compound (18) of Scheme II with a compound of formula “R⁸-L” and separating from any undesired regioisomers by preparative HPLC. The resultant compound may then be substituted for compound (6) of Scheme I or compound (18) of Scheme II to yield the desired compound.

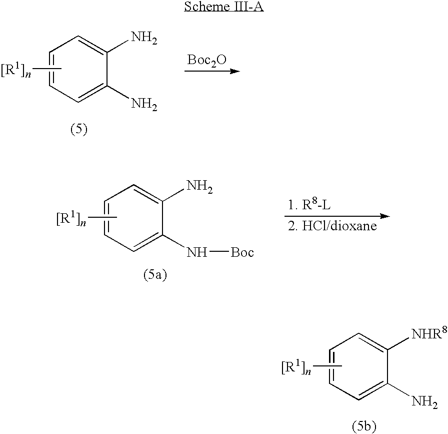

Compounds of Formula I, wherein X is —O— may be formed by the methods such as those shown in Scheme IV. The benzylated compound (25) may be reacted with compound (26) to form compound (27). The benzylated compound (25) may formed by benzylating the corresponding hydroxyl compound under standard conditions (Greene's Protective Groups in Organic Synthesis, 4^{th} Ed. (2007). The benzyl protecting group of compound (27) may be removed to form compound (28), followed by cyclization to form compound (29) by reacting compound (28) with triphosgene. After removal of the BOC protecting group of compound (29), R² group may be added by the methods analogous to those illustrated in Schemes I-A to I-B and the surrounding text.

Compounds of Formula I, wherein X is —S— may be formed by the methods analogous to those shown in Scheme IV and the surrounding text, except starting from a protected thiol compound. Appropriate protecting groups for thiol groups are summarized in Greene's Protecting Groups in Organic Synthesis, 4^{th} Ed. (2007), chapter 6. Alternatively, the compounds may be synthesized from compounds (28) of Schemes IV by appropriate substitution chemistry. For example, the amine group of (28) may be first protected. The hydroxyl group of the protected (28) may then converted to a thiol group by reaction of sodium hydrogen sulfide.

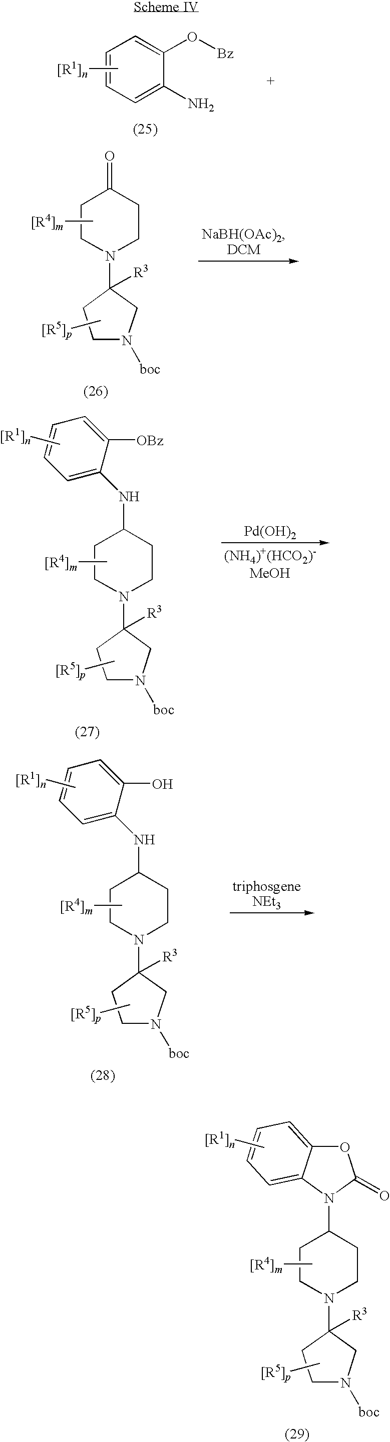

Compounds of Formula I, wherein X is —CR⁶R⁷— may be formed by the methods such as those shown in Scheme V. A BOC protected compound (30) may be first reacted to form compound (31) by converting the hydroxyl group to a better leaving group such as mesyl group. Compound (31) may then be reacted with cyanide ion to form the nitrile (32). The BOC-protected compound (30) may be synthesized starting from the corresponding unprotected amine compound by reacting it with di(t-butyl) dicarbonate. The unprotected amine compound, in turn, may be synthesized by converting the corresponding 2-amino-benzene-1-carboxylic acid to methyl or ethyl ester under standard esterification conditions, followed by reduction with a reducing agent such as lithium aluminum hydride. Substituents can be protected if necessary prior to reductions by methods such as those in Greene (supra). The nitrile (32) can be reacted with compound (33) to give compound (34). The cyano group of compound (34) can then be hydrolyzed under basic conditions to give the carboxylic acid (35). The carboxylic acid (35) may then be cyclized to give compound (36). After removal of the BOC protecting group, R² group may be added by the methods analogous to those illustrated in Schemes I-A to I-B and the surrounding text.

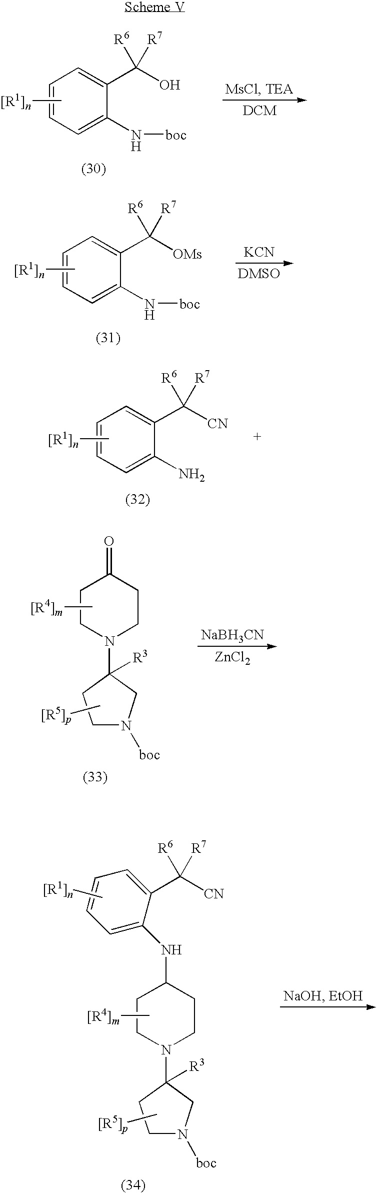

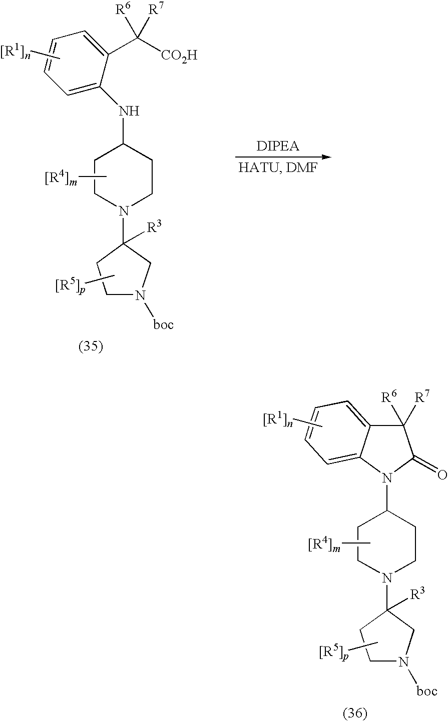

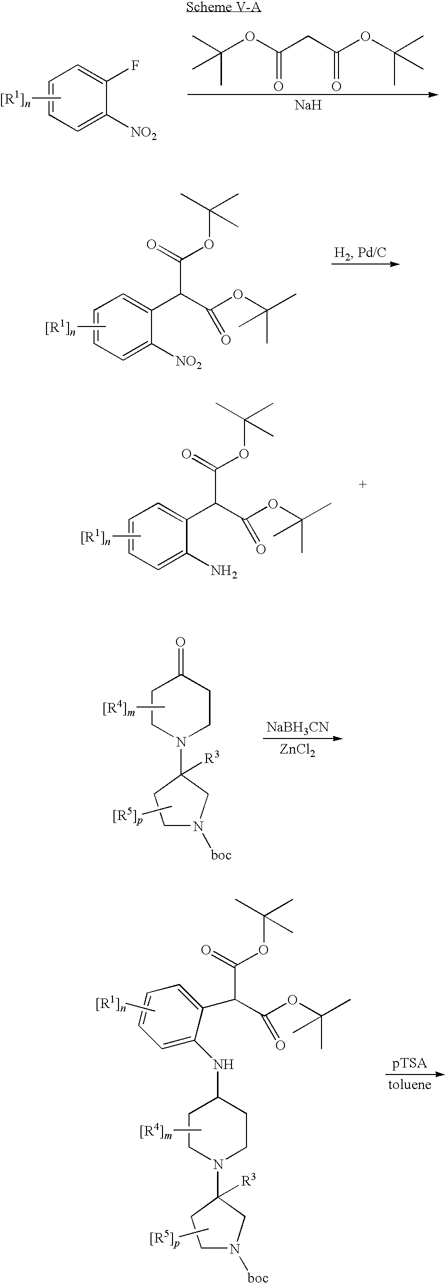

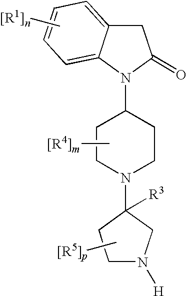

In accordance with the syntheses described above and in the examples, the present invention further provides processes for preparing the compounds of the invention.

Hence, the present invention further provides a process for preparing a compound of Formula I:

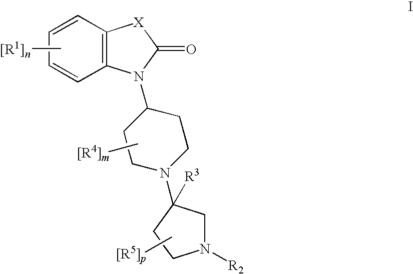

or pharmaceutically acceptable salt thereof, comprising reacting a compound of Formula X:

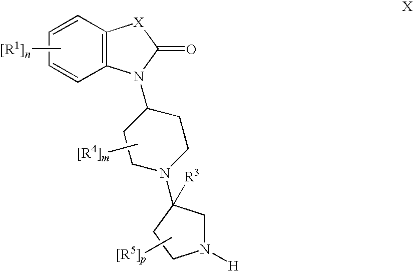

or pharmaceutically acceptable salt thereof, with a compound of Formula R^{a}OC(O)-L¹, or salt thereof, wherein L¹ is a leaving group, under conditions and for a time sufficient to form a compound of Formula I; wherein:

X is —CR⁶R⁷—, —NR⁸—, —O—, or —S—;

each R¹ is, independently, hydrogen, halogen, cyano, nitro, C₁₋₆alkyl, C₁₋₆haloalkyl, C₂₋₆alkenyl, C₂₋₆ alkynyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀aryl-C₁₋₃alkyl, C₃₋₉ heteroaryl, C₃₋₉heteroaryl-C₁₋₃alkyl, —SR, —OR^{f}, —O(CH₂)ᵣ—OR^{f}, —C(═O)—R^{e}, —C(═O)OR^{f}, —C(═O)NR^{g}R^{h}, —SO₂R^{e}, —SO₂NR^{g}R^{h}, —NR^{g}R^{h}, or —(CH₂)ᵣNR^{g}R^{h};

R² is —C(═O)OR^{a};

R³ is C₁₋₆ alkyl or C₁₋₆ haloalkyl;

each R⁴ is, independently, halogen, C₁₋₆alkyl, C₁₋₆haloalkyl, C₁₋₆alkoxy, hydroxyl-C₁₋₆alkyl-, —CH₂—OR, -or —C(═O)NR₂; or

any two of R⁴ are linked together to form a C₁₋₄ alkylene bridge and the other R⁴, if any, are each, independently, halogen, C₁₋₆alkyl, C₁₋₆haloalkyl, C₁₋₆alkoxy, hydroxyl-C₁₋₆alkyl-, —CH₂—OR, -or —C(═O)NR₂;

each R⁵ is, independently, halogen, C₁₋₆alkyl, C₁₋₆haloalkyl, C₁₋₆alkoxy, hydroxyl-C₁₋₆alkyl-, —CH₂—OR, -or —C(═O)NR₂;

R⁶, R⁷, and R⁸ are each, independently, hydrogen, C₁₋₆alkyl, C₂₋₆alkenyl, or C₁₋₆haloalkyl;

each R⁹, R¹⁰, and R¹¹ is, independently, phenyl, C₃₋₆ cycloalkyl, C₂₋₅ heterocycloalkyl, C₃₋₅ heteroaryl, halogen, cyano, nitro, —SR^{w}, —OR^{x}, —O(CH₂)ᵣ—OR^{x}, R^{x}, —C(═O)—R^{w}, —C(═O)OR^{x}, —C(═O)NR^{y}R^{z}, —SO₂R^{w}, —SO₂NR^{y}R^{z}, —NR^{y}R^{z}, or —(CH₂)ᵣNR^{y}R^{z};

R^{a} is C₁₋₇ alkyl, C₁₋₇ haloalkyl, C₂₋₆ alkenyl, C₂₋₆ alkynyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein the C₁₋₇ alkyl, C₂₋₆ alkenyl, C₂₋₆ alkynyl, and C₁₋₇ haloalkyl are each optionally substituted by 1, 2, or 3 independently selected R⁹ groups; wherein the C₃₋₇ cycloalkyl, C₃₋₇ cycloalkyl-C₁₋₃ alkyl, C₃₋₇ heterocycloalkyl, and C₃₋₇ heterocycloalkyl-C₁₋₃ alkyl are each optionally substituted by 1, 2, 3, or 4 independently selected R¹⁰ groups; and wherein the C₆₋₁₀aryl, C₆₋₁₀aryl-C₁₋₃alkyl, C₃₋₉ heteroaryl, and C₃₋₉heteroaryl-C₁₋₃ alkyl are each optionally substituted by 1, 2, 3, or 4 independently selected R¹¹ groups;

each R^{e}, R^{f}, R^{g}, R^{h}, R^{w}, R^{x}, R^{y}, R^{z}, and R is, independently hydrogen, C₁₋₆ alkyl, C₂₋₆ alkenyl, or C₁₋₆ haloalkyl;

r is 1, 2, 3, or 4;

n is 1, 2, 3, or 4;

m is 0, 1, 2, 3, 4, 5, 6, 7, or 8; and

p is an integer from 0 to 6.

In some embodiments, L¹ is halogen. In some embodiments, L¹ is chloro. In some embodiments, the conditions comprise use of a base such as a tertiary amine (e.g., triethylamine or diisopropylethylamine), imidazole, N,N-dimethyl-4-aminopyridine, or the like. The process may be used to prepare any of the preceding embodiments of the compounds of Formula I wherein R² is —C(═O)OR^{a}.

The present invention further provides a compound of Formula X:

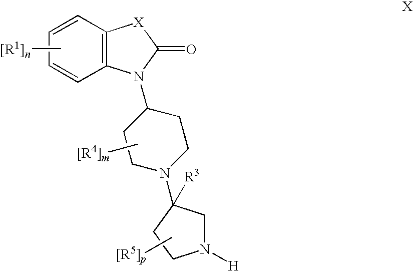

or pharmaceutically acceptable salt thereof, wherein:

X is —CR⁶R⁷—, —NR⁸—, —O—, or —S—;

each R¹ is, independently, hydrogen, halogen, cyano, nitro, C₁₋₆alkyl, C₁₋₆haloalkyl, C₂₋₆alkenyl, C₂₋₆ alkynyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀aryl-C₁₋₃alkyl, C₃₋₉ heteroaryl, C₃₋₉heteroaryl-C₁₋₃alkyl, —SR^{e}, —OR^{f}, —O(CH₂)ᵣ—OR^{f}, —C(═O)—R^{e}, —C(═O)OR^{f}, —C(═O)NR^{g}R^{h}, —SO₂R^{e}, —SO₂NR^{g}R^{h}, —NR^{g}R^{h}, or —(CH₂)ᵣNR^{g}R^{h};

R is —C(═O)OR^{a};

R³ is C₁₋₆ alkyl or C₁₋₆ haloalkyl;

each R⁴ is, independently, halogen, C₁₋₆alkyl, C₁₋₆haloalkyl, C₁₋₆alkoxy, hydroxyl-C₁₋₆alkyl-, —CH₂— OR, -or —C(═O)NR₂; or

any two of R⁴ are linked together to form a C₁₋₄ alkylene bridge and the other R⁴, if any, are each, independently, halogen, C₁₋₆alkyl, C₁₋₆haloalkyl, C₁₋₆alkoxy, hydroxyl-C₁₋₆alkyl-, —CH₂—OR, -or —C(═O)NR₂;

each R⁵ is, independently, halogen, C₁₋₆alkyl, C₁₋₆haloalkyl, C₁₋₆alkoxy, hydroxyl-C₁₋₆alkyl-, —CH₂—OR, -or —C(═O)NR₂;

R⁶, R⁷, and R³ are each, independently, hydrogen, C₁₋₆alkyl, C₂₋₆alkenyl, or C₁₋₆haloalkyl;

each R⁹, R¹⁰, and R¹¹ is, independently, phenyl, C₃₋₆ cycloalkyl, C₂₋₅ heterocycloalkyl, C₃₋₅ heteroaryl, halogen, cyano, nitro, —SR^{w}, —OR^{x}, —O(CH₂)ᵣ—OR^{x}, R^{x}, —C(═O)—R^{w}, —C(═O)OR^{x}, —C(═O)NR^{y}R^{z}, —SO₂R^{w}, —SO₂NR^{y}R^{z}, —NR^{y}R^{z}, or —(CH₂)ᵣNR^{y}R^{z};

R^{a} is C₁₋₇ alkyl, C₁₋₇ haloalkyl, C₂₋₆ alkenyl, C₂₋₆ alkynyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₂₀aryl, C₆₋₁₀ aryl-C₁₋₃alkyl, C₃₋₉ heteroaryl, or C₃₋₉ heteroaryl-C₁₋₃alkyl; wherein the C₁₋₇ alkyl, C₂₋₆ alkenyl, C₂₋₆ alkynyl, and C₁₋₇ haloalkyl are each optionally substituted by 1, 2, or 3 independently selected R⁹ groups; wherein the C₃₋₇ cycloalkyl, C₃₋₇ cycloalkyl-C₁₋₃ alkyl, C₃₋₇ heterocycloalkyl, and C₃₋₇ heterocycloalkyl-C₁₋₃ alkyl are each optionally substituted by 1, 2, 3, or 4 independently selected R¹⁰ groups; and wherein the C₆₋₁₀aryl, C₆₋₁₀aryl-C₁₋₃alkyl, C₃₋₉ heteroaryl, and C₃₋₉heteroaryl-C₁₋₃ alkyl are each optionally substituted by 1, 2, 3, or 4 independently selected R¹¹ groups;

each R^{e}, R^{f}, R^{g}, R^{h}, R^{w}, R^{x}, R^{y}, R^{z}, and R is, independently hydrogen, C₁₋₆ alkyl, C₂₋₆ alkenyl, or C₁₋₆ haloalkyl;

r is 1, 2, 3, or 4;

n is 1, 2, 3, or 4;

m is 0, 1, 2, 3, 4, 5, 6, 7, or 8; and

p is an integer from 0 to 6.

The present invention further provides compounds of Formula X corresponding to each of the embodiments for the compounds of Formula I, or suitable combination thereof. Compounds of Formula X may be useful as intermediates for producing the compounds of Formula I.

Further embodiments of the invention are the products obtainable by the process/es and/or specific Example/s disclosed herein

### Biological Evaluation

Human M1, rat M1, human M3 and human M5 Calcium Mobilization FLIPR™ Assay

The compound activity in the present invention (EC50 or IC50) is measured using a 384 plate-based imaging assay that monitors drug induced intracellular Ca² release in whole cells. Activation of hM1 (human Muscarinic receptor subtype 1, gene bank access NM_{—}000738), rM1 (rat Muscarinic receptor subtype 1, gene bank access NM_{—}080773), hM3 (human Muscarinic receptor subtype 3, gene bank access NM_{—}000740NM_{—}000740) and hM5 (human Muscarinic receptor subtype 5, gene bank access NM_{—}0121258), receptors expressed in CHO cells (Chinese hamster ovary cells, ATCC) is quantified in a Molecular Devices FLIPR II™ instrument as an increase in fluorescent signal. Inhibition of hM3 and hM5 by compounds is determined by the decrease in fluorescent signal in response to 2 nM acetylcholine activation.

CHO cells are plated in 384-well black/clear bottom poly-D-lysine plates (Becton Dickinson, 4663) at 8000 cells/well/50 μl for 24 hours in a humidified incubator (5% CO2 and 37° C.) in DMEM/F12 medium (Wisent 319-075-CL) without selection agent. Prior to experiment, the cell culture medium is removed from the plates by inversion. A loading solution of 25 μl of Hank's balanced salt solution 1× (Wisent 311-506-CL), 10 mM Hepes (Wisent 330-050-EL) and 2.5 mM Probenicid at pH 7.4 (Sigma Aldrich Canada P8761-100g) with 2 μM calcium indicator dye (FLUO-4AM, Molecular Probes F14202) and Pluronic acid F-127 0.002% (Invitrogen P3000MP) is added to each well. Plates are incubated at 37° C. for 60 minutes prior to start the experiment. The incubation is terminated by washing the cells four times in assay buffer, leaving a residual 25 μl buffer per well. Cell plates are then transferred to the FLIPR, ready for compound additions.

The day of experiment, acetylcholine and compounds are diluted in assay buffer in three-fold concentration range (10 points serial dilution) for addition by FLIPR instrument. For all calcium assays, a baseline reading is taken for 10 seconds followed by the addition of 12.5 μl of compounds, resulting in a total well volume of 37.5 μl. Data is collected every second for 60 pictures and then every 6 seconds for 20 pictures prior to the addition of agonist. For hM3 and hM5, before agonist addition, a second baseline reading is taken for 10 seconds followed by the addition of 12.5 μl of agonist or buffer, producing a final volume of 50 μl. After agonist stimulation, the FLIPR continues to collect data every second for 60 pictures and then every 6 seconds for 20 pictures. The fluorescence emission is read using filter 1 (emission 510-570 nm) by the FLIPR on board CCD camera.

Calcium mobilization output data are calculated as the maximal relative fluorescence unit (RFU) minus the minimal value for both compound and agonist reading frame (except for hM1 and rM1 using only the maximal RFU). Data are analyzed using sigmoidal fits of a non-linear curve-fitting program (XLfit version 4.2.2 Excel add-in version 4.2.2 build 18 math 1Q version 2.1.2 build 18). All pEC50 and plC50 values are reported as arithmetic means ±standard error of mean of ‘n’ independent experiments.

### hM2 Receptor GTPγS Binding

Membranes produced from Chinese hamster ovary cells (CHO) expressing the cloned human M2 receptor (human Muscarinic receptor subtype 2, gene bank access NM_{—}000739), are obtained from Perkin-Elmer (RBHM2M). The membranes are thawed at 37° C., passed 3 times through a 23-gauge blunt-end needle, diluted in the GTPγS binding buffer (50 mM Hepes, 20 mM NaOH, 100 mM NaCl, 1 mM EDTA, 5 mM MgCl₂, pH 7.4, 100 μM DTT). The EC₅₀, IC₅₀ and Eₘₐₓ of the compounds of the invention are evaluated from 10-point dose-response curves (three fold concentration range) done in 60 μl in 384-well non-specific binding surface plate (Corning). Ten microliters from the dose-response curves plate (5× concentration) are transferred to another 384 well plate containing 25 μl of the following: 5 μg of hM2 membranes, 500 μg of Flashblue beads (Perkin-Elmer) and GDP 25 μM. An additional 15 μl containing 3.3× (60,000 dpm) of GTPγ³⁵S (0.4 nM final) are added to the wells resulting in a total well volume of 50 μl. Basal and maximal stimulated [³⁵S]GTPγS binding are determined in absence and presence of 30 μM final of acetylcholine agonist. The membranes/beads mix are pre-incubated for 15 minutes at room temperature with 25 μM GDP prior to distribution in plates (12.5 μM final). The reversal of acetylcholine-induced stimulation (2 μM final) of [³⁵S]GTPγS binding is used to assay the antagonist properties (IC₅₀) of the compounds. The plates are incubated for 60 minutes at room temperature then centrifuged at 400 rpm for 5 minutes. The radioactivity (cpm) is counted in a Trilux (Perkin-Elmer).

Values of EC₅₀, IC₅₀ and Eₘₐₓ are obtained using sigmoidal fits of a non-linear curve-fitting program (XLfit version 4.2.2 Excel add-in version 4.2.2 build 18 math 1Q version 2.1.2 build 18) of percent stimulated [³⁵S]GTPγS binding vs. log(molar ligand). All pEC50 and plC50 values are reported as arithmetic means ±standard error of mean of ‘n’ independent experiments.

### hM4 Receptor GTPγS Binding

Membranes produced from Chinese hamster ovary cells (CHO) expressing the cloned human M4 receptor (human Muscarinic receptor subtype 4, gene bank access NM_{—}000741), are obtained from Perkin-Elmer (RBHM4M). The membranes are thawed at 37° C., passed 3 times through a 23-gauge blunt-end needle, diluted in the GTPγS binding buffer (50 mM Hepes, 20 mM NaOH, 100 mM NaCl, 1 mM EDTA, 5 mM MgCl₂, pH 7.4, 100 μM DTT). The EC₅₀, IC₅₀ and Eₘₐₓ of the compounds of the invention are evaluated from 10-point dose-response curves (three fold concentration range) done in 60 μl in 384-well non-specific binding surface plate (Corning). Ten microliters from the dose-response curves plate (5× concentration) are transferred to another 384 well plate containing 25 μl of the following: 10 μg of hM4 membranes, 500 μg of Flashblue beads (Perkin-Elmer) and GDP 40 μM. An additional 15 μl containing 3.3× (60,000 dpm) of GTPγ³⁵S (0.4 nM final) are added to the wells resulting in a total well volume of 50 μl. Basal and maximal stimulated [³⁵S]GTPγS binding are determined in absence and presence of 30 μM final of acetylcholine agonist. The membranes/beads mix are pre-incubated for 15 minutes at room temperature with 40 μM GDP prior to distribution in plates (20 μM final). The reversal of acetylcholine-induced stimulation (10 μM final) of [³⁵S]GTPγS binding is used to assay the antagonist properties (IC₅₀) of the compounds. The plates are incubated for 60 minutes at room temperature then centrifuged at 400 rpm for 5 minutes. The radioactivity (cpm) is counted in a Trilux (Perkin-Elmer).

Values of EC₅₀, IC₅₀ and Eₘₐₓ are obtained using sigmoidal fits of a non-linear curve-fitting program (XLfit version 4.2.2 Excel add-in version 4.2.2 build 18 math 1Q version 2.1.2 build 18) of percent stimulated [³⁵S]GTPγS binding vs. log(molar ligand). All pEC50 and plC50 values are reported as arithmetic means ±standard error of mean of ‘n’ independent experiments.

Certain biological properties of certain compounds of the invention measured using one or more assays described above are listed in Table 1 below.

TABLE 1
Certain Biological Properties of the Certain Compounds of the Invention

| Example | hM1 EC50 (nM) | hM2 EC50 (nM) | hM3 EC50 (nM) | hM4 EC50 (nM) | hM5 EC50 (nM) |
|---|---|---|---|---|---|
| 1 | 4.2 | 498 | 1404 | 1696 | 136 |
| 2 | 13 | 865 | | 2484 | |
| 3 | 108.3 | | | | |
| 4 | 13.5 | 1864 | >40000 | 4380 | >9599 |
| 5 | 43.3 | 5128 | >40000 | >16028 | >2963 |
| 6 | <1.4 | 588 | | 1731 | |
| 7 | <6.6 | 4503 | >40000 | >24440 | 3102 |
| 8 | 18.3 | 1278 | | 5209 | |
| 9 | 123.9 | | | | |
| 10 | 26.3 | 2144 | >40000 | 5168 | 537 |
| 11 | 2 | 149 | 1127 | 714 | |
| 12 | 10.1 | 771 | | 2005 | |
| 13 | 70.1 | | | | |
| 14 | 48.3 | 1502 | | >90000 | |
| 15 | 94.9 | 2849 | >40000 | >90000 | |
| 16 | 8.1 | 418 | | 1703 | |
| 17 | 9.7 | 709 | | 3147 | |
| 18 | 158.2 | | | | |
| 19 | 324.1 | | | | |
| 20 | 14.2 | 61 | 535 | 97 | 720 |
| 21 | 12.5 | 94 | | 262 | |
| 22 | 2.8 | 126 | | 421 | |
| 23 | 9.4 | 668 | | 3868 | |
| 24 | 10.4 | 1352 | >26456 | 5094 | 1574 |
| 25 | 1.8 | 146 | | 684 | |
| 26 | 7.8 | 146 | | 857 | |
| 27 | 10.8 | 620 | | 3089 | |
| 28 | 10 | 2402 | >24336 | 8394 | 2652 |
| 29 | 166.4 | | | | |
| 30 | 108.1 | | | | |
| 31 | 13.1 | 997 | | 1193 | |
| 32 | 52.1 | 5835 | | 33715 | |
| 33 | 1.7 | 104 | | 316 | |
| 34 | 14.7 | 897 | | 1674 | |
| 35 | 56.9 | | | | |
| 36 | 32 | 1563 | | 6828 | |
| 37 | 76.5 | | | | |
| 38 | 111.6 | | | | |
| 39 | 26.7 | >12782 | | >90000 | |
| 40 | 36.3 | 2805 | | >22340 | |
| 41 | 23.5 | 2215 | >40000 | 8798 | >40000 |
| 42 | 235.1 | | | | |
| 43 | 2.5 | 109 | 553 | 460 | 205 |
| 44 | <2.3 | 26 | | 107 | |
| 45 | 14.1 | 1640 | >40000 | 11187 | 1075 |
| 46 | 132.4 | | | | |
| 47 | <3.2 | 127 | | 907 | |
| 48 | 8.5 | 571 | >40000 | 3478 | >40000 |
| 49 | 15.1 | 4550 | >40000 | 9478 | >6213 |
| 50 | 2.2 | 498 | >40000 | 2017 | 3562 |
| 51 | 57.1 | | | | |
| 52 | 14.7 | 1242 | >40000 | 5089 | >9635 |
| 53 | 11.5 | 1622 | >40000 | 5575 | >6160 |
| 54 | 92.8 | | | | |
| 55 | 4.9 | 319 | >40000 | 1713 | 1822 |
| 56 | 3.5 | 24 | >3541 | 238 | 1282 |
| 57 | 2.6 | 428 | 3553 | 2203 | 356 |
| 58 | 17.7 | 2103 | >40000 | 9810 | 2208 |
| 59 | 77.6 | | | | |
| 60 | 13.5 | 1034 | >40000 | 5075 | >7266 |
| 61 | 14.1 | 1707 | >40000 | 4418 | >2705 |
| 62 | 79.8 | | | | |
| 63 | 343.7 | | | | |
| 64 | 80.1 | | | | |
| 65 | 104.1 | | | | |
| 66 | 50.2 | | | | |
| 67 | 20.9 | 4649 | >33483 | 15129 | >6711 |
| 68 | 13.3 | 1150 | 2330 | 6214 | >4085 |
| 69 | 11 | 1861 | >40000 | 8924 | 1373 |
| 70 | 9.8 | 448 | | 1769 | |
| 71 | 122.6 | | | | |
| 72 | 9.5 | 950 | >40000 | >90000 | >40000 |
| 73 | 31 | 6157 | >40000 | >90000 | >40000 |
| 74 | 10 | 1345 | >40000 | 3604 | >21208 |
| 75 | 47.5 | 2623 | >40000 | >18920 | >27033 |
| 76 | 40.1 | 3504 | | >90000 | |
| 77 | 94.5 | | | | |
| 78 | 76.2 | | | | |
| 79 | 9.2 | 1126 | >40000 | 4331 | 2539 |
| 80 | 69.4 | 3256 | | >21767 | |
| 81 | 8.7 | 1675 | >13333 | 7067 | 1241 |
| 82 | 8.7 | 270 | | 937 | |
| 83 | 26.1 | 2843 | >18404 | 11289 | 3563 |
| 84 | 7.7 | 684 | | 2539 | |
| 85 | 1465.8 | | | | |
| 86 | 1416 | | | | |
| 87 | 124.7 | | | | |

### Rat SNL Heat Hyperalgesia Assay

Rats undergo spinal nerve ligation surgery as described in Kim and Chung (1992) (reference 1). Briefly, rats are anesthetized with isoflurane, the left L5 and L6 are isolated and tightly ligated with 4-0 silk thread. The wound is closed by suturing and applying tissue adhesive. Compound testing is performed at day 9 to day 36 post-surgery.

For behavioral testing, the animals are acclimatized to the test room environment for a minimum of 30 min. In order to assess the degree of hyperalgesia, the animals are placed on a glass surface (maintained at 30° C.), and a heat-source is focused onto the plantar surface of the left paw. The time from the initiation of the heat until the animal withdraws the paw is recorded. Each animal is tested twice (with an interval of 10 min between the two tests). A decrease in Paw Withdrawal Latency (PWL, average of the two tests) relative to naïve animals indicates a hyperalgesic state. The rats with a PWL of at least 2 seconds less than average PWL of Naive group are selected for compound testing.

Each individual experiment consists of several groups of SNL rats, one group receiving vehicle while the other groups receive different doses of the test article. In all experiments, animals are tested for heat hyperalgesia using the plantar test before drug or vehicle administration to ensure stable heat-hyperalgesia baseline and rats are evenly divided into groups for compound testing. At a suitable interval after vehicle or drug administration, another test is performed to measure PWL. Generally, results from 2 individual experiments are pooled together and the data are presented as the mean paw withdrawal latency (PWL) (s) ±standard error of mean (SEM).

A combination containing a compound of the present invention and morphine at a predetermined ratio (e.g., 0.64:1) may be tested using this instant model. The combination drugs may be administered to the rats subcutaneously, orally or combination thereof, simultaneously or sequentially. The results (expressed as ED₅₀) for the combination may be compared with results obtained singly for the compound of the instant invention and morphine at the same or similar dosage range. If the ED₅₀ of the combination is significantly lower than the theoretical ED₅₀ calculated based on the ED₅₀ measured using the compound of the invention and morphine singly, then a synergy for the combination is indicated.

### Prophetic Assays for Measuring the Effects of the Compound on Intraocular Pressure (IOP), Pupil Diameter (PD) in Rabbits and Monkeys

### Drug Preparation and Administration

A compound to be tested is dissolved in physiological saline at 0.1, 0.3, 1.0% for rabbit study and 0.5, 1.0% for monkey studies. Drug or vehicle aliquots (25 ul) are administered topically unilaterally or bilaterally. In unilateral applications, the contralateral eyes receive an equal volume of saline. Proparacaine (0.5%) is applied to the cornea prior to tonometry to minimize discomfort. Intraocular pressure (IOP) is recorded using a pneumatic tonometer (Alcon Applanation Pneumatonograph) or equivalent.

### Analysis

The results are expressed as the changes in IOP from the basal level measured just prior to administration of drug or vehicle and represent the mean, plus or minus standard deviation. Statistical comparisons are made using the Student's t-test for non-paired data between responses of drug-treated and vehicle-treated animals and for paired data between ipsilateral and contralateral eyes at comparable time intervals. The significance of the date is also determined as the difference from the “t-0” value using Dunnett's “t” test. Asterisks represent a significance level of p<0.05.

### Intraocular Pressure Measurement in Rabbits

Male Dutch Belted rabbits weighing 2.5-4.0 kg are maintained on a 12-hour light/dark cycle and rabbit chow. All experiments are performed at the same time of day to minimize variability related to diurnal rhythm. IOP is measured before treatment then the test compound or vehicle (saline) is instilled (one drop of 25 ul) into one or both eyes and IOP is measured at 30, 60, 120, 180, 240, 300, and 360 minutes after instillation. In some cases, equal number of animals treated bilaterally with vehicle only are evaluated and compared to drug treated animals as parallel controls.

### Pupil Diameter (PD) Measurement in Rabbits

Male Dutch Belted rabbits weighing 2.5-4.0 kg are maintained on a 12-hour light/dark cycle and rabbit chow. All experiments are performed at the same time of day to minimize variability related to diurnal rhythm. PD is measured before treatment then the test compound or vehicle (saline) is instilled (one drop of 25 μl) into one or both eyes and PD is measured at 30, 60, 120, 180, 240, 300, and 360 minutes after instillation. In some cases, equal number of animals treated bilaterally with vehicle only are evaluated and compared to drug treated animals as parallel controls.

### Intraocular Pressure Measurements in Monkeys

Unilateral ocular hypertension of the right eye is induced in female cynomolgus monkeys weighing between 2 and 3 kg by photocoagulation of the trabecular meshwork with an argon laser system (Coherent NOVUS 2000, Palo Alto, USA) using the method of Lee at al. (1985). The prolonged increase in intraocular pressure (IOP) results in changes to the optic nerve head that are similar to those found in glaucoma patients.

For IOP measurements, the monkeys are kept in a sitting position in restraint chairs for the duration of the experiment. Animals are lightly anesthetized by the intramuscular injection of ketamine hydrochloride (3-5 mg/kg) approximately five minutes before each IOP measurement and one drop of 0.5% proparacaine is instilled prior to recording IOP. IOP is measured using a pneumatic tonometer (Alcon Applanation Tonometer) or a Digilab pneumatonometer (Bio-Rad Ophthalmic Division, Cambridge, Mass., USA).

IOP is measured before treatment and generally at 30, 60, 124, 180, 300, and 360 minutes after treatment. Baseline values are also obtained at these time points generally two or three days prior to treatment. Treatment consisted of instilling one drop of 25 μl of the test compound (0.5 and 1.0%) or vehicle (saline). At least one-week washout period is employed before testing on the same animal. The normotensive (contralateral to the hypertensive) eye is treated in an exactly similar manner to the hypertensive eye. IOP measurements for both eyes are compared to the corresponding baseline values at the same time point. Results are expressed as mean plus-or-minus standard deviation in mm Hg.

### Pupil Diameter (PD) Measurement in Monkeys

All experiments are performed at the same time of day to minimize variability related to diurnal rhythm. PD is measured with a pupillometer before treatment then the test compound or vehicle (saline) is instilled (one drop of 25 ul) into one or both eyes and PD is measured at 30, 60, 120, 180, 240, 300, and 360 minutes after instillation. In some cases, equal number of animals treated bilaterally with vehicle only are evaluated and compared to drug treated animals as parallel controls.

### EXAMPLES

In order that the invention disclosed herein may be more efficiently understood, examples are provided below. It should be understood that these examples are for illustrative purposes only and are not to be construed as limiting the invention in any manner.

The following abbreviations are used herein: “RT” or “rt” means room temperature.

Preparative LCMS Conditions: High pH LCMS purifications are run on Xbridge column with the following specification: XBridge Prep C18 OBD, 30×50, 5 um, run time: 10 min, mobile phases for high pH preparative LCMS are pH˜10 water and acetonitrile. pH˜10 water is prepared in the following fashion: dissolve 3.16 g NH₄HCO₃ (final concentration of 10 mM), 15 mL concentrated ammonium hydroxide for every 4 L water. The gradient description in the experimental part, such as “High pH, 30-50% CH₃CN” means that the starting gradient for the run is 30% CH₃CN,/70% water for 1 minute, and then it goes to 50% CH₃CN/50% water in 7 minutes followed by a 2 minutes wash at 100% CH₃CN.

The compounds described in this application may be named with ChembridgeSoft naming program (Chemoffice 9.0.7)

Chiral Super Critical Fluid Chromatography conditions: Chiral SFC are run on ChiralPak AD-H or ChiralPak AS-H with the following specifications: Dimensions of 10×250 mm, particle size 5 uM, Main eluent is CO₂ with mixture of co-eluents such as methanol, isopropanol and dimethylethylamine (DMEA). Column temperature: 35° C., back pressure 100 Bar. Detection by UV at 215 nM wavelength.

“HRMS” means high resolution mass spectra.

“HATU” means O-(7-Azabenzotriazol-1-yl)-N,N,N′,N′-tetramethyluronium hexafluorophosphate.

“CDI” means 1,1′-Carbonyldiimidazole.

“DIPEA” means Diisopropylethylamine.

### Example 1

### ethyl 3-methyl-3-(4-(2-oxo-2,3-dihydro-1H-benzo[d]imidazol-1-yl)piperidin-1-yl)pyrrolidine-1-carboxylate

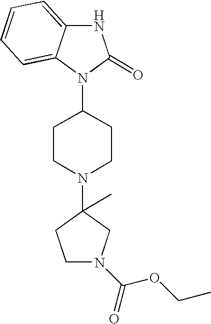

### Step A: Preparation of tert-butyl 3-cyano-3-(4-hydroxypiperidin-1-yl)pyrrolidine-1-carboxylate

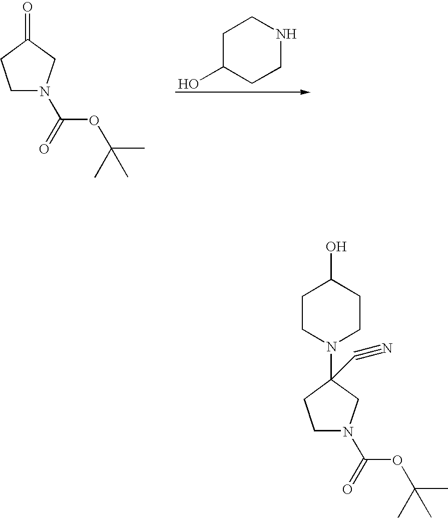

To a mixture of piperidin-4-ol (5.06 g, 0.05 mol) and tert-butyl 3-oxopyrrolidine-1-carboxylate (7.72 g, 0.04 mol) in ClCH₂CH₂Cl (200 mL) was added tetraisopropoxytitanium (0.012 kg, 0.04 mol). The reaction mixture was stirred at room temperature for 24 hours. 1M solution of cyanodiethylaluminum (100 mL, 0.10 mol) in toluene was added and the mixture was stirred at room temperature for 24 hours. The solution was then diluted with dichloromethane (250 mL) and quenched with saturated aqueous NH₄Cl solution (100 mL) at 0° C. The mixture was filtered through a small pad of celite, and the filtrate was concentrated in vacuo to give the title product as pale yellow solid, which was used in the subsequent step without further purification. 1H NMR (400 MHz, CHLOROFORM-D) δ ppm 1.47 (s, 9H) 1.55-1.70 (m, 4H) 1.87-2.12 (m, 3H) 2.29-2.53 (m, 3H) 2.65-2.77 (m, 1H) 2.88 (d, J=8.59 Hz, 1H) 3.28 (d, J=9.37 Hz, 1H) 3.48-3.84 (m, 2H) 3.99 (dd, J=42.77, 10.74 Hz, 1H).

### Step B: Preparation of tert-butyl 3-(4-hydroxypiperidin-1-yl)-3-methylpyrrolidine-1-carboxylate

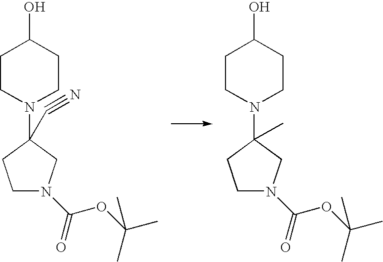

To a solution of tert-butyl 3-cyano-3-(4-hydroxypiperidin-1-yl)pyrrolidine-1-carboxylate (1 g, 3.39 mmol) in dry THF (20 mL) and was added a 1.0 M solution of methylmagnesium bromide (13.5 mL, 13.54 mmol) in butylether at 0° C. The reaction mixture was stirred at room temperature for 4 hours. The reaction mixture was quenched with saturated aqueous NH₄Cl solution (30 mL) at 0° C. and diluted with ethyl acetate (50 mL). The layers were separated and the organic layer was washed with brine, dried over Na₂SO₄, filtered and filtrate was concentrated in vacuo to give the title compound (1.069 g), which was used in the subsequent step without further purification.

### Step C: Preparation of tert-butyl 3-methyl-3-(4-oxopiperidin-1-yl)pyrrolidine-1-carboxylate

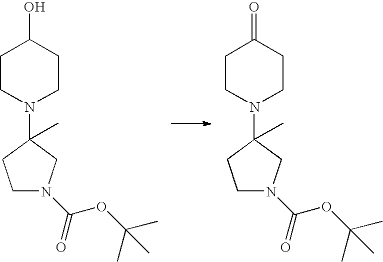

To a solution of oxalyl dichloride (2M, 2.5 mL, 5.09 mmol) in dichloromethane was added dropwise DMSO (0.722 mL, 10.17 mmol) at −78° C. under an nitrogen atmosphere. The reaction flask was kept in a −78° C. bath and after stirring for 10 minutes, a solution of tert-butyl 3-(4-hydroxypiperidin-1-yl)-3-methylpyrrolidine-1-carboxylate (0.964 g, 3.39 mmol) in dichloromethane (2 mL) was added and stirred for another 10 minutes. Triethylamine (1.890 mL, 13.56 mmol) was added and stirred at −78° C. for 30 minutes and then the reaction mixture was allowed to warm to 0° C. over 30 minutes. The reaction was quenched with saturated aqueous NH₄Cl (10 mL) and extracted with dichloromethane (3×10 mL). The combined the organic extract was washed with brine, dried over MgSO₄, filtered and concentrated in vacuo to give the title compound (0.856 g, 89%) as pale yellow solid, which was used in the subsequent step without further purification.

### Step D: Preparation of tert-butyl 3-(4-(2-aminophenylamino)piperidin-1-yl)-3-methylpyrrolidine-1-carboxylate

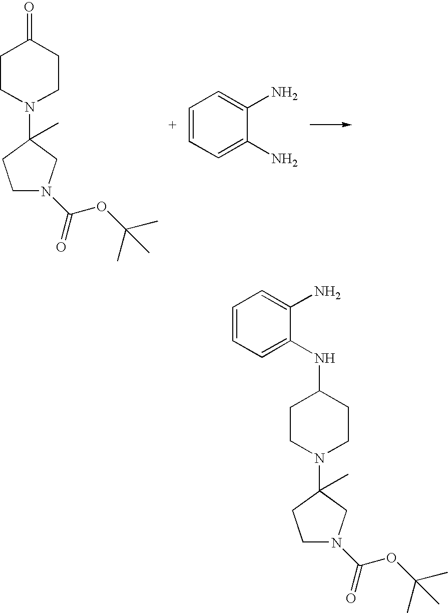

To a mixture of tert-butyl 3-methyl-3-(4-oxopiperidin-1-yl)pyrrolidine-1-carboxylate (1.5 g, 5.31 mmol) and benzene-1,2-diamine (0.574 g, 5.31 mmol) in dichloromethane (20 mL) was added sodium triacetoxyhydroborate (3.38 g, 15.94 mmol) followed by acetic acid (1.520 mL, 26.56 mmol). The reaction mixture was stirred at room temperature for 2 hours. The reaction was quenched with water and extracted with dichloromethane (3×10 mL). The combined organic extract was washed with brine, dried over MgSO₄, filtered and concentrated in vacuo. The residue was purified by high pH preparative HPLC (40-60% MeCN in water) to give the title compound (0.838 g, 42.1%) as pale yellow solid. 1H NMR (400 MHz, METHANOL-D4) δ ppm 1.08 (s, 3H) 1.39-1.55 (m, 12H) 1.79-1.92 (m, 2H) 2.04 (d, J=12.50 Hz, 2H) 2.38 (t, J=11.13 Hz, 1H) 2.47 (t, J=11.33 Hz, 1H) 2.62-2.73 (m, 1H) 2.82-2.91 (m, 1H) 3.14 (t, J=9.96 Hz, 1H) 3.18-3.31 (m, 2H) 3.31-3.36 (m, 2H) 3.44-3.53 (m, 1H) 6.51-6.56 (m, 1H) 6.59-6.67 (m, 2H) 6.69 (d, J=7.42 Hz, 1H). MS (M+1): 375.33.

### Step E: Preparation of tert-butyl 3-methyl-3-(4-(2-oxo-2,3-dihydro-1H-benzo[d]imidazol-1-yl)piperidin-1-yl)pyrrolidine-1-carboxylate

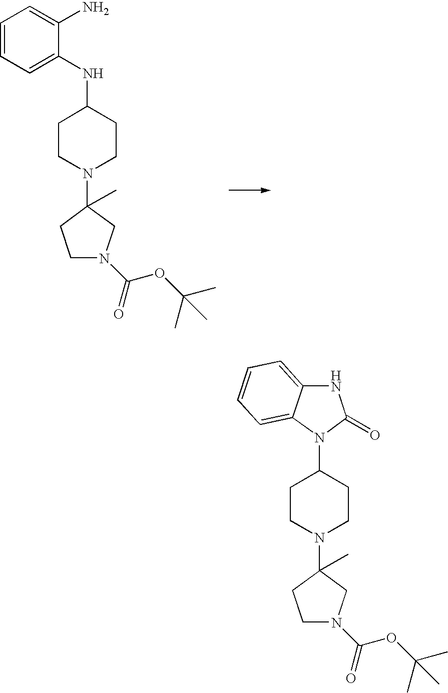

To a solution of tert-butyl 3-(4-(2-aminophenylamino)piperidin-1-yl)-3-methylpyrrolidine-1-carboxylate (0.838 g, 2.24 mmol) and triethylamine (0.468 mL, 3.36 mmol) in dichloromethane (15 mL) was added a solution of bis(trichloromethyl) carbonate (0.199 g, 0.67 mmol) in dichloromethane (2 mL) drop wise at 0° C. The reaction mixture was stirred for 30 minutes at 0° C. The reaction was quenched with water and extracted with dichloromethane (3×10 mL). Combined organic extract was washed with brine, dried over MgSO₄, filtered and concentrated in vacuo. The residue was purified by high pH preparative HPLC (30-50% MeCN in water) to give the title compound as white solid. 1H NMR (400 MHz, METHANOL-D4) δ ppm 1.12 (s, 3H) 1.44 (s, 9H) 1.70-1.80 (m, 2H) 1.87-1.96 (m, 2H) 2.39-2.62 (m, 4H) 2.81 (t, J=8.79 Hz, 1H) 2.94-3.06 (m, 1H) 3.16-3.25 (m, 1H) 3.29-3.41 (m, 2H) 3.46-3.57 (m, 1H) 4.17-4.32 (m, 1H) 6.98-7.07 (m, 3H) 7.33-7.40 (m, 1H). MS (M+1): 401.3.

### Step F: Preparation of ethyl 3-methyl-3-(4-(2-oxo-2,3-dihydro-1H-benzo[d]imidazol-1-yl)piperidin-1-yl)pyrrolidine-1-carboxylate

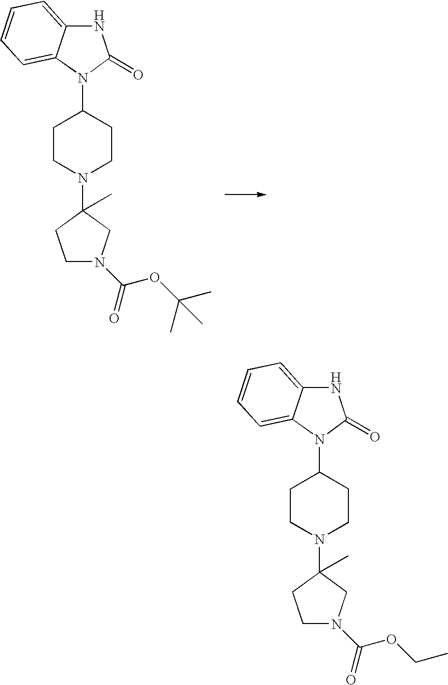

A solution of tert-butyl 3-methyl-3-(4-(2-oxo-2,3-dihydro-1H-benzo[d]imidazol-1-yl)piperidin-1-yl)pyrrolidine-1-carboxylate (37.1 mg, 0.09 mmol) and 2,2,2-trifluoroacetic acid (0.5 mL, 6.73 mmol) in dichloromethane (2 mL) was stirred at room temperature for 30 minutes and concentrated in vacuo. Triethylamine (0.065 mL, 0.46 mmol) and dichloromethane (3 mL) was added followed by ethyl carbonochloridate (8.83 μL, 0.09 mmol) at 0° C. The reaction mixture was stirred for 30 minutes at 0° C. The reaction was quenched with water and extracted with dichloromethane (3×10 mL). Combined the organic extract was washed with brine, dried over MgSO₄, filtered and concentrated in vacuo. The residue was added with t-butylamine (94 uL, 0.9 mmol) and MeOH (5 mL) and the mixture was heated at 60° C. for 1 hour. Concentrated in vacuo and the residue was purified by high pH preparative HPLC (20-40% MeCN in water) to give the title compound (35.0 mg, 92%) as white solid. 1H NMR (400 MHz, METHANOL-D4) δ ppm 1.13 (s, 3H) 1.20-1.27 (m, 3H) 1.72-1.82 (m, 2H) 1.87-2.02 (m, 2H) 2.38-2.62 (m, 4H) 2.78-2.88 (m, 1H) 2.97-3.08 (m, 1H) 3.24 (dd, J=10.16, 5.08 Hz, 1H) 3.32-3.48 (m, 2H) 3.51-3.64 (m, 1H) 4.10 (q, J=6.77 Hz, 2H) 4.19-4.33 (m, 1H) 6.98-7.07 (m, 3H) 7.33-7.40 (m, 1H). MS (M+1): 373.3, HRMS (M+1): 373.2236.

### Example 2

### 3-methyl -3-(4-(2-oxo-2,3-dihydro-1H-benzo[d]imidazol-1-yl)piperidin-1-yl)pyrrolidine-1-carboxylate

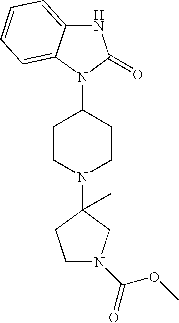

### Preparation of 3-methyl-3-(4-(2-oxo-2,3-dihydro-1H-benzo[d]imidazol-1-yl)piperidin-1-yl)pyrrolidine-1-carboxylate

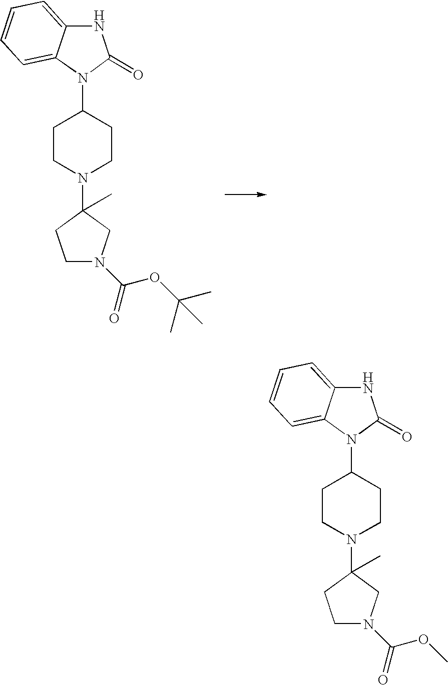

Following an analogous procedure to that described in the Step F of the Example 1, the title compound was made from tert-butyl 3-methyl-3-(4-(2-oxo-2,3-dihydro-1H-benzo[d]imidazol-1-yl)piperidin-1-yl)pyrrolidine-1-carboxylate (42.5 mg, 0.11 mmol) and methyl carbonochloridate (8.20 μL, 0.11 mmol). After the purification by high pH preparative HPLC (20-40% MeCN in water), the title compound was obtained as white solid (39.6 mg, 95%). 1H NMR (400 MHz, METHANOL-D4) δ ppm 1.12 (s, 3H) 1.69-1.82 (m, 2H) 1.88-2.04 (m, 2 H) 2.36-2.63 (m, 4H) 2.82 (t, J=8.20 Hz, 1H) 2.94-3.09 (m, 1H) 3.24 (d, J=10.16 Hz, 1H) 3.31-3.47 (m, 2 H) 3.52-3.61 (m, 1H) 3.67 (d, J=1.56 Hz, 3H) 4.18-4.32 (m, 1H) 6.98-7.08 (m, 3H) 7.32-7.40 (m, 1H). MS (M+1): 359.2, HRMS (M+1): 359.2075.

### Example 3

### isopropyl 3-methyl-3-(4-(2-oxo-2,3-dihydro-1H-benzo[d]imidazol-1-yl)piperidin-1-yl)pyrrolidine-1-carboxylate

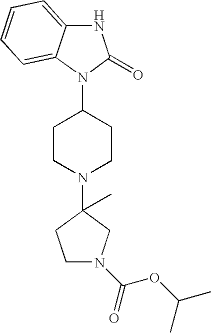

### Preparation of isopropyl 3-methyl-3-(4-(2-oxo-2,3-dihydro-1H-benzo[d]imidazol-1-yl)piperidin-1-yl)pyrrolidine-1-carboxylate

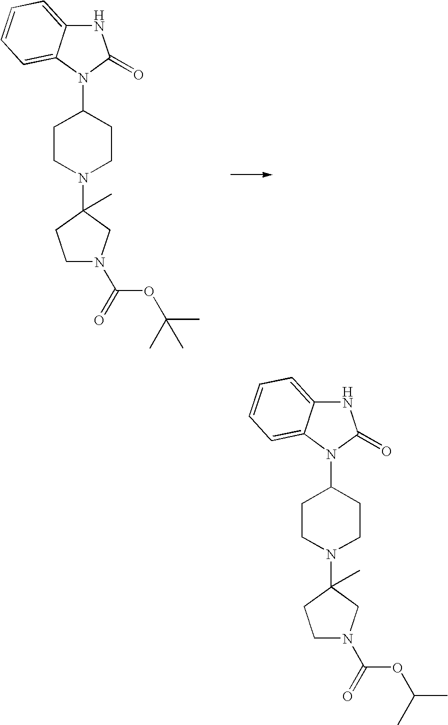

Following an analogous procedure to that described in the Step F of the Example 1, the title compound was made from tert-butyl 3-methyl-3-(4-(2-oxo-2,3-dihydro-1H-benzo[d]imidazol-1-yl)piperidin-1-yl)pyrrolidine-1-carboxylate (42.5 mg, 0.11 mmol) and isopropyl carbonochloridate (13.00 mg, 0.11 mmol). After the purification by high pH preparative HPLC (30-50% MeCN in water), the title compound was obtained as white solid (41.5 mg, 92%). 1H NMR (400 MHz, METHANOL-D4) δ ppm 1.12 (s, 3H) 1.19-1.28 (m, 6H) 1.70-1.81 (m, 2H) 1.88-1.98 (m, 2H) 2.38-2.64 (m, 4H) 2.77-2.87 (m,1H) 2.96-3.07 (m, 1H) 3.23 (dd, J=10.16, 1.95 Hz, 1H) 3.31-3.45 (m, 2H) 3.50-3.61 (m,1H) 4.17-4.33 (m, 1H) 4.76-4.92 (m, 1H) 6.98-7.07 (m, 3H) 7.34-7.40 (m, 1H). MS (M+1): 387.2, HRMS (M+1): 387.2387.

### Example 4

### 2-fluoroethyl 3-methyl-3-(4-(2-oxo-2,3-dihydro-1H-benzo[d]imidazol-1-yl)piperidin-1-yl)pyrrolidine-1-carboxylate

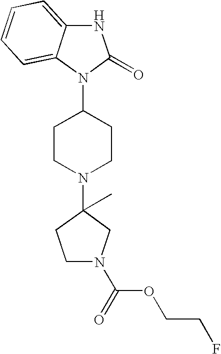

### Preparation of 2-fluoroethyl 3-methyl-3-(4-(2-oxo-2,3-dihydro-1H-benzo[d]imidazol-1-yl)piperidin-1-yl)pyrrolidine-1-carboxylate

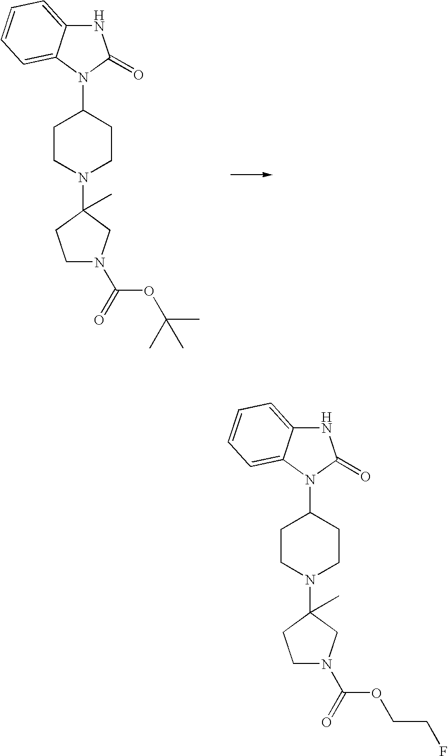

Following an analogous procedure to that described in the Step F of the Example 1, the title compound was made from tert-butyl 3-methyl-3-(4-(2-oxo-2,3-dihydro-1H-benzo[d]imidazol-1-yl)piperidin-1-yl)pyrrolidine-1-carboxylate (42.5 mg, 0.11 mmol) and 2-fluoroethyl carbonochloridate (10.02 μL, 0.11 mmol). After the purification by high pH preparative HPLC (20-40% MeCN in water), the title compound was obtained as white solid (40.5 mg, 89%).1H NMR (400 MHz, METHANOL-D4) δ ppm 1.13 (s, 3H) 1.70-1.82 (m, 2H) 1.88-1.99 (m, 2H) 2.38-2.63 (m, 4H) 2.78-2.90 (m, 1H) 3.02 (dd, J=3.91, 1.95 Hz, 1H) 3.34-3.52 (m, 3H) 3.53-3.69 (m, 1H) 4.19-4.36 (m, 3H) 4.47-4.53 (m, 1H) 4.60-4.67 (m, 1H) 6.97-7.09 (m, 3H) 7.33-7.39 (m, 1H). MS (M+1): 391.2, HRMS (M+1): 391.2142.

### Example 5

### N-ethyl-3-methyl-3-(4-(2-oxo-2,3-dihydro-1H-benzo[d]imidazol-1-yl)piperidin-1-yl)pyrrolidine-1-carboxamide

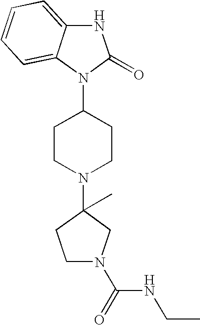

### Preparation of N-ethyl-3-methyl-3-(4-(2-oxo-2,3-dihydro-1H-benzo[d]imidazol-1-yl)piperidin-1-yl)pyrrolidine-1-carboxamide

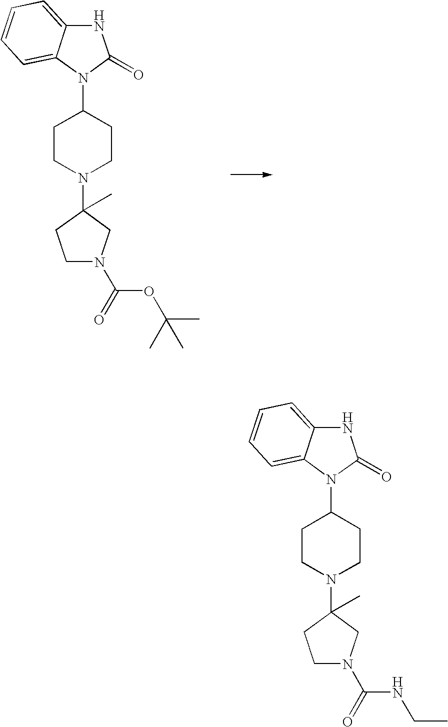

Following an analogous procedure to that described in the Step F of the Example 1, the title compound was made from tert-butyl 3-methyl-3-(4-(2-oxo-2,3-dihydro-1H-benzo[d]imidazol-1-yl)piperidin-1-yl)pyrrolidine-1-carboxylate (42.5 mg, 0.11 mmol) and isocyanatoethane (8.40 μL, 0.11 mmol). After the purification by high pH preparative HPLC (20-40% MeCN in water), the title compound was obtained as white solid (27.2 mg, 62.8%). 1H NMR (400 MHz, METHANOL-D4) δ ppm 1.09 (t, J=7.23 Hz, 3H) 1.12 (s, 3H) 1.70-1.83 (m, 2H) 1.91-2.02 (m, 2H) 2.39-2.62 (m, 4H) 2.85 (dd, J=10.74, 2.15 Hz, 1H) 2.96-3.08 (m, 1H) 3.12-3.24 (m, 3H) 3.29-3.45 (m, 2H) 3.46-3.59 (m, 1H) 4.19-4.34 (m, 1H) 6.96-7.10 (m, 3H) 7.33-7.43 (m, 1H). MS (M+1): 372.3, HRMS (M+1): 372.2389.

### Example 6 (isomer 1) and Example 7 (Isomer 2)

### ethyl 3-(4-(5-fluoro-2-oxo-2,3-dihydro-1H-benzo[d]imidazol-1-yl)piperidin-1-yl)-3-methylpyrrolidine-1-carboxylate(enantiomers)

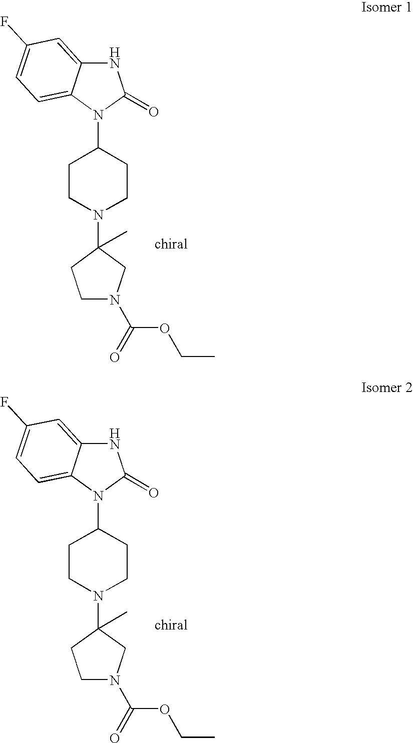

### Step A: Preparation of ethyl 3-cyano-3-(4-hydroxypiperidin-1-yl)pyrrolidine-1-carboxylate

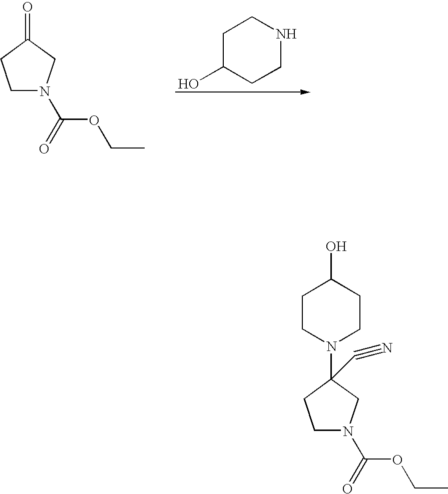

Following an analogous procedure to that described in the Step A of the Example 1, the title compound was made from piperidin-4-ol (4.68 g, 46.27 mmol) and ethyl 3-oxopyrrolidine-1-carboxylate (5.81 g, 37 mmol). The crude product was used in the subsequent step without further purification. 1H NMR (400 MHz, CHLOROFORM-D) δ ppm 1.27 (t, J=7.03 Hz, 3H) 1.55-1.71 (m, 2H) 1.89-2.16 (m, 3H) 2.28-2.51 (m, 3H) 2.73 (d, J=5.47 Hz, 1H) 2.83-2.96 (m, 1H) 3.33 (dd, J=10.55, 3.52 Hz, 1H) 3.53-3.90 (m, 4H) 4.04 (dd, J=27.93, 10.74 Hz, 1H) 4.15 (q, J=7.03 Hz, 2H). MS (M+1): 268.22.

### Step B: Preparation of ethyl 3-(4-hydroxypiperidin-1-yl)-3-methylpyrrolidine-1-carboxylate

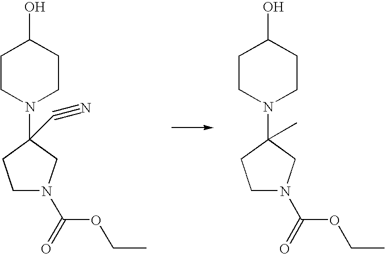

Following an analogous procedure to that described in the Step B of the Example 1, the title compound was made from ethyl 3-cyano-3-(4-hydroxypiperidin-1-yl)pyrrolidine-1-carboxylate (1 g, 3.74 mmol). The title compound was obtained as colorless oil (0.853 g, 89% yield), which was used in the subsequent step without further purification. MS (M+1): 257.28.

### Step C: Preparation of ethyl 3-methyl-3-(4-oxopiperidin-1-yl)pyrrolidine-1-carboxylate

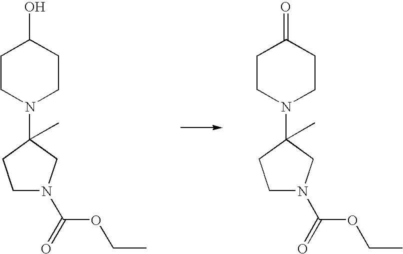

Following an analogous procedure to that described in the Step C of the Example 1, the title compound was made from ethyl 3-(4-hydroxypiperidin-1-yl)-3-methylpyrrolidine-1-carboxylate (0.853 g, 3.33 mmol). After the purification by high pH preparative HPLC (10-30% MeCN in water), the title compound was obtained as colorless oil (12.6 mg. 1.5% yield). 1H NMR (400 MHz, CHLOROFORM-D) δ ppm 0.99-1.10 (m, 3H) 1.16-1.30 (m, 3H) 1.74-2.05 (m, 4H) 2.37-2.46 (m, 2H) 2.65-2.76 (m,1H) 2.77-2.89 (m, 1H) 3.19-3.69 (m, 6H) 4.02-4.23 (m, 2H). MS (M+1): 255.26.

### Step D: Preparation of ethyl 3-(4-(2-amino-4-fluorophenylamino)piperidin-1-yl)-3-methylpyrrolidine-1-carboxylate

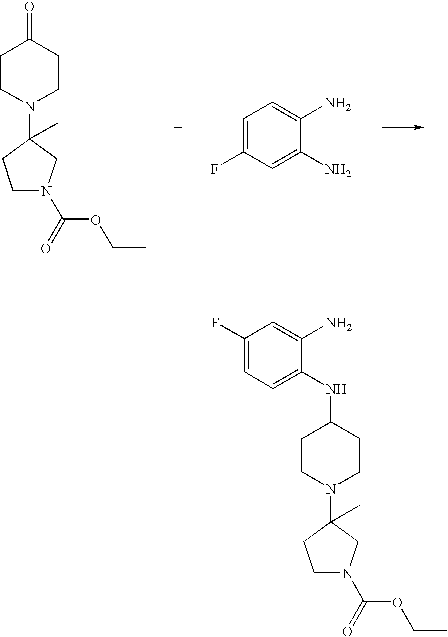

Following an analogous procedure to that described in the Step D of the Example 1, the title compound was made from ethyl 3-methyl-3-(4-oxopiperidin-1-yl)pyrrolidine-1-carboxylate (75.6 mg, 0.30 mmol) and 4-fluorobenzene-1,2-diamine (37.5 mg, 0.30 mmol). After the purification by high pH preparative HPLC (30-50% MeCN in water), the title compound was obtained as a mixture of regioisomers (regio isomer: 3-(4-(2-amino-5-fluorophenylamino)piperidin-1-yl)-3-methylpyrrolidine-1-carboxylate) as pale yellow solid (66.7 mg, 61.7% yield). 1H NMR (400 MHz, CHLOROFORM-D) δ ppm 1.06 (d, J=1.95 Hz, 3H) 1.26 (t, J=7.03 Hz, 3H) 1.38-1.56 (m, 2H) 1.76-2.14 (m, 4H) 2.26-2.51 (m, 2H) 2.66 (dd, J=6.45, 4.49 Hz, 1H) 2.76-2.93 (m, 1H) 3.05-3.15 (m, 1H) 3.22 (t, J=9.96 Hz, 1H) 3.29-3.75 (m, 6H) 4.02-4.22 (m, 2H) 6.39-6.50 (m, 2H) 6.53-6.68 (m, 1H). MS (M+1): 365.3.

### Step E: Preparation of ethyl 3-(4-(5-fluoro-2-oxo-2,3-dihydro-1H-benzo[d]imidazol-1-yl)piperidin-1-yl)-3-methylpyrrolidine-1-carboxylate

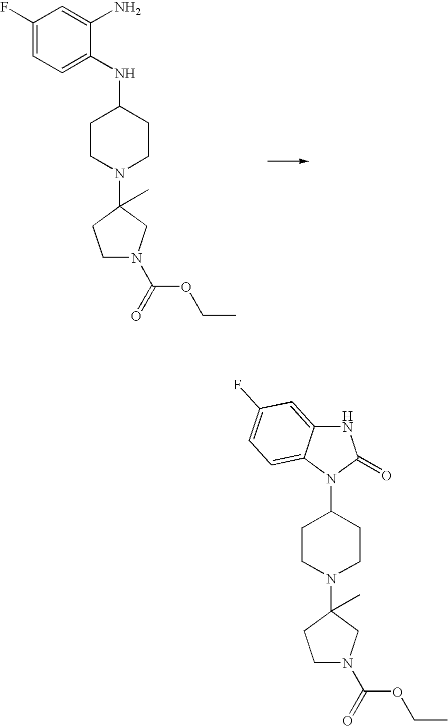

To a solution of ethyl 3-(4-(2-amino-4-fluorophenylamino)piperidin-1-yl)-3-methylpyrrolidine-1-carboxylate (96 mg, 0.26 mmol) (which also contains the regio isomer 3-(4-(2-amino-5-fluorophenylamino)piperidin-1-yl)-3-methylpyrrolidine-1-carboxylate) and triethylamine (0.066 mL, 0.47 mmol) in dichloromethane (5 mL) was added bis(trichloromethyl)carbonate (31.0 mg, 0.10 mmol) in dichloromethane (1 mL) drop wise at 0° C. The reaction mixture was stirred at 0° C. for 30 minutes. The reaction was quenched with water and extracted with dichloromethane (3×10 mL). Combined the organic extract was washed with brine, dried over MgSO4, filtered and concentrated in vacuo. The residue was purified by high pH preparative HPLC (40-60% MeCN in water) to afford the title compound as white solid (72 mg, 59.5% yield) (also contains the regio isomer).

### Step F: Chiral separation of racemic ethyl 3-(4-(5-fluoro-2-oxo-2,3-dihydro-1H-benzo[d]imidazol-1-yl)piperidin-1-yl)-3-methylpyrrolidine-1-carboxylate

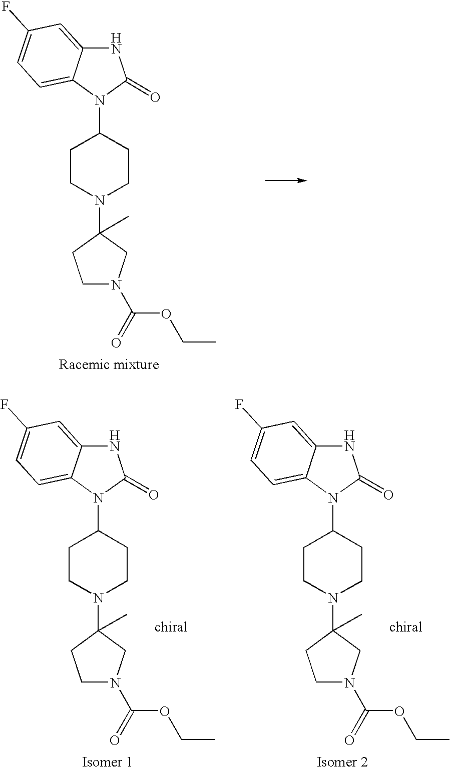

Enantiomers (isomer 1 and isomer 2) of ethyl 3-(4-(5-fluoro-2-oxo-2,3-dihydro-1H-benzo[d]imidazol-1-yl)piperidin-1-yl)-3-methylpyrrolidine-1-carboxylate (72 mg, 0.184 mmol) were separated by chiral column chromatography (Chiralpak AD column, 20% isopropyl alcohol/methanol 50/50 containing 0.1% diethylamine in hexane).

Isomer 1 of ethyl 3-(4-(5-fluoro-2-oxo-2,3-dihydro-1H-benzo[d]imidazol-1-yl)piperidin-1-yl)-3-methylpyrrolidine-1-carboxylate (Example 6) was the first fraction (0.030 g). Retention time: 13.62 minutes (ChiralPak AD, 7.5% EtOH/7.5% MeOH/85% hexane). 1H NMR (400 MHz, METHANOL-D4) δ ppm 1.09-1.15 (m, 3H) 1.17-1.28 (m, 3H) 1.75 (d, J=8.59 Hz, 2H) 1.84-2.00 (m, 2H) 2.34-2.62 (m, 4H) 2.75-2.86 (m, 1H) 3.00 (d, J=5.47 Hz, 1H) 3.23 (dd, J=9.96, 4.88 Hz, 1H) 3.31-3.48 (m, 2H) 3.50-3.62 (m, 1H) 4.09 (q, J=7.03 Hz, 2H) 4.17-4.30 (m, 1H) 6.73-6.84 (m, 2H) 7.31 (dd, J=8.79, 4.49 Hz, 1H). MS (M+1): 391.2.

Isomer 2 of ethyl 3-(4-(5-fluoro-2-oxo-2,3-dihydro-1H-benzo[d]imidazol-1-yl)piperidin-1-yl)-3-methylpyrrolidine-1-carboxylate (Example 7) was the second fraction (0.034 g). Retention time: 21.5 minutes (ChiralPak AD, 7.5% EtOH/7.5% MeOH/85% hexane). 1H NMR (400 MHz, METHANOL-D4) δ ppm 1.12 (s, 3H) 1.20-1.27 (m, 3H) 1.68-1.80 (m, 2H) 1.85-1.99 (m, 2H) 2.35-2.61 (m, 4H) 2.72-2.89 (m, 1H) 2.94-3.06 (m, 1H) 3.23 (dd, J=9.96, 4.88 Hz, 1H) 3.30-3.48 (m, 2H) 3.50-3.63 (m, 1H) 4.09 (q, J=7.03 Hz, 2H) 4.17-4.32 (m, 1H) 6.72-6.84 (m, 2H) 7.31 (dd, J=8.59, 4.30 Hz, 1H). MS (M+1): 391.2.

### Example 8

### methyl 3-(4-(5-fluoro-2-oxo-2,3-dihydro-1H-benzo[d]imidazol-1-yl)piperidin-1-yl)-3-methylpyrrolidine-1-carboxylate

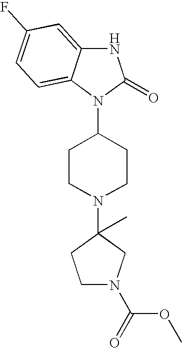

### Step A: Preparation of tert-butyl 3-(4-(2-amino-4-fluorophenylamino)piperidin-1-yl)-3-methylpyrrolidine-1-carboxylate

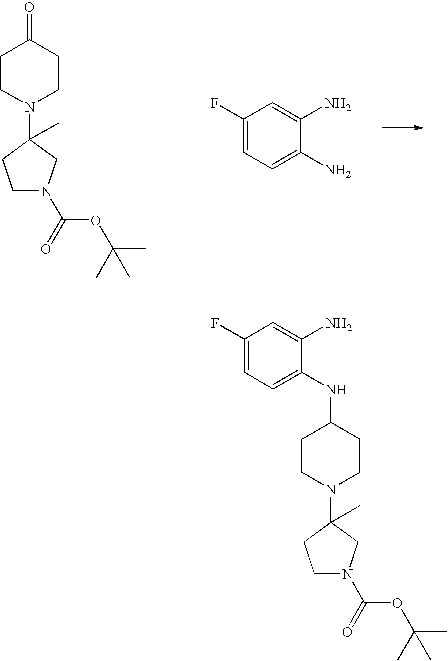

Following an analogous procedure to that described in the Step D of the Example 1, the title compound was made from tert-butyl 3-methyl-3-(4-oxopiperidin-1-yl)pyrrolidine-1-carboxylate (1.618 g, 5.73 mmol) and 4-fluorobenzene-1,2-diamine (0.723 g, 5.73 mmol). After the purification by high pH preparative HPLC (30-50% MeCN in water), the title compound was obtained as white solid (0.894 g, 39.8%). 1H NMR (400 MHz, CHLOROFORM-D) δ ppm 1.06 (d, J=3.52 Hz, 3H) 1.46 (s, 9H) 1.54-1.73 (m, 2H) 1.75-1.93 (m, 2H) 2.03 (d, J=12.89 Hz, 2H) 2.23-2.48 (m, 2H) 2.67 (d, J=10.94 Hz, 1H) 2.82 (dd, J=10.16, 2.73 Hz, 1 H) 3.04-3.22 (m, 2H) 3.25-3.38 (m, 1H) 3.40-3.69 (m, 3H) 6.38-6.49 (m, 2H) 6.60 (dd, J=7.81, 5.86 Hz, 1H). MS (M+1): 393.34.

### Step B: Preparation of tert-butyl 3-(4-(5-fluoro-2-oxo-2,3-dihydro-1H-benzo[d]imidazol-1-yl)piperidin-1-yl)-3-methylpyrrolidine-1-carboxylate

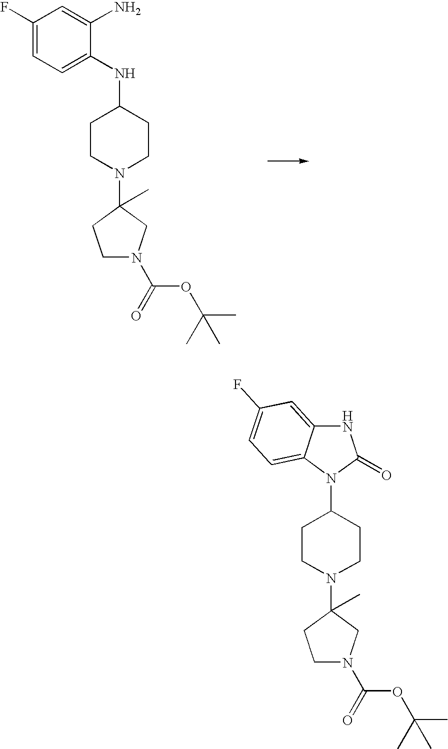

Following an analogous procedure to that described in the Step E of the Example 7, the title compound was made from tert-butyl 3-(4-(2-amino-4-fluorophenylamino)piperidin-1-yl)-3-methylpyrrolidine-1-carboxylate (0.894 g, 2.28 mmol). After the purification by high pH preparative HPLC (30-50% MeCN in water), the title compound was obtained as white solid (0.478 g, 50.1%). 1H NMR (400 MHz, METHANOL-D4) δ ppm 1.12 (s, 3H) 1.44 (s, 9H) 1.76 (d, J=8.98 Hz, 2H) 1.86-1.97 (m, 2H) 2.32-2.62 (m, 3H) 2.75-2.87 (m, 1H) 2.95-3.08 (m, 1H) 3.20 (t, J=11.13 Hz, 1H) 3.29-3.42 (m, 3H) 3.45-3.60 (m, 1H) 4.13-4.34 (m, 1H) 6.73-6.85 (m, 2H) 7.33 (dd, J=8.59, 4.30 Hz, 1H). MS (M+1): 419.15.

### Step C: Preparation of methyl 3-(4-(5-fluoro-2-oxo-2,3-dihydro-1H-benzo[d]imidazol-1-yl)piperidin-1-yl)-3-methylpyrrolidine-1-carboxylate

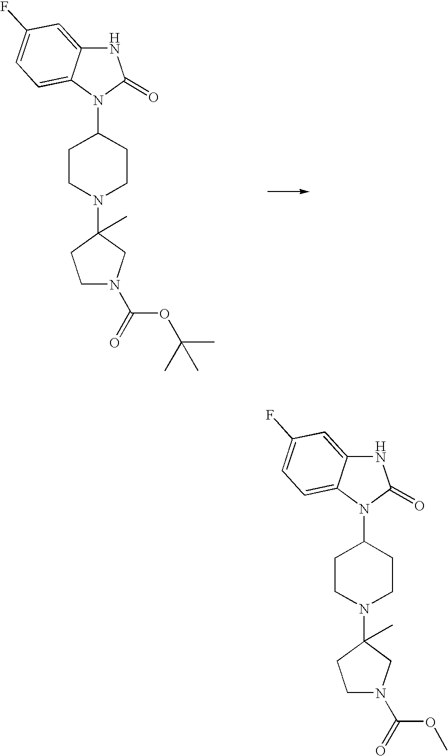

Following an analogous procedure to that described in the Step F of the Example 1, the title compound was made from tert-butyl 3-(4-(5-fluoro-2-oxo-2,3-dihydro-1H-benzo[d]imidazol-1-yl)piperidin-1-yl)-3-methylpyrrolidine-1-carboxylate (95.5 mg, 0.23 mmol) and methyl carbonochloridate (0.018 mL, 0.23 mmol). After the purification by high pH preparative HPLC (30-50% MeCN in water), the title compound was obtained as white solid (81 mg, 86%).1H NMR (400 MHz, METHANOL-D4) δ ppm 1.07-1.17 (m, 3H) 1.68-1.80 (m, 2H) 1.85-1.98 (m, 2H) 2.31-2.60 (m, 4H) 2.81 (t, J=8.59 Hz, 1H) 3.00 (dd, J=7.42, 1.95 Hz, 1H) 3.23 (d, J=10.55 Hz, 1 H) 3.32-3.46 (m, 2H) 3.52-3.61 (m, 1H) 3.66 (d, J=1.95 Hz, 3H) 4.09-4.32 (m, 1H) 6.72-6.86 (m, 2H) 7.31 (dd, J=8.59, 4.30 Hz, 1H). MS (M+1): 377.2, HRMS (M+1): 377.1985.

### Example 9

### isopropyl 3-(4-(5-fluoro-2-oxo-2,3-dihydro-1H-benzo[d]imidazol-1-yl)piperidin-1-yl)-3-methylpyrrolidine-1-carboxylate

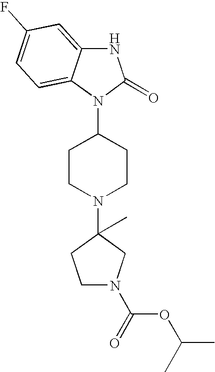

### Preparation of isopropyl 3-(4-(5-fluoro-2-oxo-2,3-dihydro-1H-benzo[d]imidazol-1-yl)piperidin-1-yl)-3-methylpyrrolidine-1-carboxylate

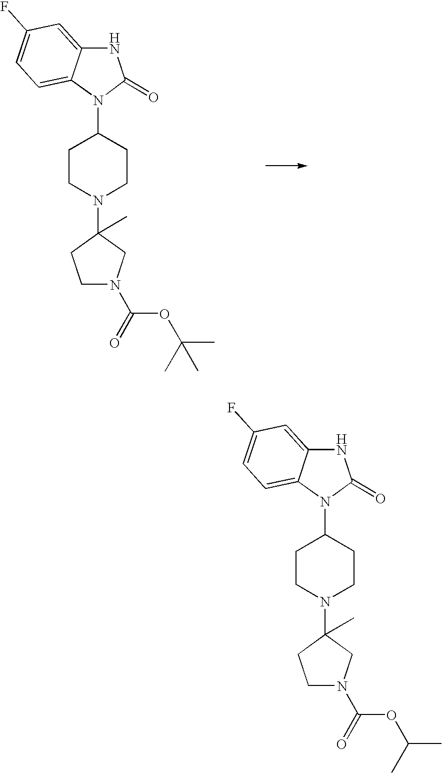

Following an analogous procedure to that described in the Step F of the Example 1, the title compound was made from tert-butyl 3-(4-(5-fluoro-2-oxo-2,3-dihydro-1H-benzo[d]imidazol-1-yl)piperidin-1-yl)-3-methylpyrrolidine-1-carboxylate (95.5 mg, 0.23 mmol) and isopropyl carbonochloridate (28.0 mg, 0.23 mmol). After the purification by high pH preparative HPLC (30-50% MeCN in water), the title compound was obtained as white solid (86 mg, 85%). 1H NMR (400 MHz, METHANOL-D4) δ ppm 1.11 (s, 3H) 1.18-1.28 (m, 6H) 1.67-1.83 (m, 2H) 1.87-2.01 (m, 2H) 2.34-2.62 (m, 4H) 2.74-2.87 (m, 1H) 2.97-3.06 (m, 1H) 3.22 (dd, J=10.35, 2.54 Hz, 1H) 3.31-3.45 (m, 2H) 3.50-3.63 (m, 1H) 4.16-4.31 (m, 1H) 4.78-4.90 (m, 1H) 6.73-6.84 (m, 2H) 7.31 (dd, J=8.59, 4.30 Hz, 1H). MS (M+1): 405.1, HRMS (M+1): 405.2294.

### Example 10

### 2-fluoroethyl 3-(4-(5-fluoro-2-oxo-2,3-dihydro-1H-benzo[d]imidazol-1-yl)piperidin-1-yl)-3-methylpyrrolidine-1-carboxylate

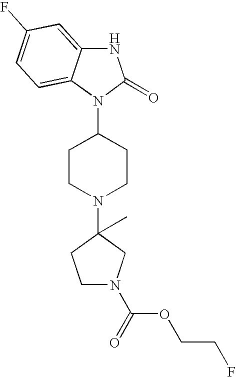

### Preparation of 2-fluoroethyl 3-(4-(5-fluoro-2-oxo-2,3-dihydro-1H-benzo[d]imidazol-1-yl)piperidin-1-yl)-3-methylpyrrolidine-1-carboxylate

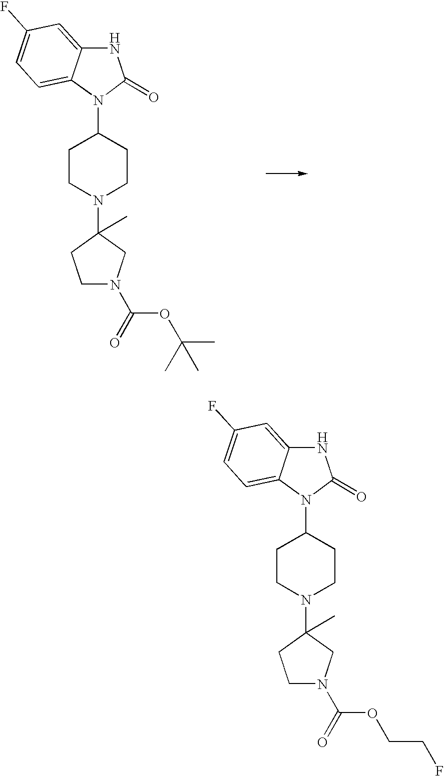

Following an analogous procedure to that described in the Step F of the Example 1, the title compound was made from tert-butyl 3-(4-(5-fluoro-2-oxo-2,3-dihydro-1H-benzo[d]imidazol-1-yl)piperidin-1-yl)-3-methylpyrrolidine-1-carboxylate (95.5 mg, 0.23 mmol) and 2-fluoroethyl carbonochloridate (0.022 ml, 0.23 mmol). After the purification by high pH preparative HPLC (20-40% MeCN in water), the title compound was obtained as white solid (100 mg, 99%). 1H NMR (400 MHz, METHANOL-D4) δ ppm 1.12 (s, 3H) 1.70-1.81 (m, 2H) 1.86-2.02 (m, 2H) 2.35-2.62 (m, 4H) 2.76-2.86 (m, 1H) 3.01 (dd, J=7.42, 1.95 Hz, 1H) 3.22-3.31 (m, 1H) 3.33-3.51 (m, 2H) 3.53-3.66 (m, 1H) 4.15-4.28 (m, 2H) 4.32 (dd, J=5.08, 3.12 Hz, 1H) 4.47-4.53 (m, 1H) 4.59-4.65 (m, 1H) 6.73-6.85 (m, 2H) 7.31 (dd, J=8.79, 4.49 Hz, 1H). MS (M+1): 409.2, HRMS (M+1): 409.2042.

### Example 11

### prop-2-ynyl 3-(4-(5-fluoro-2-oxo-2,3-dihydro-1H-benzo[d]imidazol-1-yl)piperidin-1-yl)-3-methylpyrrolidine-1-carboxylate

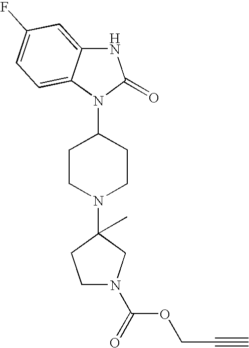

### Preparation of prop-2-ynyl 3-(4-(5-fluoro-2-oxo-2,3-dihydro-1H-benzo[d]imidazol-1-yl)piperidin-1-yl)-3-methylpyrrolidine-1-carboxylate

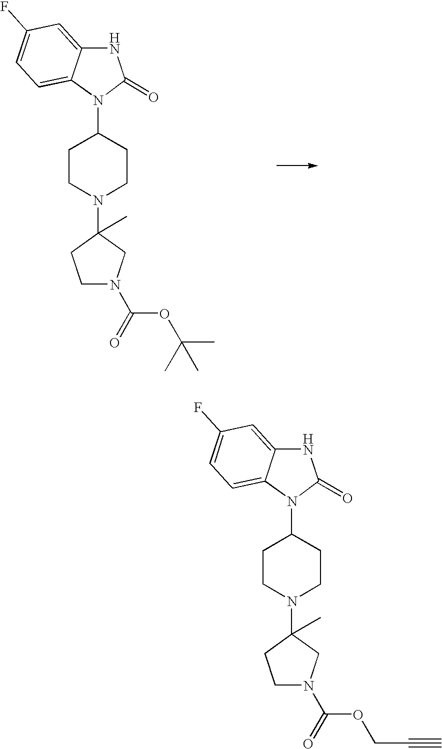

Following an analogous procedure to that described in the Step F of the Example 1, the title compound was made from tert-butyl 3-(4-(5-fluoro-2-oxo-2,3-dihydro-1H-benzo[d]imidazol-1-yl)piperidin-1-yl)-3-methylpyrrolidine-1-carboxylate (95.5 mg, 0.23 mmol) and prop-2-ynyl carbonochloridate (27 mg, 0.23 mmol). After the purification by high pH preparative HPLC (30-50% MeCN in water), the title compound was obtained as white solid (59.2 mg, 64.8%). 1H NMR (400 MHz, METHANOL-D4) δ ppm 1.13 (s, 3H) 1.71-1.80 (m, 2H) 1.85-2.05 (m, 2H) 2.31-2.63 (m, 4H) 2.82 (d, J=10.94 Hz, 1H) 2.89 (s, 1H) 3.01 (dd, J=7.23, 2.15 Hz, 1H) 3.22-3.31 (m, 1H) 3.33-3.51 (m, 2H) 3.52-3.67 (m, 1H) 4.11-4.30 (m, 1H) 4.67 (d, J=1.95 Hz, 2 H) 6.72-6.84 (m, 2H) 7.31 (dd, J=8.59, 4.30 Hz, 1H). MS (M+1): 401.2, HRMS (M+1): 401.1981.

### Example 12 (isomer 1) and Example 13 (Isomer 2)

### ethyl 3-methyl-3-(4-(5-methyl-2-oxo-2,3-dihydro-1H-benzo[d]imidazol-1-yl)piperidin-1-yl)pyrrolidine-1-carboxylate (enantiomers)

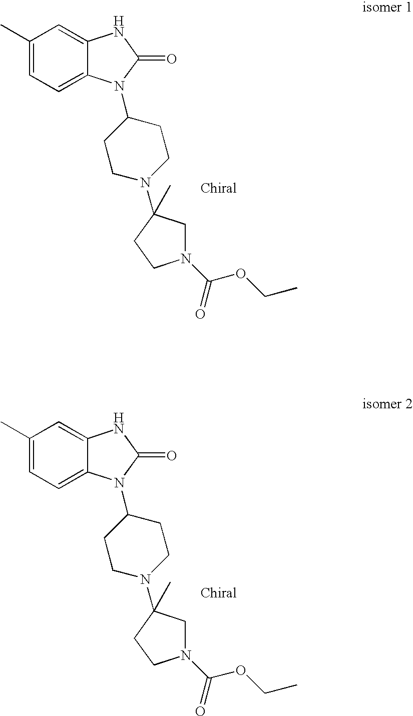

### Step A: Preparation of tert-butyl 3-(4-aminopiperidin-1-yl)-3-methylpyrrolidine-1-carboxylate

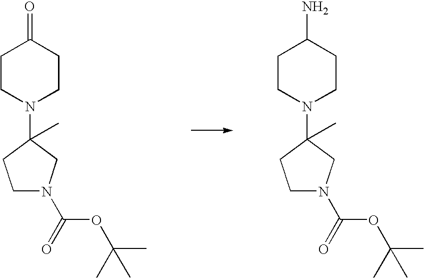

To a solution of tert-butyl 3-methyl-3-(4-oxopiperidin-1-yl)pyrrolidine-1-carboxylate (1 g, 3.54 mmol) in ethanol (15 mL) was added titanium(IV) isopropoxide (2.075 mL, 7.08 mmol) and ammonia (5.31 mL, 10.62 mmol). The reaction mixture was stirred at room temperature overnight. Sodium borohydride (0.201 g, 5.31 mmol) was added and stirred at room temperature overnight. 2N aqueous NaOH solution (4 mL) was added, stirred for 1 hour and precipitate was filtered through a pad of celite. The filtrate was concentrated in vacuo to give the title compound (1.480 g), which was used in the subsequent step without further purification. MS (M+1): 284.3.

### Step B: Preparation of tert-butyl 3-methyl-3-(4-(4-methyl-2-nitrophenylamino)piperidin-1-yl)pyrrolidine-1-carboxylate

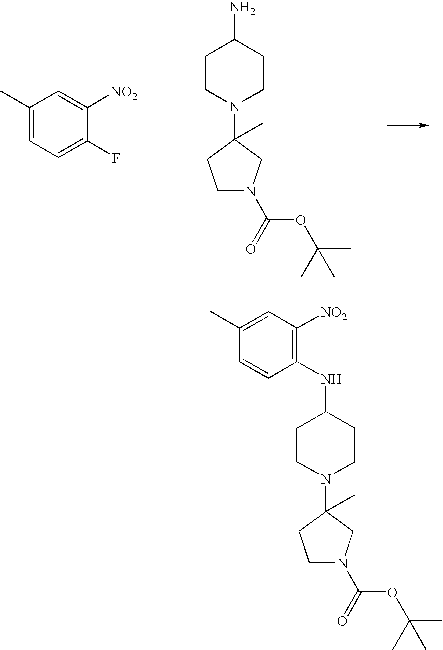

A mixture of 1-fluoro-4-methyl-2-nitrobenzene (411 mg, 2.65 mmol), tert-butyl 3-(4-aminopiperidin-1-yl)-3-methylpyrrolidine-1-carboxylate (500 mg, 1.76 mmol), and K₂CO₃ (244 mg, 1.76 mmol) in acetonitrile and water (3:1, 7 mL) was heated at 60° C. for 24 hours and heated further at 80° C. overnight. Concentrated in vacuo and the residue was purified by flash chromatography (60% to 70% ethyl acetate in Hexane) to provide the title compound (17.0 mg, 2.3%). MS (M+1): 419.4.

### Step C: Preparation of tert-butyl 3-(4-(2-amino-4-methylphenylamino)piperidin-1-yl)-3-methylpyrrolidine-1-carboxylate

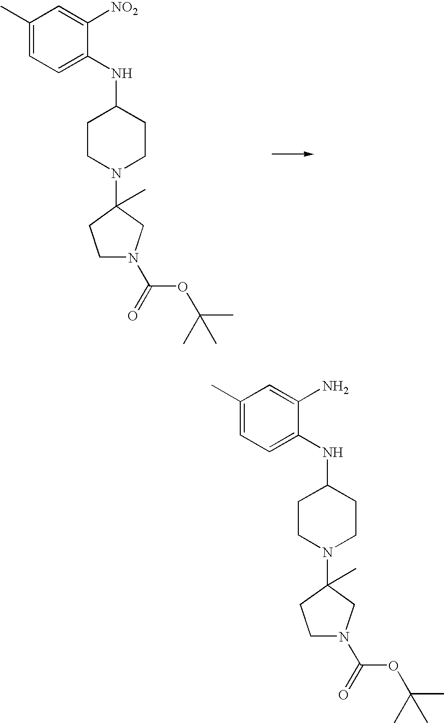

A solution of tert-butyl 3-methyl-3-(4-(4-methyl-2-nitrophenylamino)piperidin-1-yl)pyrrolidine-1-carboxylate (17 mg, 0.04 mmol) in MeOH (5 mL) was purged with nitrogen and added with 10% Pd/C (excess). The reaction mixture was purged with H₂ and was stirred under 40 psi H₂ atmosphere at room temperature overnight. Filtered through celite pad to remove the solids and the filtrate was concentrated in vacuo to give the title compound (36.1 mg), which was used in the subsequent step without further purification. MS (M+1): 388.9.

### Step D: Preparation of tert-butyl 3-methyl-3-(4-(5-methyl-2-oxo-2,3-dihydro-1H-benzo[d]imidazol-1-yl)piperidin-1-yl)pyrrolidine-1-carboxylate

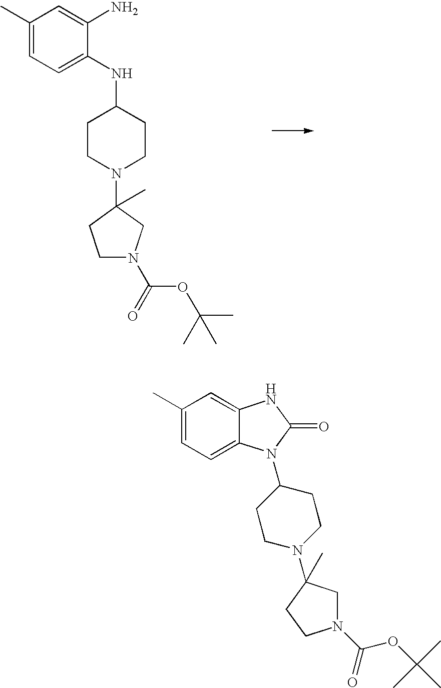

To a solution of tert-butyl 3-(4-(2-amino-4-methylphenylamino)piperidin-1-yl)-3-methylpyrrolidine-1-carboxylate (36.1 mg, 0.09 mmol) and DIPEA (catalytic amount) in dichloromethane (5 mL) at 0° C. was added triphosgene (13.82 mg, 0.05 mmol). The reaction mixture was allowed to warm to room temperature over 2.5 hours with stirring. Additional amount of triphosgene (13.82 mg, 0.05 mmol) was added and stirred at room temperature for another 2 hours. Aqueous solution of NaOH (2 mL, 2M) was added, stirred for 10 minutes and poured into Hydrometrix varian chem elut column. Column was rinsed with dichloromethane and concentrated in vacuo to give the title compound, which was used in the subsequent step without further purification (43.0 mg). MS (M+1): 414.8.

### Step E: Preparation of 5-methyl-1-(4-(3-methylpyrrolidin-3-yl)piperidin-1-yl)-1H-benzo[d]imidazol-2(3H)-one

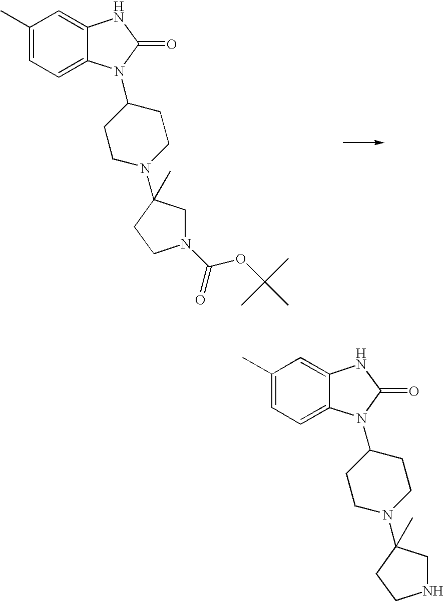

To a solution of tert-butyl 3-methyl-3-(4-(5-methyl-2-oxo-2,3-dihydro-1H-benzo[d]imidazol-1-yl)cyclohexyl)pyrrolidine-1-carboxylate (43 mg, 0.10 mmol) in MeOH (5 mL) was added 4M hydrogen chloride in dioxane (0.156 mL, 0.62 mmol). The reaction mixture was stirred at room temperature overnight. Additional amount of 4N HCl in dioxane (0.5 mL) was added and stirred for another 5 hours. Concentrated in vacuo to give the title compound (HCl salt, 66.9 mg), which was used in the subsequent step without further purification. MS (M+1): 314.9.

### Step F: Preparation of ethyl 3-methyl-3-(4-(5-methyl-2-oxo-2,3-dihydro-1H-benzo[d]imidazol-1-yl)piperidin-1-yl)pyrrolidine-1-carboxylate

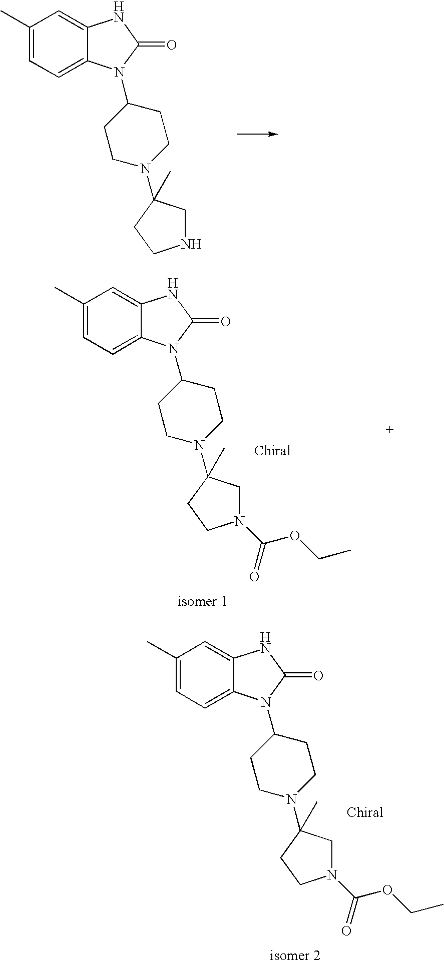

To a solution of 5-methyl-1-(4-(3-methylpyrrolidin-3-yl)cyclohexyl)-1H-benzo[d]imidazol-2(3H)-one and potassium carbonate (0.029 g, 0.21 mmol) in water (2.000 mL) was added a solution of ethyl carbonochloridate (0.02 mL, 0.21 mmol) in dichloromethane (2 mL). The reaction mixture was stirred at room temperature overnight. Aqueous solution of NaOH (2 mL, 2M) was added and stirred for 10 minutes. Poured into Hydrometrix varian chem elut column, rinsed with dichloromethane and concentrated in vacuo. The residue was purified by low pH preparative LC/MS (15%-35% MeCN in water) to give the racemic mixture of the title compound (TFA salt, 76.4 mg). Enantiomers (Isomer 1 and Isomer 2) were separated by chiral preparative HPLC (Chiralpak AD column, 1:140% iso propanol in methanol and heptane). Both enantiomers were further purified by low pH preparative LC/MS (15%-35% MeCN in water).

Isomer 1 of the title product (Example 12) (TFA salt, 3.20 mg, 6.09%) 1H NMR (400 MHz, METHANOL-D4) δ ppm 1.18-1.36 (m, 5H), 1.53 (s, 3H), 2.05-2.19 (m, 2H), 2.20-2.34 (m, 2H), 2.35 (s, 3H), 2.75-2.95 (m, 2H), 3.38-3.80 (m, 8H), 4.15 (q, J=7.03 Hz, 2H), 4.55 (tt, J=12.01, 4.30, 4.10 Hz, 1H), 6.84-6.96 (m, 2H), 7.16 (d, J=8.59 Hz, 1H) MS (M+1): 387.2 [M+H]+:387.23991.

Isomer 2 of the title product (Example 13) (TFA salt, 3.83 mg, 7.29%). 1H NMR (400 MHz, METHANOL-D4) δ ppm 1.15 (s, 3H), 1.21-1.33 (m, 5H), 1.78 (d, J=9.37 Hz, 2H), 1.91-2.04 (m, 2H), 2.34 (s, 3H), 2.39-2.47 (m, 1H), 2.49 (d, J=8.59 Hz, 2H), 2.51-2.63 (m, 1H), 2.79-2.90 (m, 1H), 2.96-3.08 (m, 1H), 3.37-3.44 (m, 1H), 3.47 (dd, J=9.96, 5.66 Hz, 1H), 3.52-3.72 (m, 2H), 4.12 (q, J=7.03 Hz, 2H), 4.20-4.34 (m, 1H), 6.81-6.97 (m, 2H), 7.26 (d, J=8.59 Hz, 1H) MS (M+1): 387.2 HRMS:387.23995.

### Example 14 (isomer 1)

### isopropyl 3-methyl-3-(4-(5-methyl-2-oxo-2,3-dihydro-1H-benzo[d]imidazol-1-yl)piperidin-1-yl)pyrrolidine-1-carboxylate(enantiomer 1)

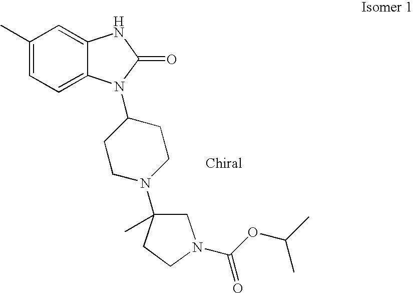

### Step A: Chiral separation of tert-butyl 3-methyl-3-(4-(5-methyl-2-oxo-2,3-dihydro-1H-benzo[d]imidazol-1-yl)piperidin-1-yl)pyrrolidine-1-carboxylate

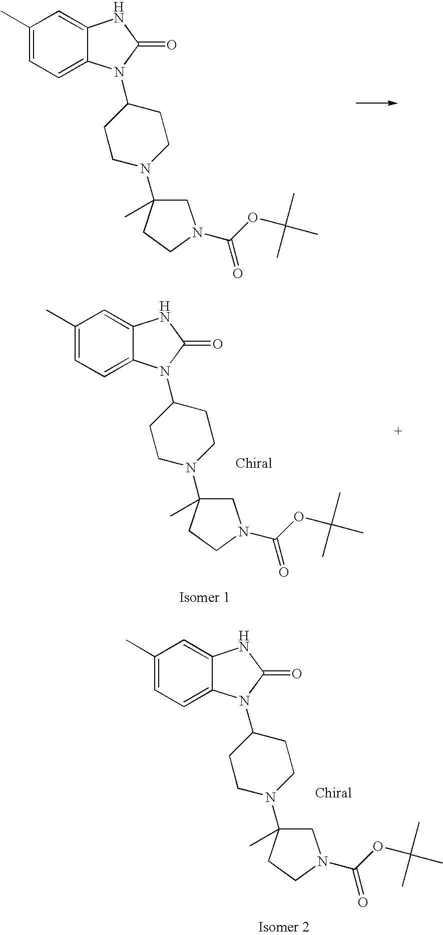

### Enantiomers (isomer 1 and isomer 2) of isopropyl 3-methyl-3-(4-(5-methyl-2-oxo-2,3-dihydro-1H-benzo[d]imidazol-1-yl)piperidin-1-yl)pyrrolidine-1-carboxylate (72 mg, 0.184 mmol) were separated by chiral super critical fluid chromatography (AS Column with 30% EtOH+0.1% N,N-dimethylethylamine, Main eluent: CO₂, Flow: 10 mL/min, Column Temperature: 35° C.).

Isomer 1 of isopropyl 3-methyl-3-(4-(5-methyl-2-oxo-2,3-dihydro-1H-benzo[d]imidazol-1-yl)piperidin-1-yl)pyrrolidine-1-carboxylate (retention time: 2.93 minutes): (26.8 mg, 83%). MS (M+1): 414.8.

Isomer 2 of isopropyl 3-methyl-3-(4-(5-methyl-2-oxo-2,3-dihydro-1H-benzo[d]imidazol-1-yl)piperidin-1-yl)pyrrolidine-1-carboxylate (retention time 4.08 minutes): (25.2 mg, 78%). MS (M+1): 414.8.

### Step B: Preparation of isopropyl 3-methyl-3-(4-(5-methyl-2-oxo-2,3-dihydro-1H-benzo[d]imidazol-1-yl)piperidin-1-yl)pyrrolidine-1-carboxylate (Isomer 1)

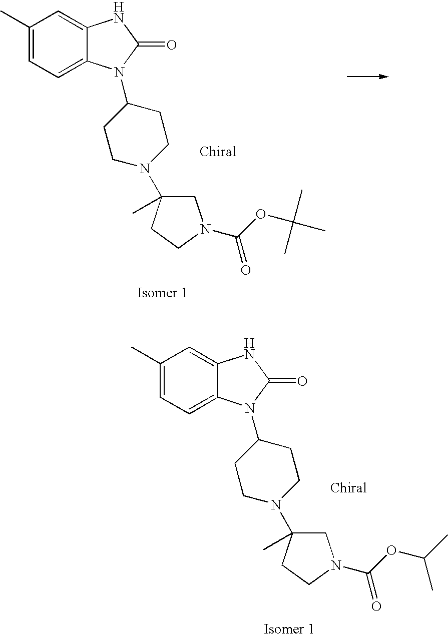

To a solution of isomer 1 of tert-butyl 3-methyl-3-(4-(5-methyl-2-oxo-2,3-dihydro-1H-benzo[d]imidazol-1-yl)piperidin-1-yl)pyrrolidine-1-carboxylate (26.8 mg, 0.06 mmol) in MeOH (1 mL) was added 4N HCl in dioxane (2 mL, 8.0 mmol). The reaction mixture was stirred at room temperature overnight and concentrated in vacuo. The residue dissolved in water (1.0 mL) and K₂CO₃ (17.87 mg, 0.13 mmol) was added. A solution of 1M isopropyl carbonochloridate in toluene (0.065 mL, 0.06 mmol) and dichloromethane (1.0 mL) was added and stirred at room temperature for 24 hours. Aqueous solution of NaOH (2 mL, 2M) was added and stirred at room temperature for 1 hour. Poured into Hydrometrix varian chem elut column, rinsed with dichloromethane and concentrated in vacuo. The residue was purified by low pH preparative LC/MS (25%-45% MeCN in water) to give the title compound (TFA salt, 31.1 mg, 93%).1H NMR (400 MHz, METHANOL-D4) δ ppm 1.26 (d, J=6.25 Hz, 6H), 1.54 (s, 3H), 2.00-2.19 (m, 2H), 2.20-2.37 (m, 2H), 2.39 (s, 3H), 2.88 (qd, J=13.22, 4.10 Hz, 2H), 3.39-3.57 (m, 5H), 3.56-3.81 (m, 4H), 4.58 (tt, J=12.21, 4.10, 3.91 Hz, 1H), 6.88-6.93 (m, 1H), 6.93-6.98 (m,1H), 7.12 (s, 1H) MS (M+1): 401.2, HRMS (M+1): 401.25473.

### Example 15 (Isomer 2)

### isopropyl 3-methyl-3-(4-(5-methyl-2-oxo-2,3-dihydro-1H-benzo[d]imidazol-1-yl)piperidin-1-yl)pyrrolidine-1-carboxylate(enantiomer 2)

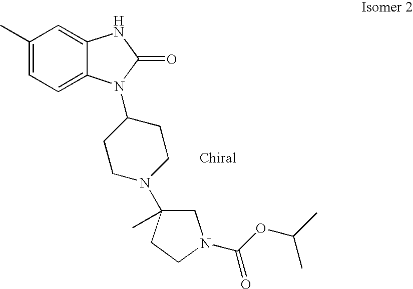

### Preparation of isopropyl 3-methyl-3-(4-(5-methyl-2-oxo-2,3-dihydro-1H-benzo[d]imidazol-1-yl)piperidin-1-yl)pyrrolidine-1-carboxylate (enantiomer 2)

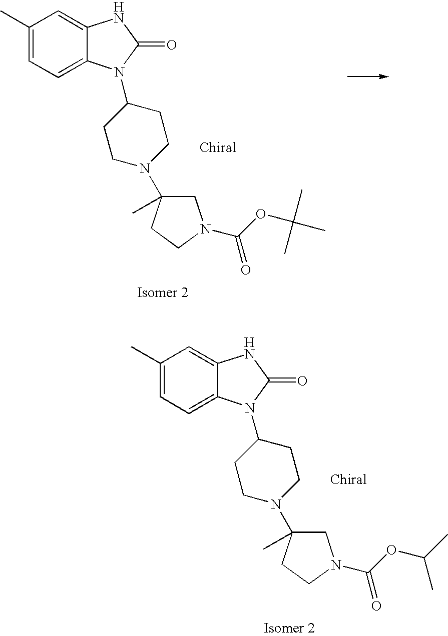

Following an analogous procedure to that described in the Step B of the Example 14, the title compound was made from isomer 2 of tert-butyl 3-methyl-3-(4-(5-methyl-2-oxo-2,3-dihydro-1H-benzo[d]imidazol-1-yl)piperidin-1-yl)pyrrolidine-1-carboxylate (25.2 mg, 0.06 mmol) and isopropyl carbonochloridate (0.061 mL, 0.06 mmol, 1M in toluene). The residue was purified by low pH preparative LC/MS (25%-45% MeCN in water) to give the title compound (TFA salt, 24.30 mg, 78%). 1H NMR (400 MHz, Methanol-D4) δ ppm 1.27 (d, J=6.25 Hz, 6H), 1.54 (s, 3H), 2.13 (d, J=7.81 Hz, 2H), 2.27 (s, 2H), 2.39 (s, 3H), 2.78-2.98 (m, 2H), 3.39-3.81 (m, 9H), 4.49-4.63 (m, 1H), 6.87-6.92 (m, 1H), 6.93-7.00 (m, 1H), 7.12 (s, 1H). MS (M+1): 401.2, HRMS (M+1): 401.25432.

### Example 16

### ethyl 3-methyl-3-(4-(6-methyl-2-oxo-2,3-dihydro-1H-benzo[d]imidazol-1-yl)piperidin-1-yl)pyrrolidine-1-carboxylate

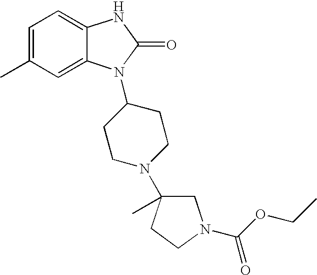

### Step A: Preparation of tert-butyl 3-methyl-3-(4-(6-methyl-2-oxo-2,3-dihydro-1H-benzo[d]imidazol-1-yl)piperidin-1-yl)pyrrolidine-1-carboxylate

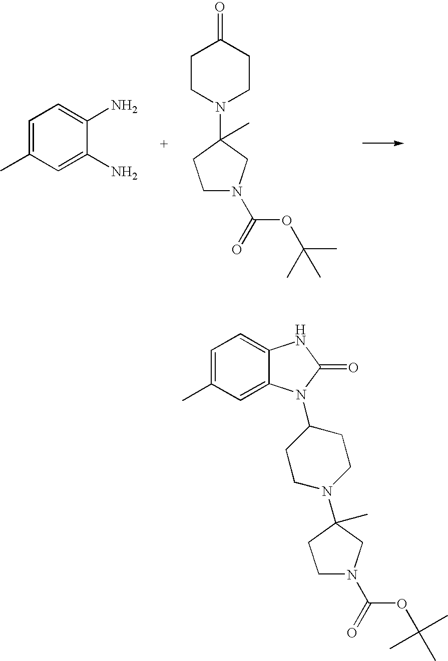

Sodium triacetoxyborohydride (37.5 mg, 0.18 mmol) was added to a mixture of tert-butyl 3-methyl-3-(4-oxopiperidin-1-yl)pyrrolidine-1-carboxylate (50 mg, 0.18 mmol) and 4-methylbenzene-1,2-diamine (108 mg, 0.89 mmol) in dichloromethane (5 mL). The resulting mixture was stirred at room temperature overnight. An aqueous solution of 2N NaOH (2 mL) was added and stirred for 5 minutes. The reaction mixture was concentrated to dryness. The residue was dissolved in dichloromethane (10 mL) and DIPEA (0.046 mL, 0.27 mmol) was added to the solution. The solution was cooled to 0° C. Triphosgene (26.3 mg, 0.09 mmol) was added to the solution and stirred for 2 hours. The reaction mixture was allowed to warm to room temperature and additional amount of triphosgene (26.3 mg, 0.09 mmol) was added to the reaction mixture. The resulting mixture was stirred at room temperature for another 2 hours. 2N NaOH aqueous solution was added to the reaction mixture and poured into Hydrometrix column. The eluent was concentrated under reduced pressure. The crude product was purified by flash chromatography to give a mixture of tert-butyl 3-methyl-3-(4-(6-methyl-2-oxo-2,3-dihydro-1H-benzo[d]imidazol-1-yl)piperidin-1-yl)pyrrolidine-1-carboxylate and tert-butyl 3-methyl-3-(4-(5-methyl-2-oxo-2,3-dihydro-1H-benzo[d]imidazol-1-yl)piperidin-1-yl)pyrrolidine-1-carboxylate (24.2 mg). The mixture was purified further by chiral HPLC (Chiralpak AD column, 20% EtOH/80% Hexane) to give the title compound.

### Step B: Preparation of 6-methyl-1-(1-(3-methylpyrrolidin-3-yl)piperidin-4-yl)-1H-benzo[d]imidazol-2(3H)-one

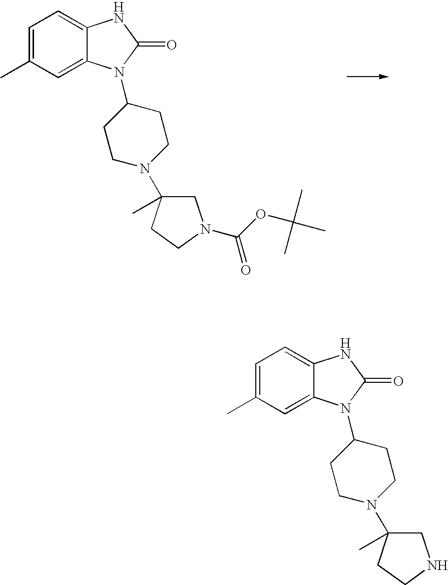

4N HCl in dioxane (0.6 mL, 2.40 mmol) was added to a mixture of tert-butyl 3-methyl-3-(4-(6-methyl-2-oxo-2,3-dihydro-1H-benzo[d]imidazol-1-yl)piperidin-1-yl)pyrrolidine-1-carboxylate (159.7 mg, 0.39 mmol) in methanol (3 mL). The resulting mixture was stirred at room temperature overnight. Concentrated under reduced pressure to give the title compound (HCl salt, 174 mg). The crude product was used in the subsequent step without further purification. MS (M+1): 314.9.

### Step C: Preparation of ethyl 3-methyl-3-(4-(6-methyl-2-oxo-2,3-dihydro-1H-benzo[d]imidazol-1-yl)piperidin-1-yl)pyrrolidine-1-carboxylate

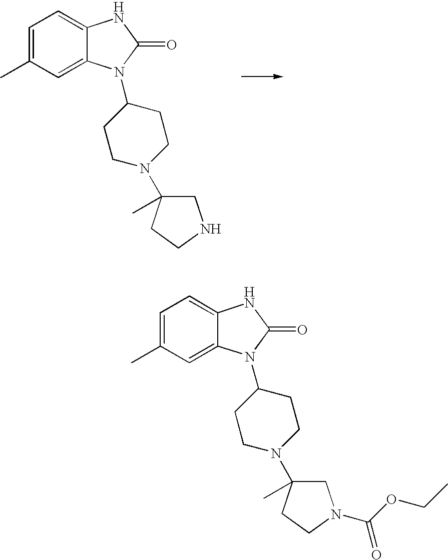

Following an analogous procedure to that described in Step F of the Example 12, the title compound was made from 6-methyl-1-(1-(3-methylpyrrolidin-3-yl)piperidin-4-yl)-1H-benzo[d]imidazol-2(3H)-one (HCl salt, 87 mg, 0.25 mmol) and ethyl carbonochloridate (0.035 mL, 0.37 mmol, 1M in toluene). The residue was purified by low pH preparative LC/MS (15%-35% MeCN in water) to give the title compound (TFA salt, 7.20 mg, 5.80%). 1H NMR (400 MHz, METHANOL-D4) δ ppm 1.28 (t, J=7.23 Hz, 3H), 1.55 (s, 3H), 2.01-2.19 (m, 2H), 2.20-2.33 (m, 2H), 2.36 (s, 3H), 2.78-2.96 (m, 2H), 3.41-3.57 (m, 5H), 3.57-3.81 (m, 4H), 4.16 (q, J=7.03 Hz, 2H), 4.48-4.63 (m,1H), 6.88-6.97 (m, 2H), 7.16 (d, J=8.59 Hz, 1H). MS (M+1):387.2, HRMS (M+1): 387.23876.

### Example 17

### methyl 3-methyl-3-(4-(6-methyl-2-oxo-2,3-dihydro-1H-benzo[d]imidazol-1-yl)piperidin-1-yl)pyrrolidine-1-carboxylate

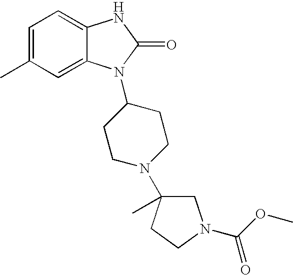

### Preparation of methyl 3-methyl-3-(4-(6-methyl-2-oxo-2,3-dihydro-1H-benzo[d]imidazol-1-yl)piperidin-1-yl)pyrrolidine-1-carboxylate

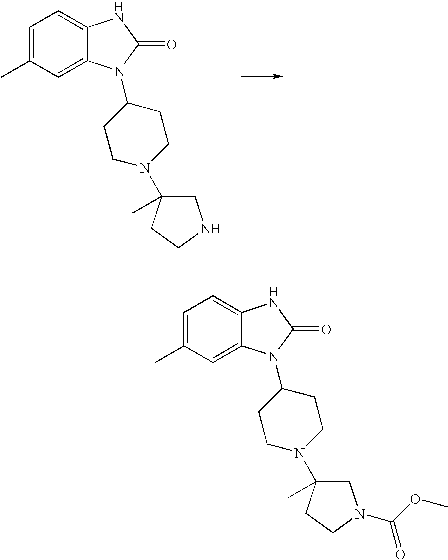

Following an analogous procedure to that described in Step F of the Example 12, the title compound was made from 6-methyl-1-(1-(3-methylpyrrolidin-3-yl)piperidin-4-yl)-1H-benzo[d]imidazol-2(3H)-one (HCl salt, 87 mg, 0.25 mmol) and methyl carbonochloridate (0.029 mL, 0.37 mmol). The residue was purified by low pH preparative LC/MS (15%-35% MeCN in water) to give the title compound (TFA salt, 62.3 mg, 51.6%). 1H NMR (400 MHz, MeOD₄) δ ppm 1.53 (s, 3H), 2.05-2.15 (m, 2H), 2.19-2.29 (m, 1H), 2.31-2.47 (m, 1H), 2.34 (s, 3H), 2.74-2.99 (m, 2H), 3.38-3.58 (m, 4H), 3.59-3.79 (m, 4H), 3.71 (s, 3H), 4.49-4.68 (m, 1 H), 6.84-6.93 (m, 2H), 7.20 (d, J=7.81 Hz, 1H). MS (M+1): 373.3, HRMS (M+1): 373.22394.

### Example 18 (Isomer 1), Example 19 (Isomer 2), Example 20 (Isomer 3) and Example 21 (Isomer 4)

### Enantiomers of but-2-ynyl 3-methyl-3-(4-(6-methyl-2-oxo-2,3-dihydro-1H-benzo[d]imidazol-1-yl)piperidin-1-yl)pyrrolidine-1-carboxylate (Isomer 1 and Isomer 3) and enantiomers of but-2-ynyl 3-methyl-3-(4-(5-methyl-2-oxo-2,3-dihydro-1H-benzo[d]imidazol-1-yl)piperidin-1-yl)pyrrolidine-1-carboxylate (Isomer 2 and Isomer 4)

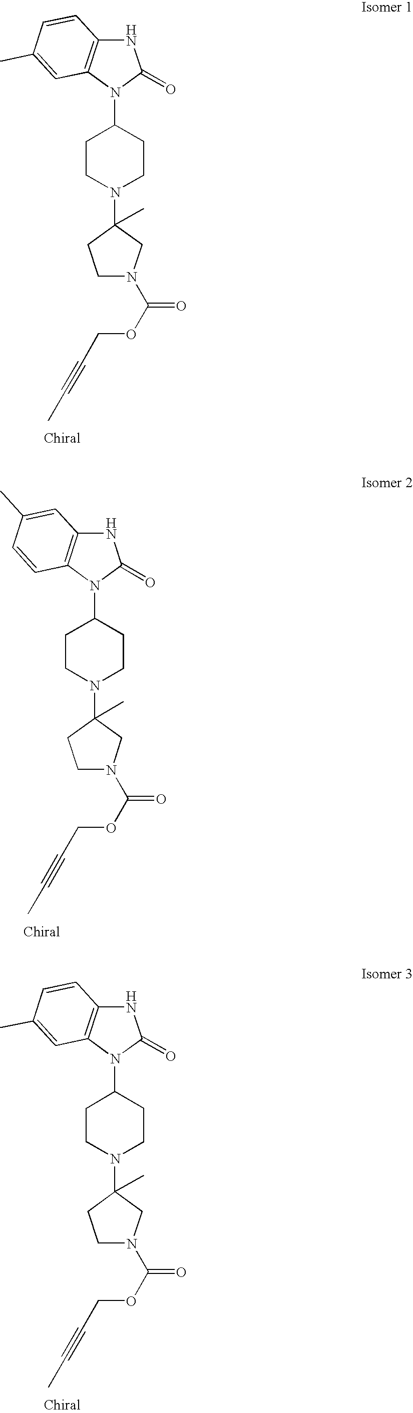

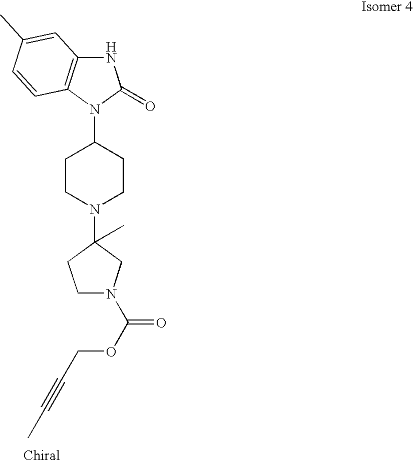

### Step A: Preparation of tert-butyl 2-amino-4-methylphenylcarbamate and tert-butyl 2-amino-5-methylphenylcarbamate

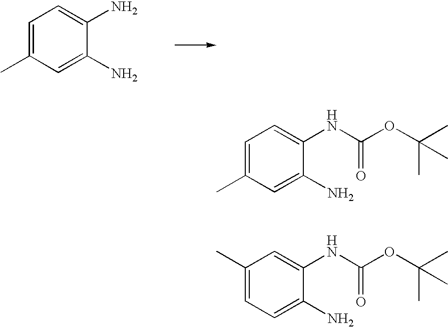

A solution of di-tert-butyl dicarbonate (3.39 g, 15.55 mmol) in dichloromethane (20 mL) was added slowly to a mixture of 4-methylbenzene-1,2-diamine (2 g, 16.37 mmol) in water (40.0 mL). The reaction mixture was stirred at room temperature overnight. Layers were separated and aqueous layer was extracted with dichloromethane. Combined organic extract was dried, filtered and concentrated in vacuo. The residue was purified by flash chromatography (20% ethylacetate in heptane) to give the title product as a mixture of regioisomers (2.53 g, 69.5%). MS (M+1): 223.

### Step B: Preparation of tert-butyl 3-(4-(2-(tert-butoxycarbonylamino)-5-methylphenylamino)piperidin-1-yl)-3-methylpyrrolidine-1-carboxylate and tert-butyl 3-(4-(2-(tert-butoxycarbonylamino)-4-methylphenylamino)piperidin-1-yl)-3-methylpyrrolidine-1-carboxylate (mixture)

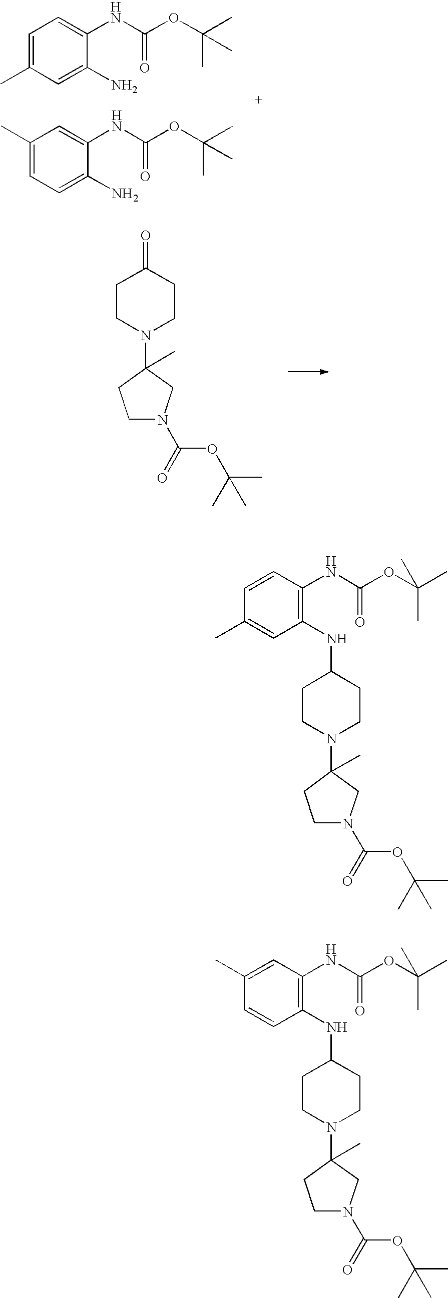

A solution of tert-butyl 2-amino-4-methylphenylcarbamate and tert-butyl 2-amino-5-methylphenylcarbamate (mixture of two isomers) (2.53 g, 11.38 mmol) and tert-butyl 3-methyl-3-(4-oxopiperidin-1-yl)pyrrolidine-1-carboxylate (3.21 g, 11.38 mmol) in MeOH (20 mL) was stirred at room temperature for 5 minutes. A solution of sodium cyanoborohydride (0.755 g, 12.01 mmol) and zinc chloride (0.786 g, 5.77 mmol) in methanol (20.0 mL) was then added. The reaction mixture was stirred at room temperature overnight. Additional amount of sodium cyanoborohydride (0.755 g, 12.01 mmol) was added and stirred at room temperature for 6 hours. Concentrated under reduced pressure, the residue dissolved in ethyl acetate and washed with 2N NaOH. Aqueous layer was extracted with ethyl acetate. Combined organic extract was dried, filtered and concentrated in under reduced pressure to give the title compounds as a mixture of two isomers (5.84 g), which was used in the subsequent step without further purification. MS (M+1): 489.3.

### Step C: Preparation of tert-butyl 3-methyl-3-(4-(6-methyl-2-oxo-2,3-dihydro-1H-benzo[d]imidazol-1-yl)piperidin-1-yl)pyrrolidine-1-carboxylate and 3-methyl-3-(4-(5-methyl-2-oxo-2,3-dihydro-1H-benzo[d]imidazol-1-yl)piperidin-1-yl)pyrrolidine-1-carboxylate (mixture)

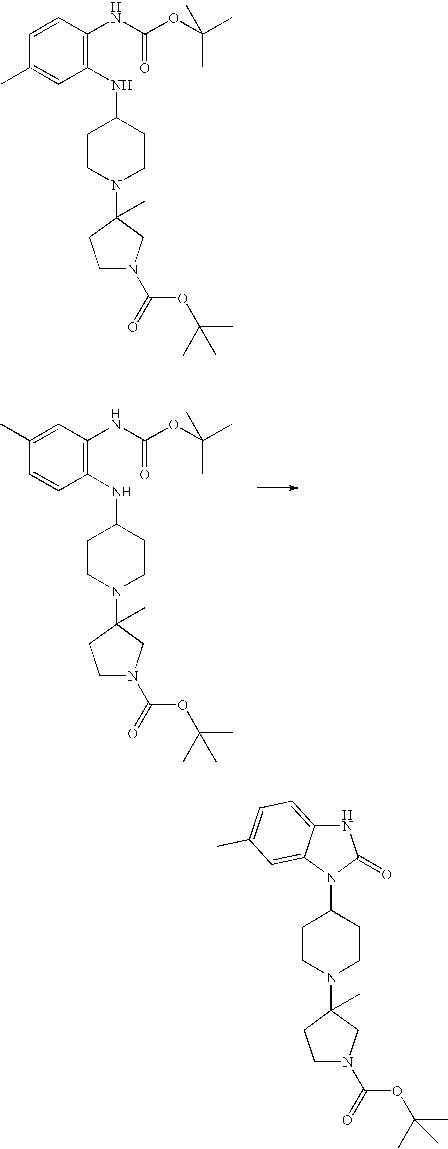

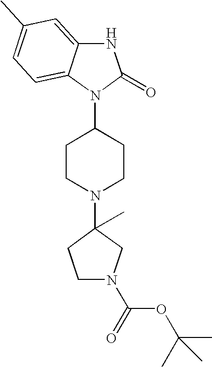

Potassium tert-butoxide (4 g, 35.65 mmol) was added to a solution of crude tert-butyl 3-(4-(2-(tert-butoxycarbonylamino)-5-methylphenylamino)piperidin-1-yl)-3-methylpyrrolidine-1-carboxylate and tert-butyl 3-(4-(2-(tert-butoxycarbonylamino)-4-methylphenylamino)piperidin-1-yl)-3-methylpyrrolidine-1-carboxylate (mixture of isomers from previous step) in THF (15 mL). The reaction mixture was heated at reflux for 5 hours. Additional amount of potassium tert-butoxide (4 g, 35.65 mmol) was added and heated at reflux for 5 hours. The reaction was cooled to room temperature. Water (20 mL) was added and stirred for 5 minutes at room temperature. Layers were separated, and aqueous layer was extracted with ethyl acetate. Combined organic extract was dried, filtered and concentrated in under reduced pressure. The residue was purified by flash chromatography (10% Methanol in dichloromethane) to give the title compound as a mixture of two isomers (2.103 g, 44.6%). MS (M+1): 415.4.

### Step D: Preparation of 6-methyl-1-(1-(3-methylpyrrolidin-3-yl)piperidin-4-yl)-1H-benzo[d]imidazol-2(3H)-one and 5-methyl-1-(4-(3-methylpyrrolidin-3-yl)piperidin-1-yl)-1H-benzo[d]imidazol-2(3H)-one (mixture)

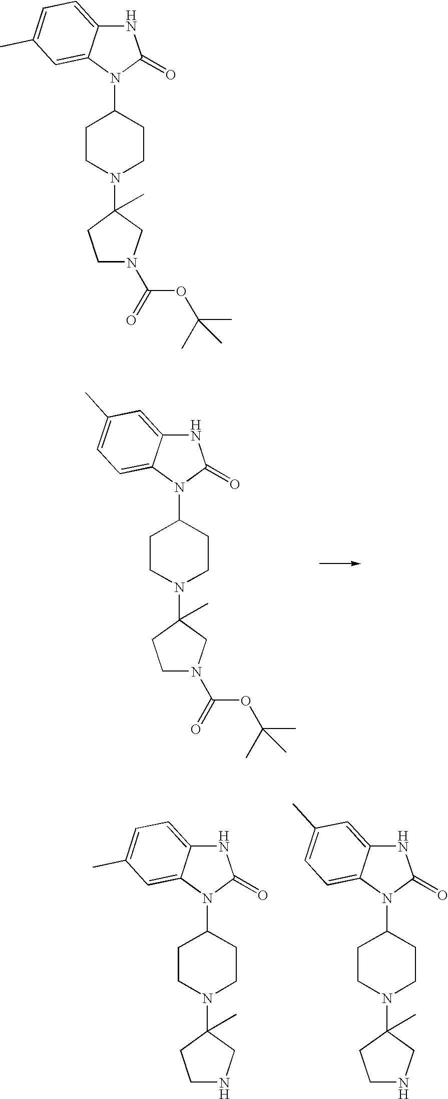

4N HCl in dioxane (15.26 mL, 61.02 mmol) was added to a mixture of tert-butyl 3-methyl-3-(4-(6-methyl-2-oxo-2,3-dihydro-1H-benzo[d]imidazol-1-yl)piperidin-1-yl)pyrrolidine-1-carboxylate and 3-methyl-3-(4-(5-methyl-2-oxo-2,3-dihydro-1H-benzo[d]imidazol-1-yl)piperidin-1-yl)pyrrolidine-1-carboxylate (mixture of isomers from previous step) (2.108 g, 5.09 mmol). The reaction mixture was stirred at room temperature overnight. Concentrated in under reduced pressure to give the title compound mixture (HCl salt, 2.82 g, 158%), which was used in the subsequent step without further purification. MS (M+1): 315.3.

### Step E. The preparation of racemic but-2-ynyl 3-methyl-3-(4-(6-methyl-2-oxo-2,3-dihydro-1H-benzo[d]imidazol-1-yl)piperidin-1-yl)pyrrolidine-1-carboxylate and but-2-ynyl 3-methyl-3-(4-(5-methyl-2-oxo-2,3-dihydro-1H-benzo[d]imidazol-1-yl)piperidin-1-yl)pyrrolidine-1-carboxylate (mixture)

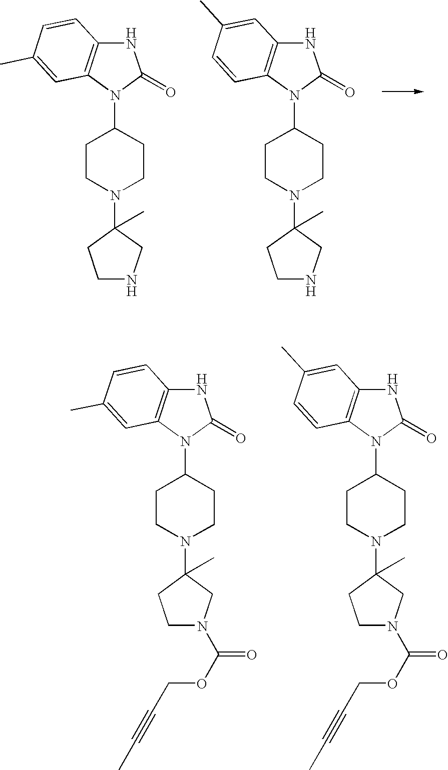

A mixture of crude 6-methyl-1-(1-(3-methylpyrrolidin-3-yl)piperidin-4-yl)-1H-benzo[d]imidazol-2(3H)-one (HCl salt) and 5-methyl-1-(1-(3-methylpyrrolidin-3-yl)piperidin-4-yl)-1H-benzo[d]imidazol-2(3H)-one (HCl salt) (6:4) (0.4819 g, 1.37 mmol) and K₂CO₃ (0.190 g, 1.37 mmol) in water (3.00 mL) was stirred for 5 minutes at 40° C. under nitrogen. A solution of but-2-ynyl carbonochloridate (0.171 mL, 1.51 mmol) in DCM (3 mL) was dropwise added. The mixture was stirred at room temperature for 2 hours and phases were separated. The aqueous phase was extracted with dichloromethane. The organic phases were combined, dried, filtered and concentrated under reduced pressure. The residue was purified by preperative LC/MS (high pH, 35-55% CH₃CN) to afford the mixture of but-2-ynyl 3-methyl-3-(4-(6-methyl-2-oxo-2,3-dihydro-1H-benzo[d]imidazol-1-yl)piperidin-1-yl)pyrrolidine-1-carboxylate and but-2-ynyl 3-methyl-3-(4-(5-methyl-2-oxo-2,3-dihydro-1H-benzo[d]imidazol-1-yl)piperidin-1-yl)pyrrolidine-1-carboxylate (6:4) (0.182 g, 32.2%) MS (M+1):411.4

### Step F. Separation of enantiomers of but-2-ynyl 3-methyl-3-(4-(6-methyl-2-oxo-2,3-dihydro-1H-benzo[d]imidazol-1-yl)piperidin-1-yl)pyrrolidine-1-carboxylate (Isomer s 1 & 3) and enantiomers of but-2-ynyl 3-methyl-3-(4-(5-methyl-2-oxo-2,3-dihydro-1H-benzo[d]imidazol-1-yl)piperidin-1-yl)pyrrolidine-1-carboxylate (Isomers 2 & 4)

The mixture of isomers was separated by chiral SFC (OJ column, 15% EtOH/0.1% DMEA/CO₂) to afford 4 isomers:

Isomer 1 (Example 18): Enantiomer 1 of but-2-ynyl 3-methyl-3-(4-(6-methyl-2-oxo-2,3-dihydro-1H-benzo[d]imidazol-1-yl)piperidin-1-yl)pyrrolidine-1-carboxylate (0.036 g). Retention time: 3.05 minutes (Chiral SFC, OJ Column, 20% EtOH/0.1% DMEA/CO₂) (1H NMR (400 MHz, Methanol-D4) δ ppm 1.17 (s, 3H), 1.73-1.86 (m, 5H), 1.92-2.07 (m, 2H), 2.38 (s, 3H), 2.43-2.66 (m, 4H), 2.87 (d, J=10.16 Hz, 1H), 3.05 (s, 1H), 3.35-3.54 (m, 3H), 3.55-3.69 (m, 1H), 4.21-4.36 (m, 1H), 4.64 (d, J=1.95 Hz, 2H), 6.87 (d, J=7.80 Hz 1H), 6.93 (d, J=7.80 Hz, 1H), 7.27 (s, 1H). HRMS [M+1]: 411.23860.

Isomer 2 (Example 19): Enantiomer 1 of but-2-ynyl 3-methyl-3-(4-(5-methyl-2-oxo-2,3-dihydro-1H-benzo[d]imidazol-1-yl)piperidin-1-yl)pyrrolidine-1-carboxylate (0.025 g). Retention time: 3.29 minutes (Chiral SFC, OJ Column, 20% EtOH/0.1% DMEA/CO₂). 1HNMR (400 MHz, Methanol-D4) δ ppm 1.22 (s, 3H), 1.73-1.91 (m, 5H), 1.95-2.08 (m, 2H), 2.34 (s, 3H), 2.43-2.80 (m, 4H), 2.87-2.99 (m, 1H), 3.05-3.21 (m, 1H), 3.37-3.57 (m, 3H), 3.58-3.68 (m, 1H), 4.29 (s, 1H), 4.65 (s, 2H), 6.84-6.93 (m, 2H) 7.24 (d, J=8.20 Hz, 1H). HRMS [M+1]: 411.23848.

Isomer 3 (Example 20): Enantiomer 2 of but-2-ynyl 3-methyl-3-(4-(6-methyl-2-oxo-2,3-dihydro-1H-benzo[d]imidazol-1-yl)piperidin-1-yl)pyrrolidine-1-carboxylate (0.044 g). Retention time: 3.60 minutes (Chiral SFC, OJ Column, 20% EtOH/0.1% DMEA/CO₂). 1HNMR (400 MHz, Methanol-D4) δ ppm 1.17 (s, 3H), 1.71-1.85 (m, 5H), 1.93-2.03 (m, 2H), 2.38 (s, 3H), 2.43-2.67 (m, 4H), 2.87 (d, J=10.16 Hz, 1H), 3.05 (s, 1H), 3.34-3.53 (m, 3H), 3.56-3.67 (m, 1H), 4.16-4.36 (m, 1H), 4.64 (d, J=1.95 Hz, 2H), 6.87 (d, J=8.20 Hz, 1H), 6.93 (d, J=8.20 Hz, 1H), 7.27 (s, 1H). HRMS [M+1]: 411.23927.

Isomer 4 (Example 21): Enantiomer 2 of but-2-ynyl 3-methyl-3-(4-(5-methyl-2-oxo-2,3-dihydro-1H-benzo[d]imidazol-1-yl)piperidin-1-yl)pyrrolidine-1-carboxylate (0.028 g). Retention time: 4.25 minutes (Chiral SFC, OJ Column, 20% EtOH/0.1% DMEA/CO₂). 1H NMR (400 MHz, Methanol-D4) δ ppm 1.16 (s, 3H), 1.70-1.86 (m, 5H), 1.91-2.05 (m, 2H), 2.34 (s, 3H), 2.40-2.65 (m, 4H), 2.85 (d, J=10.16 Hz, 1H), 3.01-3.09 (m, 1H), 3.35-3.53 (m, 3H), 3.54-3.72 (m, 1H), 4.15-4.35 (m, 1H), 4.64 (s, 2H), 6.82-6.95 (m, 2H), 7.26 (d, J=8.59 Hz, 1H). HRMS [M+1]: 411.23886.

### Example 22 (Isomer 1)

### but-2-ynyl 3-(4-(5-fluoro-2-oxo-2,3-dihydro-1H-benzo[d]imidazol-1-yl)piperidin-1-yl)-3-methylpyrrolidine-1-carboxylate

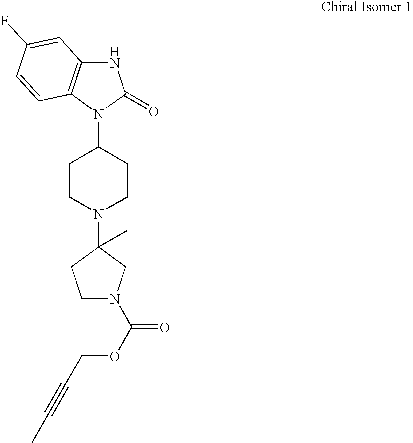

### Step A: Separation of enantiomers of tert-butyl 3-(4-(5-fluoro-2-oxo-2,3-dihydro-1H-benzo[d]imidazol-1-yl)piperidin-1-yl)-3-methylpyrrolidine-1-carboxylate

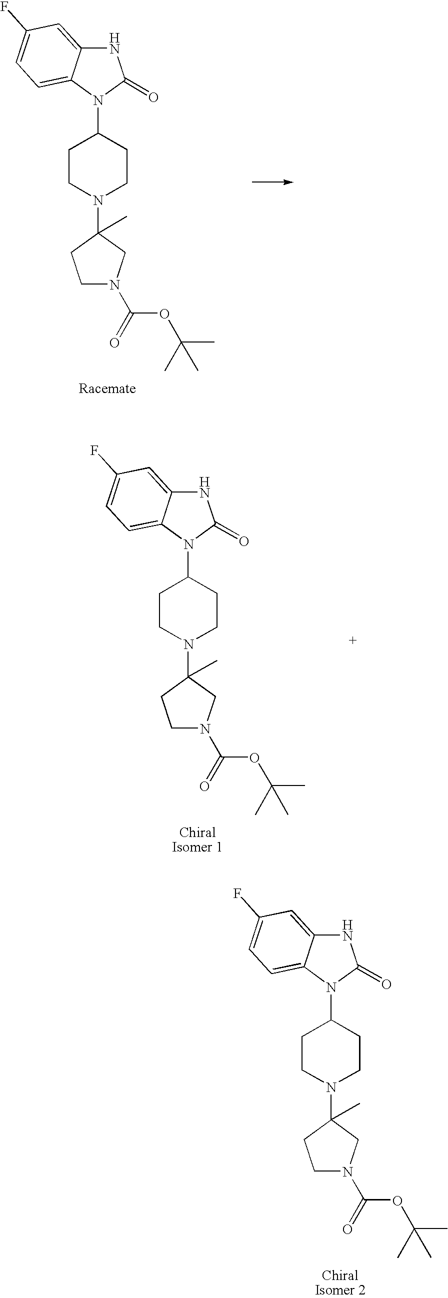

Racemic mixture of tert-butyl 3-(4-(5-fluoro-2-oxo-2,3-dihydro-1H-benzo[d]imidazol-1-yl)piperidin-1-yl)-3-methylpyrrolidine-1-carboxylate (0.892 g, 2.13 mmol) was separated by chiral chromatography (Chiralpak AD column, 10% iPrOH/10% MeOH/80% heptane). Isomer 1 of tert-butyl 3-(4-(5-fluoro-2-oxo-2,3-dihydro-1H-benzo[d]imidazol-1-yl)piperidin-1-yl)-3-methylpyrrolidine-1-carboxylate (0.400 g, 44.8%) was the first eluent. Retention time: 8.20 minutes (Chiralpak AD column, 10% iPrOH/10% MeOH/80% heptane).

Isomer 2 of tert-butyl 3-(4-(5-fluoro-2-oxo-2,3-dihydro-1H-benzo[d]imidazol-1-yl)piperidin-1-yl)-3-methylpyrrolidine-1-carboxylate (0.360 g, 40.4%) was the second fraction. Retention time: 14.03 minutes (Chiralpak AD column, 10% iPrOH/10% MeOH/80% heptane).

### Step B: Preparation of but-2-ynyl 3-(4-(5-fluoro-2-oxo-2,3-dihydro-1H-benzo[d]imidazol-1-yl)piperidin-1-yl)-3-methylpyrrolidine-1-carboxylate

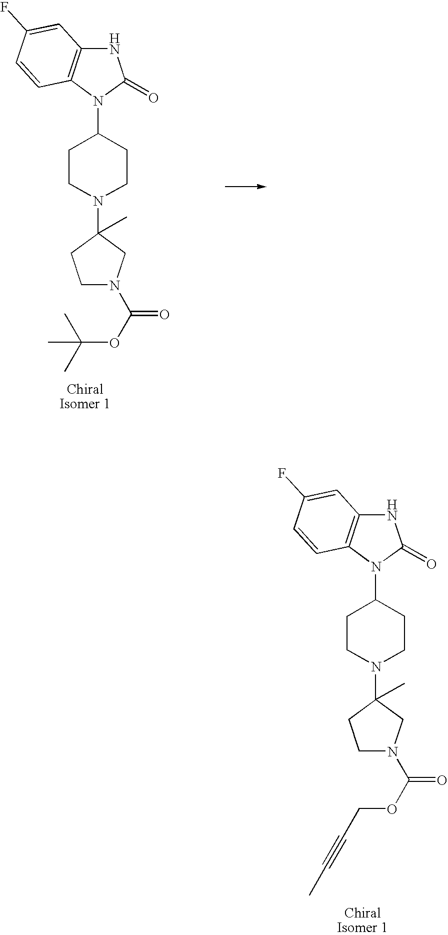

Following an analogous procedure to that described in Step F of the Example 1, the title compound was made from isomer 1 of tert-butyl 3-(4-(5-fluoro-2-oxo-2,3-dihydro-1H-benzo[d]imidazol-1-yl)piperidin-1-yl)-3-methylpyrrolidine-1-carboxylate (100 mg, 0.24 mmol) and but-2-ynyl carbonochloridate (0.027 mL, 0.24 mmol). The crude product was purified by high pH preparative HPLC (20-40% MeCN in water) to give the title compound (76 mg, 76%) as white solid, 1H NMR (400 MHz, METHANOL-D4) δ ppm 1.08-1.13 (m, 3H) 1.67-1.82 (m, 5H) 1.85-2.01 (m, 2H) 2.34-2.58 (m, 4H) 2.81 (d, J=10.94 Hz, 1H) 2.94-3.07 (m, 1H) 3.21-3.30 (m, 1H) 3.33-3.49 (m, 2H) 3.50-3.65 (m, 1H) 4.16-4.30 (m, 1H) 4.61 (d, J=2.34 Hz, 2H) 6.71-6.86 (m, 2H) 7.31 (dd, J=8.59, 4.30 Hz, 1H). HRMS [M+1]: 415.2146.

### Example 23

### methyl 3-(4-(5-fluoro-2-oxo-2,3-dihydro-1H-benzo[d]imidazol-1-yl)piperidin-1-yl)-3-methylpyrrolidine-1-carboxylate

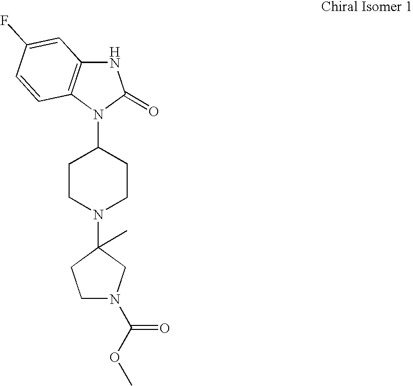

### Preparation of methyl 3-(4-(5-fluoro-2-oxo-2,3-dihydro-1H-benzo[d]imidazol-1-yl)piperidin-1-yl)-3-methylpyrrolidine-1-carboxylate

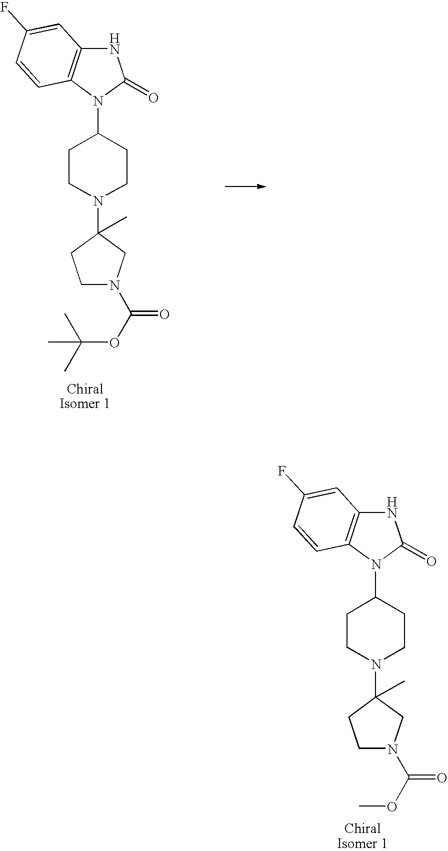

Following an analogous procedure to that described in Step F of the Example 1, the title compound was made from isomer 1 of tert-butyl 3-(4-(5-fluoro-2-oxo-2,3-dihydro-1H-benzo[d]imidazol-1-yl)piperidin-1-yl)-3-methylpyrrolidine-1-carboxylate (100 mg, 0.24 mmol) and methyl carbonochloridate (0.018 mL, 0.24 mmol). The crude product was purified by high pH preparative HPLC (20-40% MeCN in water) to give the title compound (61.9 mg, 68.8%) as solid. 1H NMR (400 MHz, METHANOL-D4) δ ppm 1.11 (s, 3H) 1.69-1.80 (m, 2H) 1.87-1.97 (m, 2H) 2.34-2.61 (m, 4H) 2.81 (t, J=8.40 Hz, 1H) 2.94-3.06 (m, 1H) 3.23 (d, J=10.16 Hz, 1H) 3.31-3.47 (m, 2H) 3.51-3.60 (m, 1H) 3.66 (d, J=1.95 Hz, 3H) 4.14-4.30 (m, 1H) 6.73-6.83 (m, 2H) 7.31 (dd, J=8.79, 4.49 Hz, 1H). HRMS [M+1]: 377.1984.

### Example 24

### 2-fluoroethyl 3-(4-(5-fluoro-2-oxo-2,3-dihydro-1H-benzo[d]imidazol-1-yl)piperidin-1-yl)-3-methylpyrrolidine-1-carboxylate

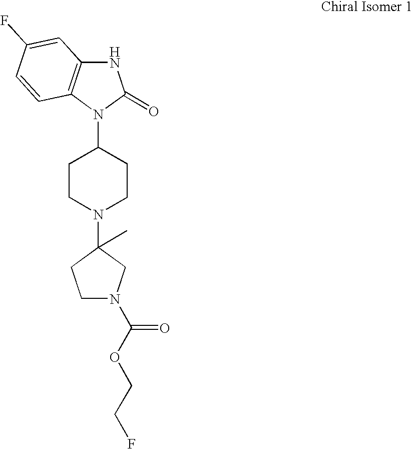

### Preparation of 2-fluoroethyl 3-(4-(5-fluoro-2-oxo-2,3-dihydro-1H-benzo[d]imidazol-1-yl)piperidin-1-yl)-3-methylpyrrolidine-1-carboxylate

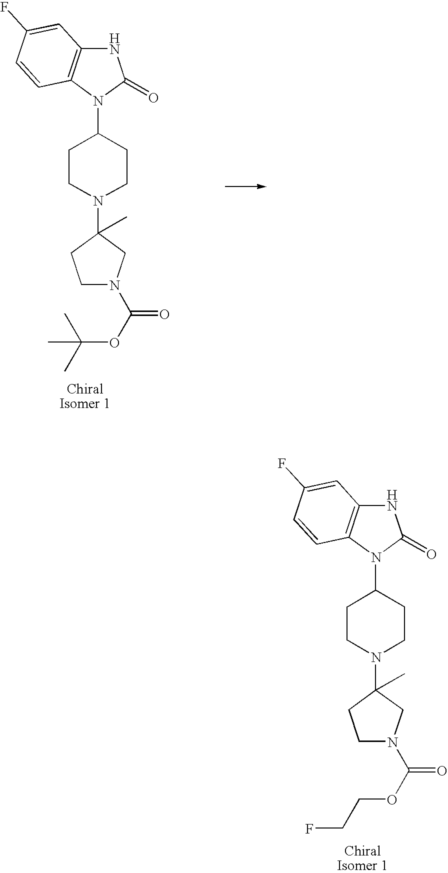

Following an analogous procedure to that described in Step F of the Example 1, the title compound was made from isomer 1 of tert-butyl 3-(4-(5-fluoro-2-oxo-2,3-dihydro-1H-benzo[d]imidazol-1-yl)piperidin-1-yl)-3-methylpyrrolidine-1-carboxylate (100 mg, 0.24 mmol) and 2-fluoroethyl carbonochloridate (0.020 mL, 0.22 mmol). The crude product was purified by high pH preparative HPLC (20-40% MeCN in water) to give the title compound (70.1 mg, 80%) as solid. 1H NMR (400 MHz, METHANOL-D4) δ ppm 1.12 (s, 3H) 1.69-1.81 (m, 2H) 1.87-1.98 (m, 2H) 2.34-2.61 (m, 4H) 2.82 (d, J=10.94 Hz, 1H) 3.00 (dd, J=7.62, 1.76 Hz, 1H) 3.22-3.31 (m, 1H) 3.33-3.51 (m, 2H) 3.54-3.66 (m, 1H) 4.17-4.28 (m, 2H) 4.30-4.35 (m, 1H) 4.47-4.54 (m, 1H) 4.58-4.65 (m, 1H) 6.73-6.84 (m, 2H) 7.28-7.34 (m, J=8.98, 4.30 Hz, 1 H). HRMS [M+1]: 409.2047.

### Example 25

### prop-2-ynyl 3-(4-(5-fluoro-2-oxo-2,3-dihydro-1H-benzo[d]imidazol-1-yl)piperidin-1-yl)-3-methylpyrrolidine-1-carboxylate

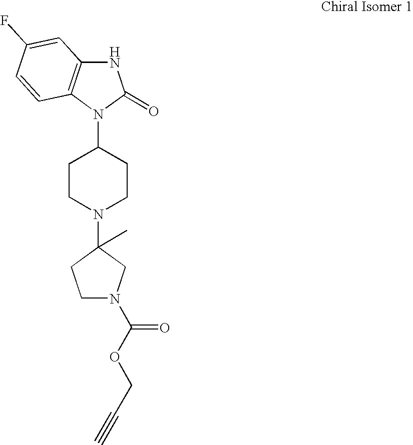

### Preparation of: prop-2-ynyl 3-(4-(5-fluoro-2-oxo-2,3-dihydro-1H-benzo[d]imidazol-1-yl)piperidin-1-yl)-3-methylpyrrolidine-1-carboxylate

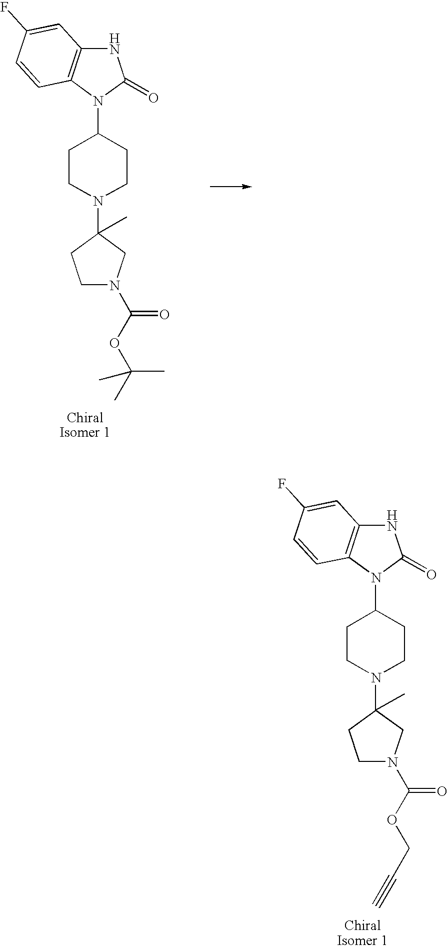

Following an analogous procedure to that described in Step F of the Example 1, the title compound was made from isomer 1 of tert-butyl 3-(4-(5-fluoro-2-oxo-2,3-dihydro-1H-benzo[d]imidazol-1-yl)piperidin-1-yl)-3-methylpyrrolidine-1-carboxylate (100 mg, 0.24 mmol) and prop-2-ynyl carbonochloridate (0.023 mL, 0.24 mmol). The crude product was purified by high pH preparative HPLC (20-40% MeCN in water) to give the title compound (52.2 mg, 54.6%) as solid. 1H NMR (400 MHz, METHANOL-D4) δ ppm 1.13 (s, 3H) 1.70-1.81 (m, 2H) 1.89-2.02 (m, 2H) 2.35-2.61 (m, 4H) 2.82 (d, J=10.94 Hz, 1H) 2.89 (t, J=2.93 Hz, 1H) 2.95-3.07 (m, 1H) 3.22-3.31 (m, 1H) 3.33-3.50 (m, 2H) 3.53-3.68 (m, 1H) 4.16-4.31 (m, 1H) 4.67 (d, J=1.95 Hz, 2H) 6.71-6.84 (m, 2H) 7.31 (dd, J=8.79, 4.49 Hz, 1H). HRMS [M+1]: 401.1987.

### Example 26 (Isomer 1), Example 27 (Isomer 2), Example 28 (Isomer 3) and Example 29

### Enantiomers of 2-fluoroethyl 3-methyl-3-(4-(6-methyl-2-oxo-2,3-dihydro-1H-benzo[d]imidazol-1-yl)piperidin-1-yl)pyrrolidine-1-carboxylate (Example 26 and Example 28) and enantiomers of 2-fluoroethyl 3-methyl-3-(4-(5-methyl-2-oxo-2,3-dihydro-1H-benzo[d]imidazol-1-yl)piperidin-1-yl)pyrrolidine-1-carboxylate (Example 27 and Example 29)

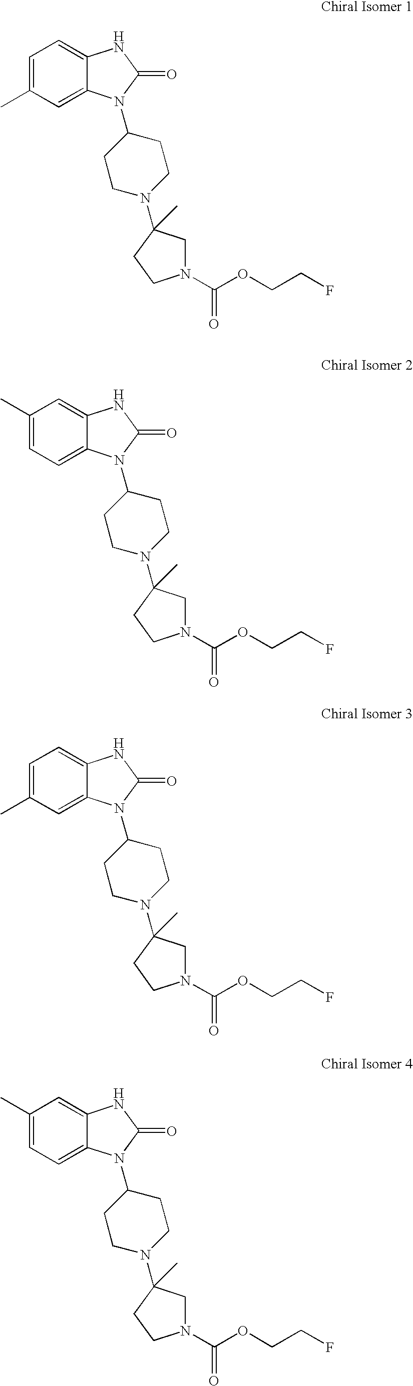

### Step A. The preparation of racemic 2-fluoroethyl 3-methyl-3-(4-(6-methyl-2-oxo-2,3-dihydro-1H-benzo[d]imidazol-1-yl)piperidin-1-yl)pyrrolidine-1-carboxylate and racemic 2-fluoroethyl 3-methyl-3-(4-(5-methyl-2-oxo-2,3-dihydro-1H-benzo[d]imidazol-1-yl)piperidin-1-yl)pyrrolidine-1-carboxylate (mixture)

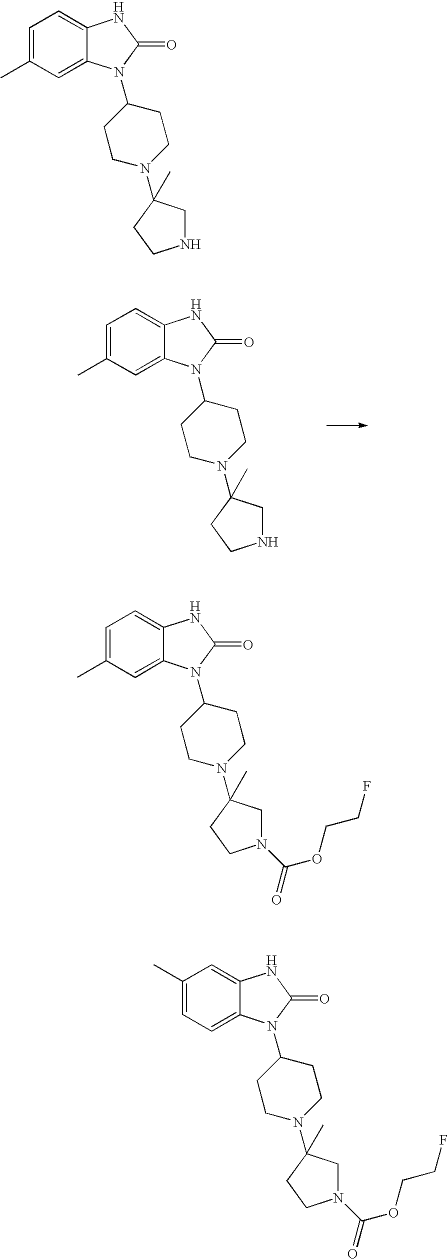

Following an analogous procedure to that described in Step E of the Example 18: The mixture of racemic 2-fluoroethyl 3-methyl-3-(4-(6-methyl-2-oxo-2,3-dihydro-1H-benzo[d]imidazol-1-yl)piperidin-1-yl)pyrrolidine-1-carboxylate and racemic 2-fluoroethyl 3-methyl-3-(4-(5-methyl-2-oxo-2,3-dihydro-1H-benzo[d]imidazol-1-yl)piperidin-1-yl)pyrrolidine-1-carboxylate (151 mg) was prepared from crude 6-methyl-1-(1-(3-methylpyrrolidin-3-yl)piperidin-4-yl)-1H-benzo[d]imidazol-2(3H)-one (HCl salt) and 5-methyl-1-(1-(3-methylpyrrolidin-3-yl)piperidin-4-yl)-1H-benzo[d]imidazol-2(3H)-one (HCl salt) (0.4962 g, 1.41 mmol), K₂CO₃ (0.280 g, 2.03 mmol) 2-fluoroethyl carbonochloridate (0.2 mL, 2.12 mmol). MS (M+1): 405.3

### Step B. Chiral separation of enantiomers of 2-fluoroethyl 3-methyl-3-(4-(6-methyl-2-oxo-2,3-dihydro-1H-benzo[d]imidazol-1-yl)piperidin-1-yl)pyrrolidine-1-carboxylate and enantiomers of 2-fluoroethyl 3-methyl-3-(4-(5-methyl-2-oxo-2,3-dihydro-1H-benzo[d]imidazol-1-yl)piperidin-1-yl)pyrrolidine-1-carboxylate

The mixture of isomers was separated by chiral SFC (AS chiral column, 50% MeOH/0.1% DMEA)/CO₂). First two isomers (Isomer 1 and Isomer 2) were collected together as a mixture and Isomer 3 and Isomer 4 were collected separately. Mixture of the isomer 1 and Isomer 2 were separated as individual isomers by a second purification by chiral SFC (AS chiral column, 30% MeOH/0.1% DMEA)/CO₂).

Isomer 1 (Example 26): Enantiomer 1 of 2-fluoroethyl 3-methyl-3-(4-(6-methyl-2-oxo-2,3-dihydro-1H-benzo[d]imidazol-1-yl)piperidin-1-yl)pyrrolidine-1-carboxylate (0.038 g). Retention time: 2.09 minutes (Chiral SFC, AS Column, 60% MeOH/0.1% DMEA/CO₂). 1HNMR (400 MHz, Methanol-D4) δ ppm 1.17 (s, 3H), 1.77 (d, J=9.37 Hz, 2H), 1.92-2.02 (m, 2H), 2.38 (s, 3H), 2.43-2.65 (m, 4H), 2.87 (d, J=10.94 Hz, 1H), 3.06 (d, J=4.30 Hz, 1H), 3.35-3.57 (m, 3H), 3.57-3.71 (m, 1H), 4.23-4.32 (m, 2H), 4.33-4.38 (m, 1H), 4.51-4.55 (m, 1H), 4.63-4.66 (m, 1H), 6.87 (d, J=7.8 Hz, 1H), 6.93 (d, J=7.8 Hz, 1H), 7.27 (s, 1H). HRMS [M+1]: 405.23037.

Isomer 2 (Example 27): Enantiomer 1 of 2-fluoroethyl 3-methyl-3-(4-(5-methyl-2-oxo-2,3-dihydro-1H-benzo[d]imidazol-1-yl)piperidin-1-yl)pyrrolidine-1-carboxylate (0.025 g, 81%). Retention time: 2.26 minutes (Chiral SFC, AS Column, 60% MeOH/0.1% DMEA/CO₂). 1HNMR (400 MHz, Methanol-D4) δ ppm 1.16 (s, 3H), 1.74-1.81 (m, 2H), 1.93-2.04 (m, H), 2.34 (s, 3H), 2.39-2.64 (m, 4H), 2.86 (d, J=10.94 Hz, 1H), 3.00-3.09 (m, 1H), 3.35-3.54 (m, 3H), 3.56-3.70 (m, 1H), 4.20-4.31 (m, 2H), 4.32-4.37 (m, 1H), 4.46-4.58 (m, 1), 4.60-4.71 (m, 1), 6.78-6.99 (m, 2H), 7.26 (d, J=8.59 Hz, 1H). HRMS [M+1]: 405.23012.

Isomer 3 (Example 28): Enantiomer 2 of 2-fluoroethyl 3-methyl-3-(4-(6-methyl-2-oxo-2,3-dihydro-1H-benzo[d]imidazol-1-yl)piperidin-1-yl)pyrrolidine-1-carboxylate (0.043 g). Retention time: 3.29 minutes (Chiral SFC, AS Column, 60% MeOH/0.1% DMEA/CO₂). 1HNMR (400 MHz, Methanol-D4) δ ppm 1.17 (s, 3H) 1.77 (d, J=9.37 Hz, 2H) 1.98 (t, J=6.25 Hz, 2H) 2.38 (s, 3H) 2.44-2.65 (m, 4H) 2.87 (d, J=10.55 Hz, 1H) 3.05 (s, 1H) 3.35-3.57 (m, 3H) 3.57-3.70 (m, 1H) 4.22-4.32 (m, 2H) 4.32-4.39 (m, 1H) 4.52 (d, J=2.73 Hz, 1H) 4.64 (d, J=3.13 Hz, 1H) 6.87 (d, J=7.80 Hz, 1H) 6.93 (d, J=8.20 Hz, 1H) 7.27 (s, 1H). HRMS [M+1]: 405.22992

Isomer 4 (Example 29): Enantiomer 2 of 2-fluoroethyl 3-methyl-3-(4-(5-methyl-2-oxo-2,3-dihydro-1H-benzo[d]imidazol-1-yl)piperidin-1-yl)pyrrolidine-1-carboxylate (0.029 g). Retention time: 4.01 minutes (Chiral SFC, AS Column, 60% MeOH/0.1% DMEA/CO₂). 1HNMR (400 MHz, Methanol-D4) δ ppm 1.16 (s, 3H), 1.72-1.83 (m, 2H), 1.93-2.08 (m, 2H), 2.34 (s, 3H), 2.39-2.64 (m, 4H), 2.79-2.92 (m, 1H), 3.04 (d, J=7.03 Hz, 1H), 3.36-3.54 (m, 3H), 3.57-3.70 (m, 1H), 4.18-4.31 (m, 2H), 4.31-4.41 (m, 1H), 4.48-4.55 (m, 1H), 4.60-4.72 (m,1H), 6.78-7.03 (m, 2H), 7.26 (d, J=8.59 Hz, 1H). HRMS [M+1]: 405.22970

### Example 30 (Isomer 1)

### methyl 3-methyl-3-(4-(2-oxo-2,3-dihydro-1H-benzo[d]imidazol-1-yl)piperidin-1-yl)pyrrolidine-1-carboxylate

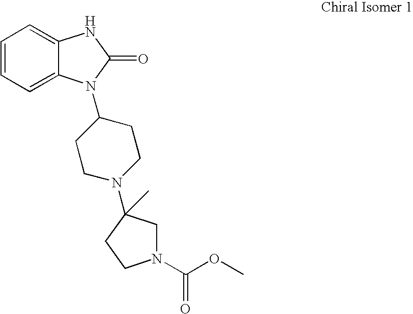

Racemic mixture of methyl 3-methyl-3-(4-(2-oxo-2,3-dihydro-1H-benzo[d]imidazol-1-yl)piperidin-1-yl)pyrrolidine-1-carboxylate (Example 2) (175.8 mg, 0.49 mmol) was separated by chiral chromatography (Chiralpak AD column, 40% EtOH/60% Hexanes) to give isomer 1 and isomer 2 of the title compound.

Isomer 1 (Example 30) was the first fraction: Enantiomer 1 of methyl 3-methyl-3-(4-(2-oxo-2,3-dihydro-1H-benzo[d]imidazol-1-yl)piperidin-1-yl)pyrrolidine-1-carboxylate (81 mg). Retention time: 10.7 minutes (Chiral HPLC, Chiralpak AD column, 40% EtOH/60% Hexanes).1H NMR (400 MHz, CHLOROFORM-D) δ ppm 1.03 (s, 3H) 1.71-2.02 (m, 4H) 2.28-2.52 (m, 4H) 2.65-2.80 (m, 1H) 2.85-3.00 (m, 1H) 3.20 (t, J=11.13 Hz, 1H) 3.26-3.61 (m, 3H) 3.64 (d, J=3.52 Hz, 3H) 4.17-4.34 (m, 1H) 6.94-7.07 (m, 3H) 7.18-7.29 (m, 1H) 9.02 (s, 1H). HRMS [M+1]: 359.2079.

### Example 31 (Isomer 1) and Example 32 (Isomer 2)

### 2-fluoroethyl 3-methyl-3-(4-(2-oxo-2,3-dihydro-1H-benzo[d]imidazol-1-yl)piperidin-1-yl)pyrrolidine-1-carboxylate

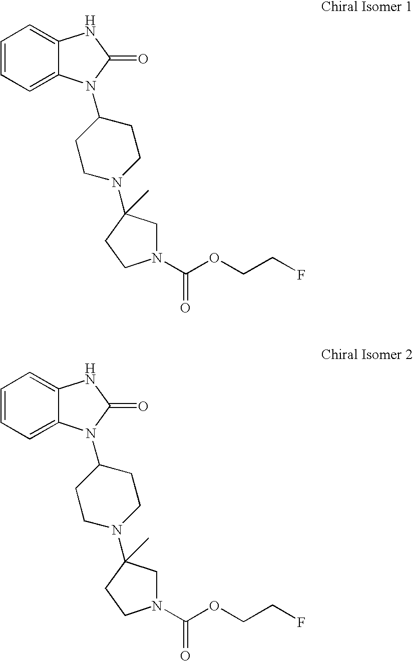

Racemic mixture of 2-fluoroethyl 3-methyl-3-(4-(2-oxo-2,3-dihydro-1H-benzo[d]imidazol-1-yl)piperidin-1-yl)pyrrolidine-1-carboxylate (Example 4) (200 mg, 0.51 mmol) was separated by chiral chromatography (Chiralpak AD column, 20% ⁱPrOH/80% Hexanes) to give isomer 1 and isomer 2 of the title compound.

Isomer 1 (Example 31) was the first fraction: Enantiomer 1 of 2-fluoroethyl 3-methyl-3-(4-(2-oxo-2,3-dihydro-1H-benzo[d]imidazol-1-yl)piperidin-1-yl)pyrrolidine-1-carboxylate (62.0 mg). Retention time: 18.48 minutes (Chiralpak AD column, 20% ⁱPrOH/80% Hexanes). 1H NMR (400 MHz, CHLOROFORM-D) δ ppm 1.04 (s, 3H) 1.70-1.97 (m, 4H) 2.27-2.54 (m, 4H) 2.68-2.78 (m, 1H) 2.89 (t, J=7.03 Hz, 1H) 3.23 (d, J=9.77 Hz, 1H) 3.30-3.48 (m, 2H) 3.52-3.67 (m, 1H) 4.19-4.36 (m, 3H) 4.45-4.53 (m, 1H) 4.58-4.64 (m, 1H) 6.92-7.04 (m, 3H) 7.15-7.30 (m, 1H) 8.10 (s, 1H). HRMS [M+1]: 391.2135.

Isomer 2 (Example 32) was the second fraction: Enantiomer 2 of 2-fluoroethyl 3-methyl-3-(4-(2-oxo-2,3-dihydro-1H-benzo[d]imidazol-1-yl)piperidin-1-yl)pyrrolidine-1-carboxylate (74.4 mg). Retention time: 23.99 minutes (Chiralpak AD column, 20% ⁱPrOH/80% Hexanes). 1H NMR (400 MHz, CHLOROFORM-D) δ ppm 1.04 (s, 3H) 1.70-1.97 (m, 4H) 2.27-2.54 (m, 4H) 2.68-2.78 (m, 1H) 2.89 (t, J=7.03 Hz, 1H) 3.23 (d, J=9.77 Hz, 1H) 3.30-3.48 (m, 2H) 3.52-3.67 (m, 1H) 4.19-4.36 (m, 3H) 4.45-4.53 (m, 1H) 4.58-4.64 (m, 1H) 6.92-7.04 7.04 (m, 3H) 7.15-7.30 (m, 1H) 8.10 (s, 1H). HRMS [M+1]: 391.2142.

### Example 33 (Isomer 1) and Example 34 (Isomer 2)

### but-2-ynyl 3-methyl-3-(4-(2-oxo-2,3-dihydro-1H-benzo[d]imidazol-1-yl)piperidin-1-yl)pyrrolidine-1-carboxylate

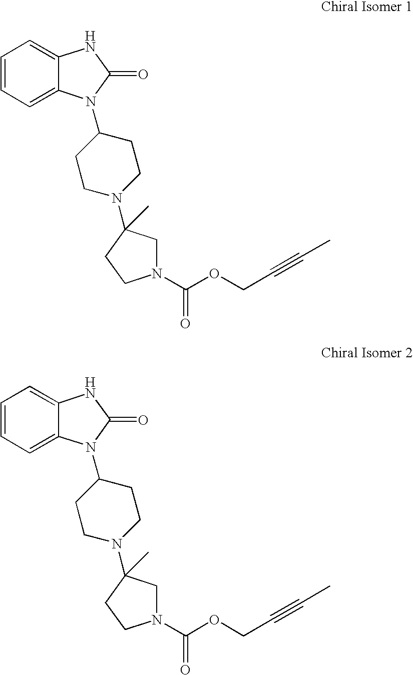

Step A: Preparation of but-2-ynyl 3-methyl-3-(4-(2-oxo-2,3-dihydro-1H-benzo[d]imidazol-1-yl)piperidin-1-yl)pyrrolidine-1-carboxylate

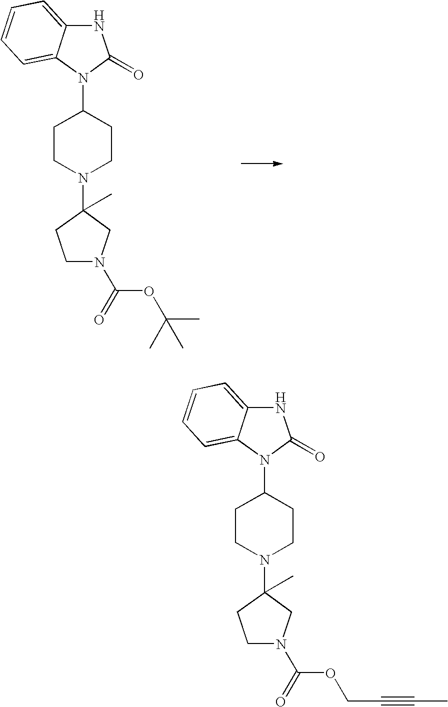

Following an analogous procedure to that described in Step F of the Example 1, the title compound was made from tert-butyl 3-methyl-3-(4-(2-oxo-2,3-dihydro-1H-benzo[d]imidazol-1-yl)piperidin-1-yl)pyrrolidine-1-carboxylate (237 mg, 0.59 mmol) and but-2-ynyl carbonochloridate (0.067 mL, 0.59 mmol). The crude product was purified by high pH preparative HPLC (20-40% MeCN in water) to give the title compound (204 mg, 87%) as solid.

### Step B: Separation of enantiomers of but-2-ynyl 3-methyl-3-(4-(2-oxo-2,3-dihydro-1H-benzo[d]imidazol-1-yl)piperidin-1-yl)pyrrolidine-1-carboxylate

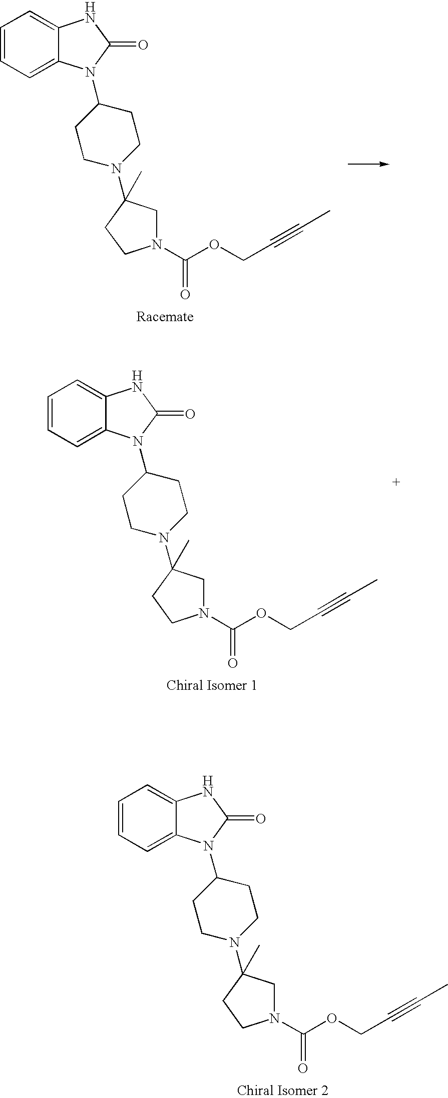

Racemic mixture of but-2-ynyl 3-methyl-3-(4-(2-oxo-2,3-dihydro-1H-benzo[d]imidazol-1-yl)piperidin-1-yl)pyrrolidine-1-carboxylate (204 mg, 0.51 mmol) was separated by chiral chromatography (Chiralpak AD column, 20% ⁱPrOH/80% Hexanes) to give isomer 1 and isomer 2 of the title compound

Isomer 1 (Example 33) was the first fraction: Enantiomer 1 of but-2-ynyl 3-methyl-3-(4-(2-oxo-2,3-dihydro-1H-benzo[d]imidazol-1-yl)piperidin-1-yl)pyrrolidine-1-carboxylate (98 mg). Retention time: 18.48 minutes (Chiralpak AD column, 20% ⁱPrOH/80% Hexanes). 1H NMR (400 MHz, CHLOROFORM-D) δ ppm 1.00-1.12 (m, 3H) 1.69-2.06 (m, 7H) 2.21-2.59 (m, 4H) 2.69-3.08 (m, 2H) 3.15-3.72 (m, 4H) 4.30 (s, 1H) 4.64 (s, 2H) 6.90-7.16 (m, 3H) 7.24 (d, J=8.59 Hz, 1H). HRMS [M+1]: 397.2236.

Isomer 2 (Example 34) was the second fraction: Enantiomer 2 of but-2-ynyl 3-methyl-3-(4-(2-oxo-2,3-dihydro-1H-benzo[d]imidazol-1-yl)piperidin-1-yl)pyrrolidine-1-carboxylate. Retention time: 21.41 minutes (Chiralpak AD column, 20% ⁱPrOH/80% Hexanes). 1H NMR (400 MHz, CHLOROFORM-D) δ ppm 1.00-1.12 (m, 3H) 1.69-2.06 (m, 7H) 2.21-2.59 (m, 4H) 2.69-3.08 (m, 2H) 3.15-3.72 (m, 4H) 4.30 (s, 1H) 4.64 (s, 2H) 6.90-7.16 (m, 3H) 7.24 (d, J=8.59 Hz, 1H). HRMS [M+1]: 397.2230.

### Example 35

### but-2-ynyl 3-(4-(5-fluoro-2-oxo-2,3-dihydro-1H-benzo[d]imidazol-1-yl)piperidin-1-yl)-3-methylpyrrolidine-1-carboxylate

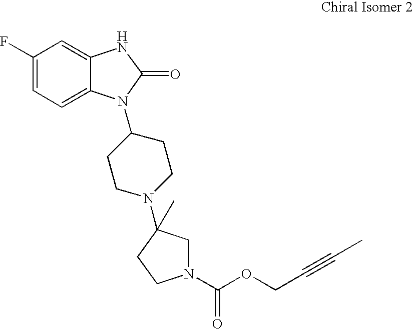

### Preparation of but-2-ynyl 3-(4-(5-fluoro-2-oxo-2,3-dihydro-1H-benzo[d]imidazol-1-yl)piperidin-1-yl)-3-methylpyrrolidine-1-carboxylate

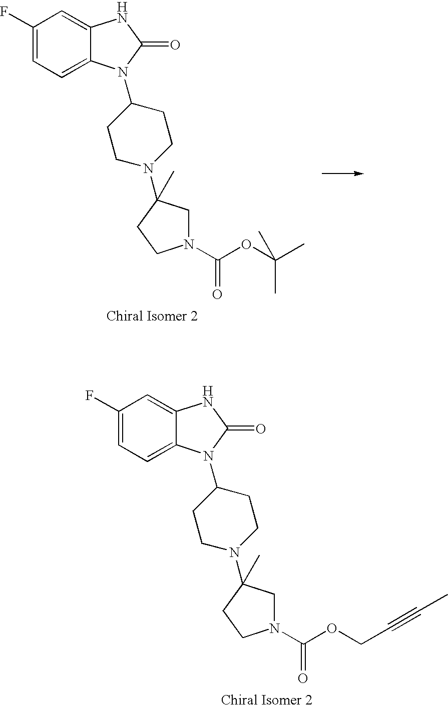

Following an analogous procedure to that described in Step F of the Example 1, the title compound was made from isomer 2 of tert-butyl 3-(4-(5-fluoro-2-oxo-2,3-dihydro-1H-benzo[d]imidazol-1-yl)piperidin-1-yl)-3-methylpyrrolidine-l-carboxylate (Example 22, Step A) (90 mg, 0.22 mmol) and but-2-ynyl carbonochloridate (0.024 mL, 0.22 mmol). The crude product was purified by high pH preparative HPLC (20-40% MeCN in water) to give the title compound (61.7 mg, 69.2%) as solid. 1H NMR (400 MHz, CHLOROFORM-D) δ ppm 1.07 (s, 3H) 1.71-2.10 (m, 7H) 2.27-2.54 (m, 4H) 2.77 (d, J=10.94 Hz, 1H) 2.89-2.99 (m, 1H) 3.26 (d, J=10.16 Hz, 1H) 3.33-3.51 (m, 2H) 3.54-3.69 (m, 1H) 4.29 (t, J=10.94 Hz, 1H) 4.57-4.72 (m, 2H) 6.70-6.78 (m, 1H) 6.86 (d, J=8.20 Hz, 1H) 7.14 (dd, J=8.79, 4.49 Hz, 1H) 10.54 (s, 1H). HRMS [M+1]: 415.2141.

### Example 36 (Isomer 2)

### prop-2-ynyl 3-(4-(5-fluoro-2-oxo-2,3-dihydro-1H-benzo[d]imidazol-1-yl)piperidin-1-yl)-3-methylpyrrolidine-1-carboxylate

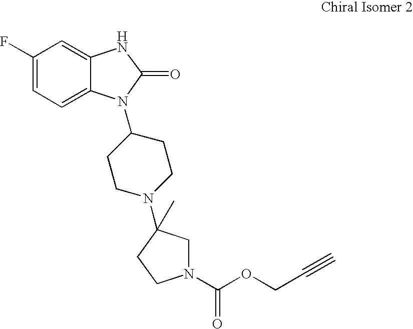

### Preparation of prop-2-ynyl 3-(4-(5-fluoro-2-oxo-2,3-dihydro-1H-benzo[d]imidazol-1-yl)piperidin-1-yl)-3-methylpyrrolidine-1-carboxylate

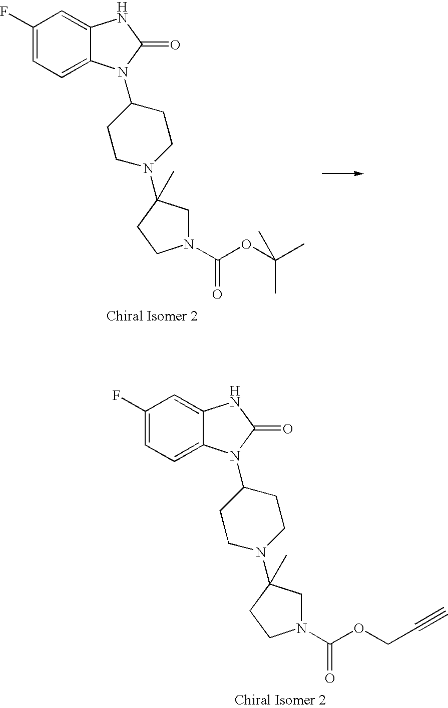

Following an analogous procedure to that described in Step F of the Example 1, the title compound was made from isomer 2 of tert-butyl 3-(4-(5-fluoro-2-oxo-2,3-dihydro-1H-benzo[d]imidazol-1-yl)piperidin-1-yl)-3-methylpyrrolidine-1-carboxylate (90 mg, 0.22 mmol) and prop-2-ynyl carbonochloridate (0.021 mL, 0.22 mmol). The crude product was purified by high pH preparative HPLC (20-40% MeCN in water) to give the title compound (48.7 mg, 56.6%) as solid. 1H NMR (400 MHz, CHLOROFORM-D) δ ppm 1.07 (d, J=2.34 Hz, 3H) 1.69-2.10 (m, 5H) 2.26-2.54 (m, 4H) 2.71-2.82 (m, 1H) 2.93 (dd, J=6.25, 3.12 Hz, 1H) 3.27 (d, J=9.77 Hz, 1H) 3.32-3.51 (m, 2H) 3.55-3.69 (m, 1H) 4.21-4.38 (m, 1H) 4.56-4.82 (m, 2H) 6.68-6.78 (m, 1H) 6.85 (d, J=8.59 Hz, 1H) 7.15 (dd, J=8.59, 4.30 Hz, 1H) 10.40 (s, 1H). HRMS [M+1]: 401.1981.

### Example 37 (Isomer 2)

### methyl 3-(4-(5-fluoro-2-oxo-2,3-dihydro-1H-benzo[d]imidazol-1-yl)piperidin-1-yl)-3-methylpyrrolidine-1-carboxylate

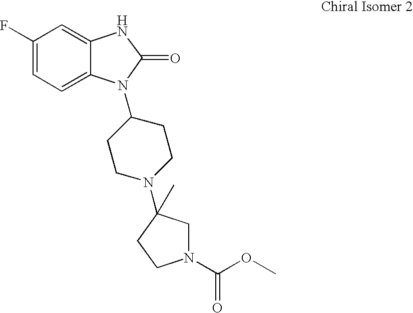

### Preparation of methyl 3-(4-(5-fluoro-2-oxo-2,3-dihydro-1H-benzo[d]imidazol-1-yl)piperidin-1-yl)-3-methylpyrrolidine-1-carboxylate

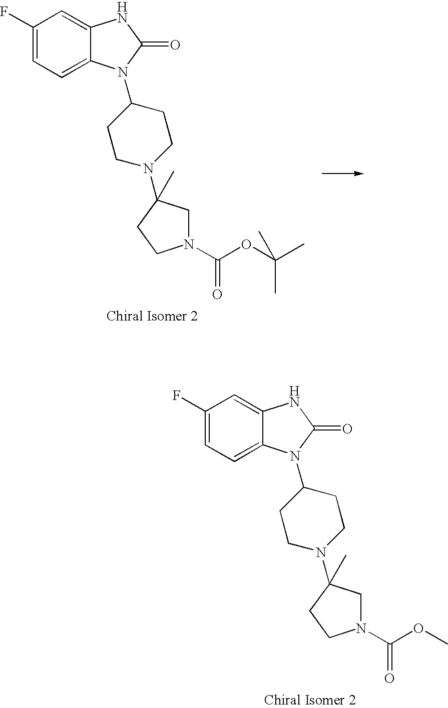

Following an analogous procedure to that described in Step F of the Example 1, the title compound was made from isomer 2 of tert-butyl 3-(4-(5-fluoro-2-oxo-2,3-dihydro-1H-benzo[d]imidazol-1-yl)piperidin-1-yl)-3-methylpyrrolidine-1-carboxylate (90 mg, 0.22 mmol) and methyl carbonochloridate (0.017 mL, 0.22 mmol). The crude product was purified by high pH preparative HPLC (20-40% MeCN in water) to give the title compound (50.8 mg, 62.8%) as solid. 1H NMR (400 MHz, CHLOROFORM-D) δ ppm 1.07 (s, 3H) 1.72-2.00 (m, 5H) 2.26-2.55 (m, 4H) 2.68-2.83 (m, 1H) 2.94 (d, J=2.73 Hz, 1H) 3.24 (t, J=11.33 Hz, 1H) 3.30-3.65 (m, 3H) 3.68 (d, J=3.52 Hz, 3H) 4.29 (t, J=11.72 Hz, 1H) 6.70-6.79 (m, 1H) 6.85 (dd, J=8.59, 2.34 Hz, 1H) 7.15 (dd, J=8.98, 4.30 Hz, 1H) 10.27 (s, 1H). HRMS [M+1]: 377.1979.

### Example 38 (Isomer 2)

### 2-fluoroethyl 3-(4-(5-fluoro-2-oxo-2,3-dihydro-1H-benzo[d]imidazol-1-yl)piperidin-1-yl)-3-methylpyrrolidine-1-carboxylate

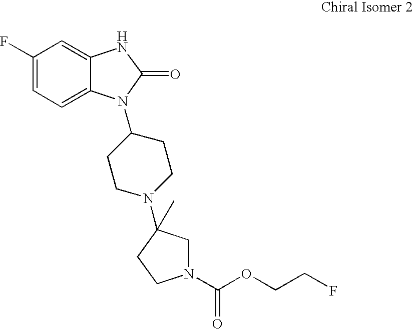

### Preparation of 2-fluoroethyl 3-(4-(5-fluoro-2-oxo-2,3-dihydro-1H-benzo[d]imidazol-1-yl)piperidin-1-yl)-3-methylpyrrolidine-1-carboxylate

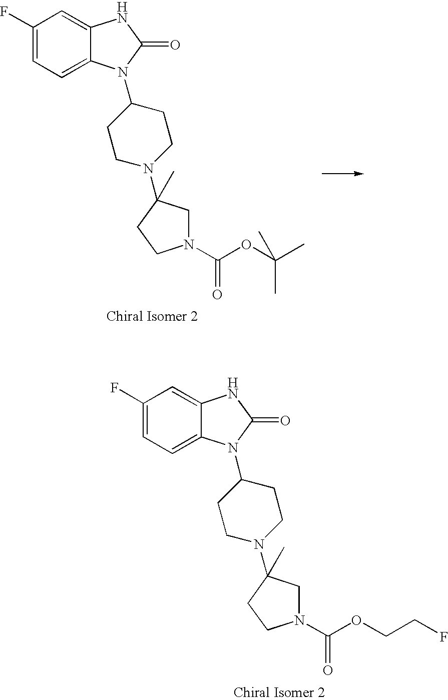

Following an analogous procedure to that described in Step F of the Example 1, the title compound was made from isomer 2 of tert-butyl 3-(4-(5-fluoro-2-oxo-2,3-dihydro-1H-benzo[d]imidazol-1-yl)piperidin-1-yl)-3-methylpyrrolidine-1-carboxylate (90 mg, 0.22 mmol) and 2-fluoroethyl carbonochloridate (0.020 mL, 0.22 mmol). The crude product was purified by high pH preparative HPLC (20-40% MeCN in water) to give the title compound (66.3 mg, 75%) as solid. 1H NMR (400 MHz, CHLOROFORM-D) δ ppm 1.08 (s, 3H) 1.76-2.03 (m, 5H) 2.27-2.55 (m, 4H) 2.78 (d, J=10.55 Hz, 1H) 2.88-3.00 (m, 1H) 3.27 (d, J=10.16 Hz, 1H) 3.34-3.51 (m, 2H) 3.61 (q, J=9.24 Hz, 1H) 4.21-4.41 (m, 3H) 4.49-4.56 (m, 1H) 4.61-4.68 (m, 1H) 6.69-6.78 (m, 1H) 6.82-6.90 (m, 1H) 7.14 (dd, J=8.59, 4.30 Hz, 1H) 10.49 (d, J=3.52 Hz, 1H). HRMS [M+1]: 409.2041.

### Example 39 (Isomer 1) and Example 40 (Isomer 3)

### isopropyl 3-methyl-3-(4-(6-methyl-2-oxo-2,3-dihydro-1H-benzo[d]imidazol-1-yl)piperidin-1-yl)pyrrolidine-1-carboxylate

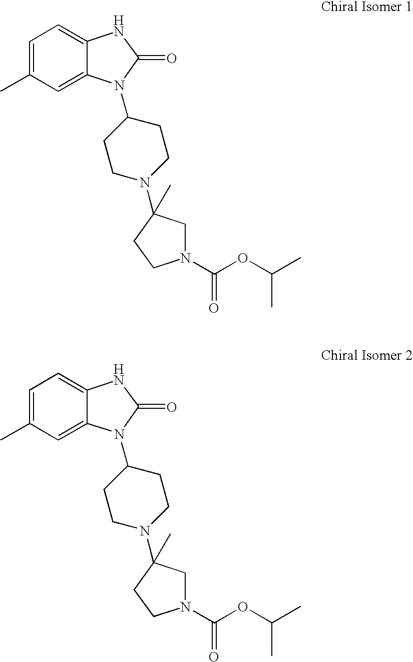

### Step A. The preparation of racemic isopropyl 3-methyl-3-(4-(6-methyl-2-oxo-2,3-dihydro-1H-benzo[d]imidazol-1-yl)piperidin-1-yl)pyrrolidine-1-carboxylate and racemic isopropyl 3-methyl-3-(4-(5-methyl-2-oxo-2,3-dihydro-1H-benzo[d]imidazol-1-yl)piperidin-1-yl)pyrrolidine-1-carboxylate (mixture)

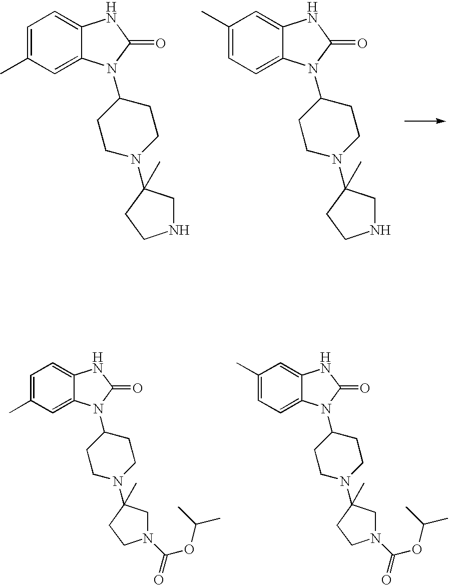

Following an analogous procedure to that described in Step E of the Example 18, a mixture of isopropyl 3-methyl-3-(4-(6-methyl-2-oxo-2,3-dihydro-1H-benzo[d]imidazol-1-yl)piperidin-1-yl)pyrrolidine-1-carboxylate and racemic isopropyl 3-methyl-3-(4-(5-methyl-2-oxo-2,3-dihydro-1H-benzo[d]imidazol-1-yl)piperidin-1-yl)pyrrolidine-1-carboxylate (241 mg) was prepared from crude 6-methyl-1-(1-(3-methylpyrrolidin-3-yl)piperidin-4-yl)-1H-benzo[d]imidazol-2(3H)-one (HCl salt) and 5-methyl-1-(1-(3-methylpyrrolidin-3-yl)piperidin-4-yl)-1H-benzo[d]imidazol-2(3H)-one (HCl salt) (0.4927 g, 1.40 mmol), and isopropyl carbonochloridate (1M, 1.40 mL, 1.40 mmol). MS (M+1): 401.2

### Step B. The separation of enantiomers of isopropyl 3-methyl-3-(4-(6-methyl-2-oxo-2,3-dihydro-1H-benzo[d]imidazol-1-yl)piperidin-1-yl)pyrrolidine-1-carboxylate and enantiomers of isopropyl 3-methyl-3-(4-(5-methyl-2-oxo-2,3-dihydro-1H-benzo[d]imidazol-1-yl)piperidin-1-yl)pyrrolidine-1-carboxylate

The mixture of isomers was separated by chiral SFC (AS column, 40% MeOH/0.1% DMEA/CO₂) to give 4 isomers (Isomer 1 (2.37 min), Isomer 2 (2.58 min), Isomer 3 (3.73 min), Isomer 4 (4.48 min).

Isomer 1 (Example 39): Enantiomer 1 of isopropyl 3-methyl-3-(4-(6-methyl-2-oxo-2,3-dihydro-1H-benzo[d]imidazol-1-yl)piperidin-1-yl)pyrrolidine-1-carboxylate (57.7 mg,). Retention time: 2.37 minutes (Chiral SFC, AS Column, 40% MeOH/0.1% DMEA/CO₂). 1H NMR (400 MHz, Methanol-D4) δ ppm 1.16 (s, 3H), 1.25 (d, J=6.25 Hz, 6H), 1.73-1.85 (m, 2H), 1.92-2.03 (m, 2H), 2.38 (s, 3H), 2.42-2.66 (m, 4H), 2.82-2.93 (m, 1H), 3.04 (s, 1H), 3.20-3.52 (m, 4H), 3.54-3.66 (m, 1H), 4.22-4.37 (m, 1H), 6.87 (d, J=8.2 Hz, 1H), 6.93 (d, J=7.8 Hz, 1H), 7.28 (s, 1H). HRMS [M+1]: 401.25464

Isomer 3 (Example 40): Enantiomer 2 of isopropyl 3-methyl-3-(4-(5-methyl-2-oxo-2,3-dihydro-1H-benzo[d]imidazol-1-yl)piperidin-1-yl)pyrrolidine-1-carboxylate (60.9 mg). Retention time: 3.73 minutes (Chiral SFC, AS Column, 40% MeOH/0.1% DMEA/CO₂). 1H NMR (400 MHz, Methanol-D4). δ ppm 1.16 (s, 3H), 1.25 (d, J=6.25 Hz, 6H), 1.78 (s, 2H), 1.88-2.09 (m, 2H), 2.38 (s, 3H), 2.42-2.66 (m, 4H), 2.86 (s, 1H), 3.05 (s, 1H), 3.22-3.51 (m, 4H), 3.52-3.67 (m, 1H), 4.05-4.35 (m, 1H), 6.87 (d, J=8.2 Hz, 1H), 6.93 (d, J=7.8 Hz 1H), 7.28 (s, 1H). HRMS [M+1]: 401.25478.

Isomer 2 and Isomer 4 were identical to examples 14 and 15, which are regio isomers of Examples 39 and Example 40.

### Example 41 (Isomer 1), Example 42 (Isomer 2), Example 43 (Isomer 3) and Example 44 (Isomer 4)

### Enantiomers of prop-2-ynyl 3-methyl-3-(4-(6-methyl-2-oxo-2,3-dihydro-1H-benzo[d]imidazol-1-yl)piperidin-1-yl)pyrrolidine-1-carboxylate and enantiomers of prop-2-ynyl 3-methyl-3-(4-(5-methyl-2-oxo-2,3-dihydro-1H-benzo[d]imidazol-1-yl)piperidin-1-yl)pyrrolidine-1-carboxylate

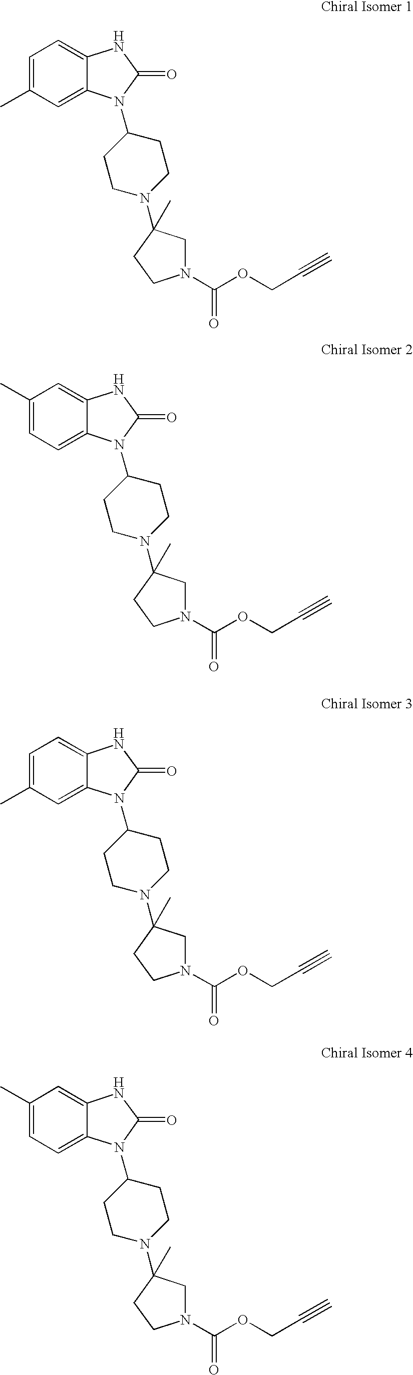

### Step A. Preparation of the mixture of racemic prop-2-ynyl 3-methyl-3-(4-(6-methyl-2-oxo-2,3-dihydro-1H-benzo[d]imidazol-1-yl)piperidin-1-yl)pyrrolidine-1-carboxylate and racemic prop-2-ynyl 3-methyl-3-(4-(5-methyl-2-oxo-2,3-dihydro-1H-benzo[d]imidazol-1-yl)piperidin-1-yl)pyrrolidine-1-carboxylate

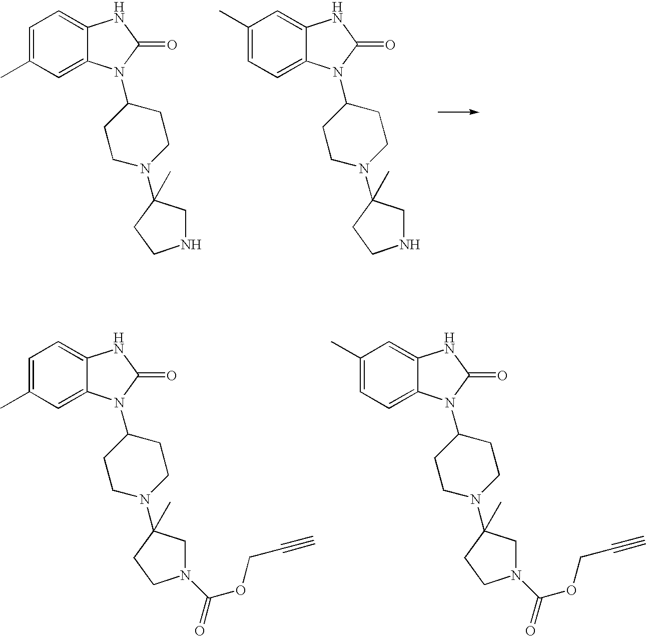

Following an analogous procedure to that described in Step E of the Example 18: The mixture of racemic prop-2-ynyl 3-methyl-3-(4-(6-methyl-2-oxo-2,3-dihydro-1H-benzo[d]imidazol-1-yl)piperidin-1-yl)pyrrolidine-1-carboxylate and racemic prop-2-ynyl 3-methyl-3-(4-(5-methyl-2-oxo-2,3-dihydro-1H-benzo[d]imidazol-1-yl)piperidin-1-yl)pyrrolidine-1-carboxylate (0.216 g) was prepared from a mixture of 6-methyl-1-(1-(3-methylpyrrolidin-3-yl)piperidin-4-yl)-1H-benzo[d]imidazol-2(3H)-one (HCl salt) (40% of 5-methyl-1-(1-(4-methylpyrrolidin-3-yl)piperidin-4-yl)-1H-benzo[d]imidazol-2(3H)-one (HCl salt) (0.5 g, 1.42 mmol) and prop-2-ynyl carbonochloridate (0.338 g, 2.85 mmol). MS (M+1): 397.2

### Step B. The separation of Enantiomers of prop-2-ynyl 3-methyl-3-(4-(6-methyl-2-oxo-2,3-dihydro-1H-benzo[d]imidazol-1-yl)piperidin-1-yl)pyrrolidine-1-carboxylate and enantiomers of prop-2-ynyl 3-methyl-3-(4-(5-methyl-2-oxo-2,3-dihydro-1H-benzo[d]imidazol-1-yl)piperidin-1-yl)pyrrolidine-1-carboxylate

The mixture of isomers was separated by chiral SFC (AS column, 50% MeOH/0.1% DMEA/CO₂)

Isomer 1 (Example 41): Enantiomer 1 of prop-2-ynyl 3-methyl-3-(4-(6-methyl-2-oxo-2,3-dihydro-1H-benzo[d]imidazol-1-yl)piperidin-1-yl)pyrrolidine-1-carboxylate (37.3 mg). Retention time: 2.76 minutes (AS column, 50% MeOH/0.1% DMEA/CO₂). 1H NMR (400 MHz, METHANOL-D4) δ ppm 1.17 (s, 1H) 2.38 (s, 3H) 2.44-2.55 (m, 4H) 2.53-2.67 (m, 2H) 2.81-2.97 (m, 2H) 3.00-3.09 (m, J=6.25 Hz, 2H) 3.12 (d, J=4.69 Hz, 2H) 3.39-3.56 (m, J=21.68, 11.91 Hz, 2H) 3.56-3.70 (m, 2H) 4.22-4.34 (m, 2H) 4.66-4.73 (m, J=2.34 Hz, 3H) 6.86 (d, J=11.33 Hz, 1H) 6.93 (d, J=10.94 Hz, 1H) 7.27 (s, 1H). HRMS [M+1]: 397.22366.

Isomer 2 (Example 42): Enantiomer 1 of prop-2-ynyl 3-methyl-3-(4-(5-methyl-2-oxo-2,3-dihydro-1H-benzo[d]imidazol-1-yl)piperidin-1-yl)pyrrolidine-1-carboxylate (34.6 mg). Retention time: 3.05 minutes (AS column, 50% MeOH/0.1%DMEA/CO₂). 1H NMR (400 MHz, METHANOL-D4) δ ppm 1.14 (s, 1H) 1.68-1.84 (m, 2H) 1.89-2.04 (m, 2H) 2.32 (s, 3H) 2.39-2.52 (m, 2H) 2.51-2.64 (m, 2H) 2.78-2.93 (m, 2H) 2.98-3.08 (m, 2H) 3.35-3.52 (m, 2H) 3.53-3.67 (m, 2H) 4.15-4.33 (m, 2H) 4.68 (s, 3H) 6.82-6.91 (m, 2H) 7.23 (d, J=8.59 Hz, 1H). HRMS [M+1]: 397.22359.

Isomer 3 (Example 43): Enantiomer 2 of prop-2-ynyl 3-methyl-3-(4-(6-methyl-2-oxo-2,3-dihydro-1H-benzo[d]imidazol-1-yl)piperidin-1-yl)pyrrolidine-1-carboxylate (43.2 mg). Retention time: 4.39 minutes (AS column, 50% MeOH/0.1% DMEA/CO₂). 1H NMR (400 MHz, METHANOL-D4) δ ppm 1.17 (s, 1H) 1.71-1.84 (m, 2H) 1.92-2.06 (m, 2H) 2.38 (s, 3H) 2.45-2.55 (m, J=8.20 Hz, 2H) 2.54-2.65 (m, J=13.28, 13.28 Hz, 2H) 2.83-2.94 (m, 2H) 3.02-3.08 (m, 2H) 3.35-3.55 (m, 2H) 3.57-3.72 (m, 2H) 4.21-4.36 (m, 2H) 4.70 (s, 3H) 6.87 (d, 1H) 6.93 (d, 1H) 7.27 (s, 1H). HRMS [M+1]: 397.22310.

Isomer 4 (Example 44): Enantiomer 2 of prop-2-ynyl 3-methyl-3-(4-(5-methyl-2-oxo-2,3-dihydro-1H-benzo[d]imidazol-1-yl)piperidin-1-yl)pyrrolidine-1-carboxylate (35.7 mg). Retention time: 5.26 minutes (AS column, 50% MeOH/0.1%DMEA/CO₂). 1H NMR (400 MHz, METHANOL-D4) δ ppm 1.14 (s, 1H) 1.75 (d, J=7.81 Hz, 2H) 1.88-2.01 (m, 2H) 2.32 (s, 3H) 2.38-2.51 (m, 2H) 2.50-2.63 (m, 2H) 2.77-2.92 (m, 2H) 2.97-3.09 (m, 2H) 3.36-3.51 (m, 2H) 3.54-3.66 (m, 2H) 4.15-4.32 (m, 2H) 4.68 (s, 3H) 6.82-6.89 (m, 2H) 7.23 (d, J=8.59 Hz, 1H). HRMS [M+1]: 397.22296.

### Example 45 (Isomer 1), Example 46 (Isomer 2), Example 47 (Isomer 3) and Example 48 (Isomer 4)

### Enantiomers of methyl 3-methyl-3-(4-(5-methyl-2-oxo-2,3-dihydro-1H-benzo[d]imidazol-1-yl)piperidin-1-yl)pyrrolidine-1-carboxylate and enantiomers of methyl 3-methyl -3-(4-(6-methyl-2-oxo-2,3-dihydro-1H-benzo[d]imidazol-1-yl)piperidin-1-yl)pyrrolidine-1-carboxylate

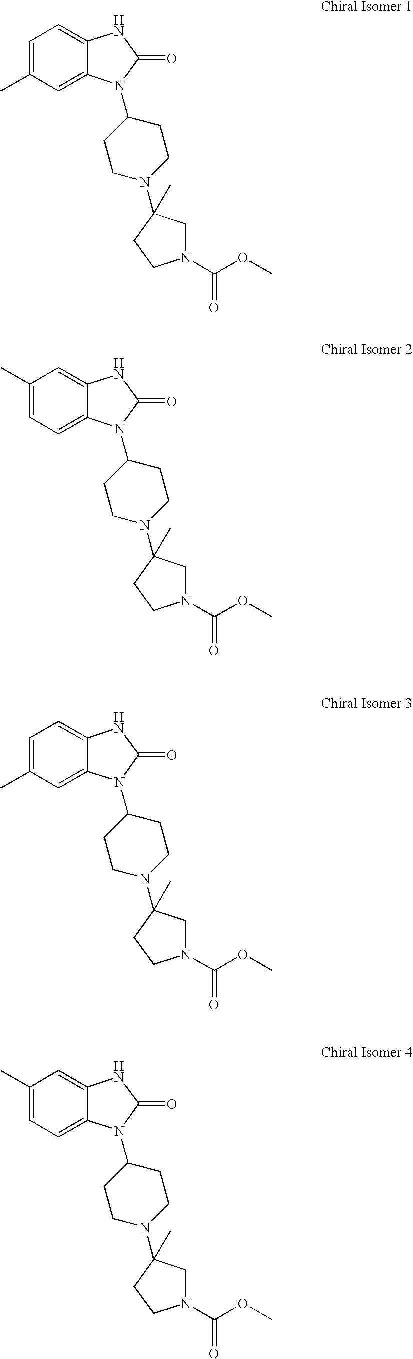

### Step A. Preparation of the mixture of racemic methyl 3-methyl-3-(4-(5-methyl-2-oxo-2,3-dihydro-1H-benzo[d]imidazol-1-yl)piperidin-1-yl)pyrrolidine-1-carboxylate and racemic methyl 3-methyl-3-(4-(6-methyl-2-oxo-2,3-dihydro-1H-benzo[d]imidazol-1-yl)piperidin-1-yl)pyrrolidine-1-carboxylate

Following an analogous procedure to that described in Step E of the Example 18, the mixture of racemic methyl 3-methyl-3-(4-(5-methyl-2-oxo-2,3-dihydro-1H-benzo[d]imidazol-1-yl)piperidin-1-yl)pyrrolidine-1-carboxylate and racemic methyl 3-methyl-3-(4-(6-methyl-2-oxo-2,3-dihydro-1H-benzo[d]imidazol-1-yl)piperidin-1-yl)pyrrolidine-1-carboxylate (115 mg) was prepared from a mixture of 6-methyl-1-(1-(3-methylpyrrolidin-3-yl)piperidin-4-yl)-1H-benzo[d]imidazol-2(3H)-one (HCl salt) (40% of 5-methyl-1-(1-(3-methylpyrrolidin-3-yl)piperidin-4-yl)-1H-benzo[d]imidazol-2(3H)-one (HCl salt)) (0.403 g, 1.15 mmol), and methyl carbonochloridate (0.106 mL, 1.38 mmol). MS (M+1): 373.1.

The mixture of isomers was separated by chiral SFC (Chiralpak AD column, 40% ⁱPrOH/0.1%DMEA/CO₂)

Isomer 1 (Example 45): Enantiomer 1 of methyl 3-methyl-3-(4-(6-methyl-2-oxo-2,3-dihydro-1H-benzo[d]imidazol-1-yl)piperidin-1-yl)pyrrolidine-1-carboxylate (0.031 g). Retention time: 3.07 minutes (Chiralpak AD column, 40% MeOH/0.1%DMEA/CO₂). 1H NMR (400 MHz, CHLOROFORM-D) δ ppm 1.11 (s, 3H) 1.85 (br. s., 2H) 1.89 (d, J=7.42 Hz, 1H) 1.92-2.02 (m, 1H) 2.40-2.41 (m, 3H) 2.41-2.46 (m, 2H) 2.48-2.58 (m, 1H) 2.80 (t, J=13.48 Hz, 1H) 2.97 (br. s., 1H) 3.25-3.33 (m, 1H) 3.33-3.49 (m, 1H) 3.52 (d, J=9.77 Hz, 1H) 3.54-3.61 (m, 1H) 3.61-3.69 (m, 1H) 3.71 (d, J=3.12 Hz, 3H) 4.31 (t, 1H) 6.86 (d, 1H) 6.94 (d, 1H) 7.08 (s, 1H) 8.25 (d, 1H). HRMS [M+1]: 373.22355.

Isomer 2 (Example 46): Enantiomer 1 of methyl 3-methyl-3-(4-(5-methyl-2-oxo-2,3-dihydro-1H-benzo[d]imidazol-1-yl)piperidin-1-yl)pyrrolidine-1-carboxylate (0.019 g, 16.43%).

Retention time: 4.09 minutes (Chiralpak AD column, 40% MeOH/0.1% DMEA/CO₂). 1H NMR (400 MHz, CHLOROFORM-D) δ ppm 1.09 (s, 3H) 1.21 (d, J=6.25 Hz, 2H) 1.60 (br. s., 1H) 1.78-1.90 (m, 4H) 1.90-2.02 (m, 1H) 2.37 (s, 3H) 2.39-2.46 (m, 2H) 2.51 (t, J=10.74 Hz, 1H) 2.72-2.86 (m, 1H) 2.95 (br. s., 1H) 3.21-3.32 (m, 1H) 3.32-3.47 (m, 1H) 3.51 (d, J=10.16 Hz, 1H) 3.56 (t, 1H) 3.64 (t, J=9.57 Hz, 0H) 3.71 (d, J=3.52 Hz, 3H) 4.32 (t, 1H) 6.87 (d, J=8.20 Hz, 1H) 6.90 (s, 1H) 7.16 (d, J=7.81 Hz, 1H) 8.47 (d, J=4.69 Hz, 1H). HRMS [M+1]: 373.22423.

Isomer 3 (Example 47): Enantiomer 2 of methyl 3-methyl-3-(4-(6-methyl-2-oxo-2,3-dihydro-1H-benzo[d]imidazol-1-yl)piperidin-1-yl)pyrrolidine-1-carboxylate (0.032 g, 92%). Retention time: 3.32 minutes (Chiralpak AD column, 40% MeOH/0.1%DMEA/CO₂). 1H NMR (400 MHz, CHLOROFORM-d) δ ppm 1.10 (s, 3H) 1.61 (br. s., 2H) 1.79-1.92 (m, 3H) 1.92-2.04 (m, 1H) 2.41 (s, 3H) 2.42-2.46 (m, 2H) 2.47-2.59 (m, 1H) 2.80 (t, J=12.89 Hz, 1H) 2.97 (br. s., 1H) 3.24-3.36 (m, 1H) 3.36-3.49 (m, 1H) 3.52 (d, J=9.77 Hz, 3.61 (m, 1H) 3.65 (t, J=9.96 Hz, 1H) 3.71 (d, J=3.52 Hz, 3H) 4.24-4.38 (m, 1H) 6.86 (d, 1H) 6.95 (d, 1H) 7.08 (s, 1H) 8.52 (d, J=5.47 Hz, 1H). HRMS [M+1]: 373.22372.

Isomer 4 (Example 48): Enantiomer 2 of methyl 3-methyl-3-(4-(5-methyl-2-oxo-2,3-dihydro-1H-benzo[d]imidazol-1-yl)piperidin-1-yl)pyrrolidine-1-carboxylate (0.021 g, 93%). Retention time: 4.63 minutes (Chiralpak AD column, 40% MeOH/0.1%DMEA/CO₂). 1H NMR (400 MHz, CHLOROFORM-d) δ ppm 1.10 (s, 3H) 1.78-1.90 (m, 3H) 1.90-2.03 (m, 1H) 2.37 (s, 3H) 2.42 (d, J=8.59 Hz, 2H) 2.46-2.59 (m, 1H) 2.73-2.86 (m, 1H) 2.96 (br. s., 1H) 3.27 (t, 1H) 3.33-3.46 (m, 1H) 3.51 (d, J=10.16 Hz, 1H) 3.57 (t, 1H) 3.57 (t, 1H) 3.65 (t, 1H) 3.71 (d, J=3.91 Hz, 3H) 4.31 (t, 1H) 6.87 (d, 1H) 6.89 (s, 1H) 7.18 (d, 1H) 8.43 (d, 1H). HRMS [M+1]: 373.22381.

### Example 49 (Isomer 1) and Example 50 (Isomer 2)

### ethyl 3-(4-(6-fluoro-2-oxo-2,3-dihydro-1H-benzo[d]imidazol-1-yl)piperidin-1-yl)-3-methylpyrrolidine-1-carboxylate

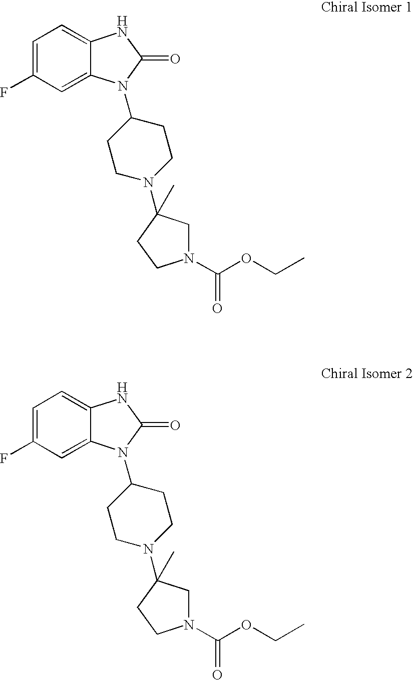

### Step A: Preparation of tert-butyl 3-(4-(hydroxyimino)piperidin-1-yl)-3-methylpyrrolidine-1-carboxylate

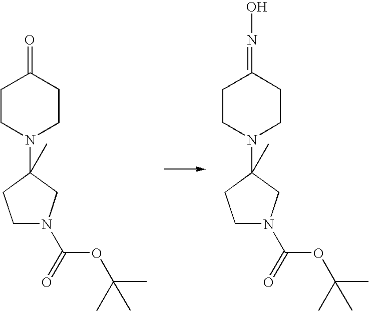

tert-butyl 3-methyl-3-(4-oxopiperidin-1-yl)pyrrolidine-1-carboxylate (100 mg, 0.35 mmol) was added to a mixture of hydroxylamine (HCl salt, 37.7 mg, 0.54 mmol) and sodium acetate (32.5 mg, 0.40 mmol) in water (2 mL) at 60° C. in one portion. The mixture was stirred at 80° C. for 2 hrs. Solid K₂CO₃ was added to neutralize the reaction followed by dichloromethane. The aqueous layer was extracted three times with dichloromethane (3×10 mL). Combined the organic layers were washed with brine, and dried over MgSO₄, filtered and concentrated under reduced pressure. The crude product was used in the subsequent step without further purification. MS (M+1) : 318.2.

### Step B : Preparation of tert-butyl 3-(4-aminopiperidin-1-yl)-3-methylpyrrolidine-1-carboxylate

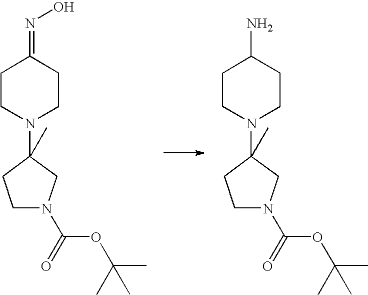

A solution of tert-butyl 3-(4-(hydroxyimino)piperidin-1-yl)-3-methylpyrrolidine-1-carboxylate (87 mg, 0.29 mmol) in 2M NH₃/MeOH (10ml) was treated with Raney Nickel (17.17 mg, 0.29 mmol) and was shaken under hydrogen atmosphere at 50 psi pressure for 12 hours. The mixture was filtered and the filtrate was concentrated under reduced pressure. The residue was purified by high pH preparative HPLC (20-40% MeCN in water) to give the title compound (52.5 mg, 63.3%) as solid. 1H NMR (400 MHz, Metanol-D4) δ ppm 1.05 (s, 3H), 1.35-1.50 (m, 10H), 1.77-1.93 (m, 4H), 2.18-2.44 (m, 2H), 2.59-2.76 (m, 2H), 2.84 (d, J=11.33 Hz, 1H), 3.11 (t, J=10.55 Hz, 1H), 3.22-3.37 (m, 3H), 3.41-3.53 (m, 1H). MS(M+1): 284.3.

### Step C: Preparation of tert-butyl 3-(4-(5-fluoro-2-nitrophenylamino)piperidin-1-yl)-3-methylpyrrolidine-1-carboxylate

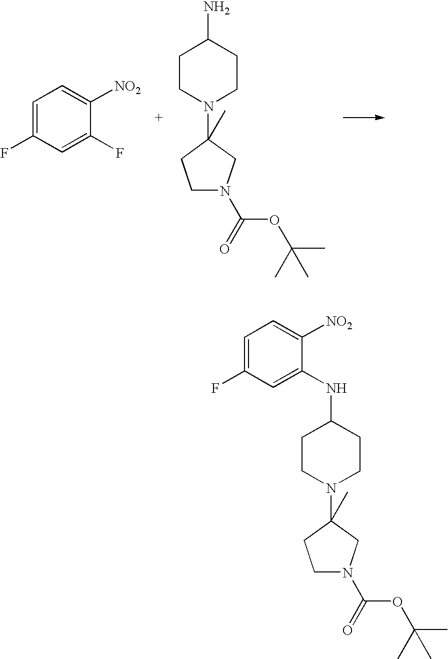

A mixture of tert-butyl 3-(4-aminopiperidin-1-yl)-3-methylpyrrolidine-1-carboxylate (56 mg, 0.20 mmol), 2,4-difluoro-1-nitrobenzene (31.4 mg, 0.20 mmol) and sodium carbonate (62.8 mg, 0.59 mmol) in DMF (5 mL) was heated at 70° C. for 12 hours. The reaction mixture was filtered and the filtrate was concentrated under reduced pressure. The residue was taken in water and was extracted three times with dichloromethane (3×20 mL). Combined organic layers were washed with brine, dried over MgSO₄, filtered and concentrated under reduced pressure. The residue was purified by high pH preparative HPLC (50-70% MeCN in water) to give the title compound (41.5 mg, 49.7%) as solid. 1H NMR (400 MHz, CDCl₃) δ ppm 1.05 (d, J=3.52 Hz, 3H), 1.36-1.48 (m, 9H), 1.55-1.70 (m, 3H), 1.73-1.92 (m, 2H), 2.05 (d, J=10.55 Hz, 2H), 2.31-2.53 (m, 2H), 2.61-2.71 (m, 1H), 2.77-2.90 (m, 1H), 3.15 (t, J=9.77 Hz, 1H), 3.23-3.36 (m, 2H), 3.37-3.61 (m, 2H), 6.28-6.35 (m, 1H), 6.45 (d, J=11.33 Hz, 1H), 8.14-8.31 (m, 2H). MS (M+1): 423.4.

### Step D: preparation of tert-butyl 3-(4-(2-amino-5-fluorophenylamino)piperidin-1-yl)-3-methylpyrrolidine-1-carboxylate

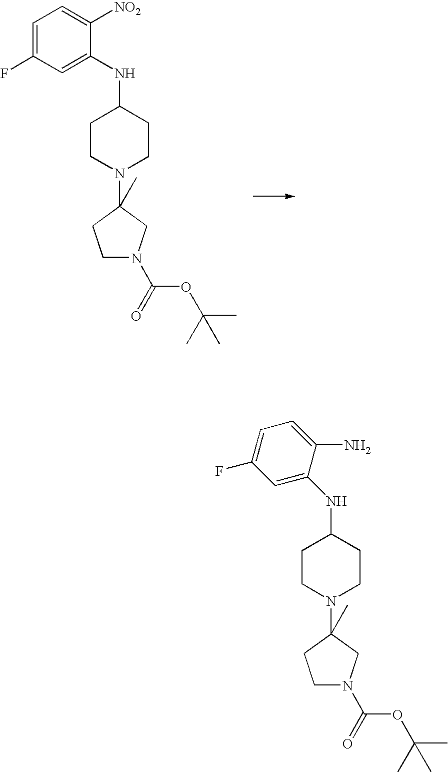

A mixture of tert-butyl 3-(4-(5-fluoro-2-nitrophenylamino)piperidin-1-yl)-3-methylpyrrolidine-1-carboxylate (41.5 mg, 0.10 mmol) in MeOH (10 mL) was treated with Palladium, 10% on Charcoal (10.45 mg, 0.10 mmol) and was shaken under hydrogen atmosphere at 50 psi pressure for 6 hours. The mixture was filtered and the filtrate was concentrated under reduced pressure. The crude product (40.0 mg, 104%) was used in the subsequent step without further purification. 1H NMR (400 MHz, CDCl₃) δ ppm 0.83 (q, J=15.49 Hz, 1H), 1.04 (d, J=3.12 Hz, 3H), 1.37-1.53 (m, 9H), 1.73-1.92 (m, 3H), 2.05 (d, J=10.55 Hz, 2H), 2.26-2.49 (m, 2H), 2.63 (d, J=3.12 Hz, 1H), 2.80 (dd, J=10.74, 4.49 Hz, 1H), 2.94-3.22 (m, 3H), 3.23-3.36 (m, 2H), 3.38-3.65 (m, 3H), 6.20-6.36 (m, 2H), 6.56-6.68 (m, 1H). MS (M+1) 393.3.

### Step E: Preparation of tert-butyl 3-(4-(6-fluoro-2-oxo-2,3-dihydro-1H-benzo[d]imidazol-1-yl)piperidin-1-yl)-3-methylpyrrolidine-1-carboxylate

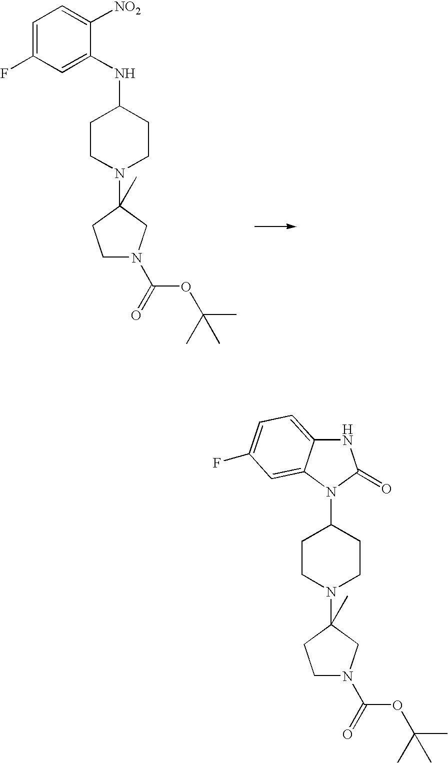

A solution of bis(trichloromethyl) carbonate (8.90 mg, 0.03 mmol) in dichloromethane (2 mL) was added slowly at 0° C. to a mixture of tert-butyl 3-(4-(2-amino-5-fluorophenylamino)piperidin-1-yl)-3-methylpyrrolidine-1-carboxylate (0.039 g, 0.1 mmol) and triethylamine (0.021 mL, 0.15 mmol) in dichloromethane (5 mL). The reaction mixture was stirred at room temperature for 0.5 hours. Water was added to the mixture and extracted with dichloromethane (3×10 mL). Combined the organic layers were washed with brine, dried over MgSO₄, filtered and concentrated under reduced pressure. The residue was purified by high pH preparative HPLC (50-70% MeCN in water) to give the title compound (0.036 g, 85%) as solid. 1H NMR (400 MHz, CDCl₃) δ ppm 1.06 (d, J=4.30 Hz, 3H), 1.39-1.51 (m, 9H), 1.72-1.94 (m, 4H), 2.23-2.54 (m, 4H), 2.77 (d, J=10.55 Hz, 1H), 2.94 (d, J=7.81 Hz, 1H), 3.19 (t, J=9.96 Hz, 1H), 3.27-3.39 (m, 1H), 3.40-3.64 (m, 2H), 4.16 4.38 (m, 1H), 6.74 (t, J=8.98 Hz, 1H), 6.93-7.08 (m, 2H), 9.95 (s, 1H). MS (M+1): 419.3.

### Step F: Preparation of ethyl 3-(4-(6-fluoro-2-oxo-2,3-dihydro-1H-benzo[d]imidazol-1-yl)piperidin-1-yl)-3-methylpyrrolidine-1-carboxylate (Racemate)

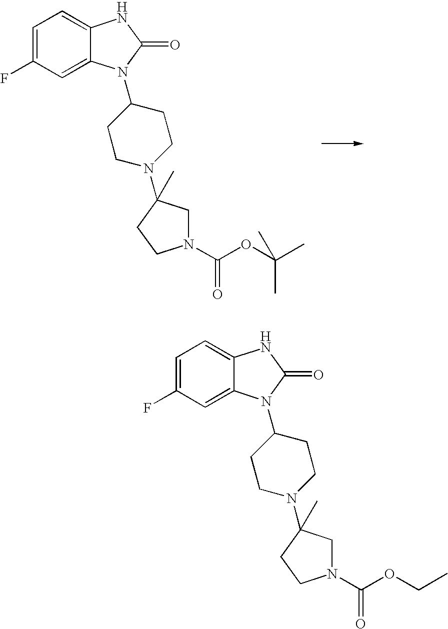

Following an analogous procedure to that described in Step F of the Example 1, the title compound was made from tert-butyl 3-(4-(6-fluoro-2-oxo-2,3-dihydro-1H-benzo[d]imidazol-1-yl)piperidin-1-yl)-3-methylpyrrolidine-1-carboxylate (36 mg, 0.09 mmol) and ethyl carbonochloridate (8.20 μL, 0.09 mmol). The crude product was purified by high pH preparative HPLC (20-40% MeCN in water) to give the title compound (19.30 mg, 57.5%) as solid.1H NMR (400 MHz, METHANOL-D4) δ ppm 1.13 (s, 3H) 1.20-1.27 (m, 3H) 1.76 (d, J=10.55 Hz, 2H) 1.88-2.02 (m, 2H) 2.35-2.64 (m, 4H) 2.76-2.89 (m, 1H) 3.02 (dd, J=7.42, 2.34 Hz, 1H) 3.20-3.30 (m, 1H) 3.33-3.48 (m, 2H) 3.52-3.65 (m, 1H) 4.10 (q, J=7.03 Hz, 2H) 4.18-4.32 (m, 1H) 6.73-6.80 (m, 1H) 6.97 (dd, J=8.59, 4.30 Hz, 1H) 7.25 (dd, J=9.77, 2.34 Hz, 1H). HRMS [M+1]: 391.2138.

### Step G: Separation of enantiomers (Isomer 1 and Isomer 2) of ethyl 3-(4-(6-fluoro-2-oxo-2,3-dihydro-1H-benzo[d]imidazol-1-yl)piperidin-1-yl)-3-methylpyrrolidine-1-carboxylate

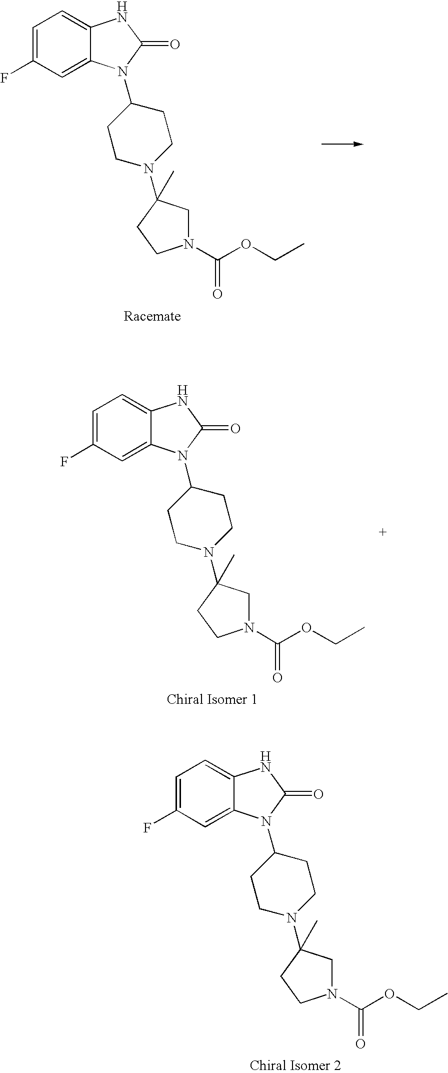

Racemic mixture of ethyl 3-(4-(6-fluoro-2-oxo-2,3-dihydro-1H-benzo[d]imidazol-1-yl)piperidin-1-yl)-3-methylpyrrolidine-1-carboxylate (105 mg, 0.27 mmol) was separated by chiral chromatography (Chiralpak AD column, 20% EtOH/80% heptane).

Isomer 1 (Example 49) was the first fraction: Enantiomer 1 of ethyl 3-(4-(6-fluoro-2-oxo-2,3-dihydro-1H-benzo[d]imidazol-1-yl)piperidin-1-yl)-3-methylpyrrolidine-1-carboxylate (35.5 mg). Retention time: 12.35 minutes (Chiralpak AD column, 20% EtOH/80% heptane). 1H NMR (400 MHz, CHLOROFORM-d) δ ppm 1.07 (d, J=3.12 Hz, 3H) 1.24 (td, J=7.13, 2.15 Hz, 3H) 1.73-2.06 (m, 4H) 2.25-2.56 (m, 4H) 2.73-2.85 (m, 1H) 2.89-3.04 (m, 1H) 3.24 (t, J=10.35 Hz, 1H) 3.30-3.68 (m, 3H) 4.03-4.18 (m, 2H) 4.27 (dddd, J=12.01, 8.11, 4.10, 3.91 Hz, 1H) 6.74 (t, J=8.98 Hz, 1H) 6.93-7.06 (m, 2H). HRMS [M+1]: 391.2145

Isomer 2 (Example 50) was the second fraction: Enantiomer 2 of ethyl 3-(4-(6-fluoro-2-oxo-2,3-dihydro-1H-benzo[d]imidazol-1-yl)piperidin-1-yl)-3-methylpyrrolidine-1-carboxylate (34.2 mg). Retention time: 21.20 minutes (Chiralpak AD column, 20% EtOH/80% heptane).1H NMR (400 MHz, CHLOROFORM-d) δ ppm 1.07 (d, J=3.12 Hz, 3H) 1.24 (td, J=7.13, 2.15 Hz, 3H) 1.73-2.06 (m, 4H) 2.25-2.56 (m, 4H) 2.73-2.85 (m, 1H) 2.89-3.04 (m, 1H) 3.24 (t, J=10.35 Hz, 1H) 3.30-3.68 (m, 3H) 4.03-4.18 (m, 2H) 4.27 (dddd, J=12.01, 8.11, 4.10, 3.91 Hz, 1H) 6.74 (t, J=8.98 Hz, 1H) 6.93-7.06 (m, 2H). HRMS [M+1]: 391.2143

### Example 51 (Isomer 1) and Example 52 (Isomer 2)

### methyl 3-(4-(6-fluoro-2-oxo-2,3-dihydro-1H-benzo[d]imidazol-1-yl)piperidin-1-yl)-3-methylpyrrolidine-1-carboxylate

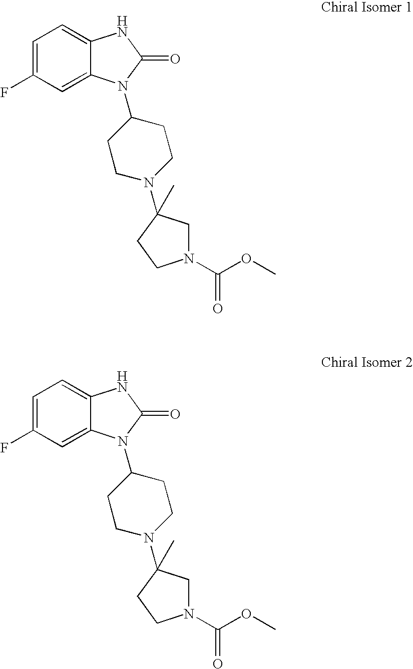

### Step A: Preparation of 6-fluoro-1-(1-(3-methylpyrrolidin-3-yl)piperidin-4-yl)-1H-benzo[d]imidazol-2(3H)-one

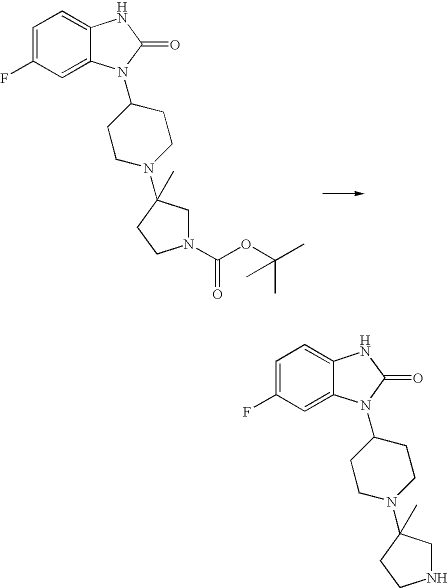

A mixture of tert-butyl 3-(4-(6-fluoro-2-oxo-2,3-dihydro-1H-benzo[d]imidazol-1-yl)piperidin-1-yl)-3-methylpyrrolidine-1-carboxylate (1.028 g, 2.46 mmol) and 2,2,2-trifluoroacetic acid (5 mL, 67.31 mmol) in dichloromethane (15 mL) was stirred at room temperature for 0.5 hours. The reaction mixture was concentrated under reduced pressure and the residue was used in the subsequent reaction without further purification.

### Step B: Preparation of methyl 3-(4-(6-fluoro-2-oxo-2,3-dihydro-1H-benzo[d]imidazol-1-yl)piperidin-1-yl)-3-methylpyrrolidine-1-carboxylate

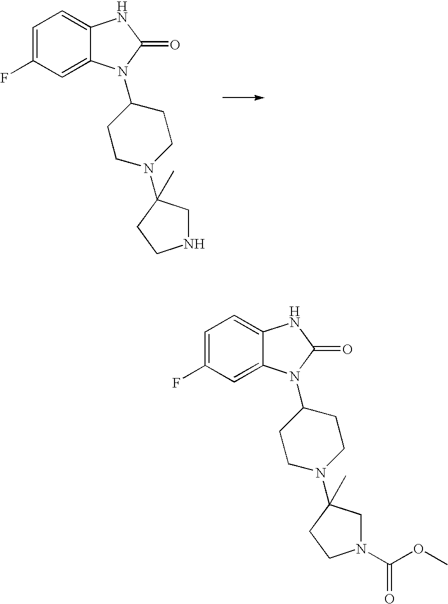

Methyl carbonochloridate (0.024 mL, 0.31 mmol) was added to a mixture of 6-fluoro-1-(1-(3-methylpyrrolidin-3-yl)piperidin-4-yl)-1H-benzo[d]imidazol-2(3H)-one (TFA salt, 0.133 g, 0.307 mmol) and triethylamine (0.428 mL, 3.07 mmol) in dichloromethane (3 mL) at 0° C. The reaction mixture was stirred at room at 0° C. for 0.5 hours. Water was added to the mixture and extracted with dichloromethane (3×10 mL). Combined organic layers were washed with brine, dried over MgSO₄, filtered and concentrated under reduced pressure. A solution of 2-Amino-2-methylpropane (0.323 mL, 3.07 mmol) in MeOH (5 ml) was added to the residue and the mixture was heated at 60° C. for 1 hour. The reaction mixture was concentrated under reduced pressure. The crude product was purified by high pH preparative HPLC (20-40% MeCN in water) to give the title compound (0.110 g, 95%) as solid. 1H NMR (400 MHz, CHLOROFORM-D) δ ppm 1.06 (d, J=1.95 Hz, 3H) 1.70-2.07 (m, 4H) 2.23-2.55 (m, 4H) 2.77 (t, J=11.33 Hz, 1H) 2.94 (d, J=7.03 Hz, 1H) 3.24 (dd, J=12.70, 10.35 Hz, 1H) 3.29-3.75 (m, 6H) 4.27 (t, J=11.72 Hz, 1H) 6.69-6.81 (m, 1H) 6.99 (dd, J=8.59, 3.52 Hz, 2H) 10.30 (s, 1H).

### Step C: Separation of enantiomers of methyl 3-(4-(6-fluoro-2-oxo-2,3-dihydro-1H-benzo[d]imidazol-1-yl)piperidin-1-yl)-3-methylpyrrolidine-1-carboxylate

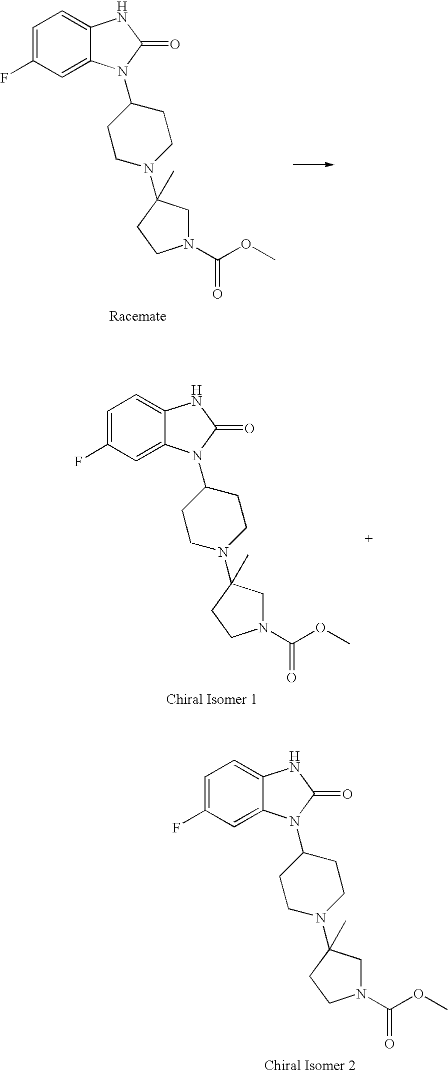

Racemic mixture of methyl 3-(4-(6-fluoro-2-oxo-2,3-dihydro-1H-benzo[d]imidazol-1-yl)piperidin-1-yl)-3-methylpyrrolidine-1-carboxylate (110 mg, 0.29 mmol) was separated by chiral chromatography (Chiralpak AD column, 20% EtOH/80% heptane).

Isomer 1 (Example 51) was the first fraction: Enantiomer 1 of methyl 3-(4-(6-fluoro-2-oxo-2,3-dihydro-1H-benzo[d]imidazol-1-yl)piperidin-1-yl)-3-methylpyrrolidine-1-carboxylate (36.6 mg). Retention time: 13.70 minutes (Chiralpak AD column, 20% EtOH/80% heptane). 1H NMR (400 MHz, CHLOROFORM-D) δ ppm 1.06 (d, J=1.95 Hz, 3H) 1.70-2.07 (m, 4H) 2.23-2.55 (m, 4H) 2.77 (t, J=11.33 Hz, 1H) 2.94 (d, J=7.03 Hz, 1H) 3.24 (dd, J=12.70, 10.35 Hz, 1H) 3.29-3.75 (m, 6H) 4.27 (t, J=11.72 Hz, 1H) 6.69-6.81 (m, 1H) 6.99 (dd, J=8.59, 3.52 Hz, 2H) 10.30 (s, 1H). HRMS [M+1]: 377.1979.

Isomer 2 (Example 52) was the second fraction: Enantiomer 2 of methyl 3-(4-(6-fluoro-2-oxo-2,3-dihydro-1H-benzo[d]imidazol-1-yl)piperidin-1-yl)-3-methylpyrrolidine-1-carboxylate (36.5 mg, 33.2%) Retention time: 24.28 minutes (Chiralpak AD column, 20% EtOH/80% heptane).1H NMR (400 MHz, CHLOROFORM-D) δ ppm 1.06 (d, J=1.95 Hz, 3H) 1.70-2.07 (m, 4H) 2.23-2.55 (m, 4H) 2.77 (t, J=11.33 Hz, 1H) 2.94 (d, J=7.03 Hz, 1H) 3.24 (dd, J=12.70, 10.35 Hz, 1H) 3.29-3.75 (m, 6H) 4.27 (t, J=11.72 Hz, 1H) 6.69-6.81 (m, 1H) 6.99 (dd, J=8.59, 3.52 Hz, 2H) 10.30 (s, 1H). HRMS [M+1]: 377.1976.

### Example 53 (Isomer 1) and Example 54 (Isomer 2)

### 2-fluoroethyl 3-(4-(6-fluoro-2-oxo-2,3-dihydro-1H-benzo[d]imidazol-1-yl)piperidin-1-yl)-3-methylpyrrolidine-1-carboxylate

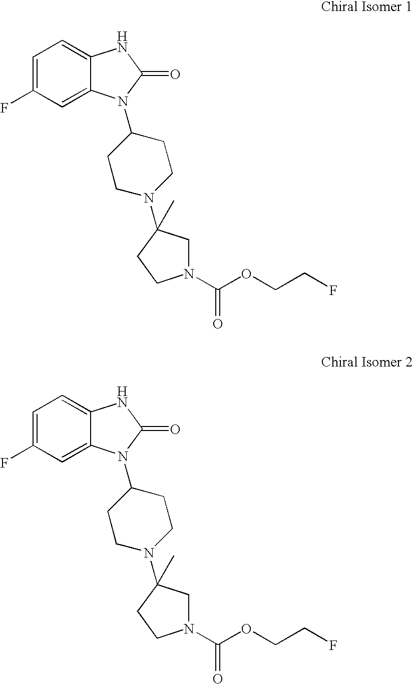

### Step A: Preparation of 2-fluoroethyl 3-(4-(6-fluoro-2-oxo-2,3-dihydro-1H-benzo[d]imidazol-1-yl)piperidin-1-yl)-3-methylpyrrolidine-1-carboxylate (racemate)

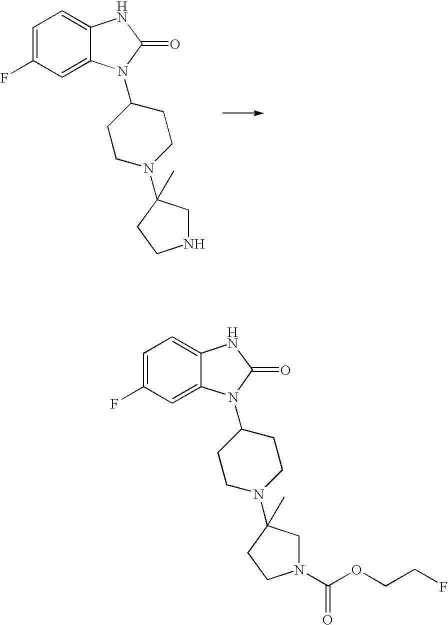

Following an analogous procedure to that described in the Step B of Example 51 and Example 52, the title compound was made from 6-fluoro-1-(1-(3-methylpyrrolidin-3-yl)piperidin-4-yl)-1H-benzo[d]imidazol-2(3H)-one (TFA salt, 0.133 g, 0.307 mmol) and 2-fluoroethyl carbonochloridate (0.029 mL, 0.31 mmol). The crude product was purified by high pH preparative HPLC (20-40% MeCN in water) to give the title compound (0.115 g, 92%) as solid. 1H NMR (400 MHz, CHLOROFORM-D) δ ppm 1.04 (d, J=1.95 Hz, 3H) 1.67-1.99 (m, 4H) 2.23-2.52 (m, 4H) 2.67-2.80 (m, 1H) 2.91 (d, J=9.77 Hz, 1H) 3.24 (d, J=10.16 Hz, 1H) 3.31-3.48 (m, 2H) 3.58 (q, J=9.37 Hz, 1H) 4.15-4.36 (m, 3H) 4.44-4.53 (m, 1H) 4.58-4.67 (m, 1H) 6.64-6.77 (m, 1H) 6.90-7.01 (m, 2H) 10.19 (br. s., 1H)

### Step B: Separation of enantiomer of 2-fluoroethyl 3-(4-(6-fluoro-2-oxo-2,3-dihydro-1H-benzo[d]imidazol-1-yl)piperidin-1-yl)-3-methylpyrrolidine-1-carboxylate

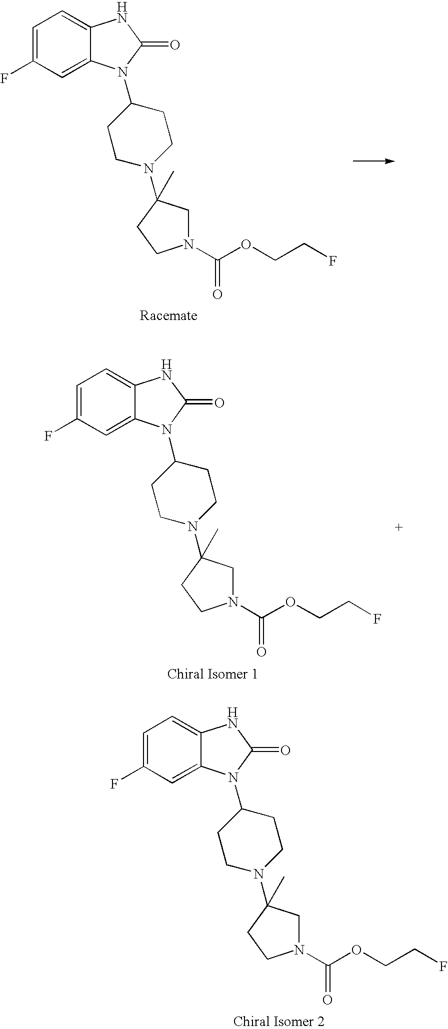

Racemic mixture of 2-fluoroethyl 3-(4-(6-fluoro-2-oxo-2,3-dihydro-1H-benzo[d]imidazol-1-yl)piperidin-1-yl)-3-methylpyrrolidine-1-carboxylate (115 mg, 0.28 mmol) was separated by chiral chromatography (Chiralpak AD column, 20% ⁱPrOH/80% heptane).

Isomer 1 (Example 53) was the first fraction: Enantiomer 1 of 2-fluoroethyl 3-(4-(6-fluoro-2-oxo-2,3-dihydro-1H-benzo[d]imidazol-1-yl)piperidin-1-yl)-3-methylpyrrolidine-1-carboxylate (37.4 mg). Retention time: 15.75 minutes (Chiralpak AD column, 40% EtOH/60% heptane). 1H NMR (400 MHz, CHLOROFORM-D) δ ppm 1.04 (d, J=1.95 Hz, 3H) 1.67-1.99 (m, 4H) 2.23-2.52 (m, 4H) 2.67-2.80 (m, 1H) 2.91 (d, J=9.77 Hz, 1H) 3.24 (d, J=10.16 Hz, 1H) 3.31-3.48 (m, 2H) 3.58 (q, J=9.37 Hz, 1H) 4.15-4.36 (m, 3H) 4.44-4.53 (m, 1H) 4.58-4.67 (m, 1H) 6.64-6.77 (m, 1H) 6.90-7.01 (m, 2H) 10.19 (br. s., 1H). HRMS [M+1]: 409.2038.

Isomer 2 (Example 54) was the second fraction: Enantiomer 2 of 2-fluoroethyl 3-(4-(6-fluoro-2-oxo-2,3-dihydro-1H-benzo[d]imidazol-1-yl)piperidin-1-yl)-3-methylpyrrolidine-1-carboxylate (40.5 mg). Retention time: 11.57 minutes (Chiralpak AD column, 40% EtOH/60% heptane). 1H NMR (400 MHz, CHLOROFORM-D) δ ppm 1.04 (d, J=1.95 Hz, 3H) 1.67-1.99 (m, 4H) 2.23-2.52 (m, 4H) 2.67-2.80 (m, 1H) 2.91 (d, J=9.77 Hz, 1H) 3.24 (d, J=10.16 Hz, 1H) 3.31-3.48 (m, 2H) 3.58 (q, J=9.37 Hz, 1H) 4.15-4.36 (m, 3H) 4.44-4.53 (m, 1H) 4.58-4.67 (m, 1H) 6.64-6.77 (m, 1H) 6.90-7.01 (m, 2H) 10.19 (br. s., 1H). HRMS [M+1]: 409.2039.

### Example 55 (Isomer 1) and Example 56 (Isomer 2)

### ethyl 3-(4-(5-fluoro-6-methyl-2-oxo-2,3-dihydro-1H-benzo[d]imidazol-1-yl)piperidin-1-yl)-3-methylpyrrolidine-1-carboxylate

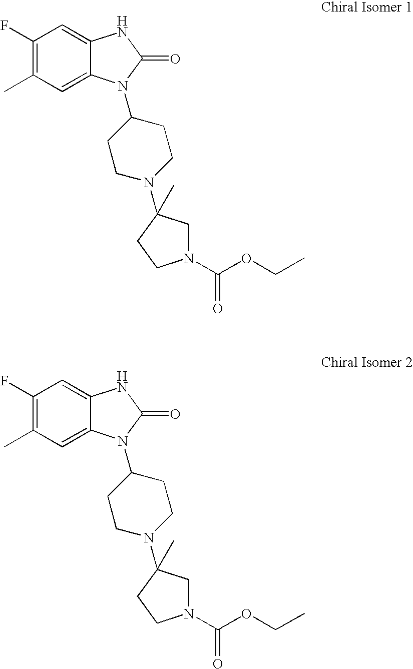

### Step A: Preparation of tert-butyl 3-(4-(4-fluoro-5-methyl-2-nitrophenylamino)piperidin-1-yl)-3-methylpyrrolidine-1-carboxylate

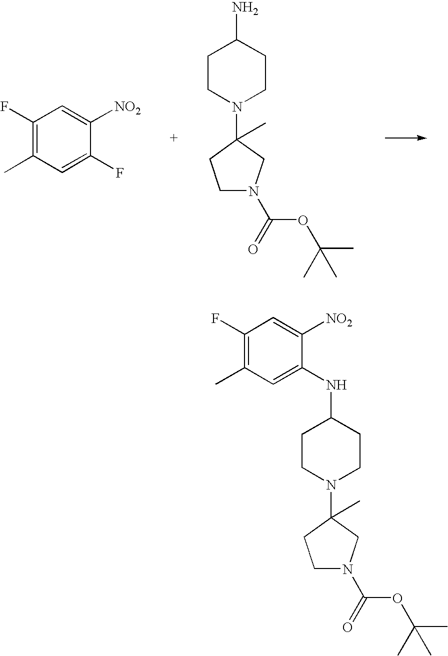

Following an analogous procedure to that described in Step C of the Example 49 and Example 50, the title compound was made from tert-butyl 3-(4-aminopiperidin-1-yl)-3-methylpyrrolidine-1-carboxylate (200 mg, 0.71 mmol) and 1,4-difluoro-2-methyl-5-nitrobenzene (122 mg, 0.71 mmol). The crude product was purified by high pH preparative HPLC (50-70% MeCN in water) to give the title compound (278 mg, 90%) as solid. MS (M+1): 437.3.

### Step B: Preparation of tert-butyl 3-(4-(2-amino-4-fluoro-5-methylphenylamino)piperidin-1-yl)-3-methylpyrrolidine-1-carboxylate

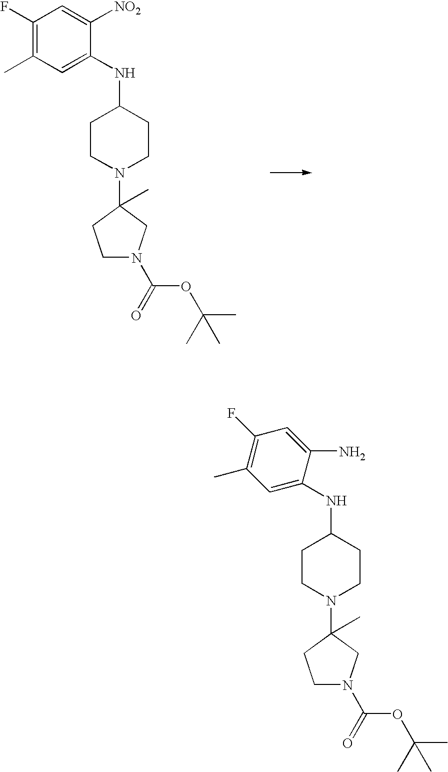

Following an analogous procedure to that described in Step D of the Example 49 and Example 50, the title compound was made from tert-butyl 3-(4-(4-fluoro-5-methyl-2-nitrophenylamino)piperidin-1-yl)-3-methylpyrrolidine-1-carboxylate. The crude product was used in the subsequent reaction without further purification. MS (M+1): 407.3.

### Step C: Preparation of tert-butyl 3-(4-(5-fluoro-6-methyl-2-oxo-2,3-dihydro-1H-benzo[d]imidazol-1-yl)piperidin-1-yl)-3-methylpyrrolidine-1-carboxylate

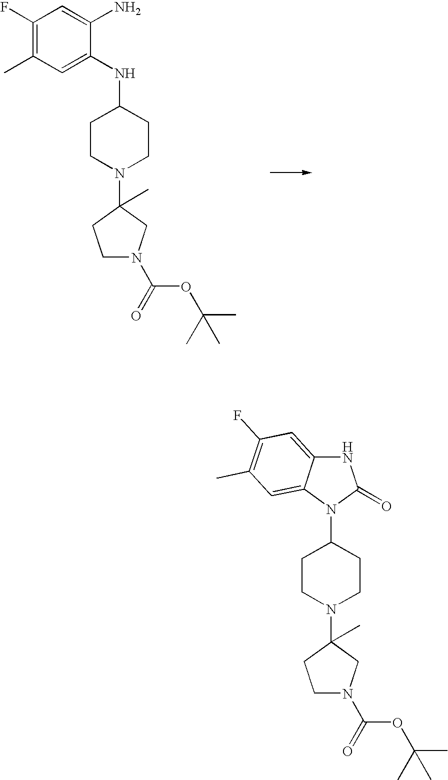

Following an analogous procedure to that described in Step E of the Example 49 and Example 50, the title compound was made from tert-butyl 3-(4-(2-amino-4-fluoro-5-methylphenylamino)piperidin-1-yl)-3-methylpyrrolidine-1-carboxylate (0.142 g, 0.35 mmol). The crude product was purified by high pH preparative HPLC (40-60% MeCN in water) to give the title compound (0.117 g, 77%) as solid. 1H NMR (400 MHz, CDCl₃) δ ppm 1.07 (d, J=3.52 Hz, 3H), 1.44 (s, 9H), 1.72-1.99 (m, 4H), 2.22-2.58 (m, 7H), 2.78 (d, J=10.55 Hz, 1H), 2.96 (d, J=19.14 Hz, 1H), 3.16-3.25 (m, 1H), 3.27-3.68 (m, 3H), 4.16-4.39 (m, 1H), 6.76 (dd, J=8.98, 1.95 Hz, 1H), 6.93-7.07 (m, 1H), 8.83 (br. s.,1H). MS (M+1): 433.3.

### Step D: Preparation of 5-fluoro-6-methyl-1-(1-(3-methylpyrrolidin-3-yl)piperidin-4-yl)-1H-benzo[d]imidazol-2(3H)-one

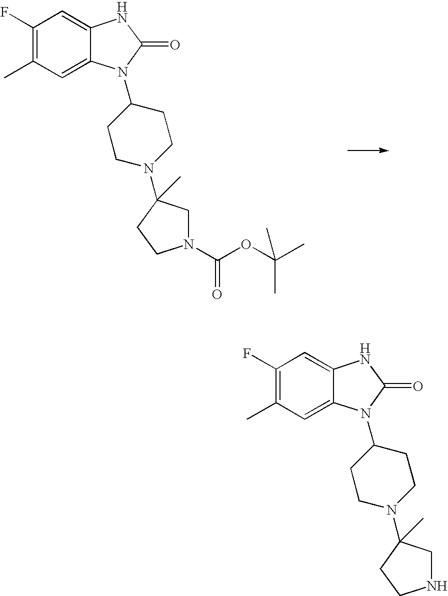

Following an analogous procedure to that described in Step A of the Example 51 and Example 52, the title compound was made from tert-butyl 3-(4-(5-fluoro-6-methyl-2-oxo-2,3-dihydro-1H-benzo[d]imidazol-1-yl)piperidin-1-yl)-3-methylpyrrolidine-1-carboxylate (0.117 g, 0.27 mmol). The crude product was used in the subsequent reaction without further purification.

### Step E: Preparation of ethyl 3-(4-(5-fluoro-6-methyl-2-oxo-2,3-dihydro-1H-benzo[d]imidazol-1-yl)piperidin-1-yl)-3-methylpyrrolidine-1-carboxylate

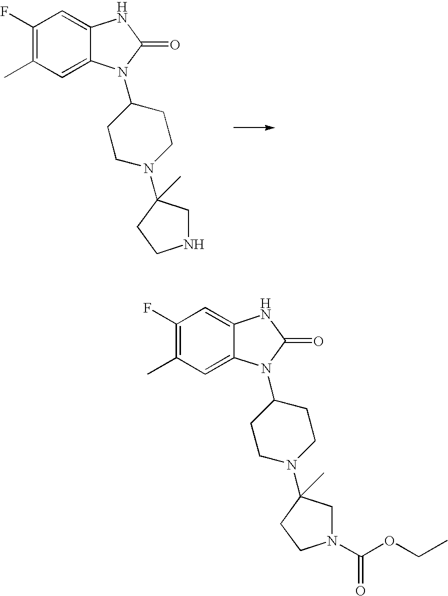

Following an analogous procedure to that described in the Step B of Example 51 and Example 52, the title compound was made from 5-fluoro-6-methyl-1-(1-(3-methylpyrrolidin-3-yl)piperidin-4-yl)-1H-benzo[d]imidazol-2(3H)-one (TFA salt, 0.121 g, 0.27 mmol) and ethyl carbonochloridate (0.026 mL, 0.27 mmol). The crude product was purified by high pH preparative HPLC (20-40% MeCN in water) to give the title compound (0.057 g, 52.2%) as solid. 1H NMR (400 MHz, CHLOROFORM-D) δ ppm 1.08 (d, J=2.34 Hz, 3H) 1.24 (td, J=7.03, 1.56 Hz, 3H) 1.68-2.07 (m, 4H) 2.21-2.58 (m, 7H) 2.72-2.85 (m, 1H) 2.95 (d, J=8.98 Hz, 1H) 3.26 (t, J=9.96 Hz, 1H) 3.31-3.68 (m, 3H) 4.03-4.17 (m, 2H) 4.20-4.35 (m, 1H) 6.80 (d, J=9.37 Hz, 1H) 6.99 (d, J=6.25 Hz, 1H) 10.03 (br. s., 1H).

### Step F: Separation of enantiomers of ethyl 3-(4-(5-fluoro-6-methyl-2-oxo-2,3-dihydro-1H-benzo[d]imidazol-1-yl)piperidin-1-yl)-3-methylpyrrolidine-1-carboxylate

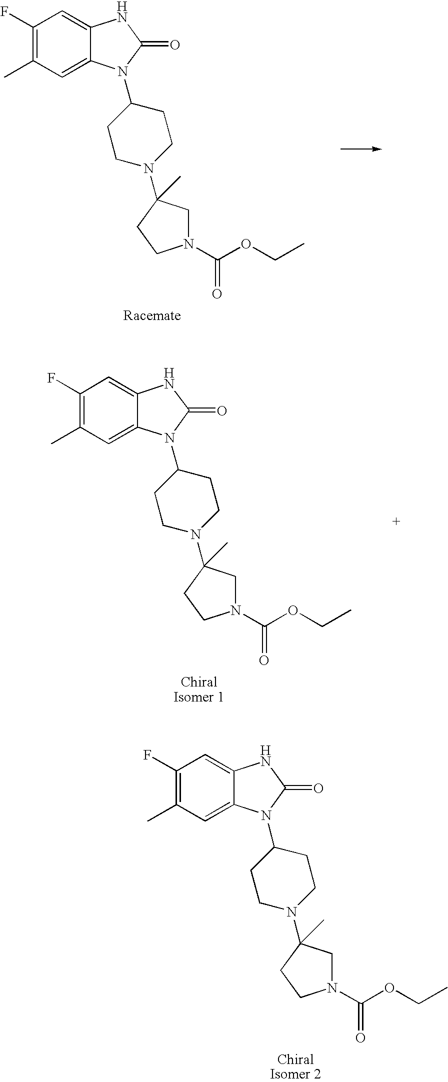

Racemic mixture of ethyl 3-(4-(5-fluoro-6-methyl-2-oxo-2,3-dihydro-1H-benzo[d]imidazol-1-yl)piperidin-1-yl)-3-methylpyrrolidine-1-carboxylate (57 mg, 0.14 mmol) was separated by chiral chromatography (Chiralpak AD column, 20% EtOH / 80% heptane).

Isomer 1 (Example 55) was the first fraction: Enantiomer 1 of ethyl 3-(4-(5-fluoro-6-methyl-2-oxo-2,3-dihydro-1H-benzo[d]imidazol-1-yl)piperidin-1-yl)-3-methylpyrrolidine-1-carboxylate (10.00 mg). Retention time: 8.70 minutes (Chiralpak AD column, 20% EtOH/80% heptane). 1H NMR (400 MHz, CHLOROFORM-D) δ ppm 1.08 (d, J=2.34 Hz, 3H) 1.24 (td, J=7.03, 1.56 Hz, 3H) 1.68-2.07 (m, 4H) 2.21-2.58 (m, 7H) 2.72-2.85 (m, 1H) 2.95 (d, J=8.98 Hz, 1H) 3.26 (t, J=9.96 Hz, 1H) 3.31-3.68 (m, 3H) 4.03-4.17 (m, 2H) 4.20-4.35 (m, 1H) 6.80 (d, J=9.37 Hz, 1H) 6.99 (d, J=6.25 Hz, 1H) 10.03 (br. s., 1H). HRMS [M+1]: 405.2296.

Isomer 2 (Example 56) was the second fraction: Enantiomer 2 of ethyl 3-(4-(5-fluoro-6-methyl-2-oxo-2,3-dihydro-1H-benzo[d]imidazol-1-yl)piperidin-1-yl)-3-methylpyrrolidine-1-carboxylate (18.30 mg). Retention time: 13.59 minutes (Chiralpak AD column, 20% EtOH/80% heptane). 1H NMR (400 MHz, CHLOROFORM-d) δ ppm 1.08 (d, J=2.34 Hz, 3H) 1.24 (td, J=7.03, 1.56 Hz, 3H) 1.68-2.07 (m, 4H) 2.21-2.58 (m, 7H) 2.72-2.85 (m, 1H) 2.95 (d, J=8.98 Hz, 1H) 3.26 (t, J=9.96 Hz, 1H) 3.31-3.68 (m, 3H) 4.03-4.17 (m, 2H) 4.20-4.35 (m, 1H) 6.80 (d, J=9.37 Hz, 1H) 6.99 (d, J=6.25 Hz, 1H) 10.03 (br. s., 1H). HRMS [M+1]: 405.2296.

### Examples 57 (Isomer 1) and Example 58 (Isomer 2)

### ethyl 3-(4-(5,6-difluoro-2-oxo-2,3-dihydro-1H-benzo[d]imidazol-1-yl)piperidin-1-yl)-3-methylpyrrolidine-1-carboxylate

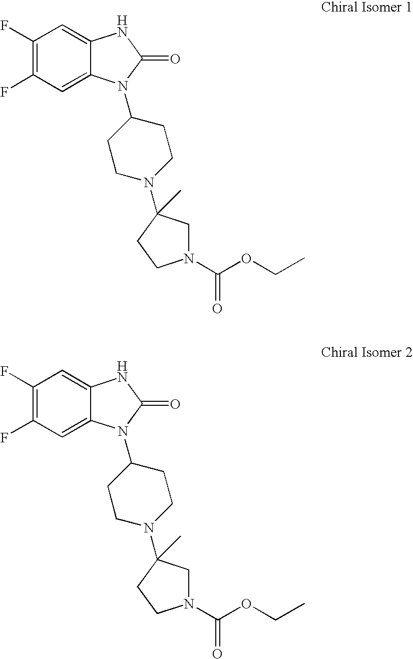

### Step A: Preparation of tert-butyl 3-(4-(2-amino-4,5-difluorophenylamino)piperidin-1-yl)-3-methylpyrrolidine-1-carboxylate

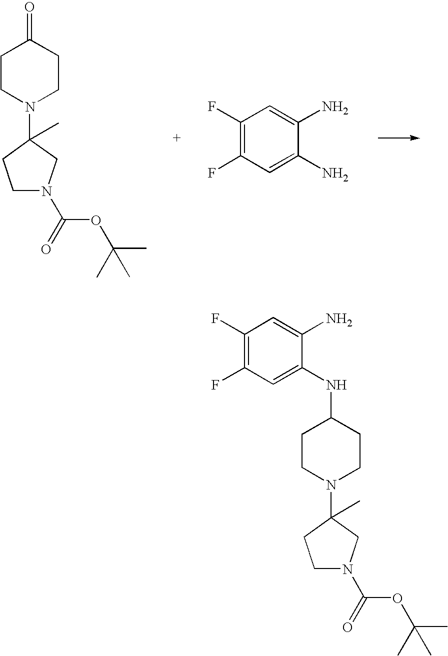

Sodium triacetoxyhydroborate (2.252 g, 10.62 mmol) was added to a mixture of tert-butyl 3-methyl-3-(4-oxopiperidin-1-yl)pyrrolidine-1-carboxylate (1 g, 3.54 mmol) and 4,5-difluorobenzene-1,2-diamine (0.510 g, 3.54 mmol) in CH₂Cl₂ (10 mL) followed by acetic acid (1.01 mL, 17.71 mmol). The reaction mixture was stirred at room temperature for 2 hours. Water was added to the mixture and the aqueous layer was extracted with dichloromethane (3×10 mL ). Combined the organic layers were washed with brine, dried over MgSO₄, filtered and concentrated under reduced pressure. The crude product was purified by high pH preparative HPLC (40-60% MeCN in water) to give the title compound (0.560 g, 38.5%) as solid. 1 H NMR (400 MHz, CDCl₃) δ ppm 1.04 (d, J=3.91 Hz, 3H), 1.42 (s, 10H), 1.74-1.87 (m, 3H), 2.01 (d, J=10.16 Hz, 2H), 2.31 (td, J,=,11.23, 2.54 Hz, 1H), 2.35-2.45 (m, 1H), 2.63 (dt, J=9.47, 2.10 Hz, 1H), 2.79 (dd, J=10.16, 5.08 Hz, 1H), 3.08 (d, J=1.95 Hz, 1H), 3.14 (t, J=9.37 Hz, 2H), 3.20-3.32 (m, 3H), 3.38-3.49 (m, 2H), 6.34-6.45 (m, 1H), 6.50 (ddd, J=10.84, 7.91, 2.34 Hz, 1H); MS (M+1): 411.3.

### Step B: Preparation of tert-butyl 3-(4-(5,6-difluoro-2-oxo-2,3-dihydro-1H-benzo[d]imidazol-1-yl)piperidin-1-yl)-3-methylpyrrolidine-1-carboxylate

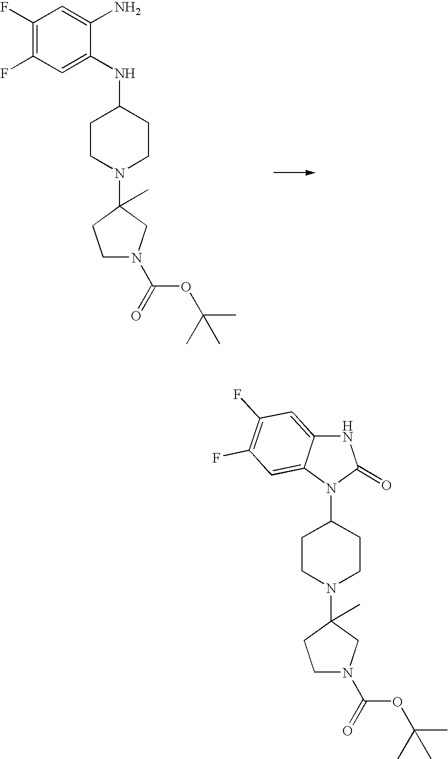

Following an analogous procedure to that described in Step E of the Example 49 and Example 50, the title compound was made from tert-butyl 3-(4-(2-amino-4,5-difluorophenylamino)piperidin-1-yl)-3-methylpyrrolidine-1-carboxylate (0.56 g, 1.36 mmol). The crude product was purified by high pH preparative HPLC (30-50% MeCN in water) to give the title compound (0.437 g, 73.4%) as solid. 1H NMR (400 MHz, CDCl₃) δ ppm 1.06 (d, J=4.30 Hz, 3H), 1.44 (s, 9H), 1.73-1.96 (m, 4H), 2.20-2.55 (m, 4H), 2.77 (d, J=11.72 Hz, 1H), 2.95 (d, J =7.03 Hz, 1H), 3.19 (t, J=10.35 Hz, 1H), 3.28-3.39 (m, 3.39-3.65 (m, 1H), 4.14-4.36 (m, 1H), 6.89 (t, J=8.40 Hz, 1H), 7.04-7.14 (m, 1H), 8.92 (s, 1H). MS (M+1): 437.2.

### Step C: Preparation of 5,6-difluoro-1-(1-(3-methylpyrrolidin-3-yl)piperidin-4-yl)-1H-benzo[d]imidazol-2(3H)-one

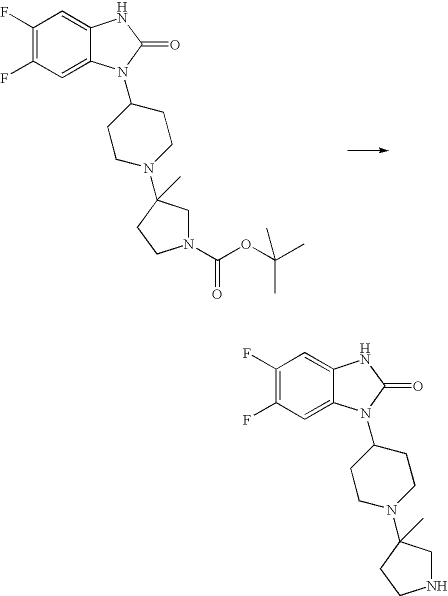

Following an analogous procedure to that described in Step A of the Example 51 and Example 52, the title compound was made from tert-butyl 3-(4-(5,6-difluoro-2-oxo-2,3-dihydro-1H-benzo[d]imidazol-1-yl)piperidin-1-yl)-3-methylpyrrolidine-1-carboxylate (0.437 g, 1.00 mmol). The crude product was used in the subsequent reaction without further purification.

### Step D: Preparation of ethyl 3-(4-(5,6-difluoro-2-oxo-2,3-dihydro-1H-benzo[d]imidazol-1-yl)piperidin-1-yl)-3-methylpyrrolidine-1-carboxylate

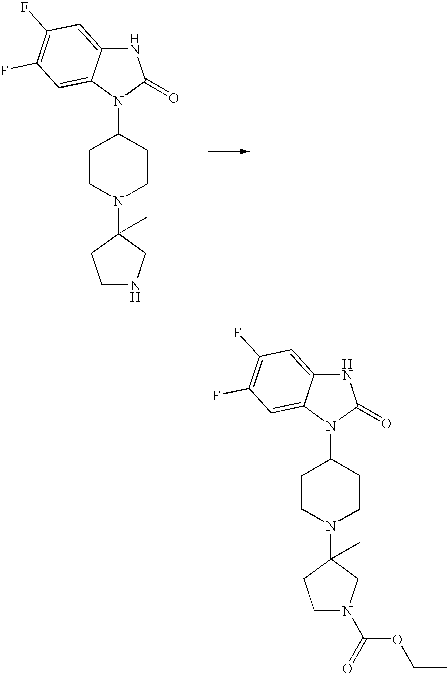

Following an analogous procedure to that described in the Step B of Example 51 and Example 52, the title compound was made from 5,6-difluoro-1-(1-(3-methylpyrrolidin-3-yl)piperidin-4-yl)-1H-benzo[d]imidazol-2(3H)-one (TFA salt, 0.150 g, 0.333 mmol) and ethyl carbonochloridate (0.032 mL, 0.33 mmol). The crude product was purified by high pH preparative HPLC (20-40% MeCN in water) to give the title compound (0.127 g, 93%). 1H NMR (400 MHz, CHLOROFORM-D) δ ppm 1.07 (d, J=2.73 Hz, 3H) 1.23 (td, J=7.13, 2.15 Hz, 3H) 1.68-1.99 (m, 4H) 2.21-2.54 (m, 4H) 2.78 (br. s.,1H) 2.95 (d, J=10.16 Hz, 1H) 3.23 (t, J=10.35 Hz, 1H) 3.29-3.70 (m, 3H) 4.04-4.17 (m, 2H) 4.24 (dddd, J=12.26, 8.25, 4.10, 3.91 Hz, 1H) 6.94 (dd, J=9.37, 7.42 Hz, 1H) 7.07 (dd, J=8.98, 7.03 Hz, 1H) 10.70 (br. s., 1H).

### Step E: Separation of enantiomers of ethyl 3-(4-(5,6-difluoro-2-oxo-2,3-dihydro-1H-benzo[d]imidazol-1-yl)piperidin-1-yl)-3-methylpyrrolidine-1-carboxylate

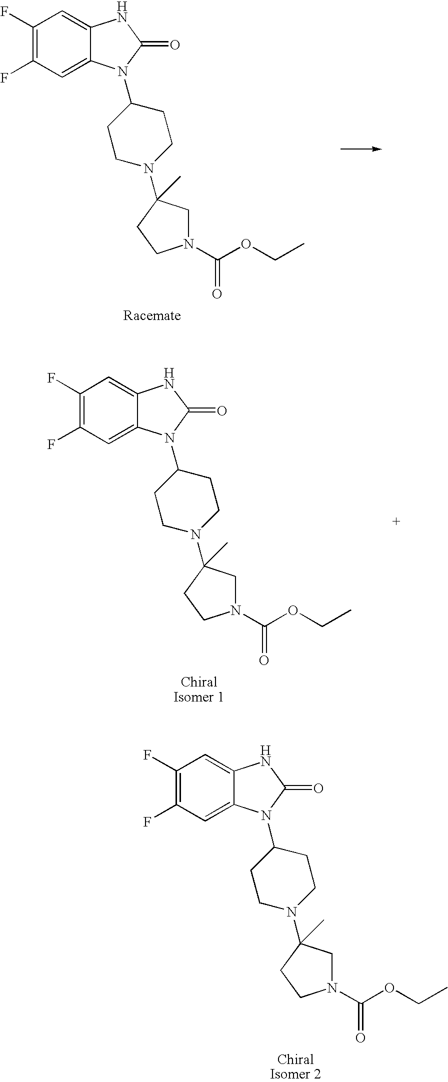

Racemic mixture of ethyl 3-(4-(5,6-difluoro-2-oxo-2,3-dihydro-1H-benzo[d]imidazol-1-yl)piperidin-1-yl)-3-methylpyrrolidine-1-carboxylate (127 mg) was separated by chiral chromatography using (Chiralpak AD column, 20% ⁱPrOH/80% heptane).

Isomer 1 (Example 57) was the first fraction: Enantiomer 1 of ethyl 3-(4-(5,6-difluoro-2-oxo-2,3-dihydro-1H-benzo[d]imidazol-1-yl)piperidin-1-yl)-3-methylpyrrolidine-1-carboxylate (17.60 mg). Retention time: 11.72 minutes (Chiralpak AD column, 20% ⁱPrOH/80% heptane).1H NMR (400 MHz, CHLOROFORM-d) δ ppm 1.07 (d, J=2.73 Hz, 3H) 1.23 (td, J=7.13, 2.15 Hz, 3H) 1.68-1.99 (m, 4H) 2.21-2.54 (m, 4H) 2.78 (br. s., 1H) 2.95 (d, J=10.16 Hz, 1H) 3.23 (t, J=10.35 Hz, 1H) 3.29-3.70 (m, 3H) 4.04-4.17 (m, 2H) 4.24 (dddd, J=12.26, 8.25, 4.10, 3.91 Hz, 1H) 6.94 (dd, J=9.37, 7.42 Hz, 1H) 7.07 (dd, J=8.98, 7.03 Hz, 1H) 10.70 (br. s., 1H). HRMS [M+1]: 409.2047.

Isomer 2 (Example 58) was the second fraction: Enantiomer 2 of ethyl 3-(4-(5,6-difluoro-2-oxo-2,3-dihydro-1H-benzo[d]imidazol-1-yl)piperidin-1-yl)-3-methylpyrrolidine-1-carboxylate (10.60 mg). Retention time: 16.10 minutes (Chiralpak AD column, 20% ⁱPrOH /80% heptane). 1H NMR (400 MHz, CHLOROFORM-D) δ ppm 1.07 (d, J=2.73 Hz, 3H) 1.23 (td, J=7.13, 2.15 Hz, 3H) 1.68-1.99 (m, 4H) 2.21-2.54 (m, 4H) 2.78 (br. s., 1H) 2.95 (d, J=10.16 Hz, 1H) 3.23 (t, J=10.35 Hz, 1H) 3.29-3.70 (m, 3H) 4.04-4.17 (m, 2H) 4.24 (dddd, J=12.26, 8.25, 4.10, 3.91 Hz, 1H) 6.94 (dd, J=9.37, 7.42 Hz, 1H) 7.07 (dd, J=8.98, 7.03 Hz, 1H) 10.70 (br. s., 1H). HRMS [M+1]: 409.2041.

### Example 59 (Isomer 1) and Example 60 (Isomer 2)

### methyl 3-(4-(5,6-difluoro-2-oxo-2,3-dihydro-1H-benzo[d]imidazol-1-yl)piperidin-1-yl)-3-methylpyrrolidine-1-carboxylate

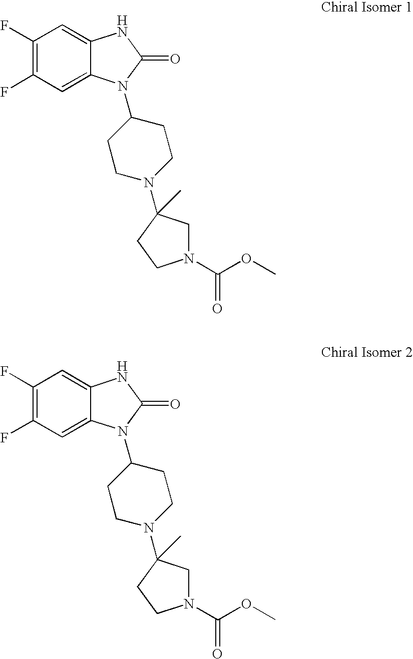

### Step A: Preparation of methyl 3-(4-(5,6-difluoro-2-oxo-2,3-dihydro-1H-benzo[d]imidazol-1-yl)piperidin-1-yl)-3-methylpyrrolidine-1-carboxylate

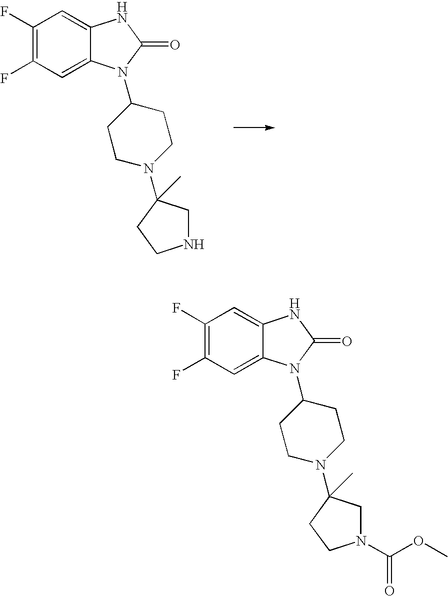

Following an analogous procedure to that described in the Step B of Example 51 and Example 52, the title compound was made from 5,6-difluoro-1-(1-(3-methylpyrrolidin-3-yl)piperidin-4-yl)-1H-benzo[d]imidazol-2(3H)-one (TFA salt, 0.150 g, 0.333 mmol) and methyl carbonochloridate (0.026 mL, 0.33 mmol). The crude product was purified by high pH preparative HPLC (20-40% MeCN in water) to give the title compound (0.110 g, 84%) as solid. 1H NMR (400 MHz, CHLOROFORM-D) δ ppm 1.06 (d, J=1.56 Hz, 3H) 1.73-1.99 (m, 4H) 2.21-2.57 (m, 4H) 2.77 (t, J=11.91 Hz, 1H) 2.94 (d, J=10.55 Hz, 1H) 3.24 (dd, J=13.09, 10.35 Hz, 1H) 3.29-3.73 (m, 6H) 4.24 (t, J=12.11 Hz, 1H) 6.94 (dd, J=9.77, 7.03 Hz, 1H) 7.07 (dd, J=10.35, 6.84 Hz, 1H) 10.72 (s, 1H).

### Step B: Separation of enantiomers of methyl 3-(4-(5,6-difluoro-2-oxo-2,3-dihydro-1H-benzo[d]imidazol-1-yl)piperidin-1-yl)-3-methylpyrrolidine-1-carboxylate

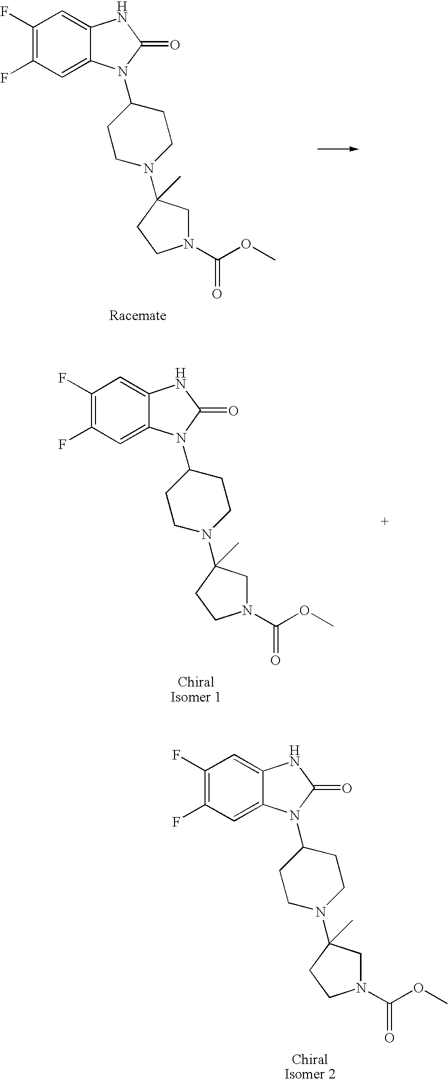

Racemic mixture of methyl 3-(4-(5,6-difluoro-2-oxo-2,3-dihydro-1H-benzo[d]imidazol-1-yl)piperidin-1-yl)-3-methylpyrrolidine-1-carboxylate (110 mg, 0.28 mmol) was separated by chiral chromatography (Chiralpak AD column, 30% EtOH/70% heptane).

Isomer 1 (Example 59) was the first fraction: Enantiomer 1 of methyl 3-(4-(5,6-difluoro-2-oxo-2,3-dihydro-1H-benzo[d]imidazol-1-yl )piperidin-1-yl)-3-methylpyrrolidine-1-carboxylate (36.1 mg). Retention time: 8.05 minutes (Chiralpak AD column, 30% EtOH/70% heptane). 1H NMR (400 MHz, CHLOROFORM-D) δ ppm 1.06 (d, J=1.56 Hz, 3H) 1.73-1.99 (m, 4H) 2.21-2.57 (m, 4H) 2.77 (t, J=11.91 Hz, 1H) 2.94 (d, J=10.55 Hz, 1H) 3.24 (dd, J=13.09, 10.35 Hz, 1H) 3.29-3.73 (m, 6H) 4.24 (t, J=12.11 Hz, 1H) 6.94 (dd, J=9.77, 7.03 Hz, 1H) 7.07 (dd, J=10.35, 6.84 Hz, 1H) 10.72 (s, 1H). HRMS [M+1]: 395.1884.

Isomer 2 (Example 60) was the second fraction: Enantiomer 2 methyl 3-(4-(5,6-difluoro-2-oxo-2,3-dihydro-1H-benzo[d]imidazol-1-yl )piperidin-1-yl)-3-methylpyrrolidine-1-carboxylate (38.5 mg). Retention time: 12.22 minutes (Chiralpak AD column, 30% EtOH/70% heptane). 1H NMR (400 MHz, CHLOROFORM-D) δ ppm 1.06 (d, J=1.56 Hz, 3H) 1.73-1.99 (m, 4H) 2.21-2.57 (m, 4H) 2.77 (t, J=11.91 Hz, 1H) 2.94 (d, J=10.55 Hz, 1H) 3.24 (dd, J=13.09, 10.35 Hz, 1H) 3.29-3.73 (m, 6H) 4.24 (t, J=12.11 Hz, 1H) 6.94 (dd, J=9.77, 7.03 Hz, 1H) 7.07 (dd, J=10.35, 6.84 Hz, 1H) 10.72 (s, 1H). HRMS [M+1]: 395.1883.

### Example 61 (Isomer 1) and Example 62 (Isomer 2)

### 2-fluoroethyl 3-(4-(5,6-difluoro-2-oxo-2,3-dihydro-1H-benzo[d]imidazol-1-yl)piperidin-1-yl)-3-methylpyrrolidine-1-carboxylate

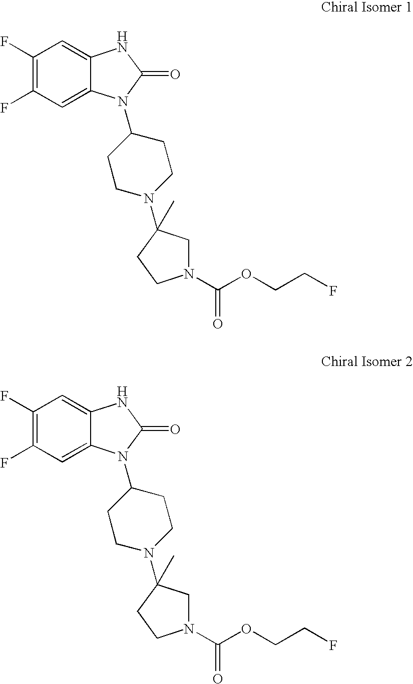

### Step A: Preparation of 2-fluoroethyl 3-(4-(5,6-difluoro-2-oxo-2,3-dihydro-1H-benzo[d]imidazol-1-yl)piperidin-1-yl)-3-methylpyrrolidine-1-carboxylate

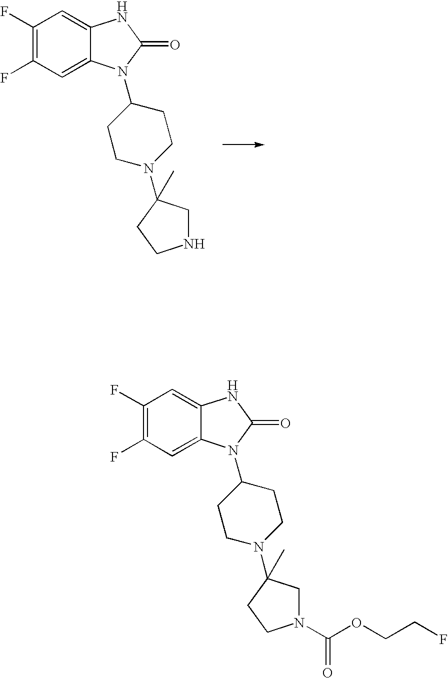

Following an analogous procedure to that described in the Step B of Example 51 and Example 52, the title compound was made from 5,6-difluoro-1-(1-(3-methylpyrrolidin-3-yl)piperidin-4-yl)-1H-benzo[d]imidazol-2(3H)-one (TFA salt, 0.150 g, 0.333 mmol) and 2-fluoroethyl carbonochloridate (0.031 mL, 0.33 mmol). The crude product was purified by high pH preparative HPLC (20-40% MeCN in water) to give the title compound (0.102 g, 72.6%) as solid. 1H NMR (400 MHz, CHLOROFORM-D) δ ppm 1.05 (s, 3H) 1.68-2.08 (m, 4H) 2.18-2.53 (m, 4H) 2.69-2.81 (m, 1H) 2.92 (d, J=10.55 Hz, 1H) 3.24 (d, J=10.16 Hz, 1H) 3.31-3.48 (m, 2H) 3.58 (q, J=9.11 Hz, 1H) 4.14-4.28 (m, 2H) 4.29-4.38 (m, 1H) 4.45-4.54 (m, 1H) 4.57-4.69 (m, 1H) 6.91 (dd, J=9.57, 6.84 Hz, 1H) 7.05 (dd, J=10.35, 6.84 Hz, 1H) 10.60 (br. s., 1H).

### Step B: separation of enantiomers of 2-fluoroethyl 3-(4-(5,6-difluoro-2-oxo-2,3-dihydro-1H-benzo[d]imidazol-1-yl)piperidin-1-yl)-3-methylpyrrolidine-1-carboxylate

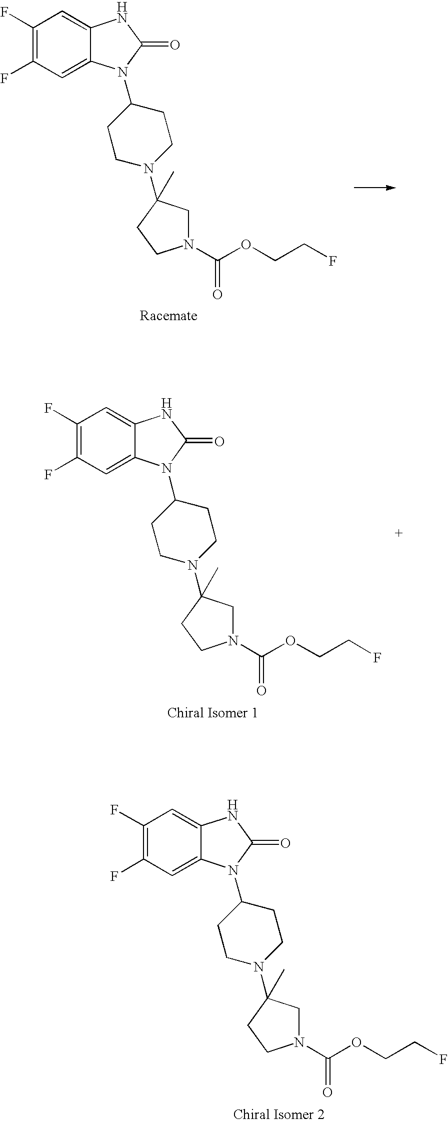

Racemic mixture of 2-fluoroethyl 3-(4-(5,6-difluoro-2-oxo-2,3-dihydro-1H-benzo[d]imidazol-1-yl)piperidin-1-yl)-3-methylpyrrolidine-1-carboxylate (102 mg, 0.24 mmol) was separated by chiral chromatography (Chiralpak AD column, 30% ⁱPrOH/70% heptane)

Isomer 1 (Example 61) was the first fraction: enantiomer 1 of 2-fluoroethyl 3-(4-(5,6-difluoro-2-oxo-2,3-dihydro-1H-benzo[d]imidazol-1-yl)piperidin-1-yl)-3-methylpyrrolidine-1-carboxylate (44.5 mg). Retention time: 10.73 minutes (Chiralpak AD column, 30% ⁱPrOH/70% heptane). 1H NMR (400 MHz, CHLOROFORM-D) δ ppm 1.05 (s, 3H) 1.68-2.08 (m, 4H) 2.18-2.53 (m, 4H) 2.69-2.81 (m, 1H) 2.92 (d, J=10.55 Hz, 1H) 3.24 (d, J=10.16 Hz, 1H) 3.31-3.48 (m, 2H) 3.58 (q, J=9.11 Hz, 1H) 4.14-4.28 (m, 2H) 4.29-4.38 (m, 1H) 4.45-4.54 (m, 1H) 4.57-4.69 (m, 1H) 6.91 (dd, J=9.57, 6.84 Hz, 1H) 7.05 (dd, J=10.35, 6.84 Hz, 1H) 10.60 (br. s., 1H). HRMS [M+1]: 427.1949.

Isomer 2 (Example 62) was the second fraction: Enantiomer 2 of 2-fluoroethyl 3-(4-(5,6-difluoro-2-oxo-2,3-dihydro-1H-benzo[d]imidazol-1-yl)piperidin-1-yl)-3-methylpyrrolidine-1-carboxylate (41.3 mg, 40.5%). Retention time: 16.30 minutes (Chiralpak AD column, 30% ⁱPrOH/70% heptane). 1H NMR (400 MHz, CHLOROFORM-D) δ ppm 1.05 (s, 3H) 1.68-2.08 (m, 4H) 2.18-2.53 (m, 4H) 2.69-2.81 (m, 1H) 2.92 (d, J=10.55 Hz, 1H) 3.24 (d, J=10.16 Hz, 1H) 3.31-3.48 (m, 2H) 3.58 (q, J=9.11 Hz, 1H) 4.14-4.28 (m, 2H) 4.29-4.38 (m, 1H) 4.45-4.54 (m, 1H) 4.57-4.69 (m, 1H) 6.91 (dd, J=9.57, 6.84 Hz, 1H) 7.05 (dd, J=10.35, 6.84 Hz, 1H) 10.60 (br. s., 1H). HRMS [M+1]: 427.1948

### Example 63 (Isomer 1) and Example 64 (Isomer 2)

### propyl 3-(4-(6-fluoro-2-oxo-2,3-dihydro-1H-benzo[d]imidazol-1-yl)piperidin-1-yl)-3-methylpyrrolidine-1-carboxylate

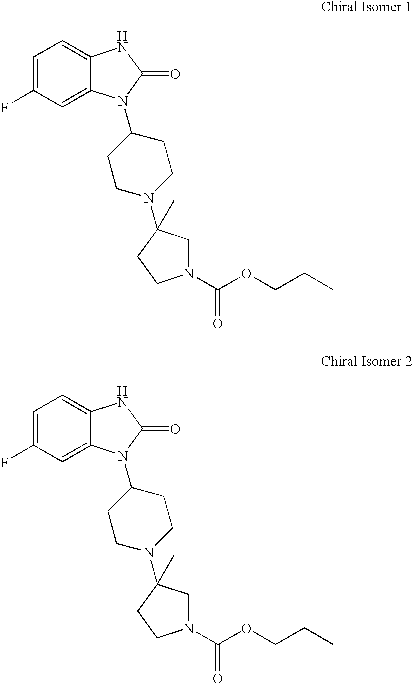

### Step A: Preparation of propyl 3-(4-(6-fluoro-2-oxo-2,3-dihydro-1H-benzo[d]imidazol-1-yl)piperidin-1-yl)-3-methylpyrrolidine-1-carboxylate (racemate)

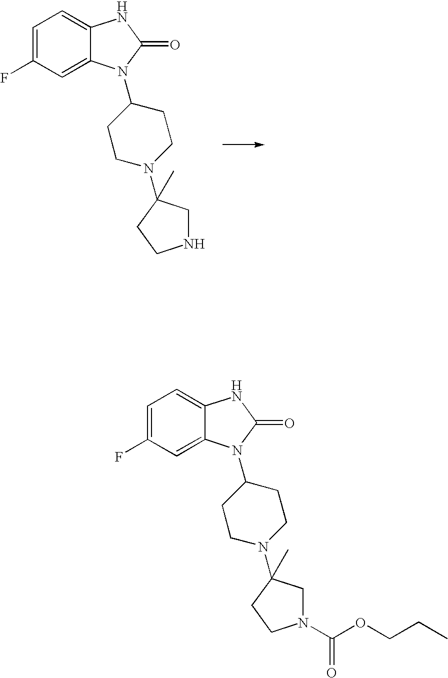

Following an analogous procedure to that described in the Step B of Example 51 and Example 52, the title compound was made from 6-fluoro-1-(1-(3-methylpyrrolidin-3-yl)piperidin-4-yl)-1H-benzo[d]imidazol-2(3H)-one (TFA salt, 0.133 g, 0.307 mmol) and propyl carbonochloridate (0.035 mL, 0.31 mmol). The crude product was purified by high pH preparative HPLC (30-50% MeCN in water) to give the title compound (0.054 g, 43.4%) as solid. 1H NMR (400 MHz, CHLOROFORM-D) δ ppm 0.92 (td, J=7.42, 2.73 Hz, 3H) 1.07 (d, J=3.12 Hz, 3H) 1.56-1.70 (m, 2H) 1.74-2.08 (m, 4H) 2.26-2.57 (m, 4H) 2.78 (t, J=8.20 Hz, 1H) 2.94 (d, J=6.25 Hz, 1H) 3.24 (t, J=9.57 Hz, 1H) 3.30-3.69 (m, 3H) 3.94-4.11 (m, 2H) 4.27 (dddd, J=12.06, 8.06, 4.30, 4.10 Hz, 1H) 6.74 (t, J=9.18 Hz, 1H) 6.92-7.08 (m, 2H) 10.12 (br. s., 1H).

### Step B: Separation of enantiomers of propyl 3-(4-(6-fluoro-2-oxo-2,3-dihydro-1H-benzo[d]imidazol-1-yl)piperidin-1-yl)-3-methylpyrrolidine-1-carboxylate

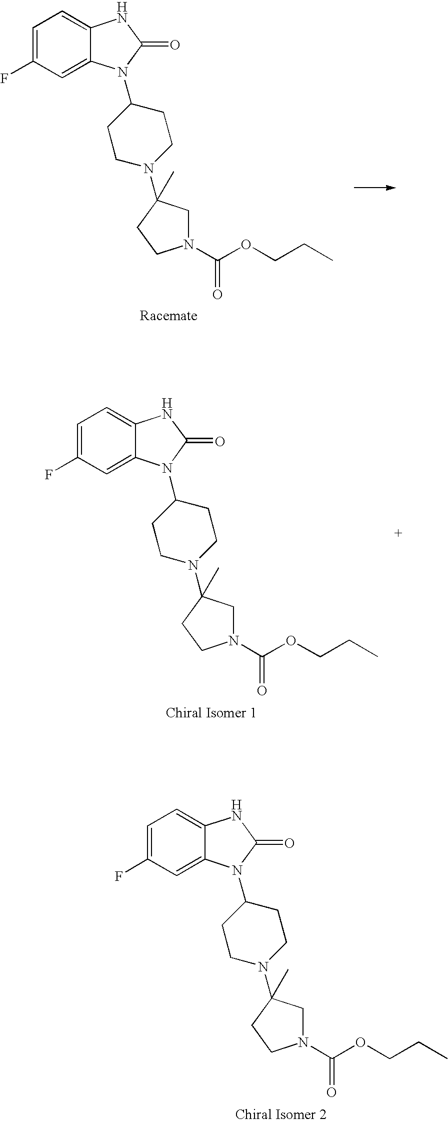

Racemic mixture of propyl 3-(4-(6-fluoro-2-oxo-2,3-dihydro-1H-benzo[d]imidazol-1-yl)piperidin-1-yl)-3-methylpyrrolidine-1-carboxylate (54 mg, 0.13 mmol) was separated by chiral chromatography (Chiralpak AD column, 20% EtOH/80% heptane).

Isomer 1 (Example 63) was the first fraction: Enantiomer 1 of propyl 3-(4-(6-fluoro-2-oxo-2,3-dihydro-1H-benzo[d]imidazol-1-yl)piperidin-1-yl)-3-methylpyrrolidine-1-carboxylate (17.30 mg). Retention time: 11.46 minutes (Chiralpak AD column, 20% EtOH/80% heptane). 1H NMR (400 MHz, CHLOROFORM-D) δ ppm 0.92 (td, J=7.42, 2.73 Hz, 3H) 1.07 (d, J=3.12 Hz, 3H) 1.56-1.70 (m, 2H) 1.74-2.08 (m, 4H) 2.26-2.57 (m, 4H) 2.78 (t, J=8.20 Hz, 1H) 2.94 (d, J=6.25 Hz, 1H) 3.24 (t, J=9.57 Hz, 1H) 3.30-3.69 (m, 3H) 3.94-4.11 (m, 2H) 4.27 (dddd, J=12.06, 8.06, 4.30, 4.10 Hz, 1H) 6.74 (t, J=9.18 Hz, 1H) 6.92-7.08 (m, 2H) 10.12 (br. s., 1H). HRMS [M+1]: 405.2295.

Isomer 2 (Example 64) was the second fraction: Enantiomer 2 of propyl 3-(4-(6-fluoro-2-oxo-2,3-dihydro-1H-benzo[d]imidazol-1-yl)piperidin-1-yl)-3-methylpyrrolidine-1-carboxylate (17.80 mg). Retention time: 23.36 minutes (Chiralpak AD column, 20% EtOH/80% heptane). 1H NMR (400 MHz, CHLOROFORM-D) δ ppm 0.92 (td, J=7.42, 2.73 Hz, 3H) 1.07 (d, J=3.12 Hz, 3H) 1.56-1.70 (m, 2H) 1.74-2.08 (m, 4H) 2.26-2.57 (m, 4H) 2.78 (t, J=8.20 Hz, 1H) 2.94 (d, J=6.25 Hz, 1H) 3.24 (t, J=9.57 Hz, 1H) 3.30-3.69 (m, 3H) 3.94-4.11 (m, 2H) 4.27 (dddd, J=12.06, 8.06, 4.30, 4.10 Hz, 1H) 6.74 (t, J=9.18 Hz, 1H) 6.92-7.08 (m, 2H) 10.12 (br. s., 1H). HRMS [M+1]: 405.2301.

### Example 65 (Isomer 1) and Example 66 (Isomer 2)

### isopropyl 3-(4-(6-fluoro-2-oxo-2,3-dihydro-1H-benzo[d]imidazol-1-yl)piperidin-1-yl)-3-methylpyrrolidine-1-carboxylate

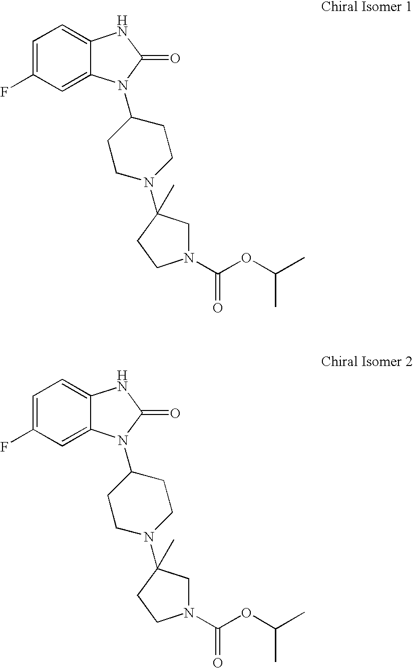

### Step A: Preparation of isopropyl 3-(4-(6-fluoro-2-oxo-2,3-dihydro-1H-benzo[d]imidazol-1-yl)piperidin-1-yl)-3-methylpyrrolidine-1-carboxylate

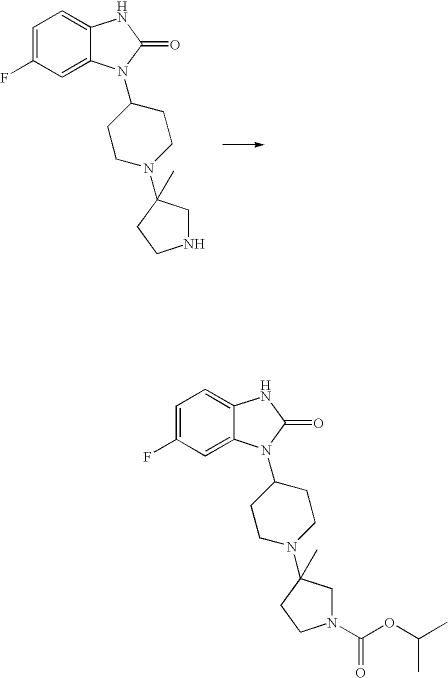

Isopropyl carbonochloridate (0.038 g, 0.31 mmol) was added to a solution of 6-fluoro-1-(1-(3-methylpyrrolidin-3-yl)piperidin-4-yl)-1H-benzo[d]imidazol-2(3H)-one (TFA salt, 0.133 g, 0.307 mmol) and triethylamine (0.428 mL, 3.07 mmol) in dichloromethane (3.000 mL) at 0° C. The resulting mixture was stirred at 0° C. for 0.5 hours. Water was added to the mixture and extracted with dichloromethane (3×10 mL ). Combined organic layers were washed with brine, dried over MgSO₄, filtered and concentrated under reduced pressure. The crude product was purified by high pH preparative HPLC (20-40% MeCN in water) to give the title compound (0.100 g, 80%) as solid. 1H NMR (400 MHz, CHLOROFORM-D) δ ppm 1.06 (d, J=2.73 Hz, 3H) 1.21 (d, J=5.86 Hz, 6H) 1.71-2.09 (m, 4H) 2.24-2.56 (m, 4H) 2.78 (d, J=10.94 Hz, 1H) 2.88-3.02 (m, 1H) 3.22 (dd, J=14.45, 10.16 Hz, 1H) 3.28-3.67 (m, 3H) 4.18-4.37 (m, 1H) 4.79-5.01 (m, 1H) 6.73 (t, J=9.18 Hz, 1H) 6.91-7.07 (m, 2H) 10.19 (br. s., 1H).

### Step B: Separation of enantiomer of isopropyl 3-(4-(6-fluoro-2-oxo-2,3-dihydro-1H-benzo[d]imidazol-1-yl)piperidin-1-yl)-3-methylpyrrolidine-1-carboxylate

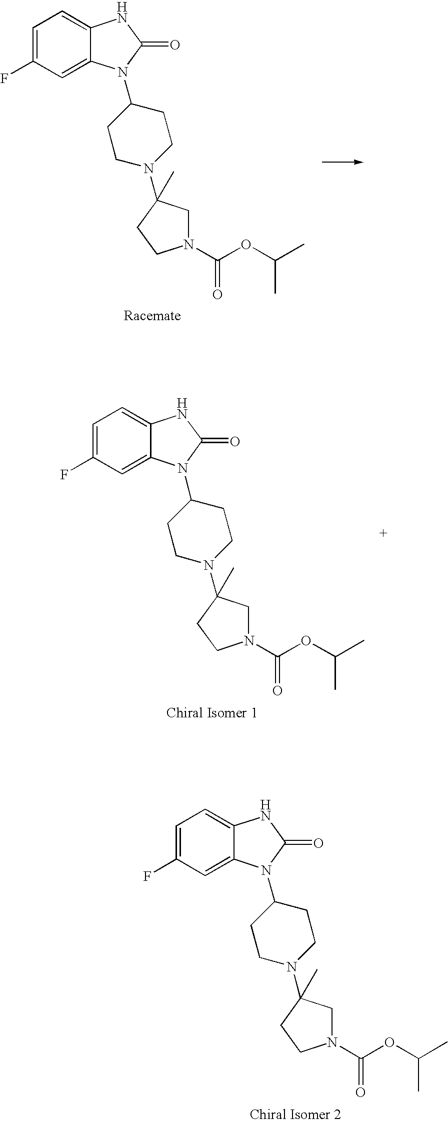

Racemic mixture of isopropyl 3-(4-(6-fluoro-2-oxo-2,3-dihydro-1H-benzo[d]imidazol-1-yl)piperidin-1-yl)-3-methylpyrrolidine-1-carboxylate (100 mg, 0.25 mmol) was separated by chiral chromatography (Chiralpak AD column, 12% EtOH/88% heptane).

Isomer 1 (Example 65) was the first fraction: Enantiomer 1 of isopropyl 3-(4-(6-fluoro-2-oxo-2,3-dihydro-1H-benzo[d]imidazol-1-yl)piperidin-1-yl)-3-methylpyrrolidine-1-carboxylate (26.8 mg). Retention time: 7.85 minutes (Chiralpak AD column, 30% EtOH/70% heptane). 1H NMR (400 MHz, CHLOROFORM-D) δ ppm 1.06 (d, J=2.73 Hz, 3H) 1.21 (d, J=5.86 Hz, 6 H) 1.71-2.09 (m, 4H) 2.24-2.56 (m, 4H) 2.78 (d, J=10.94 Hz, 1H) 2.88-3.02 (m, 1H) 3.22 (dd, J=14.45, 10.16 Hz, 1H) 3.28-3.67 (m, 3H) 4.18-4.37 (m, 1H) 4.79-5.01 (m, 1H) 6.73 (t, J=9.18 Hz, 1H) 6.91-7.07 (m, 2H) 10.19 (br. s., 1H). HRMS [M+1]: 405.2298.

Isomer 2 (Example 66) was the second fraction: Enantiomer 2 of isopropyl 3-(4-(6-fluoro-2-oxo-2,3-dihydro-1H-benzo[d]imidazol-1-yl)piperidin-1-yl)-3-methylpyrrolidine-1-carboxylate (26.0 mg). Retention time: 9.56 minutes (Chiralpak AD column, 30% EtOH/70% heptane). 1H NMR (400 MHz, CHLOROFORM-d) δ ppm 1.06 (d, J=2.73 Hz, 3H) 1.21 (d, J=5.86 Hz, 6H) 1.71-2.09 (m, 4H) 2.24-2.56 (m, 4H) 2.78 (d, J=10.94 Hz, 1H) 2.88-3.02 (m, 1H) 3.22 (dd, J=14.45, 10.16 Hz, 1H) 3.28-3.67 (m, 3H) 4.18-4.37 (m, 1H) 4.79-5.01 (m, 1H) 6.73 (t, J=9.18 Hz, 1H) 6.91-7.07 (m, 2H) 10.19 (br. s., 1H). HRMS [M+1]: 405.2301.

### Example 67 (Isomer 1) and Example 68 (Isomer 2)

### methyl 3-methyl-3-(4-(6-methyl-2-oxoindolin-1-yl)piperidin-1-yl)pyrrolidine-1-carboxylate

### Step A: Preparation of di-tert-butyl 2-(4-methyl-2-nitrophenyl)malonate

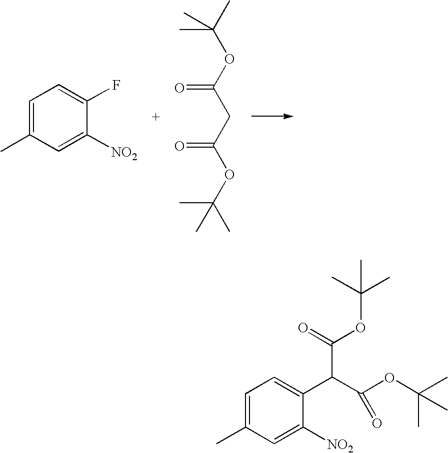

Sodium hydride hydride (2.160 g, 90.00 mmol) was added to a mixture of di-tert-butyl malonate (9.73 g, 45.00 mmol) in DMF (50 mL) and at 0° C. The reaction mixture was stirred at 0° C. for 0.5 hour. A solution of 1-fluoro-4-methyl-2-nitrobenzene (6.98 g, 45 mmol) in DMF (10 mL) was added to the mixture at room temperature. The mixture was stirred at room temperature for 12 hours. Solvent was evaporated under reduced pressure and water was added to the mixture. The aqueous layer was extracted with dichloromethane. Combined the organic layers were washed with brine, dried over MgSO₄, filtered and concentrated under reduced pressure. The crude material was purified by column chromatography using EtOAc/heptane 1:10 as eluent to give the title compound (10.36 g, 65.5%) as oil. 1H NMR (400 MHz, CDCl₃) δ ppm 1.49 (s, 18H), 2.43 (s, 3H), 5.06 (s, 1H), 7.38-7.47 (m, 2H), 7.84 (d, J=7.03 Hz, 1H).

### Step B: Preparation of di-tert-butyl 2-(2-amino-4-methylphenyl)malonate

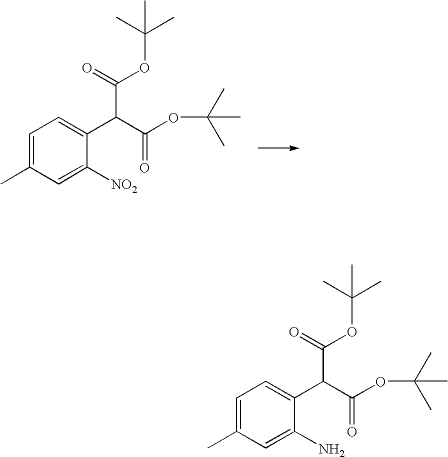

A solution of di-tert-butyl 2-(4-methyl-2-nitrophenyl)malonate (6 g, 17.07 mmol) in MeOH (100 mL) was treated with 10% palladium (0.6 g, 5.64 mmol) on charcoal and was shaken under hydrogen atmosphere at 50 psi pressure for 20 hours. The mixture was filtered and the filtrate was concentrated under reduced pressure. The crude product was purified by high pH preparative HPLC (60-80% MeCN in water) to give the title compound (3.32 g, 60.4%) as solid. MS (M+1): 322.3.

### Step C: Preparation of di-tert-butyl 2-(2-(1-(1-(tert-butoxycarbonyl)-3-methylpyrrolidin-3-yl)piperidin-4-ylamino)-4-methylphenyl)malonate

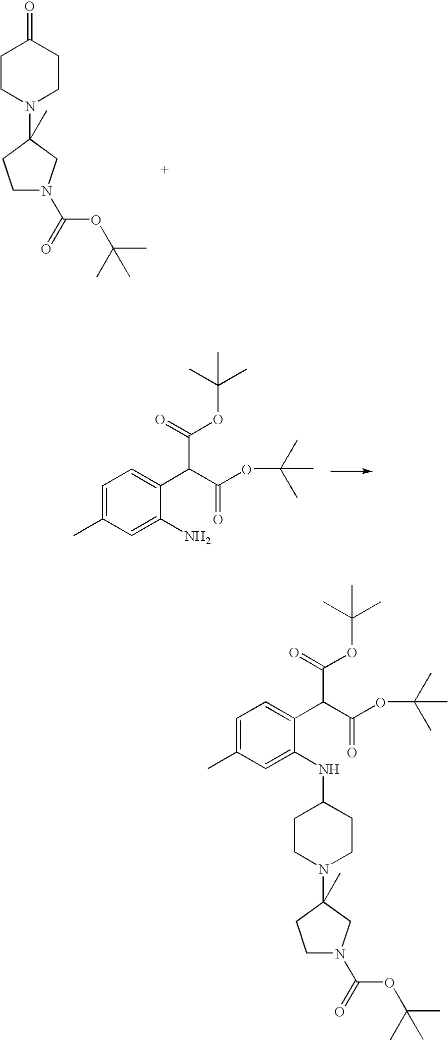

Sodium triacetoxyhydroborate (6.42 g, 30.30 mmol) was added to a mixture of di-tert-butyl 2-(2-amino-4-methylphenyl)malonate (3.246 g, 10.10 mmol) and tert-butyl 3-methyl-3-(4-oxopiperidin-1-yl)pyrrolidine-1-carboxylate (2.85 g, 10.10 mmol) in ClCH₂CH₂Cl (15 mL) followed by acetic acid (2.89 ml, 50.50 mmol). The resulting mixture was stirred at room temperature for 12 hours. Water was added to the mixture and the aqueous layer was extracted with dichloromethane. The organic layers were combined, washed with brine, dried over MgSO₄, filtered and, then concentrated under reduced pressure. The crude product was purified by high pH preparative HPLC (70-90% MeCN in water) to give the title compound (3.63 g, 61.2%).

### Step D: Preparation of 6-methyl-1-(1-(3-methylpyrrolidin-3-yl)piperidin-4-yl)indolin-2-one

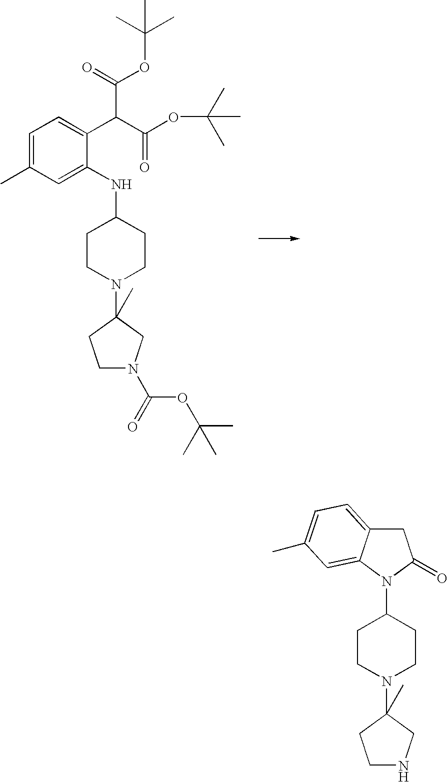

The mixture of di-tert-butyl 2-(2-(1-(1-(tert-butoxycarbonyl)-3-methylpyrrolidin-3-yl)piperidin-4-ylamino)-4-methylphenyl)malonate (3.385 g, 5.76 mmol) and 4-methylbenzenesulfonic acid (9.92 g, 57.59 mmol) in toluene (40 mL) was heated at reflux for 3 hours. The reaction mixture was concentrated under reduced pressure and was used directly in the subsequent step.

### Step E: Preparation of methyl 3-methyl-3-(4-(6-methyl-2-oxoindolin-1-yl)piperidin-1-yl)pyrrolidine-1-carboxylate

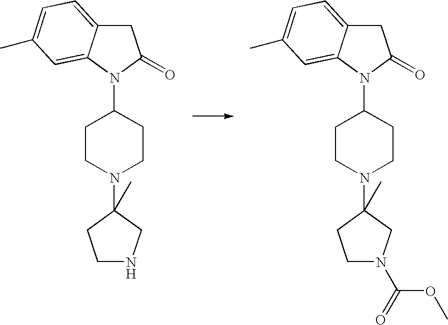

Methyl carbonochloridate (0.890 mL, 11.52 mmol) was added to a mixture of 6-methyl-1-(1-(3-methylpyrrolidin-3-yl)piperidin-4-yl)indolin-2-one (from previous step, approximately 1.805 g, 5.76 mmol) and triethylamine (16.06 mL, 115.20 mmol) in dichloromethane (20 mL) at 0° C. The reaction mixture was stirred at 0° C. Water was added to the reaction mixture and the aqueous layer was extracted with dichloromethane. Combined the organic layers were washed with brine, dried over MgSO₄, filtered and concentrated under reduced pressure. The crude product was purified by high pH preparative HPLC (40-60% MeCN in water) to give the title compound (1.290 g, 60.3%) as solid. 1H NMR (400 MHz, CDCl₃) δ ppm 1.09 (s, 3H), 1.65-1.79 (m, 2H), 1.81-2.02 (m, 2H), 2.33-2.59 (m, 7H), 2.69-2.85 (m, 1H), 2.95 (d, J=2.34 Hz, 1H), 3.28 (dd, J=13.09, 9.96 Hz, 1H), 3.33-3.68 (m, 5H), 3.71 (d, J=3.12 Hz, 3H), 4.19-4.34 (m, 1H), 6.83 (d, J=7.42 Hz, 1H), 6.95 (s, 1H), 7.11 (d, J=7.42 Hz, 1H). MS (M+1): 372.3.

### Step F: Separation of enantiomers of methyl 3-methyl-3-(4-(6-methyl-2-oxoindolin-1-yl)piperidin-1-yl)pyrrolidine-1-carboxylate

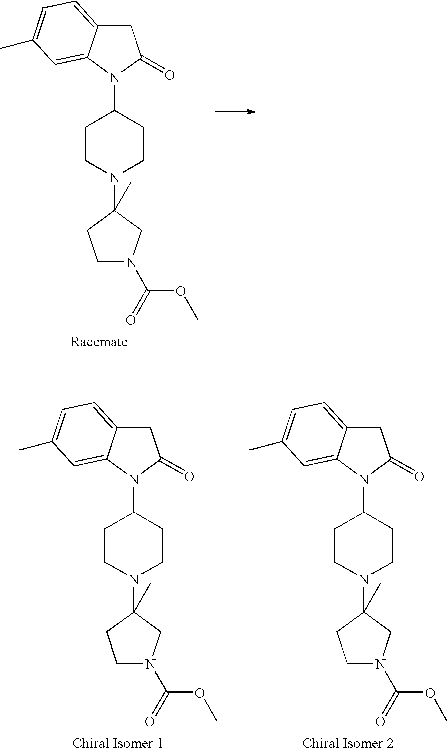

Racemic mixture of methyl 3-methyl-3-(4-(6-methyl-2-oxoindolin-1-yl)piperidin-1-yl)pyrrolidine-1-carboxylate (1.29 g, 3.47 mmol) was separated by chiral HPLC (Chiracel Chiralpak AD column with 20% EtOH/0.1% DEA in heptane).

Isomer 1 (Example 67) was the first eluting fraction: Enantiomer 1 of methyl 3-methyl-3-(4-(6-methyl-2-oxoindolin-1-yl)piperidin-1-yl)pyrrolidine-1-carboxylate (0.446 g). Retention time: 6.25 minutes (Chiracel OD column, 40% EtOH/0.1% DEA in heptane). [α]D at 25° C. in MeOH −15.4. 1H NMR (400 MHz, CDCl₃) δ ppm 1.04 (s, 3H), 1.59-1.71 (m, 2H), 1.76-2.00 (m, 2H), 2.28-2.48 (m, 7H), 2.73 (t, J=10.94 Hz, 1H), 2.91 (br. s., 1H), 3.23 (t, J=10.74 Hz, 1H), 3.28-3.63 (m, 5H), 3.65 (d, J=3.52 Hz, 3H), 4.12-4.27 (m, 1H), 6.77 (d, J=7.42 Hz, 1H), 6.92 (s, 1H), 7.06 (d, J=7.42 Hz, 1H). HRMS [M+1]: 372.2281.

Isomer 2 (Example 68) was the second eluting fraction. Enantiomer 2 of methyl 3-methyl-3-(4-(6-methyl-2-oxoindolin-1-yl)piperidin-1-yl)pyrrolidine-1-carboxylate (0.227 g). Retention time: 9.23 minutes (Chiracel OD column, 40% EtOH/0.1% DEA in heptane). [α]D at 25° C. in MeOH +19.75. 1H NMR (400 MHz, CDCl₃) δ ppm 1.04 (s, 3H), 1.59-1.71 (m, 2H), 1.76-2.00 (m, 2H), 2.28-2.48 (m, 7H), 2.73 (t, J=10.94 Hz, 1H), 2.91 (br. s., 1H), 3.23 (t, J=10.74 Hz, 1H), 3.28-3.63 (m, 5H), 3.65 (d, J=3.52 Hz, 3H), 4.12-4.27 (m, 1H), 6.77 (d, J=7.42 Hz, 1H), 6.92 (s, 1H), 7.06 (d, J=7.42 Hz, 1H). HRMS [M+1]: 372.2285.

### Example 69 (Isomer 1) and Example 70 (Isomer 2): ethyl 3-methyl-3-(4-(2-oxoindolin-1-yl)piperidin-1-yl)pyrrolidine-1-carboxylate

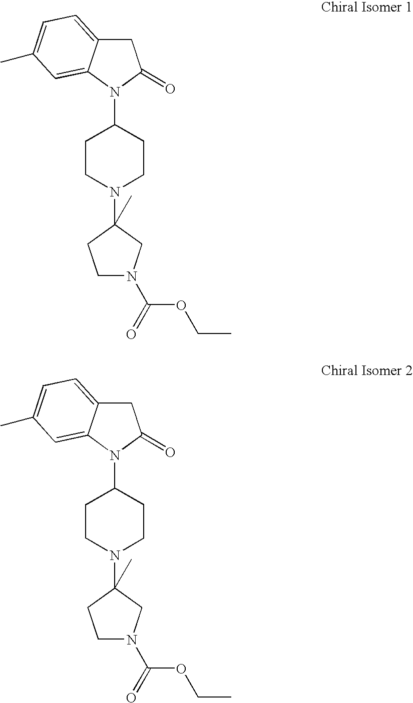

### Step A: Preparation of ethyl 3-methyl-3-(4-(2-oxoindolin-1-yl)piperidin-1-yl)pyrrolidine-1-carboxylate

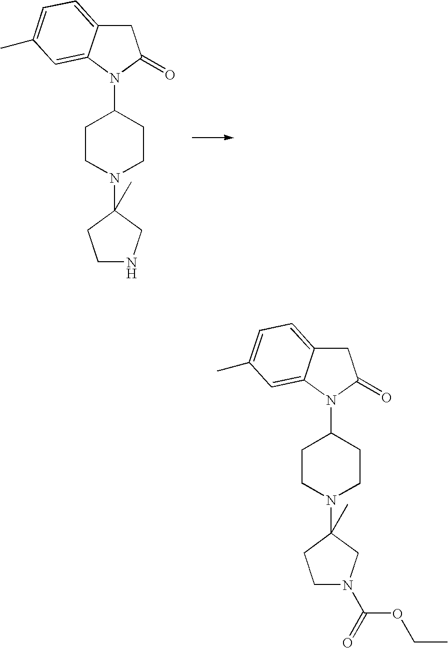

Following an analogous procedure to that described in Step E of the Example 67 and Example 68, the title compound was made from 1-(1-(3-methylpyrrolidin-3-yl)piperidin-4-yl)indolin-2-one (43.6 mg, 0.15 mmol) and ethyl carbonochloridate (0.014 mL, 0.15 mmol). The crude product was purified by high pH preparative HPLC (30-50% MeCN in water) to give the title compound (mixture of racemates) (23.70 mg, 43.8%) as solid. 1H NMR (400 MHz, CHLOROFORM-d) δ ppm 1.06 (d, J=2.34 Hz, 3H) 1.24 (td, J=7.03, 2.34 Hz, 3H) 1.63-1.75 (m, 2H) 1.77-1.97 (m, 2H) 2.27-2.53 (m, 4H) 2.70-2.81 (m, 1H) 2.86-3.02 (m, 1H) 3.22 (t, J=10.16 Hz, 1H) 3.28-3.42 (m, 2H) 3.44-3.65 (m, 3H) 4.01-4.18 (m, 2H) 4.20-4.36 (m, 1H) 6.99 (t, J=7.42 Hz, 1H) 7.13 (d, J=8.20 Hz, 1H) 7.18-7.27 (m, 2H). HRMS [M+1]: 372.2284.

### Step B: Separation of enantiomers of ethyl 3-methyl-3-(4-(2-oxoindolin-1-yl)piperidin-1-yl)pyrrolidine-1-carboxylate

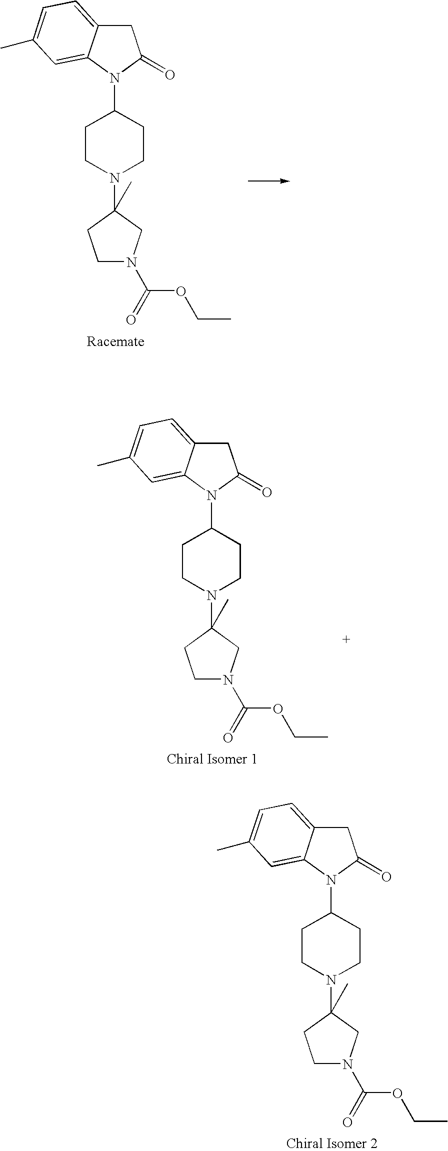

Racemic mixture of ethyl 3-methyl-3-(4-(6-methyl-2-oxoindolin-1-yl)piperidin-1-yl)pyrrolidine-1-carboxylate (0.232 g, 0.60 mmol) was separated by chiral HPLC (Chiralpak AD column with 20% EtOH/0.1% DEA in heptane).

Isomer 1 (Example 69) was the first fraction. Enantiomer 1 of ethyl 3-methyl-3-(4-(6-methyl-2-oxoindolin-1-yl)piperidin-1-yl)pyrrolidine-1-carboxylate (0.103 g). Retention time: 6.18 minutes (Chiracel OD column, 40% EtOH/0.1% DEA in heptane). 1H NMR (400 MHz, CDCl₃) δ ppm 1.02 (d, J=2.34 Hz, 3H), 1.19 (t, J=7.23 Hz, 3H), 1.65 (br. s., 2H), 1.72-1.97 (m, 2H), 2.24-2.54 (m, 7H), 2.71 (d, J=7.03 Hz, 1H), 2.88 (d, J=2.34 Hz, 1H), 3.21 (t, J=9.37 Hz, 1H), 3.26-3.63 (m, 5H), 4.07 (qd, J=7.03, 3.12 Hz, 2H), 4.19 (d, J=4.69 Hz, 1H), 6.75 (d, J=7.42 Hz, 1H), 6.89 (s, 1H), 7.04 (d, J=7.81 Hz, 1H). HRMS [M+1]: 386.2438.

Isomer 2 (Example 70) was the second fraction: Enantiomer 2 of ethyl 3-methyl-3-(4-(6-methyl-2-oxoindolin-1-yl)piperidin-1-yl)pyrrolidine-1-carboxylate (0.108 g). Retention time: 8.28 minutes (Chiracel OD column, 40% EtOH/0.1% DEA in heptane). 1H NMR (400 MHz, CDCl₃) δ ppm 1.02 (d, J=2.34 Hz, 3H), 1.19 (t, J=7.23 Hz, 3H), 1.65 (br. s., 2H), 1.72-1.97 (m, 2H), 2.24-2.54 (m, 7H), 2.71 (d, J=7.03Hz, 1H), 2.88 (d, J=2.34 Hz, 1H), 3.21 (t, J=9.37 Hz, 1H), 3.26-3.63 (m, 5H), 4.07 (qd, J=7.03, 3.12 Hz, 2H), 4.19 (d, J=4.69 Hz, 1H), 6.75 (d, J=7.42 Hz, 1H), 6.89 (s, 1H), 7.04 (d, J=7.81 Hz, 1H). HRMS [M+1]386.2437.

### Example 71

### methyl 3-methyl-3-(4-(2-oxoindolin-1-yl)piperidin-1-yl)pyrrolidine-1-carboxylate

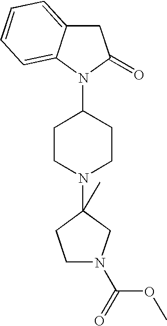

### Step A. The preparation of di-tert-butyl 2-(2-nitrophenyl)malonate

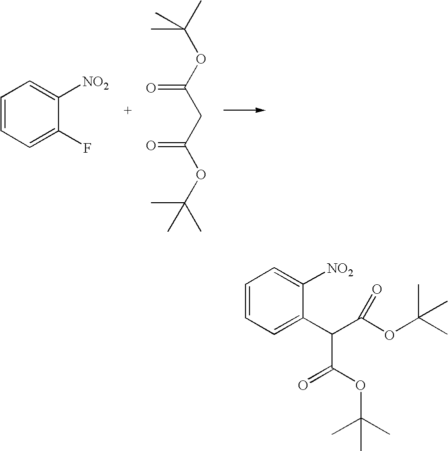

Following an analogous procedure to that described in Step A of the Example 67 and Example 68, the title compound (0.437 g, 43.3%) was prepared from 1-fluoro-2-nitrobenzene (0.423 g, 3.00 mmol) and di-tert-butyl malonate (0.672 mL, 3 mmol). 1H NMR (400 MHz, CHLOROFORM-D) δ ppm 1.47 (s, 18H), 5.08 (s, 1H), 7.46 (t, J=7.62 Hz, 1H), 7.50-7.55 (m, 1H), 7.61 (t, J=7.42 Hz, 1H), 8.01 (d, J=8.20 Hz, 1H).

### Step B. The preparation of di-tert-butyl 2-(2-aminophenyl)malonate

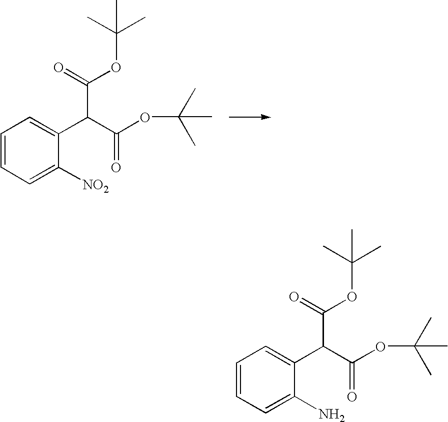

Following an analogous procedure to that described in Step B of the Example 67 and Example 68, the title compound (214 mg, 53.8%) was prepared from di-tert-butyl 2-(2-nitrophenyl)malonate (437 mg, 1.30 mmol). 1H NMR (400 MHz, CHLOROFORM-d) δ ppm 1.44 (s, 18H) 4.08 (br. s., 2H) 4.44 (s, 1H) 6.65-6.70 (m, 1H) 6.73 (td, J=7.62, 1.17 Hz, 1H) 7.08 (td, J=7.71, 1.37 Hz, 1H) 7.14 (dd, J=7.62, 1.37 Hz, 1H)

### Step C: Preparation of di-tert-butyl 2-(2-(1-(1-(tert-butoxycarbonyl)-3-methylpyrrolidin-3-yl)piperidin-4-ylamino)phenyl)malonate

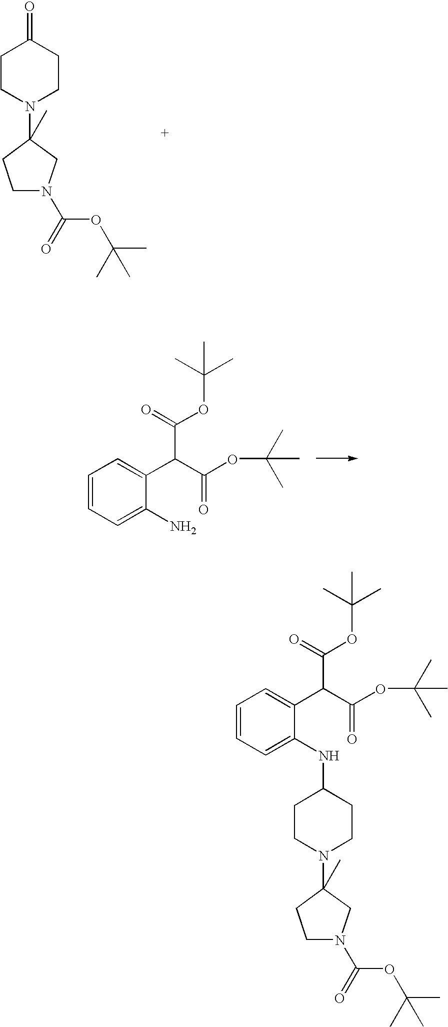

Following an analogous procedure to that described in Step C of the Example 67 and Example 68 (dichloromethane was used as a solvent instead of dichloroethane), the title compound was made from di-tert-butyl 2-(2-aminophenyl)malonate (214 mg, 0.70 mmol) and tert-butyl 3-methyl-3-(4-oxopiperidin-1-yl)pyrrolidine-1-carboxylate (197 mg, 0.70 mmol). The crude product was purified by high pH preparative HPLC (60-80% MeCN in water) to give the title compound (88 mg, 22.03%) as pale yellow solid. 1H NMR (400 MHz, CDCl₃) δ ppm 1.05 (d, J=2.73 Hz, 3H), 1.31-1.64 (m, 28H), 1.69-1.91 (m, 2H), 1.94-2.10 (m, 2H), 2.24-2.47 (m, 2H), 2.64 (d, J=4.30 Hz, 1H), 2.79 (d, J=5.86 Hz, 1H), 3.14 (t, J=9.18 Hz, 1H), 3.22-3.36 (m, 3H), 3.37-3.59 (m, 2H), 4.40 (s, 1H), 4.62 (br. s., 1H), 6.59-6.69 (m, 2H), 7.06-7.17 (m, 2H); MS (M+1): 574.4.

### Step D: Preparation of 1-(1-(3-methylpyrrolidin-3-yl)piperidin-4-yl)indolin-2-one

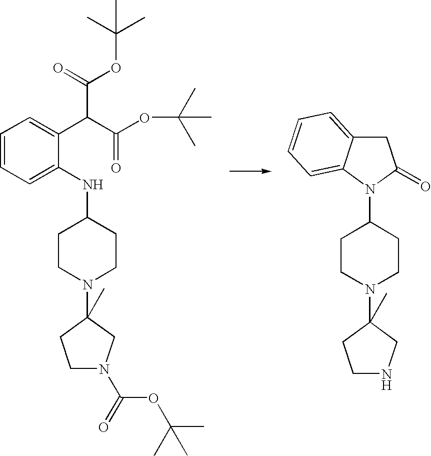

The mixture of di-tert-butyl 2-(2-(1-(1-(tert-butoxycarbonyl)-3-methylpyrrolidin-3-yl)piperidin-4-ylamino)phenyl)malonate (88 mg, 0.15 mmol) and 4-methylbenzenesulfonic acid (264 mg, 1.53 mmol) in toluene (15 mL) was heated at reflux for 3 hours. 1N solution of NaOH was added to the reaction mixture and extracted with dichloromethane (3×10 mL). The combined organic extracts were washed with water and brine, dried over MgSO₄, and filtered. The crude product was purified by high pH preparative HPLC (10-30% MeCN in water) to give the title compound (43.6 mg, 95%) as solid. 1H NMR (400 MHz, CDCl₃) δ ppm 1.05 (s, 3H), 1.57-1.73 (m, 3H), 1.76-1.88 (m, 1H), 2.24-2.50 (m, 4H), 2.69-2.91 (m, 3H), 2.92-3.15 (m, 3H), 3.48 (s, 2H), 4.17-4.39 (m, 1H), 6.98 (t, J=7.42 Hz, 1H), 7.12-7.30 (m, 3H). MS (M+1): 300.2.

### Step E: Preparation of methyl 3-methyl-3-(4-(2-oxoindolin-1-yl)piperidin-1-yl)pyrrolidine-1-carboxylate

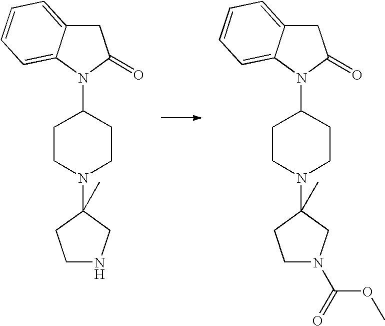

Following an analogous procedure to that described in Step E of the Example 67 and Example 68, the title compound was made from 1-(1-(3-methylpyrrolidin-3-yl)piperidin-4-yl)indolin-2-one, and methyl carbonochloridate (0.024 g, 0.26 mmol). The crude product was purified by high pH preparative HPLC (30-50% MeCN in water) to give the title compound (0.024 g, 51.6%) as solid. 1H NMR (400 MHz, CDCl₃) δ ppm 1.05 (s, 3H), 1.61-1.76 (m, 2H), 1.77-1.99 (m, 2H), 2.28-2.52 (m, 4H), 2.68-2.81 (m, 1H), 2.84-2.99 (m, 1H), 3.17-3.28 (m, 1H), 3.29-3.64 (m, 5H), 3.67 (d, J=3.52 Hz, 3H), 4.27 (t, J,=10.16 Hz, 1H), 6.99 (t, J=7.42 Hz, 1H), 7.10-7.16 (m, 1H), 7.18-7.27 (m, 2H). HRMS [M+1]: 358.2124.

### Example 72 (isomer 1) and Example 73 (isomer 2)

### ethyl 3-methyl-3-(4-(2-oxoindolin-1-yl)piperidin-1-yl)pyrrolidine-1-carboxylate

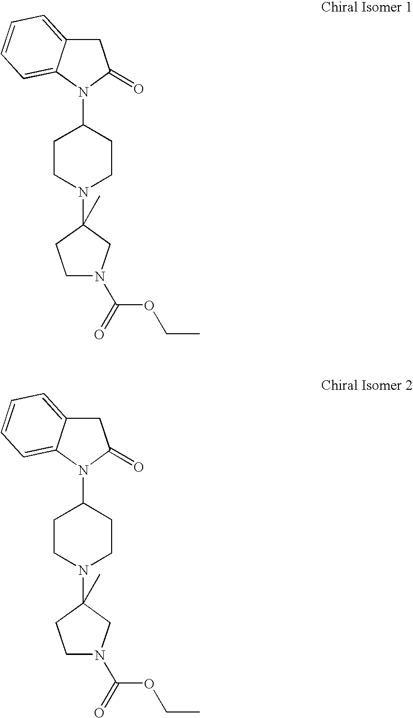

### Step A. Preparation of ethyl 3-methyl-3-(4-(2-oxoindolin-1-yl)piperidin-1-yl)pyrrolidine-1-carboxylate

Following an analogous procedure to that described in Step E of the Example 67 and Example 68, the title compound was made from 1-(1-(3-methylpyrrolidin-3-yl)piperidin-4-yl)indolin-2-one, and ethyl carbonochloridate.

### Step B. Separation of enantiomers of ethyl 3-methyl-3-(4-(2-oxoindolin-1-yl)piperidin-1-yl)pyrrolidine-1-carboxylate

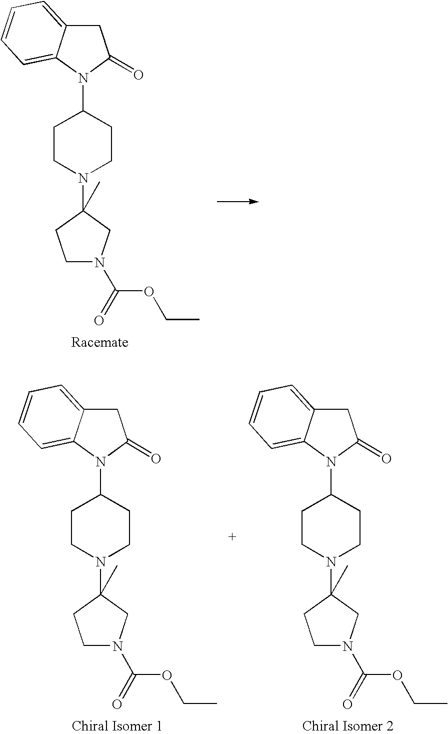

Racemic mixture of ethyl 3-methyl-3-(4-(2-oxoindolin-1-yl)piperidin-1-yl)pyrrolidine-1-carboxylate (186 mg, 0.50 mmol) was separated by chiral SFC (AS column, 40% MeOH/0.1% DMEA/CO₂).

Isomer 1 (Example 72) was the first fraction. Enantiomer 1 of ethyl 3-methyl-3-(4-(2-oxoindolin-1-yl)piperidin-1-yl)pyrrolidine-1-carboxylate (76 mg). Retention time: 2.06 minutes (Chiral SFC, AS column, 40% EtOH/0.1% DEA/CO₂). 1H NMR (400 MHz, CD₃OD) δ ppm 1.10 (s, 3H), 1.23 (td, J=7.23, 1.95 Hz, 3H), 1.60-1.72 (m, 2H), 1.84-2.01 (m, 2H), 2.37-2.56 (m, 4H), 2.77 (t, J=6.25 Hz, 1H), 2.97 (dd, J=7.42, 2.73 Hz, 1H), 3.21 (dd, J=9.96, 6.05 Hz, 1H), 3.30-3.49 (m, 3H), 3.50-3.59 (m, 1H), 4.09 (q, J=7.03 Hz, 2H), 4.13-4.24 (m, 1H), 6.96-7.04 (m, 1H), 7.17-7.28 (m, 3H). HRMS [M+1]: 372.2278.

Isomer 2 (Example 73) was the second fraction. Enantiomer 2 of ethyl 3-methyl-3-(4-(2-oxoindolin-1-yl)piperidin-1-yl)pyrrolidine-1-carboxylate (84 mg). Retention time: 3.26 minutes (Chiral SFC, AS column, 40% EtOH/0.1% DEA/CO₂). 1H NMR (400 MHz, CD₃OD) δ ppm 1.10 (s, 3H), 1.23 (td, J=7.23, 1.95 Hz, 3H), 1.60-1.72 (m, 2H), 1.84-2.01 (m, 2H), 2.37-2.56 (m, 4H), 2.77 (t, J=6.25 Hz, 1H), 2.97 (dd, J=7.42, 2.73 Hz, 1H), 3.21 (dd, J=9.96, 6.05 Hz, 1H), 3.30-3.49 (m, 3H), 3.50-3.59 (m, 1H), 4.09 (q, J=7.03 Hz, 2H), 4.13-4.24 (m, 1H), 6.96-7.04 (m, 1H), 7.17-7.28 (m, 3H). HRMS [M+1]: 372.2283.

### Examples 74 (Isomer 1) and Example 75

### ethyl 3-(4-(6-fluoro-2-oxoindolin-1-yl)piperidin-1-yl)-3-methylpyrrolidine-1-carboxylate

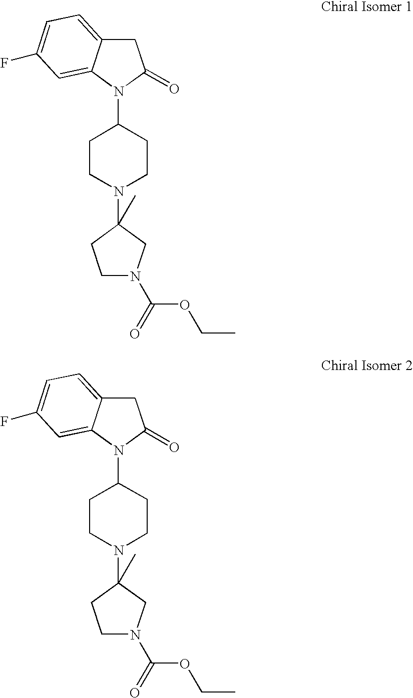

### Step A. Preparation of di-tert-butyl 2-(4-fluoro-2-nitrophenyl)malonate

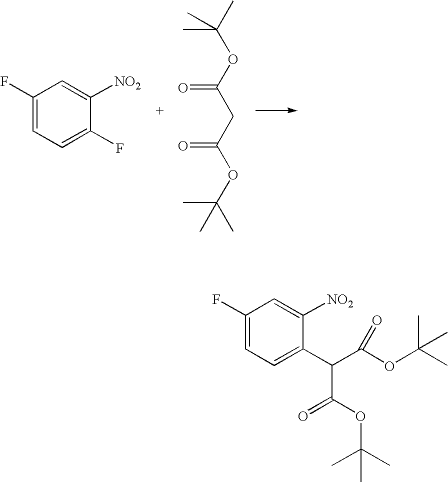

Following an analogous procedure to that described in Step A of the Example 67 and Example 68, the title compound (2.76 g, 51.8%) was prepared from 1,4-difluoro-2-nitrobenzene (2.386 g, 15 mmol) and di-tert-butyl malonate (3.24 g, 15.00 mmol). 1H NMR (400 MHz, CHLOROFORM-d) δ ppm 1.46 (s, 18H), 5.05 (s, 1H), 7.29-7.38 (m, 1H), 7.54 (dd, J=8.79, 5.27 Hz, 1H), 7.73 (dd, J=8.59, 2.73 Hz, 1H).

### Step B. Preparation of di-tert-butyl 2-(2-amino-4-fluorophenyl)malonate

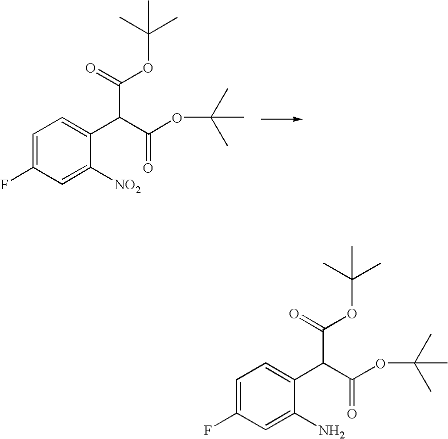

Following an analogous procedure to that described in Step B of the Example 67 and Example 68, the title compound (1.253 g, 49.6%) was prepared from di-tert-butyl 2-(4-fluoro-2-nitrophenyl)malonate (2.76 g, 7.77 mmol). 1H NMR (400 MHz, CHLOROFORM-D) δ ppm 1.44 (s, 18H), 4.24 (s, 2H), 4.38 (s, 1H), 6.31-6.49 (m, 2H), 7.07 (dd, J=8.20, 6.25 Hz, 1H).

### Step C: Preparation of di-tert-butyl 2-(2-(1-(1-(tert-butoxycarbonyl)-3-methylpyrrolidin-3-yl)piperidin-4-ylamino)-4-fluorophenyl)malonate

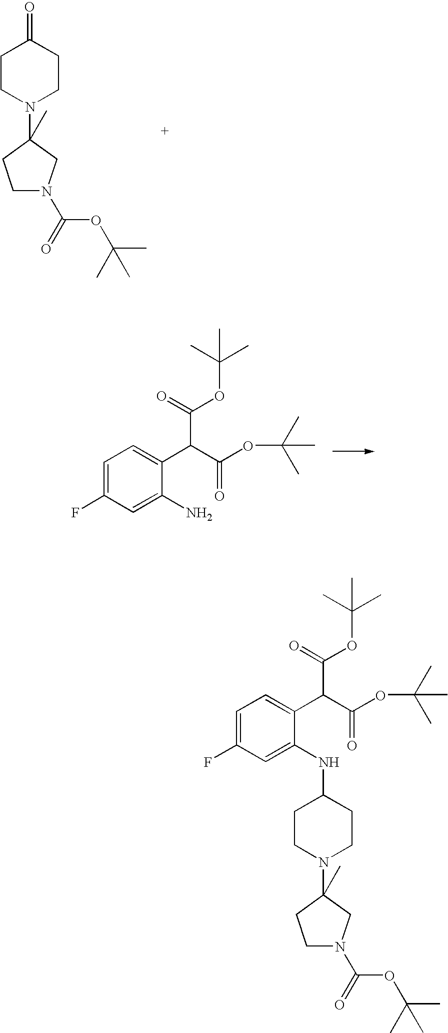

Following an analogous procedure to that described in Step C of the Example 67 and Example 68, the title compound was made from di-tert-butyl 2-(2-amino-4-fluorophenyl)malonate (0.63 g, 1.92 mmol) and tert-butyl 3-methyl-3-(4-oxopiperidin-1-yl)pyrrolidine-1-carboxylate (0.54 g, 1.92 mmol). The crude product was purified by high pH preparative HPLC (60-80% MeCN in water) to give the title compound (0.51 g, 44.5%) as solid. 1H NMR (400 MHz, CDCl₃) δ ppm 1.05 (d, J=3.12 Hz, 3H), 1.36-1.55 (m, 29H), 1.72-1.93 (m, 2H), 1.96-2.08 (m, 2H), 2.25-2.49 (m, 2H), 2.57-2.70 (m, 1H), 2.80 (d, J=10.94 Hz, 1H), 3.10-3.61 (m, 5H), 4.33 (s, 1H), 4.85-5.03 (m, 1H), 6.25-6.36 (m, 2H), 7.02 (t, J=7.62 Hz, 1H). MS (M+1): 592.5.

### Step D: Preparation of 6-fluoro-1-(1-(3-methylpyrrolidin-3-yl)piperidin-4-yl)indolin-2-one

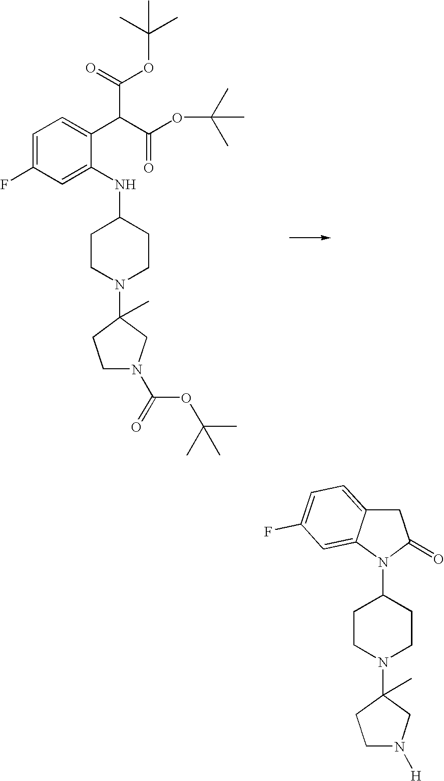

Following an analogous procedure to that described in Step D of the Example 67 and 68, the title compound was made from di-tert-butyl 2-(2-(1-(1-(tert-butoxycarbonyl)-3-methylpyrrolidin-3-yl)piperidin-4-ylamino)-4-fluorophenyl)malonate (0.59 g, 1.00 mmol). The crude product was used in the subsequent reaction without further purification. MS (M+1): 318.2.

### Step E: Preparation of ethyl 3-(4-(6-fluoro-2-oxoindolin-1-yl)piperidin-1-yl)-3-methylpyrrolidine-1-carboxylate

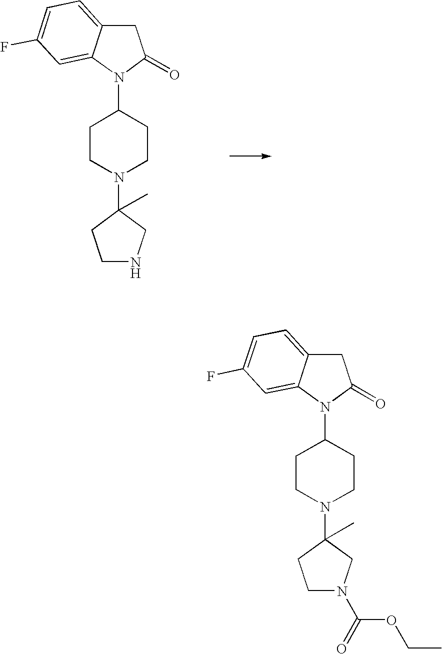

Following an analogous procedure to that described in Step E of the Example 67 and Example 68, the title compound was made from 6-fluoro-1-(1-(3-methylpyrrolidin-3-yl)piperidin-4-yl)indolin-2-one (60.3 mg, 0.19 mmol) and ethyl carbonochloridate (36.2 μl, 0.38 mmol). The crude product was purified by high pH preparative HPLC (30-50% MeCN in water) to give the title compound (31.0 mg, 41.9%) as solid. 1H NMR (400 MHz, CDCl₃) δ ppm 1.05 (br. s., 3H), 1.23 (t, J=7.03 Hz, 3H), 1.58-2.01 (m, 5H), 2.22-2.51 (m, 4H), 2.66-2.82 (m, 1H), 2.91 (br. s.,1H), 3.22 (t, J=10.16 Hz, 1H), 3.28-3.67 (m, 4H), 4.01-4.35 (m, 3H), 6.67 (t, J=8.79 Hz, 1H), 6.86 (d, J=9.77 Hz, 1H), 7.12 (t, J=6.84 Hz, 1H). MS (M+1): 390.32.

### Step F: Separation of enantiomers of ethyl 3-(4-(6-fluoro-2-oxoindolin-1-yl)piperidin-1-yl)-3-methylpyrrolidine-1-carboxylate

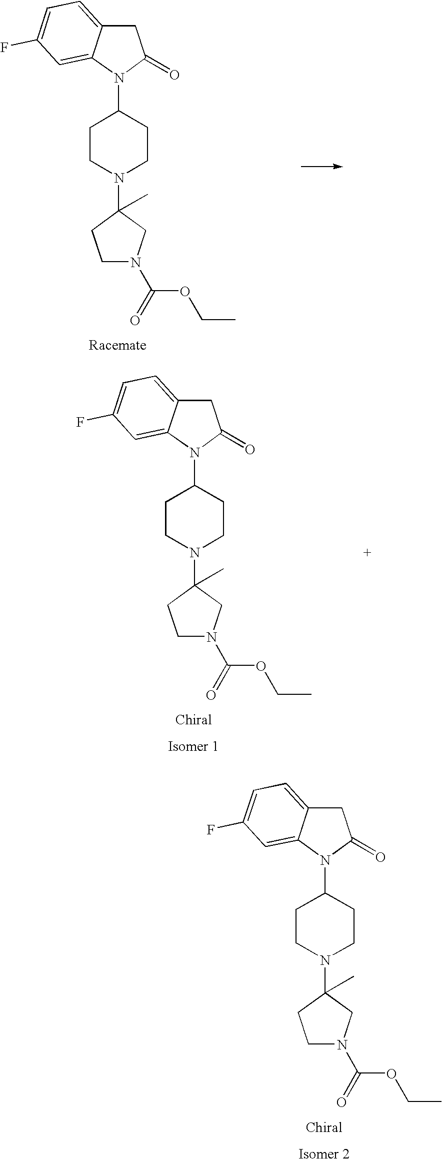

Racemic mixture of ethyl 3-(4-(6-fluoro-2-oxoindolin-1-yl)piperidin-1-yl)-3-methylpyrrolidine-1-carboxylate (235 mg, 0.60 mmol) was separated by chiral SFC (AS column, 40% EtOH/0.1% DMEA/CO₂).

Isomer 1 (Example 74) was the first fraction: Enantiomer 1 of ethyl 3-(4-(6-fluoro-2-oxoindolin-1-yl)piperidin-1-yl)-3-methylpyrrolidine-1-carboxylate (68.2 mg). Retention time: 1.89 minutes (Chiral SFC, AS column, 40% EtOH/0.1% DEA/CO₂) 1H NMR (400 MHz, CDCl₃) δ ppm 1.03 (d, 3H), 1.21 (td, J=7.03, 1.95 Hz, 3H), 1.65 (d, J=10.16 Hz, 2H), 1.75-1.97 (m, 2H), 2.08-2.49 (m, 4H), 2.73 (t, J=7.62 Hz, 1H), 2.85-2.96 (m, 1H), 3.20 (t, J=9.77 Hz, 1H), 3.26-3.64 (m, 5H), 4.01-4.26 (m, 3H), 6.65 (t, J=8.79 Hz, 1H), 6.85 (d, J=9.77 Hz, 1H), 7.06-7.15 (m, 1H). HRMS [M+1]: 390.2176.

Isomer 2 (example 75) was the second fraction. Enantiomer 2 of ethyl 3-(4-(6-fluoro-2-oxoindolin-1-yl)piperidin-1-yl)-3-methylpyrrolidine-1-carboxylate (69.9 mg). Retention time: 2.73 minutes (Chiral SFC, AS column, 40% EtOH/0.1% DEA/CO₂). 1H NMR (400 MHz, CDCl₃) δ ppm 1.03 (d, 3H), 1.21 (td, J=7.03, 1.95 Hz, 3H), 1.65 (d, J=10.16 Hz, 2H), 1.75-1.97 (m, 2H), 2.08-2.49 (m, 4H), 2.73 (t, J=7.62 Hz, 1H), 2.85-2.96 (m, 1H), 3.20 (t, J=9.77 Hz, 1H), 3.26-3.64 (m, 5H), 4.01-4.26 (m, 3H), 6.65 (t, J=8.79 Hz, 1H), 6.85 (d, J=9.77 Hz, 1H), 7.06-7.15 (m, 1H). HRMS [M+1]: 390.2181.

### Example 76 (Isomer 1) and Examples (77)

### methyl 3-(4-(6-fluoro-2-oxoindolin-1-yl)piperidin-1-yl)-3-methylpyrrolidine-1-carboxylate

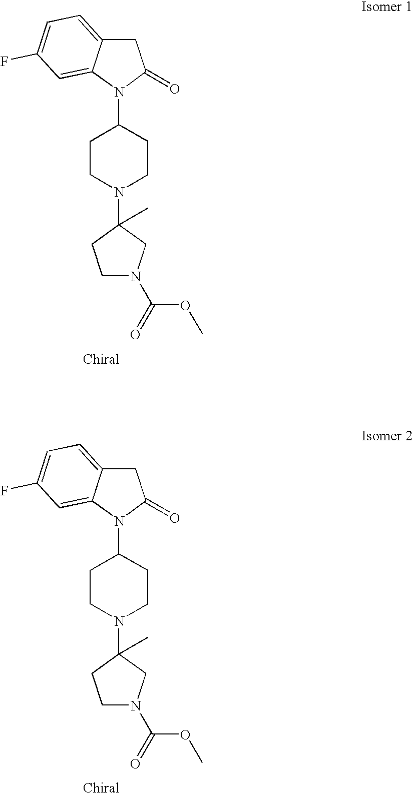

### Step A: Preparation of methyl 3-(4-(6-fluoro-2-oxoindolin-1-yl)piperidin-1-yl)-3-methylpyrrolidine-1-carboxylate (racemate)

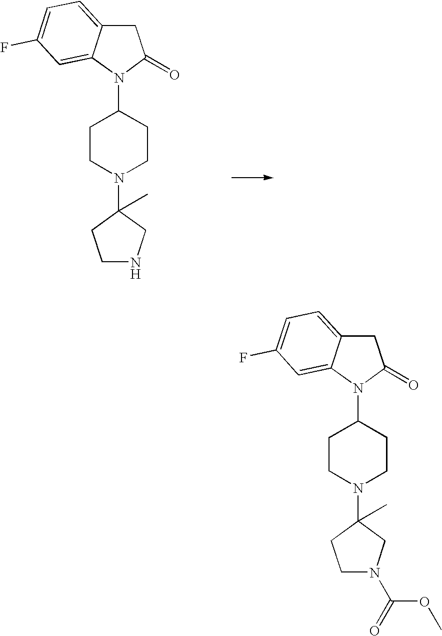

Following an analogous procedure to that described in Step E of the Example 67 and Example 68, the title compound was made from 6-fluoro-1-(1-(3-methylpyrrolidin-3-yl)piperidin-4-yl)indolin-2-one (60.3 mg, 0.19 mmol) and methyl carbonochloridate (0.053 mL, 0.68 mmol). The crude product was purified by high pH preparative HPLC (30-50% MeCN in water) to give the title compound (71.7 mg, 56.2%) as solid.1H NMR (400 MHz, CDCl₃) δ ppm 1.08 (s, 3H), 1.62-1.78 (m, 2H), 1.79-2.10 (m, 2H), 2.26-2.54 (m, 4H), 2.70-2.86 (m, 1H), 2.87-3.02 (m, 1H), 3.19-3.31 (m, 1H), 3.32-3.68 (m, 5H), 3.71 (d, J=3.12 Hz, 3H), 4.24 (t, J=11.52 Hz, 1H), 6.65-6.76 (m, 1H), 6.89 (dd, J=9.77, 1.56 Hz, 1H), 7.10-7.21 (m, 1H). MS (M+1): 376.2.

### Step B: Separation of enantiomers of methyl 3-(4-(6-fluoro-2-oxoindolin-1-yl)piperidin-1-yl)-3-methylpyrrolidine-1-carboxylate

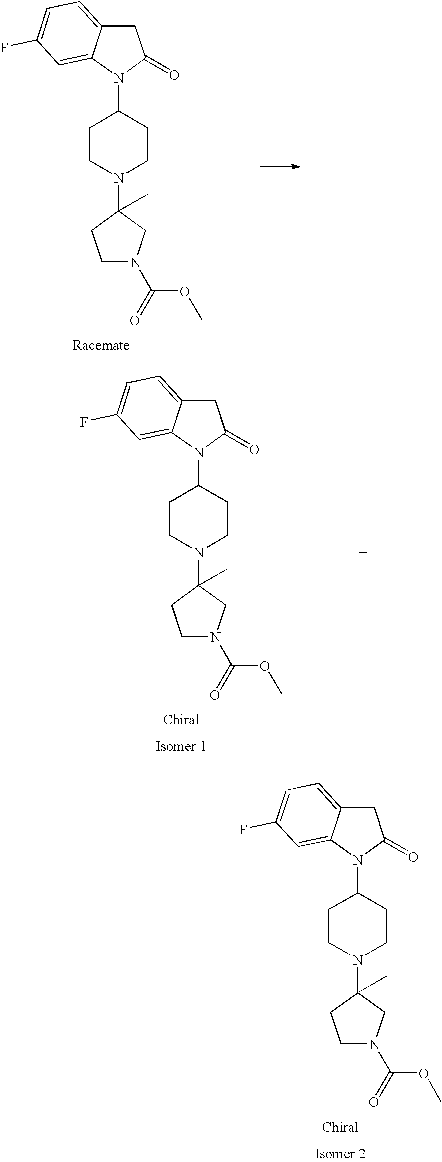

Racemic mixture of methyl 3-(4-(6-fluoro-2-oxoindolin-1-yl)piperidin-1-yl)-3-methylpyrrolidine-1-carboxylate (71 mg, 0.19 mmol) was separated by chiral SFC (AS column with 40% EtOH/0.1% DMEA/CO₂).

Isomer 1 (Example 76) was the first fraction: Enantiomer 1 of methyl 3-(4-(6-fluoro-2-oxoindolin-1-yl)piperidin-1-yl)-3-methylpyrrolidine-1-carboxylate (66.2 mg). Retention time: 2.32 minutes (Chiral SFC, AS column, 30% EtOH/0.1% DEA/CO₂) 1H NMR (400 MHz, CDCl₃) δ ppm 1.08 (s, 3H), 1.62-1.78 (m, 2H), 1.79-2.10 (m, 2H), 2.26-2.54 (m, 4H), 2.70-2.86 (m, 1H), 2.87-3.02 (m, 1H), 3.19-3.31 (m, 1H), 3.32-3.68 (m, 5H), 3.71 (d, J=3.12 Hz, 3H), 4.24 (t, J=11.52 Hz, 1H), 6.65-6.76 (m, 1H), 6.89 (dd, J=9.77, 1.56 Hz, 1H), 7.10-7.21 (m, 1H). HRMS [M+1]: 376.2024.

Isomer 2 (Example 77) was the second fraction: Enantiomer 2 of methyl 3-(4-(6-fluoro-2-oxoindolin-1-yl)piperidin-1-yl)-3-methylpyrrolidine-1-carboxylate. Retention time: 4.02 minutes (Chiral SFC, AS column, 40% EtOH/0.1% DEA/CO₂). NMR (400 MHz, CDCl₃) δ ppm 1.08 (s, 3H), 1.62-1.78 (m, 2H), 1.79-2.10 (m, 2H), 2.26-2.54 (m, 4H), 2.70-2.86 (m, 1H), 2.87-3.02 (m, 1H), 3.19-3.31 (m, 1H), 3.32-3.68 (m, 5H), 3.71 (d, J=3.12 Hz, 3H), 4.24 (t, J=11.52 Hz, 1H), 6.65-6.76 (m, 1H), 6.89 (dd, J=9.77, 1.56 Hz, 1H), 7.10-7.21 (m, 1H). HRMS [M+1]: 376.2029.

### Example 78

### methyl 3-(4-(5-fluoro-2-oxoindolin-1-yl)piperidin-1-yl)-3-methylpyrrolidine-1-carboxylate (Racemate)

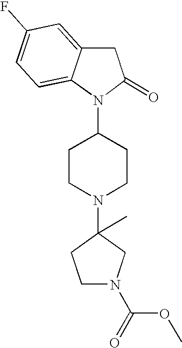

### Step A: Preparation of di-tert-butyl 2-(2-(1-(1-(tert-butoxycarbonyl)-3-methylpyrrolidin-3-yl)piperidin-4-ylamino)-5-fluorophenyl)malonate

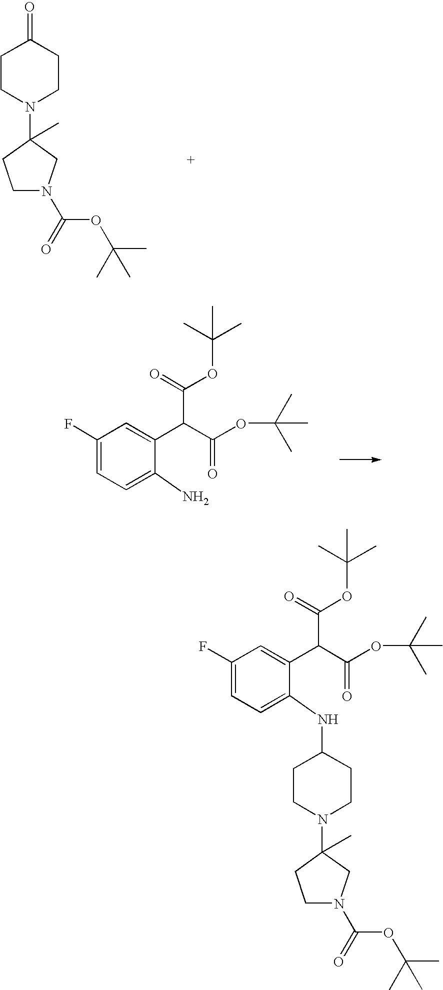

Following an analogous procedure to that described in Step C of the Example 67 and Example 68, the title compound was made from di-tert-butyl 2-(2-amino-5-fluorophenyl)malonate (0.6 g, 1.84 mmol) and tert-butyl 3-methyl-3-(4-oxopiperidin-1-yl)pyrrolidine-1-carboxylate (0.52 g, 1.84 mmol). The crude product was purified by high pH preparative HPLC (60-80% MeCN in water) to give the title compound (0.77 g, 70%) as pale yellow solid. 1H NMR (400 MHz, CDCl₃) δ ppm 1.00 (d, J=3.12 Hz, 3H), 1.30-1.51 (m, 28H), 1.67-1.87 (m, 2H), 1.95 (d, J=9.37 Hz, 2H), 2.16-2.42 (m, 2H), 2.53-2.65 (m, 1H), 2.74 (d, J=5.86 Hz, 1H), 3.05-3.31 (m, 4H), 3.32-3.56 (m, 2H), 4.20 (br. s., 1H), 4.39 (s, 1H), 6.55 (dd, J=8.79, 4.88 Hz, 1H), 6.76-6.85 (m, 1H), 6.92 (dd, J=9.57, 2.93 Hz, 1H). MS (ESI): 592.4.

### Step B: Preparation of 5-fluoro-1-(1-(3-methylpyrrolidin-3-yl)piperidin-4-yl)indolin-2-one

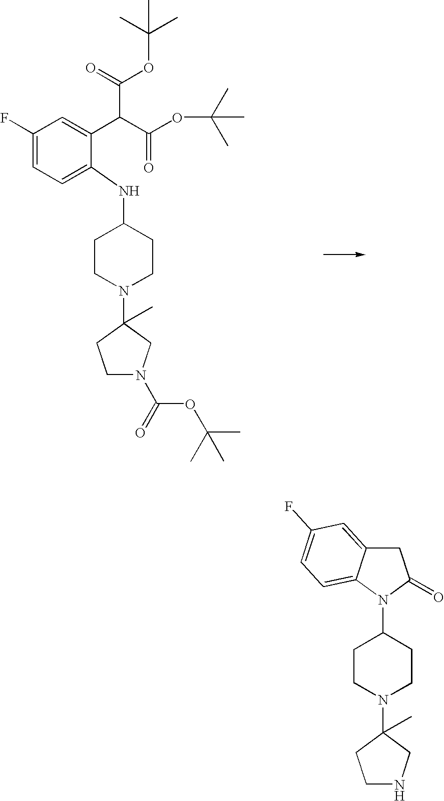

Following an analogous procedure to that described in Step D of Example 67 and Example 68, the title compound was made from di-tert-butyl 2-(2-(1-(1-(tert-butoxycarbonyl)-3-methylpyrrolidin-3-yl)piperidin-4-ylamino)-5-fluorophenyl)malonate (769 mg, 1.30 mmol). The crude product was used in the subsequent reaction without further purification. MS (M+1): 318.2.

### Step C: Preparation of methyl 3-(4-(5-fluoro-2-oxoindolin-1-yl)piperidin-1-yl)-3-methylpyrrolidine-1-carboxylate

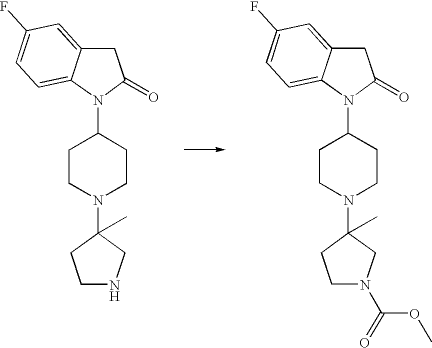

Following an analogous procedure to that described in Step E of Example 67 and Example 68, the title compound was made from 5-fluoro-1-(1-(3-methylpyrrolidin-3-yl)piperidin-4-yl)indolin-2-one (0.024 g, 0.0755 mmol) and methyl carbonochloridate (0.012 mL, 0.15 mmol). The crude product was purified by high pH preparative HPLC (30-50% MeCN in water) to give the title compound (mixture of racemates) (0.020 g, 69.9%). 1H NMR (400 MHz, CDCl₃) δ ppm 1.05 (s, 3H), 1.67 (d, J=6.25 Hz, 3H), 1.76-1.98 (m, 2H), 2.25-2.52 (m, 3H), 2.74 (t, J=11.72 Hz, 1H), 2.84-2.97 (m, 1H), 3.22 (t, J=11.13 Hz, 1H), 3.28-3.64 (m, 5H), 3.67 (d, J=3.52 Hz, 3H), 4.25 (t, J=10.16 Hz, 1H), 6.85-6.99 (m, 2H), 7.04 (dd, J=8.79, 4.10 Hz, 1H). HRMS [M+1]: 376.2022.

### Example 79 (Isomer 1) and Example 80 (Isomer 2)

### ethyl 3-(4-(5-fluoro-2-oxoindolin-1-yl)piperidin-1-yl)-3-methylpyrrolidine-1-carboxylate

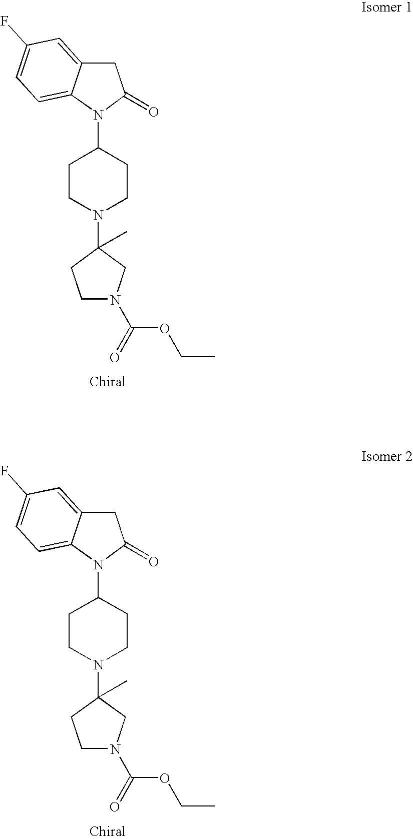

### Step A: Preparation of ethyl 3-(4-(5-fluoro-2-oxoindolin-1-yl)piperidin-1-yl)-3-methylpyrrolidine-1-carboxylate (Racemate)

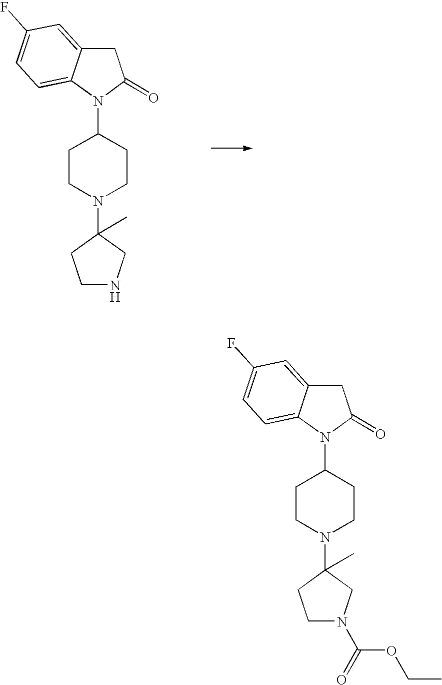

Following an analogous procedure to that described in Step E of Example 67 and Example 68, the title compound was made from 5-fluoro-1-(1-(3-methylpyrrolidin-3-yl)piperidin-4-yl)indolin-2-one (0.413 g, 1.3 mmol) and ethyl carbonochloridate (0.248 mL, 2.60 mmol). The crude product was purified by high pH preparative HPLC (30-50% MeCN in water) to give the title compound (0.380 g, 75%) as solid. 1H NMR (400 MHz, CDCl₃) δ ppm 1.01 (d, J=2.34 Hz, 3H), 1.19 (td, J=7.03, 1.95 Hz, 3H), 1.63 (d, J=9.77 Hz, 2H), 1.72-1.95 (m, 2H), 2.17-2.49 (m, 4H), 2.65-2.78 (m, 1H), 2.81-2.95 (m, 1H), 3.17 (t, J=8.79 Hz, 1H), 3.24-3.62 (m, 5H), 3.99-4.14 (m, 2H), 4.15-4.30 (m, 1H), 6.81-6.97 (m, 2H), 7.01 (m, J=8.59, 3.91 Hz, 1H).1H C NMR (101 MHz, CDCl₃) δ ppm 15.0, 28.8, 36.2, 37.6, 38.3, 45.2, 47.2, 48.2, 50.3, 58.5, 61.2, 63.4, 64.2, 110.7, 112.6, 113.8, 126.6, 139.7, 157.6, 160.0, 174.6.

### Step B: Separation of enantiomers of ethyl 3-(4-(5-fluoro-2-oxoindolin-1-yl)piperidin-1-yl)-3-methylpyrrolidine-1-carboxylate

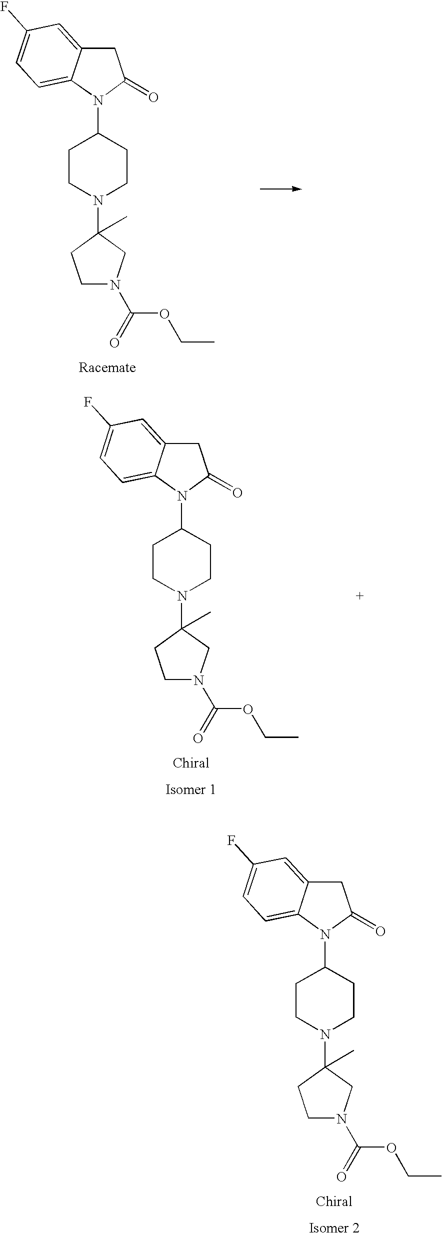

Racemic mixture of ethyl 3-(4-(5-fluoro-2-oxoindolin-1-yl)piperidin-1-yl)-3-methylpyrrolidine-1-carboxylate (380 mg, 0.98 mmol) was separated by chiral SFC (AS column, 40% EtOH/0.1% DMEA/CO₂).

Isomer 1 (Example 79) was the first fraction: Enantiomer 1 of ethyl 3-(4-(5-fluoro-2-oxoindolin-1-yl)piperidin-1-yl)-3-methylpyrrolidine-1-carboxylate (120 mg). Retention time: 2.01 minutes (Chiral SFC, AS column, 40% EtOH/0.1% DEA/CO₂). 1H NMR (400 MHz, CDCl₃) δ ppm 1.01 (d, J=2.34 Hz, 3H), 1.19 (td, J=7.03, 1.95 Hz, 3H), 1.63 (d, J=9.77 Hz, 2H), 1.72-1.95 (m, 2H), 2.17-2.49 (m, 4H), 2.65-2.78 (m, 1H), 2.81-2.95 (m, 1H), 3.17 (t, J=8.79 Hz, 1H), 3.24-3.62 (m, 5H), 3.99-4.14 (m, 2H), 4.15-4.30 (m, 1H), 6.81-6.97 (m, 2H), 7.01 (dd, J=8.59, 3.91 Hz, 1H). HRMS [M+1]: 390.2181.

Isomer 2 (Example 80) was the second fraction: Enantiomer 2 of ethyl 3-(4-(5-fluoro-2-oxoindolin-1-yl)piperidin-1-yl)-3-methylpyrrolidine-1-carboxylate (130 mg). Retention time: 3.21 minutes (Chiral SFC, AS column, 40% EtOH/0.1% DEA/CO₂). 1H NMR (400 MHz, CDCl₃) δ ppm 1.01 (d, J=2.34 Hz, 3H), 1.19 (td, J=7.03, 1.95 Hz, 3H), 1.63 (d, J=9.77 Hz, 2H), 1.72-1.95 (m, 2H), 2.17-2.49 (m, 4H), 2.65-2.78 (m, 1H), 2.81-2.95 (m, 1H), 3.17 (t, J=8.79 Hz, 1H), 3.24-3.62 (m, 5H), 3.99-4.14 (m, 2H), 4.15-4.30 (m, 1H), 6.81-6.97 (m, 2H), 7.01 (dd, J=8.59, 3.91 Hz, 1H). HRMS [M+1]: 390.2182.

### Examples 81 (Isomer 1) and Example 82 (Isomer 2)

### ethyl 3-(4-(5-fluoro-6-methyl-2-oxoindolin-1-yl)piperidin-1-yl)-3-methylpyrrolidine-1-carboxylate

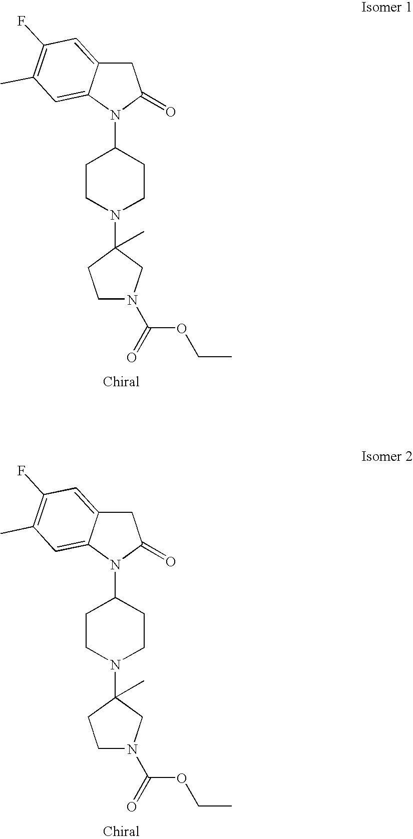

### Step A: Preparation of di-tert-butyl 2-(5-fluoro-4-methyl-2-nitrophenyl)malonate

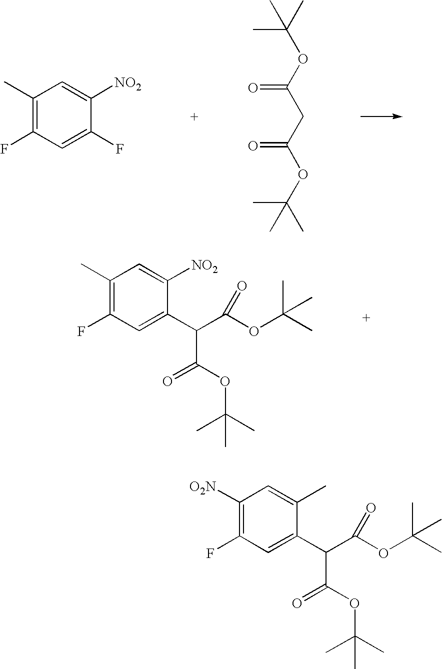

Sodium hydride (0.384 g, 16.00 mmol) was added to a mixture of di-tert-butyl malonate (1.730 g, 8.00 mmol) in DMF (10 mL) under a nitrogen atmosphere at 0° C. The resulting mixture was stirred at 0° C. for 20 minutes. 1,5-difluoro-2-methyl-4-nitrobenzene (1.385 g, 8 mmol) was added, and the mixture was stirred at room temperature for 12 hours. The reaction mixture was concentrated under reduced pressure. Water and dichloromethane was added to the residue and the phases were separated. The aqueous phase was extracted with dichloromethane. Combined organic extracts were washed with brine, dried over MgSO₄, filtered and concentrated under reduced pressure. The crude product was purified by high pH preparative HPLC (60-80% MeCN in water) to give a mixture of di-tert-butyl 2-(5-fluoro-4-methyl-2-nitrophenyl)malonate (title compound) and di-tert-butyl 2-(5-fluoro-2-methyl-4-nitrophenyl)malonate in a ratio of 3:1 (1.399 g, 47% yield). Pure di-tert-butyl 2-(5-fluoro-4-methyl-2-nitrophenyl)malonate was isolated in a small amount as white solid. 1H H NMR (400 MHz, CDCl₃) δ ppm 1.47 (s, 18H), 2.32 (d, J=1.56 Hz, 3H), 5.10 (s, 1H), 7.18 (d, J=9.77 Hz, 1H), 7.94 (d, J=7.03 Hz, 1H).

### Step B: Preparation of di-tert-butyl 2-(2-amino-5-fluoro-4-methylphenyl)malonate

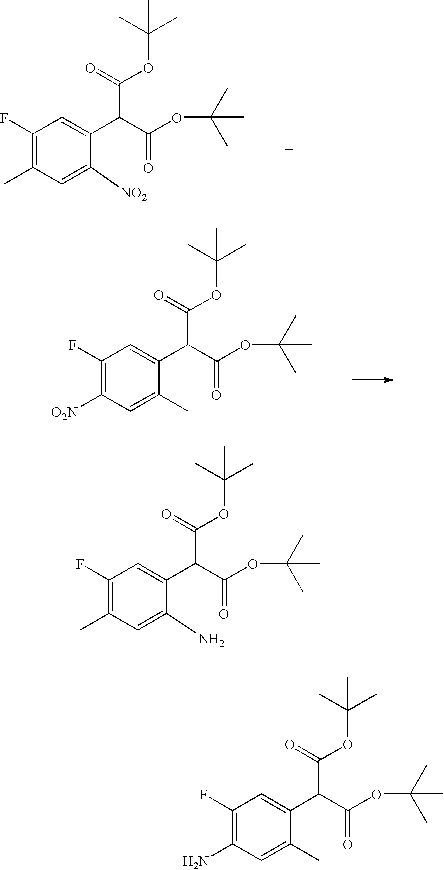

A solution of di-tert-butyl 2-(5-fluoro-4-methyl-2-nitrophenyl)malonate (1.40 g, 3.79 mmol) in MeOH (20 mL) was treated with Palladium (10% On Charcoal) (140 mg, 1.32 mmol) and was shaken under hydrogen atmosphere at 50 psi pressure for 8 hours. The mixture was filtered and the filtrate was concentrated under reduced pressure. The crude product was purified by high pH preparative HPLC (50-70% MeCN in water) to give the title product (0.697 g, 54.2%) as pale yellow oil. 1H NMR (400 MHz, CDCl3) δ ppm 1.43 (s, 18H), 2.12 (d, J=1.56 Hz, 3H), 3.81 (br. s., 2H), 4.39 (s, 1H), 6.46 (d, J=6.64 Hz, 1H), 6.89 (d, J=10.55 Hz, 1H).

Regioisomer di-tert-butyl 2-(4-amino-5-fluoro-2-methylphenyl)malonate (0.305 g, 79%) was obtained as pale yellow oil. 1H NMR (400 MHz, CDCl3) δ ppm 1.44 (s, 18H), 2.15 (s, 3H), 3.63 (s, 2H), 4.51 (s, 1H), 6.53 (d, J=9.37 Hz, 1H), 7.06 (d, J=12.50 Hz, 1H)

### Step C: Preparation of di-tert-butyl 2-(2-(1-(1-(tert-butoxycarbonyl)-3-methylpyrrolidin-3-yl)piperidin-4-ylamino)-5-fluoro-4-methylphenyl)malonate

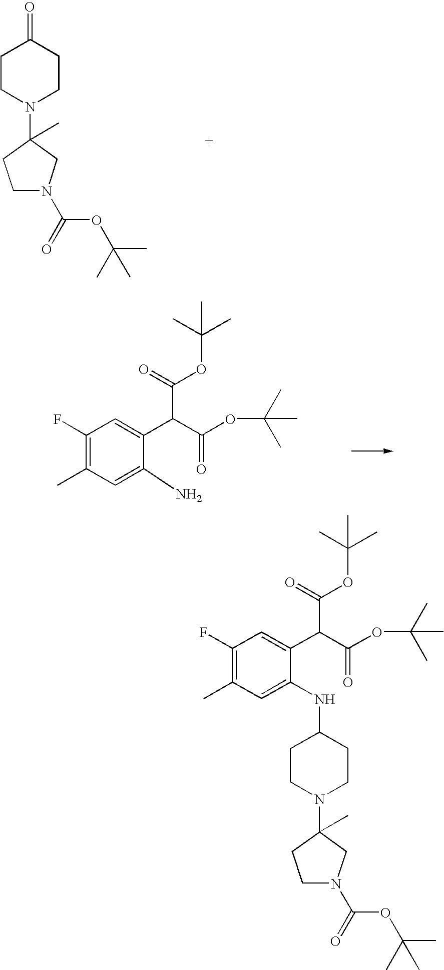

Following an analogous procedure to that described in Step C of the Example 67 and Example 68, the title compound was made from di-tert-butyl 2-(2-amino-5-fluoro-4-methylphenyl)malonate (447 mg, 1.32 mmol) and tert-butyl 3-methyl-3-(4-oxopiperidin-1-yl)pyrrolidine-1-carboxylate (372 mg, 1.32 mmol). The crude product was purified by high pH preparative HPLC (70-90% MeCN in water) to give the title compound (365 mg, 45.8%) 1H NMR (400 MHz, CDCl₃) δ ppm 1.03 (d, 3H), 1.41 (s, 29H), 1.70-1.89 (m, 2H), 1.97 (d, J=8.98 Hz, 2H), 2.16 (s, 3H), 2.23-2.46 (m, 2H), 2.61 (d, J=5.86 Hz, 1H), 2.77 (d, J=5.47 Hz, 1H), 3.04-3.61 (m, 5H), 4.14 (br. s., 1H), 4.37 (s, 1H), 6.42 (d, J=6.64 Hz, 1H), 6.87 (d, J,=,10.16 Hz, 1H).

### Step D: Preparation of 5-fluoro-6-methyl-1-(1-(3-methylpyrrolidin-3-yl)piperidin-4-yl)indolin-2-one

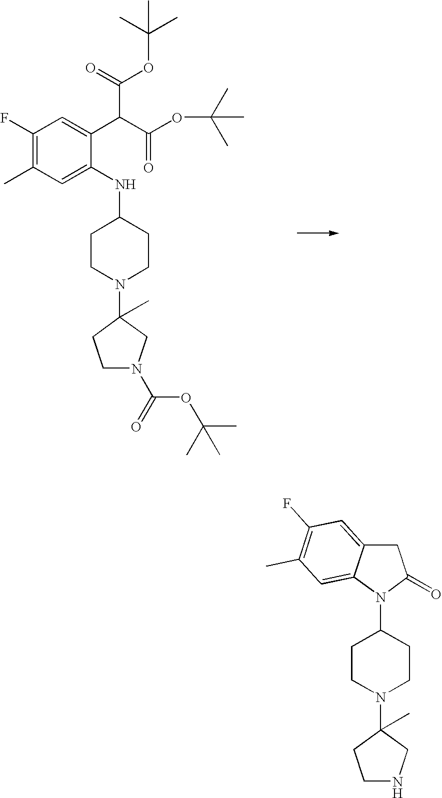

Following an analogous procedure to that described in Step D of the Example 67 and Example 68, the title compound was made from di-tert-butyl 2-(2-(1-(1-(tert-butoxycarbonyl)-3-methylpyrrolidin-3-yl)piperidin-4-ylamino)-5-fluoro-4-methylphenyl)malonate (365.3 mg, 0.60 mmol). The crude product was used for the subsequent reaction without further purification.

### Step E: Preparation of ethyl 3-(4-(5-fluoro-6-methyl-2-oxoindolin-1-yl)piperidin-1-yl)-3-methylpyrrolidine-1-carboxylate

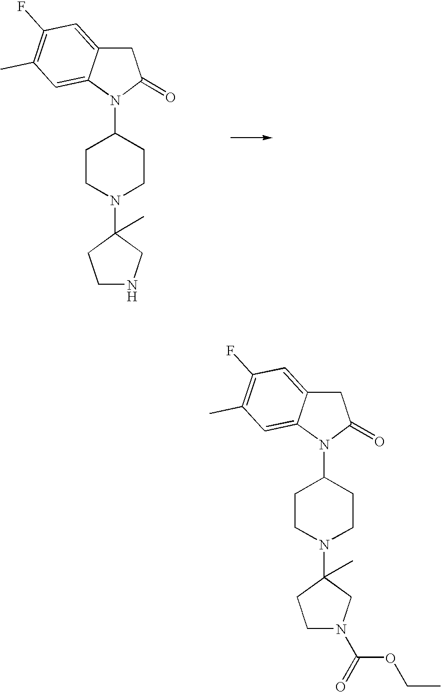

Following an analogous procedure to that described in Step E of the Example 67 and Example 68, the title compound was made from 5-fluoro-6-methyl-1-(1-(3-methylpyrrolidin-3-yl)piperidin-4-yl)indolin-2-one (0.099 g, 0.3 mmol) and ethyl carbonochloridate (0.057 mL, 0.60 mmol). The crude product was purified by high pH preparative HPLC (30-50% MeCN in water) to give the title compound (0.112 g, 93%) as solid. 1H NMR (400 MHz, CDCl₃) δ ppm 1.06 (d, J=1.95 Hz, 3H), 1.23 (td, J=7.03, 1.56 Hz, 3H), 1.67 (d, J=10.94 Hz, 2H), 1.76-2.01 (m, 3H), 2.20-2.52 (m, 6H), 2.75 (t, J=7.23 Hz, 1H), 2.84-2.99 (m, 1H), 3.23 (t, J=10.16 Hz, 1H), 3.29-3.67 (m, 5H), 4.10 (qd, J=7.03, 2.34 Hz, 2H), 4.16-4.30 (m, 1H), 6.81-6.95 (m, 2H). MS (M+1): 404.3.

### Step F: Separation of enantiomers of 3-(4-(5-fluoro-6-methyl-2-oxoindolin-1-yl)piperidin-1-yl)-3-methylpyrrolidine-1-carboxylate

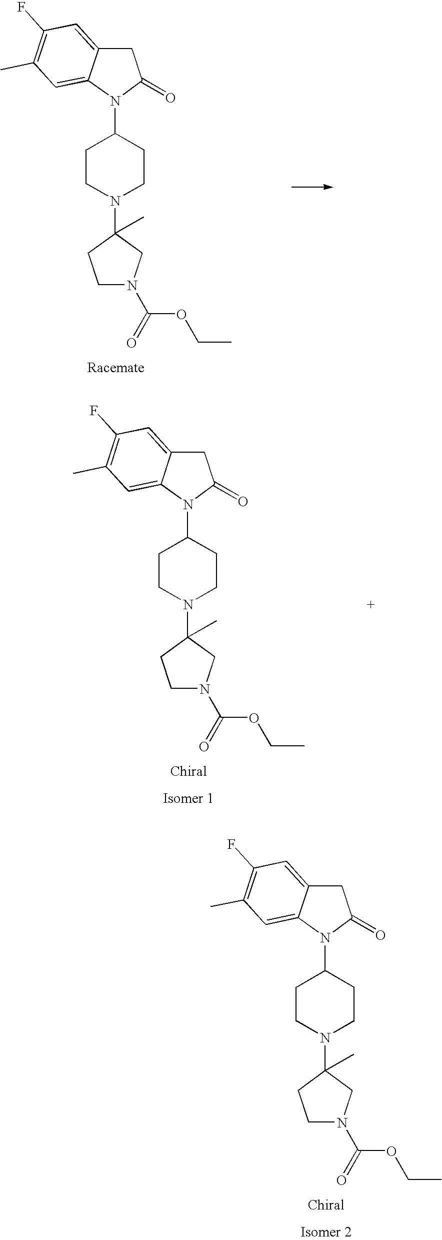

Racemic mixture of ethyl 3-(4-(5-fluoro-6-methyl-2-oxoindolin-1-yl)piperidin-1-yl)-3-methylpyrrolidine-1-carboxylate (112 mg, 0.28 mmol) was separated by chiral HPLC (Chiralpak AD column, 20% EtOH/0.1% DEA in heptane).

Isomer 1 (Example 81) was the first fraction: Enantiomer 1 of ethyl 3-(4-(5-fluoro-6-methyl-2-oxoindolin-1-yl)piperidin-1-yl)-3-methylpyrrolidine-1-carboxylate (54.6 mg). Retention time: 6.50 minutes (Chiracel OD column, 40% EtOH/0.1% DEA in heptane). 1H NMR (400 MHz, CDCl₃) δ ppm 1.06 (d, J=1.95 Hz, 3H), 1.23 (td, J=7.03, 1.56 Hz, 3H), 1.67 (d, J=10.94 Hz, 2H), 1.76-2.01 (m, 3H), 2.20-2.52 (m, 6H), 2.75 (t, J=7.23 Hz, 1H), 2.84-2.99 (m, 1H), 3.23 (t, J=10.16 Hz, 1H), 3.29-3.67 (m, 5H), 4.10 (qd, J=7.03, 2.34 Hz, 2H), 4.16-4.30 (m, 1H), 6.81-6.95 (m, 2H). HRMS [M+1]: 404.2350.

Isomer 2 (Example 82) was the second fraction: Enantiomer 2 of ethyl 3-(4-(5-fluoro-6-methyl-2-oxoindolin-1-yl)piperidin-1-yl)-3-methylpyrrolidine-1-carboxylate (52.7 mg). was obtained as solid. Retention time: 10.27 minutes (Chiracel OD column, 40% EtOH/0.1% DEA in heptane). 1H NMR (400 MHz, CDCl₃) δ ppm 1.06 (d, J=1.95 Hz, 3H), 1.23 (td, J=7.03, 1.56 Hz, 3H), 1.67 (d, J=10.94 Hz, 2H), 1.76-2.01 (m, 3H), 2.20-2.52 (m, 6H), 2.75 (t, J=7.23 Hz, 1H), 2.84-2.99 (m, 1H), 3.23 (t, J=10.16 Hz, 1H), 3.29-3.67 (m, 5H), 4.10 (qd, J=7.03, 2.34 Hz, 2H), 4.16-4.30 (m, 1H), 6.81-6.95 (m, 2H). HRMS [M+1]: 404.2353.

### Example 83 (Isomer 1) and Example 84 (Isomer 2)

### methyl 3-(4-(5-fluoro-6-methyl-2-oxoindolin-1-yl)piperidin-1-yl)-3-methylpyrrolidine-1-carboxylate

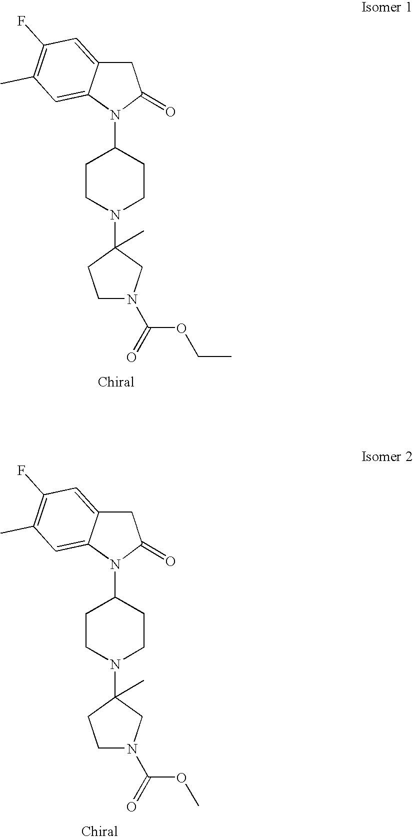

### Step A: Preparation of methyl 3-(4-(5-fluoro-6-methyl-2-oxoindolin-1-yl)piperidin-1-yl)-3-methylpyrrolidine-1-carboxylate (racemate)

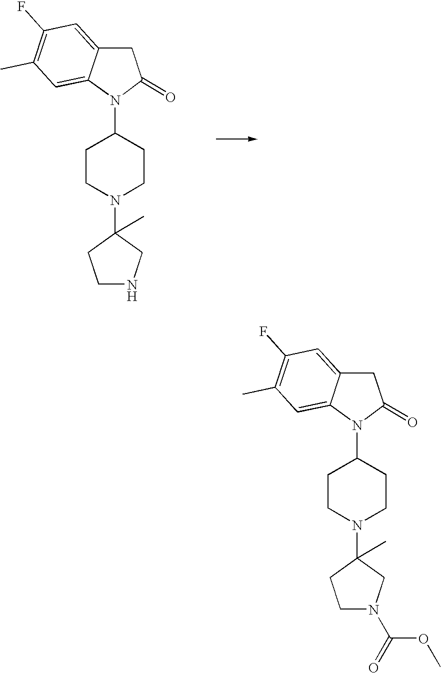

Following an analogous procedure to that described in Step E of the Example 67 and Example 68, the title compound was made from 5-fluoro-6-methyl-1-(1-(3-methylpyrrolidin-3-yl)piperidin-4-yl)indolin-2-one (0.099 g, 0.3 mmol) and methyl carbonochloridate (0.046 mL, 0.60 mmol). The crude product was purified by high pH preparative HPLC (30-50% MeCN in water) to give the title compound (0.113 g, 97%) as solid. 1H NMR (400 MHz, CDCl₃) δ ppm 1.04 (s, 3H), 1.56-1.74 (m, 2H), 1.75-1.99 (m, 2H), 2.20-2.50 (m, 7H), 2.73 (t, J=10.94 Hz, 1H), 2.89 (br. s., 1H), 3.16-3.27 (m, 1H), 3.27-3.63 (m, 5H), 3.65 (d, J=3.12 Hz, 3H). 4.19 (t, J=11.13 Hz, 1H), 6.78-6.93 (m, 2H). MS (M+1): 390.3.

### Step B: Separation of enantiomers of methyl 3-(4-(5-fluoro-6-methyl-2-oxoindolin-1-yl)piperidin-1-yl)-3-methylpyrrolidine-1-carboxylate

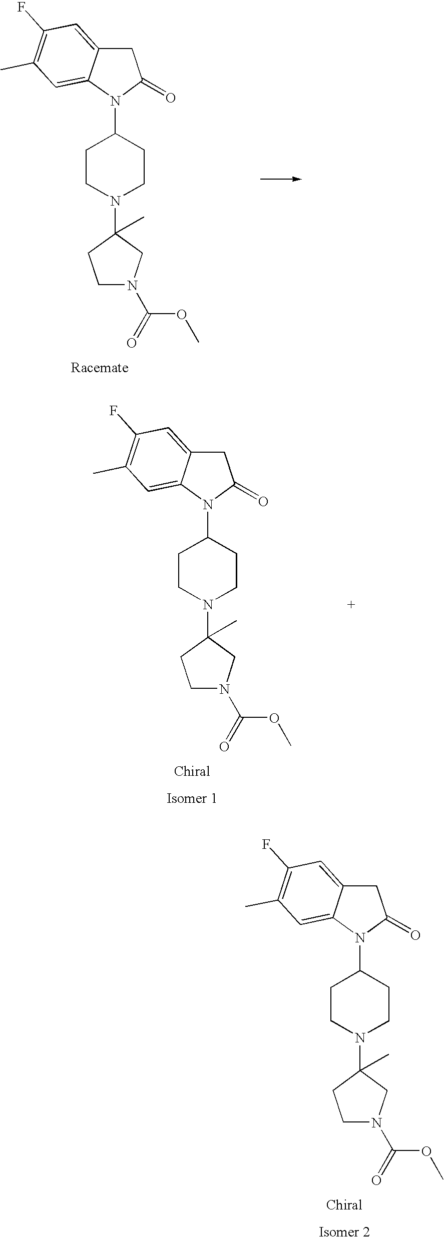

Racemic mixture of methyl 3-(4-(5-fluoro-6-methyl-2-oxoindolin-1-yl)piperidin-1-yl)-3-methylpyrrolidine-1-carboxylate (113 mg, 0.29 mmol) was separated by chiral HPLC (Chiralpak AD column, 20% EtOH/0.1% DEA in heptane).

Isomer 1 (Example 83) was the first fraction: Enantiomer 1 of methyl 3-(4-(5-fluoro-6-methyl-2-oxoindolin-1-yl)piperidin-1-yl)-3-methylpyrrolidine-1-carboxylate (40.3 mg). Retention time: 6.69 minutes (Chiracel OD column, 40% EtOH/0.1% DEA in heptane) 1H NMR (400 MHz, CDCl₃) δ ppm 1.04 (s, 3H), 1.56-1.74 (m, 2H), 1.75-1.99 (m, 2H), 2.20-2.50 (m, 7H), 2.73 (t, J=10.94 Hz, 1H), 2.89 (br. s., 1H), 3.16-3.27 (m, 1H), 3.27-3.63 (m, 5H), 3.65 (d, J=3.12 Hz, 3H), 4.19 (t, J=11.13 Hz, 1H), 6.78-6.93 (m, 2H). HRMS [M+1]: 3902188.

Isomer 2 (Example 84) was the second fraction: Enantiomer 2 of methyl 3-(4-(5-fluoro-6-methyl-2-oxoindolin-1-yl)piperidin-1-yl)-3-methylpyrrolidine-1-carboxylate (39.0 mg). Retention time: 12.27 minutes (Chiracel OD column, 40% EtOH/0.1% DEA in heptane) NMR (400 MHz, CDCl₃) δ ppm 1.04 (s, 3H), 1.56-1.74 (m, 2H), 1.75-1.99 (m, 2H), 2.20-2.50 (m, 7H), 2.73 (t, J=10.94 Hz, 1H), 2.89 (br. s.,1H), 3.16-3.27 (m, 1H), 3.27-3.63 (m, 5H), 3.65 (d, J=3.12Hz, 3H), 4.19 (t, J=11.13 Hz, 1H), 6.78-6.93 (m, 2H). HRMS [M+1]: 390.2193.

### Example 85

### ethyl 3-(4-(4-tert-butyl-6-fluoro-2-oxoindolin-1-yl)piperidin-1-yl)-3-methylpyrrolidine-1-carboxylate

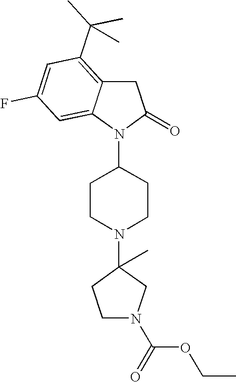

The title compound was obtained as a side product of step E of example 74 and Example 75 and was formed by the reaction of ethyl carbonochloridate in step E with the side product carried over from step D (42.6 mg). 1H NMR (400 MHz, CDCl₃) δ ppm 1.08 (d, J=2.73 Hz, 3H), 1.27 (td, J=7.13, 2.15 Hz, 3H), 1.36 (s, 9H), 1.55-1.78 (m, 2H), 1.80-2.02 (m, 2H), 2.25-2.54 (m, 4H), 2.78 (t, J=10.55 Hz, 1H), 2.89-3.00 (m, 1H), 3.25 (t, J=10.35 Hz, 1H), 3.32-3.72 (m, 5H), 4.06-4.33 (m, 3H), 6.86 (dd, J=12.89, 1.95 Hz, 1H), 7.16 (d, J=8.20 Hz, 1H). HRMS [M+1]: 446.2809.

### Example 86

### methyl 3-(4-(4-tert-butyl-6-methyl-2-oxoindolin-1-yl)piperidin-1-yl)-3-methylpyrrolidine-1-carboxylate

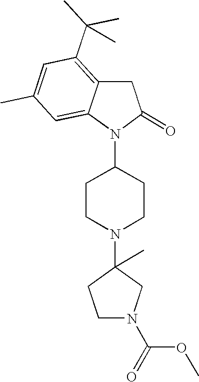

The title compound was obtained as a side product of step E of example 67 and Example 68 and was formed by the reaction of ethyl carbonochloridate in step E with the side product carried over from step D. 1H NMR (400 MHz, CDCl₃) δ ppm 1.09 (s, 3H), 1.38-1.44 (m, 9H), 1.63-1.77 (m, 2H), 1.81-2.07 (m, 2H), 2.30-2.56 (m, 4H), 2.59 (s, 3H), 2.71-2.84 (m, 1H), 2.87-3.03 (m, 1H), 3.23-3.69 (m, 6H), 3.71 (d, J=3.12 Hz, 3H), 4.25 (t, J=10.94 Hz, 1H), 6.91 (s, 1H), 7.28 (s, 1H). HRMS [M+1]: 428.2902.

### Example 87

### ethyl 3-(4-(6′-fluoro-2′-oxospiro[cyclopropane-1,3′-indoline]-1′-yl)piperidin-1-yl)-3-methylpyrrolidine-1-carboxylate

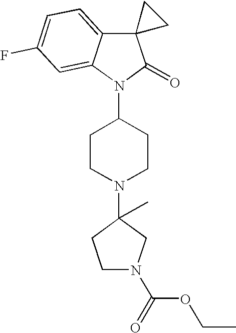

Sodium hydride (12.32 mg, 0.51 mmol) was added to a mixture of ethyl 3-(4-(6-fluoro-2-oxoindolin-1-yl)piperidin-1-yl)-3-methylpyrrolidine-1-carboxylate (20 mg, 0.05 mmol) and 1,2-dibromoethane (8.85 μL, 0.10 mmol) in DMF (3 mL) at room temperature. The resulting mixture was stirred at room temperature for 3 days. The reaction mixture was concentrated under reduced pressure and water was added to the residue. The aqueous layer was extracted three times with CH₂Cl₂. Combined the organic layers were washed with brine. The organic layer was dried over MgSO₄, filtered and concentrated. The crude product was purified by high pH preparative HPLC (40-60% MeCN in water) to give the title compound (15.50 mg, 72.6%) as solid. 1 H NMR (400 MHz, CDCI₃) δ ppm 1.09 (d, J=2.34 Hz, 3H), 1.22-1.32 (m, 3H), 1.43-1.54 (m, 2H), 1.67-1.80 (m, 4H), 1.81-1.99 (m, 2H), 2.30-2.55 (m, 4H), 2.72-2.85 (m, 1H), 2.90-3.03 (m, 1H), 3.26 (t, J=10.16 Hz, 1H), 3.33-3.69 (m, 3H), 4.05-4.22 (m, 2H), 4.25-4.40 (m, 1H), 6.60-6.81 (m, 2H), 6.99 (dt, J=9.77, 2.73 Hz, 1H); 13C NMR (101 MHz, CDCl₃) δ ppm 2.10 (s, 1C), 14.87 (s, 1C), 15.03 (s, 1C), 19.81 (s, 1C), 26.74 (s, 1C), 29.09 (s, 1C), 37.62 (s, 1C), 38.35 (s, 1C), 44.93 (s, 1C), 45.27 (s, 1C), 47.23 (s, 1C), 48.23 (s, 1C), 50.79 (d, J=5.16 Hz, 1C), 58.62 (d, J=11.05 Hz, 1C), 61.24 (br. s.,1C), 99.17 (s, 1C), 99.45 (s, 1C), 107.70 (s, 1C), 107.92 (s, 1C), 119.08 (s, 1C), 119.18 (s, 1C), 163.35 (s, 1C), 177.39 (s, 1C). HRMS [M+1]: 416.2342.

Various modifications of the invention, in addition to those described herein, will be apparent to those skilled in the art from the foregoing description. Such modifications are also intended to fall within the scope of the appended claims. Each reference, including all patents, patent applications, publications, and gene bank sequences cited in the present application, is incorporated herein by reference in its entirety.

1. A compound of Formula I: 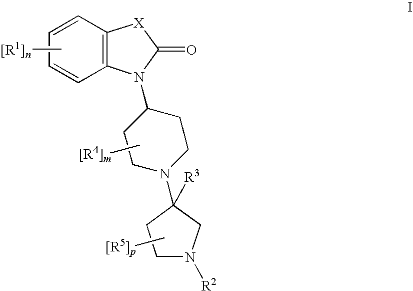
or pharmaceutically acceptable salt thereof;
wherein:
X is —CR⁶R⁷—, —NR⁸—, —O—, or —S—;
each R¹ is, independently, hydrogen, halogen, cyano, nitro, C₁₋₆alkyl, C₁₋₆haloalkyl, C₂₋₆alkenyl, C₂₋₆ alkynyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀aryl-C₁₋₃alkyl, C₃₋₉ heteroaryl-C₁₋₃alkyl, —SR^{e}, —OR^{f}, —O(CH₂)ᵣ—OR^{f}, —C(═O)—R^{e}, —C(═O)OR^{f}, —C(═O)NR^{g}R^{h}, —SO₂R^{e}, —SO₂NR^{g}R^{h}, —NR^{g}R^{h}, or —(CH₂)NR^{g}R^{h};
R² is selected from —C(═O)OR^{a}, and —C(═O)NR^{c}CR^{d};
R³ is C₁₋₆ alkyl or C₁₋₆ haloalkyl;
each R⁴ is, independently, halogen, C₁₋₆alkyl, C₁₋₆haloalkyl, C₁₋₆alkoxy, hydroxyl-C₁₋₆alkyl-, —CH₂—OR, -or —C(═O)NR₂; or
any two of R⁴ are linked together to form a C₁₋₄ alkylene bridge and the other R⁴, if any, are each, independently, halogen, C₁₋₆alkyl, C₁₋₆haloalkyl, C₁₋₆alkoxy, hydroxyl-C₁₋₆alkyl-, —CH₂—OR, -or —C(═O)NR₂;
each R⁵ is, independently, halogen, C₁₋₆alkyl, C₁₋₆haloalkyl, C₁₋₆alkoxy, hydroxyl-C₁₋₆alkyl-, —CH₂—OR, -or —C(═O)NR₂;
R⁶, R⁷, and R⁸ are each, independently, hydrogen, C₁₋₆alkyl, C₂₋₆alkenyl, or C₁₋₆haloalkyl;
each R⁹, R¹⁰, and R¹¹ is, independently, phenyl, C₃₋₆ cycloalkyl, C₂₋₅ heterocycloalkyl, C₃₋₅heteroaryl, halogen, cyano, nitro, —SR^{w}, —OR^{x}, —O(CH₂)ᵣ—OR^{x}, R^{x}, —C(═O)—R^{w}, —C(═O)OR^{x}, —C(═O)NR^{y}R^{z}, —SO₂R^{w}, —SO₂NR^{y}R^{z}, —NR^{y}R^{z}, or —(CH₂)ᵣNR^{y}R^{z};
R^{a} is selected from C₁₋₇ alkyl, C₁₋₇ haloalkyl, C₂₋₆ alkenyl, C₂₋₆ alkynyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, and C₃₋₉ heteroaryl-C₁₋₃alkyl; wherein said C₁₋₇ alkyl, C₂₋₆ alkenyl, C₂₋₆ alkynyl, and C₁₋₇ haloalkyl are each optionally substituted by 1, 2, or 3 independently selected R⁹ groups; wherein said C₃₋₇ cycloalkyl, C₃₋₇ cycloalkyl-C₁₋₃ alkyl, C₃₋₇ heterocycloalkyl, and C₃₋₇ heterocycloalkyl-C₁₋₃ alkyl are each optionally substituted by 1, 2, 3, or 4 independently selected R¹⁰ groups; and wherein said C₆₋₁₀aryl, C₆₋₁₀aryl-C₁₋₃alkyl, C₃₋₉ heteroaryl, and C₃₋₉heteroaryl-C₁₋₃ alkyl are each optionally substituted by 1, 2, 3, or 4 independently selected R¹¹ groups;
R^{c} and R^{d} are each, independently, hydrogen, C₁₋₇ alkyl, C₁₋₇ haloalkyl, C₂₋₆ alkenyl, C₂₋₆ alkynyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein said C₁₋₇ alkyl, C₂₋₆ alkenyl, C₂₋₆ alkynyl, and C₁₋₇ haloalkyl are each optionally substituted by 1, 2, or 3 independently selected R⁹ groups; wherein said C₃₋₇ cycloalkyl, C₃₋₇ cycloalkyl-C₁₋₃ alkyl, C₃₋₇ heterocycloalkyl, and C₃₋₇ heterocycloalkyl-C₁₋₃ alkyl are each optionally substituted by 1, 2, 3, or 4 independently selected R¹⁰ groups; and wherein said C₆₋₁₀aryl, C₆₋₁₀aryl-C₁₋₃alkyl, C₃₋₉ heteroaryl, and C₃₋₉heteroaryl-C₁₋₃ alkyl are each optionally substituted by 1, 2, 3, or 4 independently selected R¹¹ groups;
each R^{e}, R^{f}, R^{g}, R^{h}, R^{w}, R^{x}, R^{y}, R^{z}, and R is, independently hydrogen, C₁₋₆alkyl, C₂₋₆ alkenyl, or C₁₋₆ haloalkyl;
r is 1, 2, 3, or 4;
n is 0, 1, 2, 3, or 4;
m is 0, 1, 2, 3, 4, 5, 6, 7, or 8; and
p is an integer from 0 to 6,
with a proviso that said compound is not ethyl 3-methyl-3-(4-(2-oxobenzo[d]oxazol-3(2H)-yl)piperidin-1-yl)pyrrolidine-1-carboxylate.

2. The compound according to claim 1, or pharmaceutically acceptable salt thereof, wherein X is —CR⁶R⁷— or —NR⁸—.

3. The compound according to claim 1, or pharmaceutically acceptable salt thereof, wherein X is —NR⁸—.

4. The compound according to claim 1, or pharmaceutically acceptable salt thereof, wherein X is —NH.

5. The compound according to claim 1, or pharmaceutically acceptable salt thereof, wherein R⁶, R⁷, and R⁸ are each, independently, hydrogen, C₁₋₆alkyl, or C₁₋₆ haloalkyl.

6. The compound according to claim 1, or pharmaceutically acceptable salt thereof, wherein R⁶, R⁷, and R⁸ are each, independently, hydrogen or C₁₋₆alkyl.

7. The compound according to claim 1, or pharmaceutically acceptable salt thereof, wherein R² is —C(═O)OCH₃, —C(═O)OCH₂CH₃, —C(═O)OCH(CH₃)₂, is —C(═O)OCH₂CH₂F, —C(═O)OCH₂—C≡CH, —C(═O)OCH₂—C≡C—CH₃, or —C(═O)NHCH₂CH₃.

8. The compound according to claim 1, or pharmaceutically acceptable salt thereof, wherein R^{c}, and R^{d} are each, independently, hydrogen, C₁₋₇ alkyl, C₂₋₆ alkenyl, C₂₋₆ alkynyl, C₁₋₇ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃ alkyl, C₃₋₉ heteroaryl, or C₃₋₉ heteroaryl-C₁₋₃alkyl; and
R^{a} is selected from C₁₋₇ alkyl, C₂₋₆ alkenyl, C₂₋₆ alkynyl, C₁₋₇ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl.

9. The compound according to claim 1, or pharmaceutically acceptable salt thereof, wherein R^{c} and R^{d} are each, independently, hydrogen, C₁₋₇ alkyl, C₂₋₆ alkynyl, or C₁₋₇ haloalkyl; and R^{a} is selected from C₁₋₇ alkyl, C₂₋₆ alkynyl, or C₁₋₇ haloalkyl.

10. The compound according to claim 1, or pharmaceutically acceptable salt thereof, wherein R^{c} and R^{d} are each, independently, hydrogen, methyl, ethyl, isopropyl, prop-2-ynyl, or 2-fluoroethyl; and R^{a} is selected from methyl, ethyl, isopropyl, prop-2-ynyl, or 2-fluoroethyl.

11. The compound according to claim 1, or pharmaceutically acceptable salt thereof, wherein each R¹ is, independently, hydrogen, halogen, cyano, nitro, C₁₋₆alkyl, C₁₋₆haloalkyl, C₂₋₆alkenyl, —OR^{f}, —C(═O)OR^{f}, or —C(═O)NR^{g}R^{h}.

12. The compound according to claim 1, or pharmaceutically acceptable salt thereof, wherein each R¹ is, independently, hydrogen, halogen, cyano, C₁₋₆alkyl, C₂₋₆alkenyl, —C(═O)OR^{f}, —C(═O)NR^{g}R^{h}, hydroxyl, or C₁₋₆alkoxy.

13. The compound according to claim 1, or pharmaceutically acceptable salt thereof, wherein each R¹ is, independently, hydrogen, halogen, cyano, C₁₋₆alkyl, C₁₋₆haloalkyl, hydroxyl, or C₁₋₆alkoxy.

14. The compound according to claim 1, or pharmaceutically acceptable salt thereof, wherein each R¹ is, independently, hydrogen, halogen, or C₁₋₆alkyl.

15. The compound according to claim 1, or pharmaceutically acceptable salt thereof, wherein each R¹ is, independently, hydrogen, fluoro, or methyl.

16. The compound according to claim 1, or pharmaceutically acceptable salt thereof, wherein R³ is C₁₋₆ alkyl.

17. The compound according to claim 1, or pharmaceutically acceptable salt thereof, wherein R³ is methyl.

18. The compound according to claim 1, or pharmaceutically acceptable salt thereof, wherein each R⁴ and R⁵ is, independently, halogen, C₁₋₆alkyl, C₁₋₆haloalkyl, C₁₋₆alkoxy, hydroxyl-C₁₋₆alkyl-, —CH₂—OR, -or —C(═O)NR₂.

19. The compound according to claim 1, or pharmaceutically acceptable salt thereof, wherein each R⁴ and R⁵ is, independently, halogen, C₁₋₆alkyl, or C₁₋₆haloalkyl.

20. The compound according to claim 1, or pharmaceutically acceptable salt thereof, wherein each R⁴ and R⁵ is, independently, C₁₋₆alkyl.

21. The compound according to claim 1, or pharmaceutically acceptable salt thereof, wherein each R⁴ and R⁵ is, independently, C₁₋₃alkyl.

22. The compound according to claim 1, or pharmaceutically acceptable salt thereof, wherein each R⁴ and R⁵ is, independently, methyl.

23. The compound according to claim 1, or pharmaceutically acceptable salt thereof, wherein:
each R⁹, R¹⁰, and R¹¹ is, independently, halogen, cyano, nitro, —SR^{w}, —OR^{x}, —O(CH₂)ᵣ—OR^{x}, R^{x}, —C(═O)—R^{w}, —C(═O)OR^{x}, —C(═O)NR^{y}R^{z}, —SO₂R^{w}, —SO₂NR^{y}R^{z}, —NR^{y}R^{z}, or —(CH₂)ᵣNR^{y}R^{z}.

24. The compound according to claim 1, or pharmaceutically acceptable salt thereof, wherein:
each R⁹, R¹⁰, and R¹¹ is, independently, halogen, cyano, nitro, —OR^{x}, R^{x}, —SO₂R^{w}, —NR^{y}R^{z}, or —(CH₂)ᵣNR^{y}R^{z}.

25. The compound according to claim 1, or pharmaceutically acceptable salt thereof, wherein:
each R⁹, R¹⁰, and R¹¹ is, independently, halogen, cyano, nitro, —OR^{x}, R^{x}, or —NR^{y}R^{z}.

26. The compound according to claim 1, or pharmaceutically acceptable salt thereof, wherein:
each R⁹, R¹⁰, and R¹¹ is, independently, halogen, —OR^{x}, or R^{x}.

27. The compound according to claim 1, or pharmaceutically acceptable salt thereof, wherein m and p are each, independently, is 0, 1, or 2.

28. The compound according to claim 1, or pharmaceutically acceptable salt thereof, wherein m and p are each 0.

29. The compound according to claim 1, or pharmaceutically acceptable salt thereof, wherein n is 1 or 2.

30. The compound according to claim 1, or pharmaceutically acceptable salt thereof, wherein n is 1.

31-36. (canceled)

37. A compound selected from:
ethyl 3-methyl-3-(4-(2-oxo-2,3-dihydro-1H-benzo[d]imidazol-1-yl)piperidin-1-yl)pyrrolidine-1-carboxylate;
methyl 3-(4-(2-oxo-2,3-dihydro-1H-benzo[d]imidazol-1-yl)piperidin-1-yl)pyrrolidine-1-carboxylate;
isopropyl 3-methyl-3-(4-(2-oxo-2,3-dihydro-1H-benzo[d]imidazol-1-yl)piperidin-1-yl)pyrrolidine-1-carboxylate;
2-fluoroethyl 3-methyl-3-(4-(2-oxo-2,3-dihydro-1H-benzo[d]imidazol-1-yl)piperidin-1-yl)pyrrolidine-1-carboxylate;
N-ethyl-3-methyl-3-(4-(2-oxo-2,3-dihydro-1H-benzo[d]imidazol-1-yl)piperidin-1-yl)pyrrolidine-1-carboxamide;
ethyl 3-(4-(5-fluoro-2-oxo-2,3-dihydro-1H-benzo[d]imidazol-1-yl)piperidin-1-yl)-3-methylpyrrolidine-1-carboxylate;
methyl 3-(4-(5-fluoro-2-oxo-2,3-dihydro-1H-benzo[d]imidazol-1-yl)piperidin-1-yl)-3-methylpyrrolidine-1-carboxylate;
isopropyl 3-(4-(5-fluoro-2-oxo-2,3-dihydro-1H-benzo[d]imidazol-1-yl)piperidin-1-yl)-3-methylpyrrolidine-1-carboxylate;
2-fluoroethyl 3-(4-(5-fluoro-2-oxo-2,3-dihydro-1H-benzo[d]imidazol-1-yl)piperidin-1-yl)-3-methylpyrrolidine-1-carboxylate;
prop-2-ynyl 3-(4-(5-fluoro-2-oxo-2,3-dihydro-1H-benzo[d]imidazol-1-yl)piperidin-1-yl)-3-methylpyrrolidine-1-carboxylate;
ethyl 3-methyl-3-(4-(5-methyl-2-oxo-2,3-dihydro-1H-benzo[d]imidazol-1-yl)piperidin-1-yl)pyrrolidine-1-carboxylate;
isopropyl 3-methyl-3-(4-(5-methyl-2-oxo-2,3-dihydro-1H-benzo[d]imidazol-1-yl)piperidin-1-yl)pyrrolidine-1-carboxylate;
ethyl 3-methyl-3-(4-(6-methyl-2-oxo-2,3-dihydro-1H-benzo[d]imidazol-1-yl)piperidin-1-yl)pyrrolidine-1-carboxylate;
methyl 3-methyl-3-(4-(6-methyl-2-oxo-2,3-dihydro-1H-benzo[d]imidazol-1-yl)piperidin-1-yl)pyrrolidine-1-carboxylate;
but-2-ynyl 3-methyl-3-(4-(6-methyl-2-oxo-2,3-dihydro-1H-benzo[d]imidazol-1-yl)piperidin-1-yl)pyrrolidine-1-carboxylate;
but-2-ynyl 3-methyl-3-(4-(5-methyl-2-oxo-2,3-dihydro-1H-benzo[d]imidazol-1-yl)piperidin-1-yl)pyrrolidine-1-carboxylate;
but-2-ynyl 3-(4-(5-fluoro-2-oxo-2,3-dihydro-1H-benzo[d]imidazol-1-yl)piperidin-1-yl)-3-methylpyrrolidine-1-carboxylate;
methyl 3-(4-(5-fluoro-2-oxo-2,3-dihydro-1H-benzo[d]imidazol-1-yl)piperidin-1-yl)-3-methylpyrrolidine-1-carboxylate;
2-fluoroethyl 3-(4-(5-fluoro-2-oxo-2,3-dihydro-1H-benzo[d]imidazol-1-yl)piperidin-1-yl)-3-methylpyrrolidine-1-carboxylate;
prop-2-ynyl 3-(4-(5-fluoro-2-oxo-2,3-dihydro-1H-benzo[d]imidazol-1-yl)piperidin-1-yl)-3-methylpyrrolidine-1-carboxylate;
2-fluoroethyl 3-methyl-3-(4-(6-methyl-2-oxo-2,3-dihydro-1H-benzo[d]imidazol-1-yl)piperidin-1-yl)pyrrolidine-1-carboxylate;
2-fluoroethyl 3-methyl-3-(4-(5-methyl-2-oxo-2,3-dihydro-1H-benzo[d]imidazol-1-yl)piperidin-1-yl)pyrrolidine-1-carboxylate;
methyl 3-methyl-3-(4-(2-oxo-2,3-dihydro-1H-benzo[d]imidazol-1-yl)piperidin-1-yl)pyrrolidine-1-carboxylate;
2-fluoroethyl 3-methyl-3-(4-(2-oxo-2,3-dihydro-1H-benzo[d]imidazol-1-yl)piperidin-1-yl)pyrrolidine-1-carboxylate;
but-2-ynyl 3-methyl-3-(4-(2-oxo-2,3-dihydro-1H-benzo[d]imidazol-1-yl)piperidin-1-yl)pyrrolidine-1-carboxylate;
but-2-ynyl 3-(4-(5-fluoro-2-oxo-2,3-dihydro-1H-benzo[d]imidazol-1-yl)piperidin-1-yl)-3-methylpyrrolidine-1-carboxylate;
prop-2-ynyl 3-(4-(5-fluoro-2-oxo-2,3-dihydro-1H-benzo[d]imidazol-1-yl)piperidin-1-yl)-3-methylpyrrolidine-1-carboxylate;
methyl 3-(4-(5-fluoro-2-oxo-2,3-dihydro-1H-benzo[d]imidazol-1-yl)piperidin-1-yl)-3-methylpyrrolidine-1-carboxylate;
2-fluoroethyl 3-(4-(5-fluoro-2-oxo-2,3-dihydro-1H-benzo[d]imidazol-1-yl)piperidin-1-yl)-3-methylpyrrolidine-1-carboxylate;
isopropyl 3-methyl-3-(4-(6-methyl-2-oxo-2,3-dihydro-1H-benzo[d]imidazol-1-yl)piperidin-1-yl)pyrrolidine-1-carboxylate;
prop-2-ynyl 3-methyl-3-(4-(6-methyl-2-oxo-2,3-dihydro-1H-benzo[d]imidazol-1-yl)piperidin-1-yl)pyrrolidine-1-carboxylate;
prop-2-ynyl 3-methyl-3-(4-(5-methyl-2-oxo-2,3-dihydro-1H-benzo[d]imidazol-1-yl)piperidin-1-yl)pyrrolidine-1-carboxylate;
methyl 3-methyl-3-(4-(5-methyl-2-oxo-2,3-dihydro-1H-benzo[d]imidazol-1-yl)piperidin-1-yl)pyrrolidine-1-carboxylate;
methyl 3-methyl-3-(4-(6-methyl-2-oxo-2,3-dihydro-1H-benzo[d]imidazol-1-yl)piperidin-1-yl)pyrrolidine-1-carboxylate;
ethyl 3-(4-(6-fluoro-2-oxo-2,3-dihydro-1H-benzo[d]imidazol-1-yl)piperidin-1-yl)-3-methylpyrrolidine-1-carboxylate;
methyl 3-(4-(6-fluoro-2-oxo-2,3-dihydro-1H-benzo[d]imidazol-1-yl)piperidin-1-yl)-3-methylpyrrolidine-1-carboxylate;
2-fluoroethyl 3-(4-(6-fluoro-2-oxo-2,3-dihydro-1H-benzo[d]imidazol-1-yl)piperidin-1-yl)-3-methylpyrrolidine-1-carboxylate;
ethyl 3-(4-(5-fluoro-6-methyl-2-oxo-2,3-dihydro-1H-benzo[d]imidazol-1-yl)piperidin-1-yl)-3-methylpyrrolidine-1-carboxylate;
ethyl 3-(4-(5,6-difluoro-2-oxo-2,3-dihydro-1H-benzo[d]imidazol-1-yl)piperidin-1-yl)-3-methylpyrrolidine-1-carboxylate;
methyl 3-(4-(5,6-difluoro-2-oxo-2,3-dihydro-1H-benzo[d]imidazol-1-yl)piperidin-1-yl)-3-methylpyrrolidine-1-carboxylate;
2-fluoroethyl 3-(4-(5,6-difluoro-2-oxo-2,3-dihydro-1H-benzo[d]imidazol-1-yl)piperidin-1-yl)-3-methylpyrrolidine-1-carboxylate;
propyl 3-(4-(6-fluoro-2-oxo-2,3-dihydro-1H-benzo[d]imidazol-1-yl)piperidin-1-yl)-3-methylpyrrolidine-1-carboxylate;
isopropyl 3-(4-(6-fluoro-2-oxo-2,3-dihydro-1H-benzo[d]imidazol-1-yl)piperidin-1-yl)-3-methylpyrrolidine-1-carboxylate;
methyl 3-methyl-3-(4-(6-methyl-2-oxoindolin-1-yl)piperidin-1-yl)pyrrolidine-1-carboxylate;
ethyl 3-methyl-3-(4-(2-oxoindolin-1-yl)piperidin-1-yl)pyrrolidine-1-carboxylate;
methyl 3-methyl-3-(4-(2-oxoindolin-1-yl)piperidin-1-yl)pyrrolidine-1-carboxylate;
ethyl 3-methyl-3-(4-(2-oxoindolin-1-yl)piperidin-1-yl)pyrrolidine-1-carboxylate;
ethyl 3-(4-(6-fluoro-2-oxoindolin-1-yl)piperidin-1-yl)-3-methylpyrrolidine-1-carboxylate;
methyl 3-(4-(6-fluoro-2-oxoindolin-1-yl)piperidin-1-yl)-3-methylpyrrolidine-1-carboxylate;
methyl 3-(4-(5-fluoro-2-oxoindolin-1-yl)piperidin-1-yl)-3-methylpyrrolidine-1-carboxylate;
ethyl 3-(4-(5-fluoro-2-oxoindolin-1-yl)piperidin-1-yl)-3-methylpyrrolidine-1-carboxylate;
ethyl 3-(4-(5-fluoro-6-methyl-2-oxoindolin-1-yl)piperidin-1-yl)-3-methylpyrrolidine-1-carboxylate;
methyl 3-(4-(5-fluoro-6-methyl-2-oxoindolin-1-yl)piperidin-1-yl)-3-methylpyrrolidine-1-carboxylate;
ethyl 3-(4-(4-tert-butyl-6-fluoro-2-oxoindolin-1-yl)piperidin-1-yl)-3-methylpyrrolidine-carboxylate;
methyl 3-(4-(4-tert-butyl-6-methyl-2-oxoindolin-1-yl)piperidin-1-yl)-3-methylpyrrolidine-1-carboxylate;
ethyl 3-(4-(6′-fluoro-2′-oxospiro[cyclopropane-1,3′-indoline]-1′-yl)piperidin-1-yl)-3-methylpyrrolidine-1-carboxylate;
isolated enantiomers thereof, and pharmaceutically acceptable salts thereof.

38. A compound selected from methyl 3-methyl-3-(4-(6-methyl-2-oxoindolin-1-yl)piperidin-1-yl)pyrrolidine-1-carboxylate, isolated enantiomers thereof, and pharmaceutically acceptable salts thereof.

39. Isomer 2 of methyl 3-methyl-3-(4-(6-methyl-2-oxoindolin-1-yl)piperidin-1-yl)pyrrolidine-1-carboxylate as specified in Example 68 or a pharmaceutically acceptable salt thereof.

40. (+) Methyl 3-methyl-3-(4-(6-methyl-2-oxoindolin-1-yl)piperidin-1-yl)pyrrolidine-1-1-carboxylate or a pharmaceutically acceptable salt thereof.

41-44. (canceled)

45. A pharmaceutical composition comprising a compound according to claim 1, or pharmaceutically acceptable salt thereof, and a pharmaceutically acceptable carrier.

46. A method for the therapy of pain in a warm-blooded animal, comprising the step of administering to said animal in need of such therapy a therapeutically effective amount of a compound according to claim 1, or pharmaceutically acceptable salt thereof.

47. A method for the therapy of Alzheimer's disease in a warm-blooded animal, comprising the step of administering to said animal in need of such therapy a therapeutically effective amount of a compound according to claim 1, or pharmaceutically acceptable salt thereof.

48. A method for the therapy of schizophrenia in a warm-blooded animal, comprising the step of administering to said animal in need of such therapy a therapeutically effective amount of a compound according to claim 1, or pharmaceutically acceptable salt thereof.

49. A method for the therapy of anxiety in a warm-blooded animal, comprising the step of administering to said animal in need of such therapy a therapeutically effective amount of a compound according to claim 1, or pharmaceutically acceptable salt thereof.

50. A method for the therapy of depression in a warm-blooded animal, comprising the step of administering to said animal in need of such therapy a therapeutically effective amount of a compound according to claim 1, or pharmaceutically acceptable salt thereof.

51. A process for preparing a compound of Formula I: 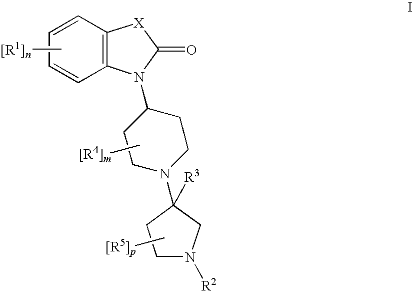
or pharmaceutically acceptable salt thereof, comprising reacting a compound of Formula X: 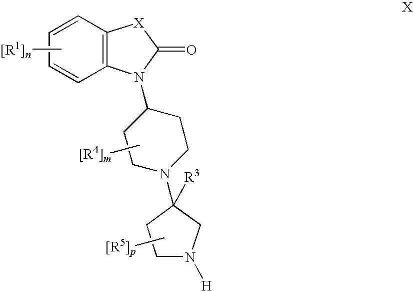
or pharmaceutically acceptable salt thereof, with a compound of Formula R^{a}OC(O)-L¹, or salt thereof, wherein L¹ is a leaving group, under conditions and for a time sufficient to form a compound of Formula I; wherein:
X is —CR⁶R⁷—, —NR⁸—, —O—, or —S—;
each R¹ is, independently, hydrogen, halogen, cyano, nitro, C₁₋₆alkyl, C₁₋₆haloalkyl, C₂₋₆alkenyl, C₂₋₆ alkynyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀aryl-C₁₋₃alkyl, C₃₋₉ heteroaryl, C₃₋₉heteroaryl-C₁₋₃alkyl, —SR^{e}, —OR^{f}, —O(CH₂)ᵣ—OR^{f}, —C(═O)—R^{e}, —C(═O)OR^{f}, —C(═O)NR^{g}R^{h}, —SO₂R , —SO₂NR^{g}R^{h}, —NR^{g}R^{h}, or —(CH₂)NR^{g}R^{h};
R² is —C(═O)OR^{a};
R³ is C₁₋₆ alkyl or C₁₋₆ haloalkyl;
each R⁴ is, independently, halogen, C₁₋₆alkyl, C₁₋₆haloalkyl, C₁₋₆alkoxy, hydroxyl-C₁₋₆alkyl-, —CH₂—OR, -or —C(═O)NR₂; or
any two of R⁴ are linked together to form a C₁₋₄ alkylene bridge and the other R⁴, if any, are each, independently, halogen, C₁₋₆alkyl, C₁₋₆haloalkyl, C₁₋₆alkoxy, hydroxyl-C₁₋₆alkyl-, —CH₂—OR, -or —C(═O)NR₂;
each R⁵ is, independently, halogen, C₁₋₆alkyl, C₁₋₆haloalkyl, C₁₋₆alkoxy, hydroxyl-C₁₋₆alkyl-, —CH₂—OR, -or —C(═O)NR₂;
R⁶, R⁷, and R⁸ are each, independently, hydrogen, C₁₋₆alkyl, C₂₋₆alkenyl, or C₁₋₆haloalkyl;
each R⁹, R¹⁰, and R¹¹ is, independently, phenyl, C₃₋₆ cycloalkyl, C₂₋₅ heterocycloalkyl, C₃₋₅heteroaryl, halogen, cyano, nitro, —SR^{w}, —OR^{x}, —O(CH₂)ᵣ—OR^{x}, R^{x}, —C(═O)—R^{w}, —C(═O)OR^{x}, —C(═O)NR^{y}R^{z}, —SO₂R^{w}, —SO₂NR^{y}R^{z}, —NR^{y}R^{z}, or —(CH₂)ᵣNR^{y}R^{z};
R^{a} is C₁₋₇ alkyl, C₁₋₇ haloalkyl, C₂₋₆ alkenyl, C₂₋₆ alkynyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein said C₁₋₇ alkyl, C₂₋₆ alkenyl, C₂₋₆ alkynyl, and C₁₋₇ haloalkyl are each optionally substituted by 1, 2, or 3 independently selected R⁹ groups; wherein said C₃₋₇ cycloalkyl, C₃₋₇ cycloalkyl-C₁₋₃ alkyl, C₃₋₇ heterocycloalkyl, and C₃₋₇ heterocycloalkyl-C₁₋₃ alkyl are each optionally substituted by 1, 2, 3, or 4 independently selected R¹⁰ groups; and wherein said C₆₋₁₀aryl, C₆₋₁₀aryl-C₁₋₃alkyl, C₃₋₉ heteroaryl, and C₃₋₉heteroaryl-C₁₋₃ alkyl are each optionally substituted by 1, 2, 3, or 4 independently selected R¹¹ groups;
each R^{e}, R^{f}, R^{g}, R^{h}, R^{w}, R^{x}, R^{y}, R^{z}, and R is, independently hydrogen, C₁₋₆alkyl, C₂₋₆ alkenyl, or C₁₋₆ haloalkyl;
r is 1, 2, 3, or 4;
n is 1, 2, 3, or 4;
m is 0, 1, 2, 3, 4, 5, 6, 7, or 8; and
p is an integer from 0 to 6.

52. A compound of formula X: 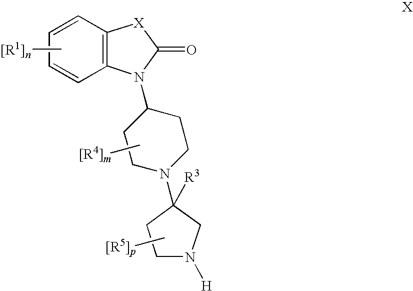
or pharmaceutically acceptable salt thereof, wherein:
X is —CR⁶R⁷—, —NR⁸—, —O—, or —S—;
each R¹ is, independently, hydrogen, halogen, cyano, nitro, C₁₋₆alkyl, C₁₋₆haloalkyl, C₂₋₆alkenyl, C₂₋₆ alkynyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀aryl-C₁₋₃alkyl, C₃₋₉ heteroaryl, C₃₋₉heteroaryl-C₁₋₃alkyl, —SR^{e}, —OR^{f}, —O(CH₂)ᵣOR^{f}, —C(═O)—R^{e}, —C(═O)OR^{f}, —C(═O)NR^{g}R^{h}, —SO₂R, —SO₂NR^{g}R^{h}, —NR^{g}R^{h}, or —(CH₂)ᵣNR^{g}R^{h};
R³ is C₁₋₆ alkyl or C₁₋₆ haloalkyl;
each R⁴ is, independently, halogen, C₁₋₆alkyl, C₁₋₆haloalkyl, C₁₋₆alkoxy, hydroxyl-C₁₋₆alkyl-, —CH₂—OR, -or —C(═O)NR₂; or
any two of R⁴ are linked together to form a C₁₋₄ alkylene bridge and the other R⁴, if any, are each, independently, halogen, C₁₋₆alkyl, C₁₋₆haloalkyl, C₁₋₆alkoxy, hydroxyl-C₁₋₆alkyl-, —CH₂—OR, -or —C(═O)NR₂;
each R⁵ is, independently, halogen, C₁₋₆alkyl, C₁₋₆haloalkyl, C₁₋₆alkoxy, hydroxyl-C₁₋₆alkyl-, —CH₂—OR, -or —C(═O)NR₂;
R⁶, R⁷, and R⁸ are each, independently, hydrogen, C₁₋₆alkyl, C₂₋₆alkenyl, or C₁₋₆haloalkyl;
each R⁹, R¹⁰, and R¹¹ is, independently, phenyl, C₃₋₆ cycloalkyl, C₂₋₅ heterocycloalkyl, C₃₋₅heteroaryl, halogen, cyano, nitro, —SR^{w}, —OR^{x}, —O(CH₂)ᵣ—OR^{x}, R^{x}, —C(═O)—R^{w}, —C(═O)OR^{x}, —C(═O)NR^{y}R^{z}, —SO₂R^{w}, —SO₂NR^{y}R^{z}, —NR^{y}R^{z}, or —(CH₂)ᵣNR^{y}R^{z};
R^{a} is C₁₋₇ alkyl, C₁₋₇ haloalkyl, C₂₋₆ alkenyl, C₂₋₆ alkynyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein said C₁₋₇ alkyl, C₂₋₆ alkenyl, C₂₋₆ alkynyl, and C₁₋₇ haloalkyl are each optionally substituted by 1, 2, or 3 independently selected R⁹ groups; wherein said C₃₋₇ cycloalkyl, C₃₋₇ cycloalkyl-C₁₋₃ alkyl, C₃₋₇ heterocycloalkyl, and C₃₋₇ heterocycloalkyl-C₁₋₃ alkyl are each optionally substituted by 1, 2, 3, or 4 independently selected R¹⁰ groups; and wherein said C₆₋₁₀aryl, C₆₋₁₀aryl-C₁₋₃alkyl, C₃₋₉ heteroaryl, and C₃₋₉heteroaryl-C₁₋₃ alkyl are each optionally substituted by 1, 2, 3, or 4 independently selected R¹¹ groups;
each R^{e}, R^{f}, R^{g}, R^{h}, R^{w}, R^{x}, R^{y}, R^{z}, and R is, independently hydrogen, C₁₋₆alkyl, C₂₋₆ alkenyl, or C₁₋₆ haloalkyl;
r is 1, 2, 3, or 4;
n is 1, 2, 3, or 4;
m is 0, 1, 2, 3, 4, 5, 6, 7, or 8; and
p is an integer from 0 to 6.

53. A method for treating ocular hypertension or glaucoma comprising administration to a patient in need of such treatment a therapeutically effective amount of a compound according to claim 1.

54. A pharmaceutical composition comprising a compound according to claim 38, or pharmaceutically acceptable salt thereof, and a pharmaceutically acceptable carrier.

55. A method for the therapy of pain in a warm-blooded animal, comprising the step of administering to said animal in need of such therapy a therapeutically effective amount of a compound according to claim 38, or pharmaceutically acceptable salt thereof.

56. A method for treating ocular hypertension or glaucoma comprising administration to a patient in need of such treatment a therapeutically effective amount of a compound according to claim 38.
